(19) United States
(12) Patent Application Publication
(10) Pub. No.: US 2024/0424109 A1
(43) Pub. Date: Dec. 26, 2024

(54) GLUCOSE-RESPONSIVE INSULIN CONJUGATES COMPRISING A TETRA-VALENT SUGAR CLUSTER FOR TREATMENT OF DIABETES

(71) Applicant: Merck Sharp & Dohme LLC, Rahway, NJ (US)

(72) Inventors: Pei Huo, Millburn, NJ (US); Songnian Lin, Holmdel, NJ (US); Christopher R. Moyes, Westfield, NJ (US); Dmitri A. Pissarnitski, Scotch Plains, NJ (US); Zhiqiang Zhao, Plainview, NY (US); David N. Hunter, Hatfield, PA (US); Yuping Zhu, Basking Ridge, NJ (US)

(73) Assignee: Merck Sharp & Dohme LLC, Rahway, NJ (US)

(21) Appl. No.: 18/711,216

(22) PCT Filed: Nov. 16, 2022
(86) PCT No.: PCT/US2022/050031

§ 371 Date: May 17, 2024

Related U.S. Application Data
(60) Provisional application No. 63/281,856, filed on Nov. 22, 2021.

(51) Int. Cl. A61K 47/54; A61K 38/28

(52) U.S. Cl. CPC A61K 47/549; A61K 38/28

(57) ABSTRACT

An insulin conjugate comprising or consisting of a tetra-valent sugar cluster is described. The tetra-valent sugar cluster is provided by tetra-dentate linker having four arms, wherein each arm of the tetra-dentate linker is independently covalently linked to a ligand comprising or consisting of a saccharide, such as a monosaccharide, disaccharide, trisaccharide, tetrasaccharide, or branched trisaccharide. In particular aspects, the insulin conjugate displays a pharmacokinetic (PK) and/or pharmacodynamic (PD) profile that is responsive to the systemic concentrations of a saccharide such as glucose or alpha-methylmannose.

### FIELD OF THE INVENTION

The present disclosure relates to an insulin conjugate comprising or consisting of a tetra-valent sugar cluster. In particular aspects, the insulin conjugate that displays a pharmacokinetic (“PK”) and/or pharmacodynamic (“PD”) profile that is responsive to the systemic concentrations of a saccharide such as glucose or alpha-methylmannose.

### BACKGROUND OF THE INVENTION

The majority of “controlled-release” drug delivery systems known in the prior art (e.g., U.S. Pat. No. 4,145,410 to Sears, which describes drug release from capsules that are enzymatically labile) are incapable of providing drugs to a patient at intervals and concentrations which are in direct proportion to the amount of a molecular indicator (e.g., a metabolite) present in the human body. The drugs in these prior art systems are thus not literally “controlled,” but simply provided in a slow release format which is independent of external or internal factors. The treatment of diabetes mellitus with injectable insulin is a well-known and studied example where uncontrolled, slow release of insulin is undesirable. In fact, it is apparent that the simple replacement of the hormone is not sufficient to prevent the pathological sequelae associated with this disease. The development of these sequelae is believed to reflect an inability to provide exogenous insulin proportional to varying blood glucose concentrations experienced by the patient. To solve this problem several biological and bioengineering approaches to develop a more physiological insulin delivery system have been suggested (e.g., see U.S. Pat. No. 4,348,387 to Brownlee et al.; U.S. Pat. Nos. 5,830,506, 5,902,603, and 6,410,053 to Taylor et al. and U.S. Patent Application Publication No. 2004-0202719 to Zion et al.).

Insulin replacement therapy for glycemic control in diabetic patients, however, is often insufficient due to the inability of exogenous insulins to function in response to the varying glucose concentration. Among approaches to develop glucose responsive insulins, conjugation of a cluster of sugars, e.g., D-mannose and L-fucose, to insulin has been reported in patent literature that potentially offer such glucose responsive insulins. See Neils C. Kaarsholm et al., Engineering Glucose Responsiveness into Insulin, 67 Diabetes 299-308 (February 2018). The cluster of sugar moieties, acting as substrate of endogenous mannose receptor, potentially affect the pharmacokinetic properties of their corresponding insulin conjugates in a way that is sensitive to the endogenous glucose concentration, rendering these insulin conjugates low risk of hypoglycemia. However, there remains a need for additional insulin replacement therapies for glycemic control in diabetic patients.

### SUMMARY OF THE INVENTION

The present disclosure provides insulin conjugates comprising a cluster of tetra-valent sugar moieties onto one, two or three amino groups of Gly^{A1}, Lys^{εB29}, or Phe^{B1} of insulin offers a balanced binding profile against both insulin receptor and mannose receptor. Such tetra-valent sugar cluster conjugates may provide glucose lowering in the presence of alpha-methylmannose, a surrogate for glucose, and may allow for improved glycemic controls in the treatment of diabetes with lower risk of hypoglycemia.

Other embodiments, aspects and features of the present disclosure are either further described in or will be apparent from the ensuing description, examples, and appended claims.

### BRIEF DESCRIPTION OF THE DRAWINGS

FIG. 1 shows blood glucose depression curves in non-diabetic male Yucatan minipigs equipped with dual vascular access ports (n=3 per study) following intravenous (“i.v.” or “IV”) injection of conjugate IOC-2 at 0.69 nmol/kg under conditions of phosphate buffered saline (“PBS”) infusion or i.v. alpha-methylmannose (“αMM”) infusion.

FIG. 2 shows blood glucose depression curves in non-diabetic male Yucatan minipigs equipped with dual vascular access ports (n=3 per study) following i.v. injection of conjugate IOC-7 at 0.69 nmol/kg under conditions of PBS infusion or i.v. αMM infusion.

FIG. 3 shows blood glucose depression curves in non-diabetic male Yucatan minipigs equipped with dual vascular access ports (n=3 per study) following i.v. injection of conjugate IOC-11 at 0.69 nmol/kg under conditions of PBS infusion or i.v. αMM infusion.

FIG. 4 shows blood glucose depression curves in non-diabetic male Yucatan minipigs equipped with dual vascular access ports (n=3 per study) following i.v. injection of conjugate IOC-12 at 0.69 nmol/kg under conditions of PBS infusion or i.v. αMM infusion.

FIG. 5 shows blood glucose depression curves in non-diabetic male Yucatan minipigs equipped with dual vascular access ports (n=3 per study) following i.v. injection of conjugate IOC-13 at 0.69 nmol/kg under conditions of PBS infusion or i.v. αMM infusion.

FIG. 6 shows blood glucose depression curves in non-diabetic male Yucatan minipigs equipped with dual vascular access ports (n=3 per study) following i.v. injection of conjugate IOC-16 at 0.69 nmol/kg under conditions of PBS infusion or i.v. αMM infusion.

FIG. 7 shows blood glucose depression curves in non-diabetic male Yucatan minipigs equipped with dual vascular access ports (n=3 per study) following i.v. injection of conjugate IOC-17 at 0.69 nmol/kg under conditions of PBS infusion or i.v. αMM infusion.

FIG. 8 shows blood glucose depression curves in non-diabetic male Yucatan minipigs equipped with dual vascular access ports (n=3 per study) following i.v. injection of conjugate IOC-18 at 0.69 nmol/kg under conditions of PBS infusion or i.v. αMM infusion.

FIG. 9 shows blood glucose depression curves in non-diabetic male Yucatan minipigs equipped with dual vascular access ports (n=3 per study) following i.v. injection of conjugate IOC-20 at 0.69 nmol/kg under conditions of PBS infusion or i.v. αMM infusion.

FIG. 10 shows blood glucose depression curves in non-diabetic male Yucatan minipigs equipped with dual vascular access ports (n=3 per study) following i.v. injection of conjugate IOC-25 at 0.69 nmol/kg under conditions of PBS infusion or i.v. αMM infusion.

FIG. 11 shows blood glucose depression curves in non-diabetic male Yucatan minipigs equipped with dual vascular access ports (n=3 per study) following i.v. injection of conjugate IOC-28 at 0.69 nmol/kg under conditions of PBS infusion or i.v. αMM infusion.

FIG. 12 shows blood glucose depression curves in non-diabetic male Yucatan minipigs equipped with dual vascular access ports (n=3 per study) following i.v. injection of conjugate IOC-31 at 0.69 nmol/kg under conditions of PBS infusion or i.v. αMM infusion.

FIG. 13 shows blood glucose depression curves in non-diabetic male Yucatan minipigs equipped with dual vascular access ports (n=3 per study) following i.v. injection of conjugate IOC-36 at 0.69 nmol/kg under conditions of PBS infusion or i.v. αMM infusion.

FIG. 14 shows blood glucose depression curves in non-diabetic male Yucatan minipigs equipped with dual vascular access ports (n=3 per study) following i.v. injection of conjugate IOC-43 at 0.69 nmol/kg under conditions of PBS infusion or i.v. αMM infusion.

FIG. 15 shows blood glucose depression curves in non-diabetic male Yucatan minipigs equipped with dual vascular access ports (n=3 per study) following i.v. injection of conjugate IOC-44 at 0.69 nmol/kg under conditions of PBS infusion or i.v. αMM infusion.

FIG. 16 shows blood glucose depression curves in non-diabetic male Yucatan minipigs equipped with dual vascular access ports (n=3 per study) following i.v. injection of conjugate IOC-47 at 0.69 nmol/kg under conditions of PBS infusion or i.v. αMM infusion.

FIG. 17 shows blood glucose depression curves in non-diabetic male Yucatan minipigs equipped with dual vascular access ports (n=3 per study) following i.v. injection of conjugate IOC-49 at 0.69 nmol/kg under conditions of PBS infusion or i.v. αMM infusion.

FIG. 18 shows blood glucose depression curves in non-diabetic male Yucatan minipigs equipped with dual vascular access ports (n=3 per study) following i.v. injection of conjugate IOC-50 at 0.69 nmol/kg under conditions of PBS infusion or i.v. αMM infusion.

FIG. 19 shows blood glucose depression curves in non-diabetic male Yucatan minipigs equipped with dual vascular access ports (n=3 per study) following i.v. injection of conjugate IOC-52 at 0.69 nmol/kg under conditions of PBS infusion or i.v. αMM infusion.

FIG. 20 shows blood glucose depression curves in non-diabetic male Yucatan minipigs equipped with dual vascular access ports (n=3 per study) following i.v. injection of conjugate IOC-55 at 0.69 nmol/kg under conditions of PBS infusion or i.v. αMM infusion.

FIG. 21 shows blood glucose depression curves in non-diabetic male Yucatan minipigs equipped with dual vascular access ports (n=3 per study) following i.v. injection of conjugate IOC-63 at 0.69 nmol/kg under conditions of PBS infusion or i.v. αMM infusion.

FIG. 22 shows blood glucose depression curves in non-diabetic male Yucatan minipigs equipped with dual vascular access ports (n=3 per study) following i.v. injection of conjugate IOC-65 at 0.69 nmol/kg under conditions of PBS infusion or i.v. αMM infusion.

FIG. 23 shows blood glucose depression curves in non-diabetic male Yucatan minipigs equipped with dual vascular access ports (n=3 per study) following i.v. injection of conjugate IOC-66 at 0.69 nmol/kg under conditions of PBS infusion or i.v. αMM infusion.

FIG. 24 shows blood glucose depression curves in non-diabetic male Yucatan minipigs equipped with dual vascular access ports (n=3 per study) following i.v. injection of conjugate IOC-67 at 0.69 nmol/kg under conditions of PBS infusion or i.v. αMM infusion.

FIG. 25 shows blood glucose depression curves in non-diabetic male Yucatan minipigs equipped with dual vascular access ports (n=3 per study) following i.v. injection of conjugate IOC-69 at 0.69 nmol/kg under conditions of PBS infusion or i.v. αMM infusion.

### DETAILED DESCRIPTION OF THE INVENTION

The present disclosure provides insulin conjugates comprising one, two, or three tetra-valent sugar cluster(s). These insulin conjugates may display a pharmacokinetic (“PK”) and/or pharmacodynamic (“PD”) profile that is responsive to the systemic concentrations of a saccharide, such as glucose or alpha-methylmannose, when administered to a subject in need thereof in the absence of an exogenous multivalent saccharide-binding molecule such as the lectin Concanavalin A (“Con A”). In general, the conjugates comprise an insulin or insulin analog molecule covalently attached at its Gly^{A1}, Lys^{εB29}, or Phe^{B1} amino acid to a linker having a tetra-valent sugar cluster thereon.

In particular embodiments, a conjugate may have a polydispersity index of one and a molecular weight (“MW”) of less than about 20,000 Da. In particular embodiments, the conjugate is long acting (i.e., exhibits a PK profile that is more sustained than soluble recombinant human insulin (“RHI”)).

The conjugates disclosed herein may display a PD or PK profile that is sensitive to the serum concentration of a serum saccharide when administered to a subject in need thereof in the absence of an exogenous saccharide binding molecule. In particular aspects, the serum saccharide is glucose or alpha-methylmannose. In further aspects, the conjugate binds an endogenous saccharide binding molecule at a serum glucose concentration of 60 or 70 mg/dL or less when administered to a subject in need thereof. The binding of the conjugate to the endogenous saccharide binding molecule is sensitive to the serum concentration of the serum saccharide. In a further aspect, the conjugate is capable of binding the insulin receptor at a serum saccharide concentration greater than 60, 70, 80, 90, or 100 mg/dL. At serum saccharide concentration at 60 or 70 mg/dL, the conjugate preferentially binds the endogenous saccharide binding molecule over the insulin receptor, and, as the serum concentration of the serum saccharide increases from 60 or 70 mg/dL, the binding of the conjugate to the endogenous saccharide binding molecule decreases, and the binding of the conjugate to the insulin receptor increases.

The present disclosure provides a conjugate comprising an insulin or insulin analog molecule covalently attached to at least one tetra-valent sugar cluster, wherein the tetra-valent sugar cluster is provided by a tetra-dentate linker having four arms, wherein each arm of the tetra-dentate linker is independently covalently linked to a ligand comprising or consisting of a saccharide, such as a monosaccharide, disaccharide, trisaccharide, tetrasaccharide, or branched trisaccharide.

In particular embodiments of the conjugate, the conjugate comprises an insulin or insulin analog molecule conjugated to at least two tetra-valent sugar clusters. In a further embodiment, the conjugate comprises an insulin or insulin analog molecule conjugated to at least three tetra-valent sugar clusters.

The present disclosure provides a conjugate comprising an insulin or insulin analog molecule covalently attached to one tetra-valent sugar cluster, wherein the tetra-valent sugar cluster is provided by a tetra-dentate linker having four arms, wherein each arm of the tetra-dentate linker is independently covalently linked to a ligand comprising or consisting of a saccharide, such as a monosaccharide, disaccharide, trisaccharide, tetrasaccharide, or branched trisaccharide.

The present disclosure provides a conjugate comprising an insulin or insulin analog molecule covalently attached to two tetra-valent sugar clusters, wherein each tetra-valent sugar cluster is provided by a tetra-dentate linker having three arms, wherein each arm of the tetra-dentate linker is independently covalently linked to a ligand comprising or consisting of a saccharide, such as a monosaccharide, disaccharide, trisaccharide, tetrasaccharide, or branched trisaccharide.

The present disclosure provides a conjugate comprising an insulin or insulin analog molecule covalently attached to three tetra-valent sugar clusters wherein each tetra-valent sugar cluster is provided by a tetra-dentate linker having three arms wherein each arm of the tetra-dentate linker is independently covalently linked to a ligand comprising or consisting of a saccharide, such as a monosaccharide, disaccharide, trisaccharide, tetrasaccharide, or branched trisaccharide.

In particular embodiments of the conjugate, the ligand comprises or consists of a saccharide selected from the group consisting of fucose, mannose, glucosamine, glucose, bimannose (also referred to herein as dimannose), trimannose, tetramannose, or branched trimannose.

In particular embodiments, the ligand comprises or consists of a saccharide and amine group. In particular embodiments, the saccharide and amine group are separated by a C₁-C₆ alkyl group, e.g., a C₁-C₃ alkyl group.

In particular embodiments, the ligand comprises or consists of a saccharide selected from the group consisting of aminoethylglucose (“AEG”), aminoethylmannose (“AEM”), aminoethylbimannose (“AEBM”), aminoethyltrimannose (“AETM”), β-aminoethyl-N-acetylglucosamine (“AEGA”), and aminoethylfucose (“AEF”). In particular embodiments, the saccharide is of the “D” configuration and in other embodiments, the saccharide is of the “L” configuration.

In particular embodiments of the conjugate, the tetra-valent sugar cluster is covalently linked to the amino acid at position A1 of the insulin or insulin analog molecule; position B1 of the insulin or insulin analog molecule; position B29 of the insulin or insulin molecule; position B28 of the insulin analog molecule; or position B3 of the insulin analog molecule.

In particular embodiments of the conjugate, the insulin analog is insulin lispro, insulin glargine, insulin aspart, insulin detemir, or insulin glulisine.

In particular embodiments of the conjugate, the conjugate displays a PD and/or PK profile that is sensitive to the serum concentration of a serum saccharide when administered to a subject in need thereof in the absence of an exogenous saccharide binding molecule.

In particular embodiments of the conjugate, the serum saccharide is glucose or alpha-methylmannose.

In particular embodiments of the conjugate, the conjugate binds an endogenous saccharide binding molecule at a serum glucose concentration of 60 mg/dL or less when administered to a subject in need thereof.

In particular embodiments of the conjugate, the endogenous saccharide binding molecule is human mannose receptor 1.

In particular embodiments of the conjugate, the conjugate has the general formula (I):

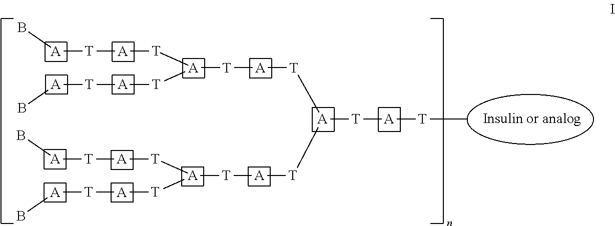

or has the general formula (II):

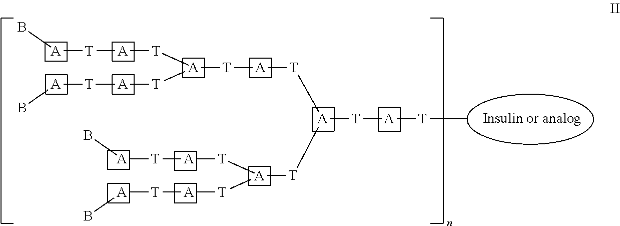

or has the general formula (III):

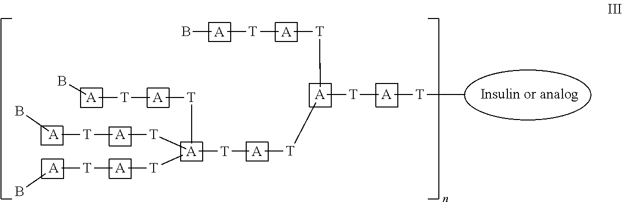

wherein:
(i) each occurrence of

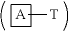

represents a repeat within a branch of the conjugate;
(ii) each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;
(iii) each occurrence of T is independently a covalent bond or a bivalent, straight or branched, saturated or unsaturated, optionally substituted C₁₋₃₀ hydrocarbon chain, wherein one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;
(iv) each occurrence of R is independently hydrogen, a suitable protecting group, an acyl moiety, arylalkyl moiety, aliphatic moiety, aryl moiety, heteroaryl moiety, or heteroaliphatic moiety;
(v) —B is -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(vi) n is 1, 2, or 3.

In further embodiments of the conjugate, the conjugate comprises or consists of the structure of conjugate I, wherein the insulin or insulin analog is conjugated to a tetra-valent linker selected from the group consisting of:

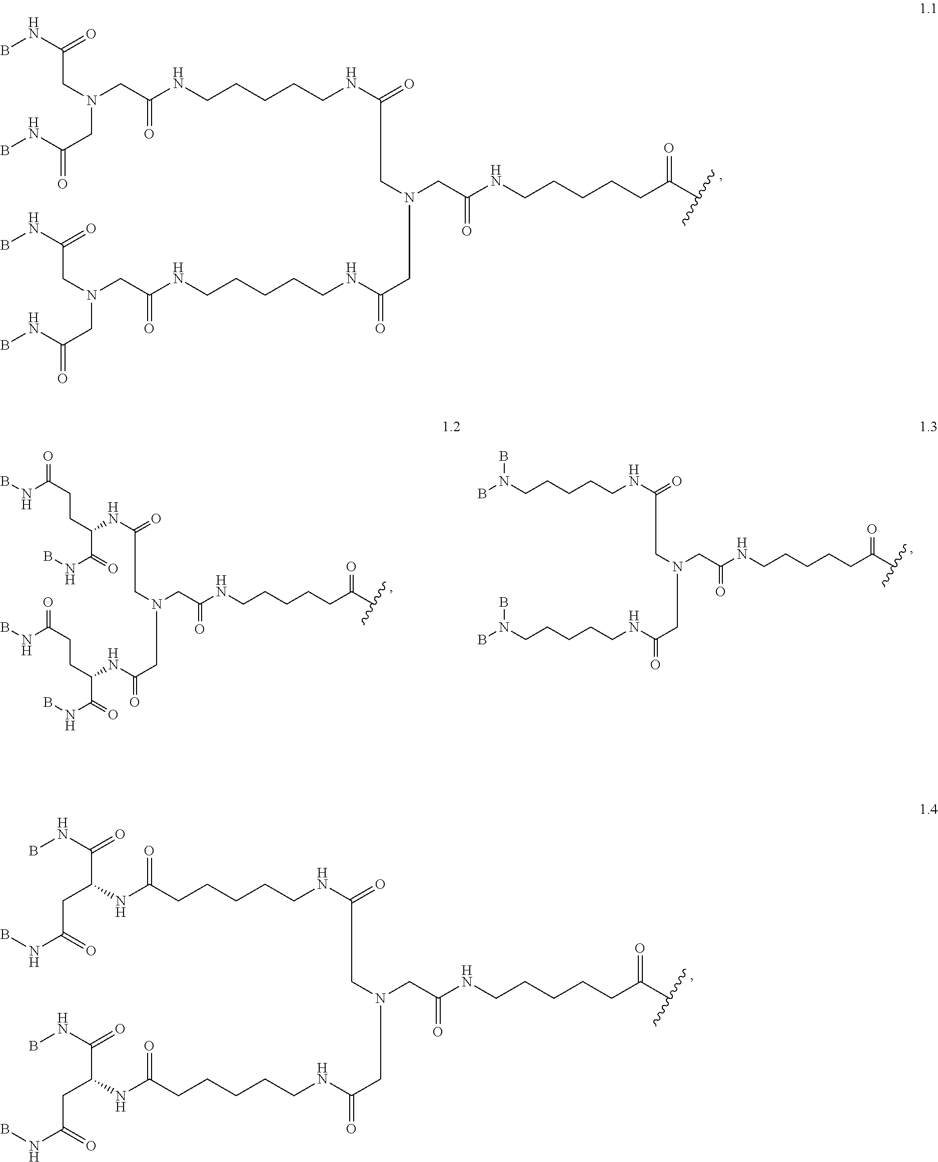

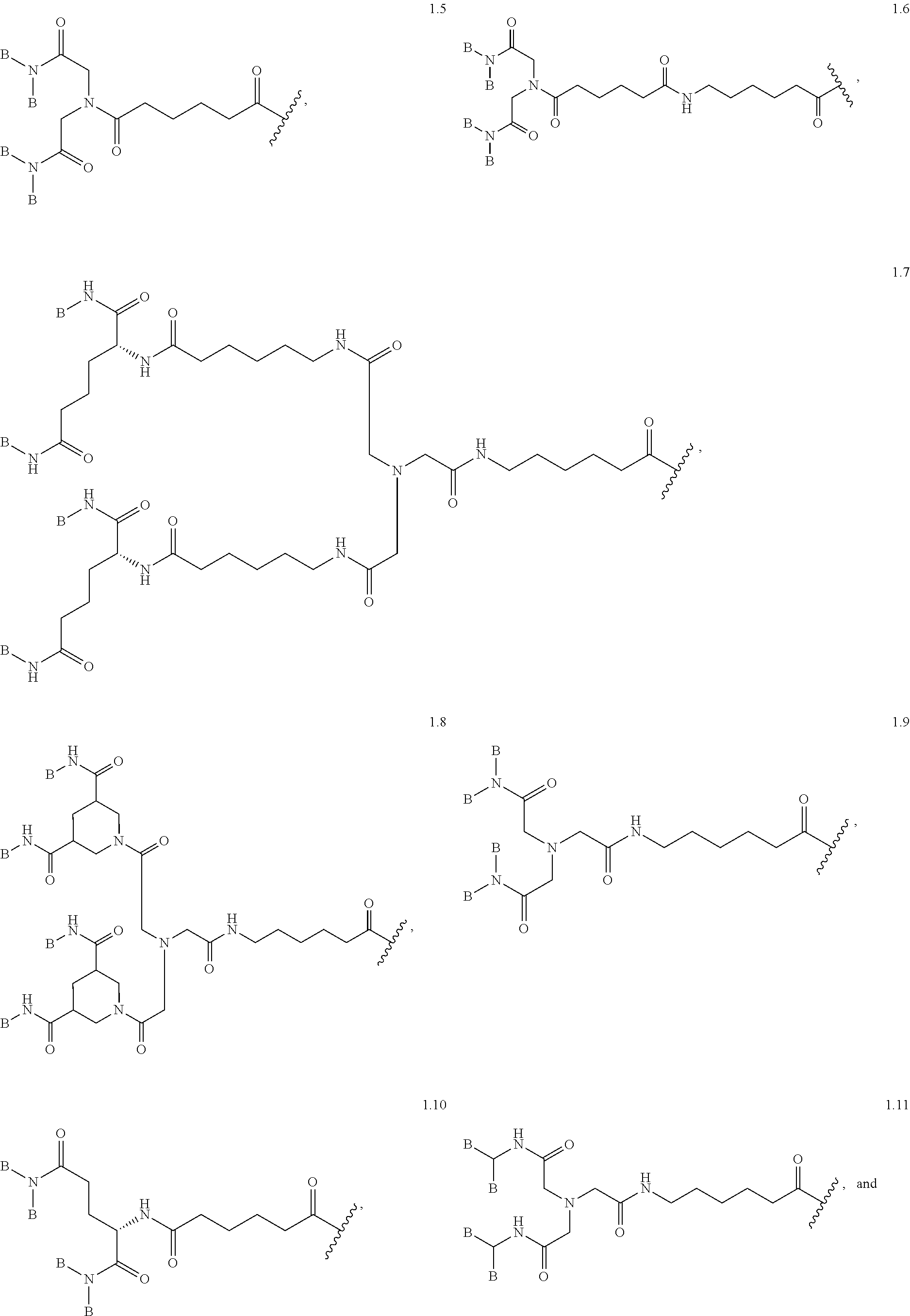

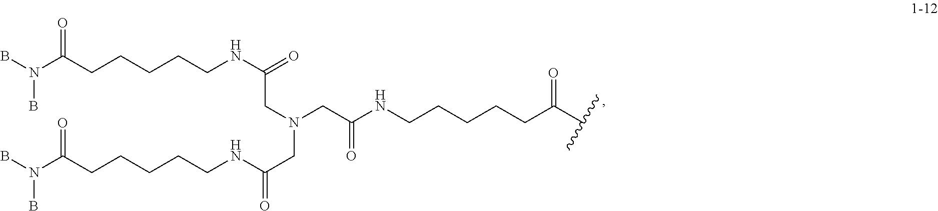

or the conjugate comprises the structure of conjugate II, wherein the insulin or insulin analog is conjugated to a tetra-valent linker selected from the group consisting of:

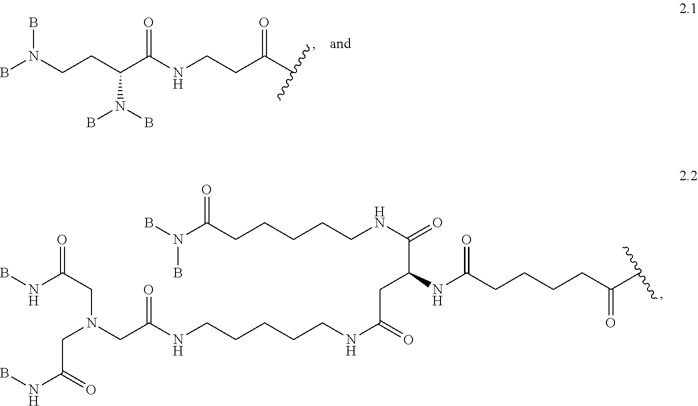

or the conjugate comprises the structure of conjugate III, wherein the insulin or insulin analog is conjugated to a tetra-valent linker selected from the group consisting of:

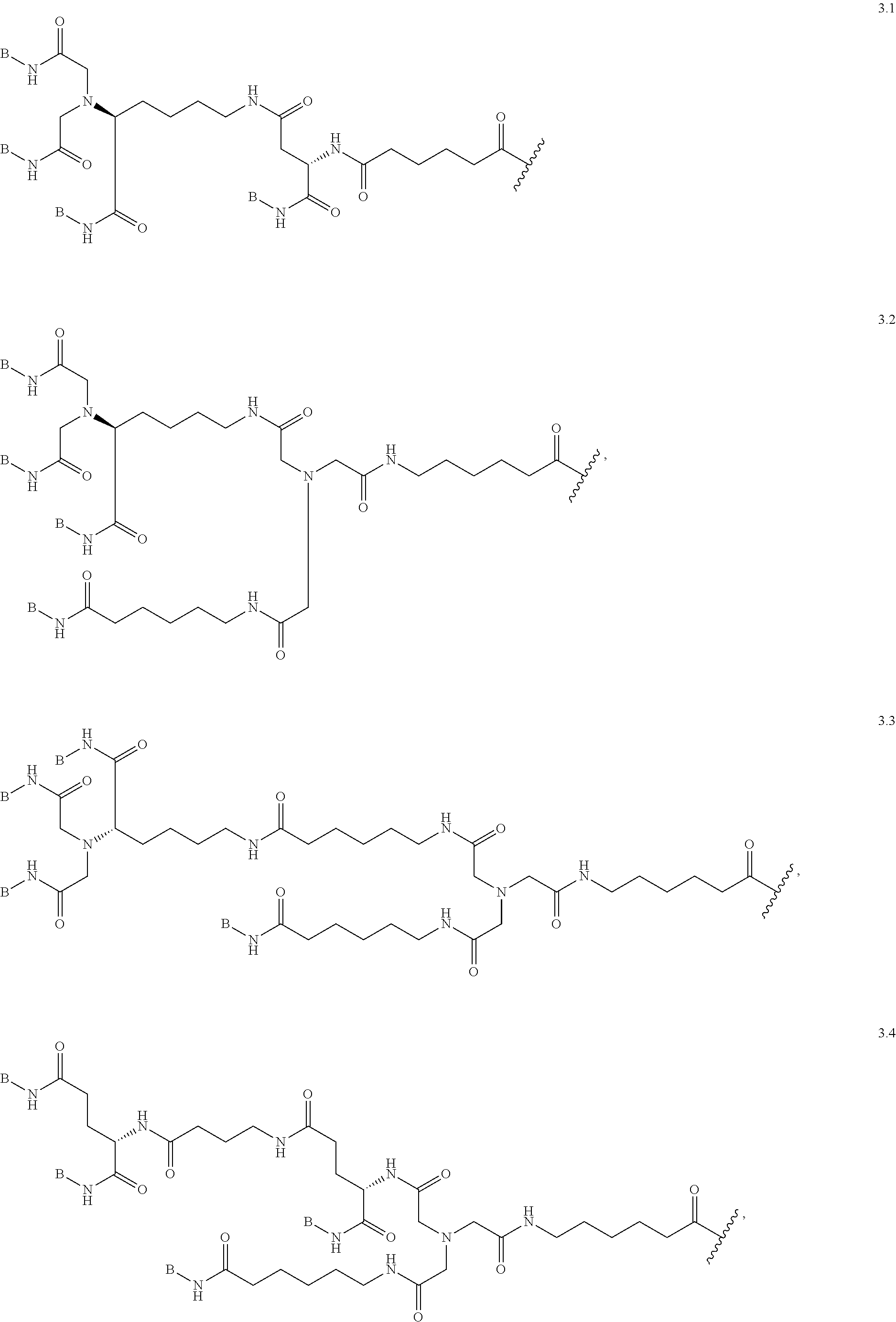

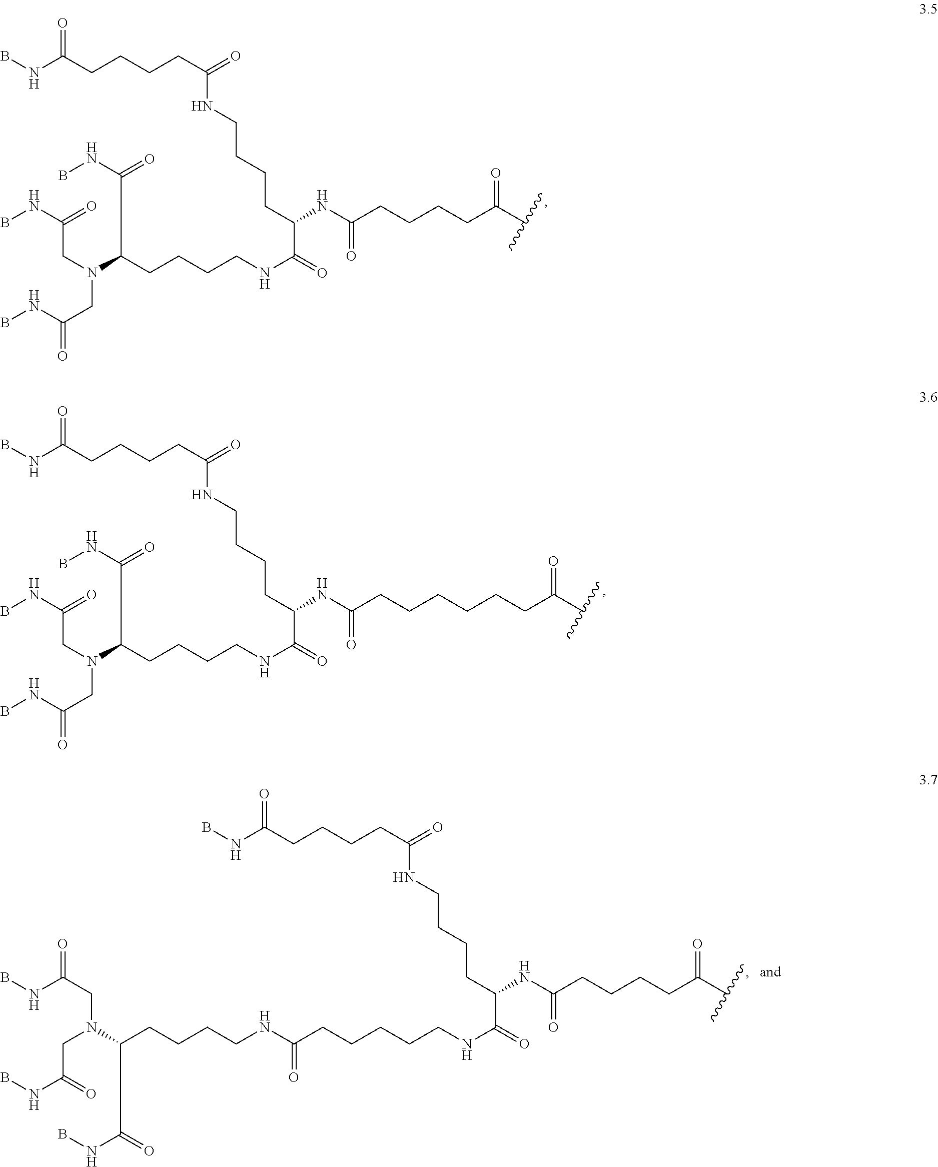

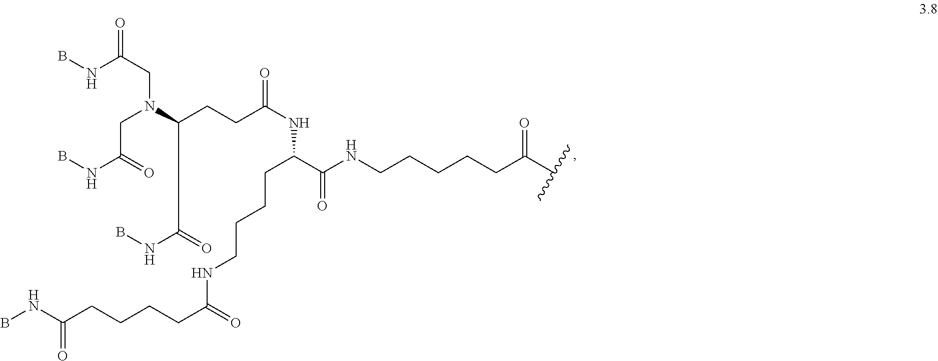

wherein a wavy line indicates the bond between the proximal end of the linker arm and amino acid on the insulin or insulin analog and wherein each B is independently -T-L^{B}-X, wherein each occurrence of X is independently the ligand and each occurrence of L^{B} is independently a covalent bond or a group derived from the covalent conjugation of a T with an X.

The present disclosure further provides a conjugate comprising an insulin or insulin analog is conjugated to a tetra-valent sugar cluster that comprises a structure selected from the group consisting of ML-1, ML-2, ML-3, ML-4, ML-5, ML-6, ML-7, ML-8, ML-9, ML-10, ML-11, ML-12, ML-13, ML-14, ML-15, ML-16, ML-17, ML-18, ML-19, ML-20, ML-21, ML-22, ML-23, ML-24, ML-25, ML-26, ML-27, ML-28, ML-29, ML-30, ML-31, ML-32, ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, ML-52, and ML-53.

In particular embodiments of the conjugate, the conjugate is selected from the group consisting of IOC-1, IOC-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-40, IOC-41, IOC-42, IOC-43, IOC-44, IOC-45, IOC-46, IOC-47, IOC-48, IOC-49, IOC-50, IOC-51, IOC-52, IOC-53, IOC-54, IOC-55, IOC-56, IOC-57, IOC-58, IOC-59, IOC-60, IOC-61, IOC-62, IOC-63, IOC-64, IOC-65, IOC-66, IOC-67, IOC-68, and IOC-69.

The present disclosure provides a composition comprising an insulin or insulin analog molecule covalently attached to at least one tetra-valent sugar cluster, wherein the tetra-valent sugar cluster is provided by a tetra-dentate linker having four arms, wherein each arm of the tetra-dentate linker is independently covalently linked to a ligand comprising or consisting of a monosaccharide, disaccharide, trisaccharide, tetrasaccharide, or branched trisaccharide, and a pharmaceutically acceptable carrier.

In particular embodiments of the composition, the ligand is selected from the group consisting of fucose, mannose, glucosamine, glucose, dimannose, trimannose, tetramannose, or branched trimannose.

In particular embodiments of the composition, the tetra-valent sugar cluster is covalently linked to the amino acid at position A1 of the insulin or insulin analog molecule; position B1 of the insulin or insulin analog molecule; or position B29 of the insulin or insulin molecule.

In particular embodiments of the composition, the insulin analog is insulin lispro, insulin glargine, insulin aspart, insulin detemir, or insulin glulisine.

In particular embodiments of the composition, the conjugate displays a PD and/or PK profile that is sensitive to the serum concentration of a serum saccharide when administered to a subject in need thereof in the absence of an exogenous saccharide binding molecule.

In particular embodiments of the composition, the serum saccharide is glucose or alpha-methylmannose.

In particular embodiments of the composition, the conjugate binds an endogenous saccharide binding molecule at a serum glucose concentration of 60 mg/dL or less when administered to a subject in need thereof.

In particular embodiments of the composition, the endogenous saccharide binding molecule is human mannose receptor 1.

The present disclosure further provides a method for treating diabetes comprising administering to an individual in need thereof a therapeutically effective amount of the conjugate or composition herein to treat the diabetes. In particular aspects, the diabetes is type I diabetes, type II diabetes, or gestational diabetes.

The present disclosure further provides for the use of the conjugate or composition herein for the treatment of diabetes. In particular aspects, the diabetes is type I diabetes, type II diabetes, or gestational diabetes.

### Definitions

Definitions of specific functional groups, chemical terms, and general terms used throughout the specification are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

As used herein, the term “acyl,” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments, aliphatic groups contain 1-3 carbon atoms. Suitable aliphatic groups include, but are not limited to, linear or branched, alkyl, alkenyl, and alkynyl groups, and hybrids thereof, such as (cycloalkyl)alkyl, (cycloalkenyl)alkyl, or (cycloalkyl)alkenyl.

As used herein, the term “alkenyl” denotes an optionally substituted monovalent group derived from a straight- or branched-chain aliphatic moiety having at least one carbon-carbon double bond. In particular embodiments, the alkenyl group employed in the disclosure contains 2-6 carbon atoms. In particular embodiments, the alkenyl group employed in the disclosure contains 2-5 carbon atoms. In some embodiments, the alkenyl group employed in the disclosure contains 2-4 carbon atoms. In another embodiment, the alkenyl group employed contains 2-3 carbon atoms. Alkenyl groups include, for example, ethenyl, propenyl, butenyl, 1-methyl-2-buten-1-yl, and the like.

As used herein, the term “alkyl” refers to optionally substituted saturated, straight- or branched-chain hydrocarbon radicals derived from an aliphatic moiety containing between 1-6 carbon atoms by removal of a single hydrogen atom. In some embodiments, the alkyl group employed in the disclosure contains 1-5 carbon atoms. In another embodiment, the alkyl group employed contains 1-4 carbon atoms. In still other embodiments, the alkyl group contains 1-3 carbon atoms. In yet another embodiment, the alkyl group contains 1-2 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

As used herein, the term “alkynyl” refers to an optionally substituted monovalent group derived from a straight- or branched-chain aliphatic moiety having at least one carbon-carbon triple bond by the removal of a single hydrogen atom. In particular embodiments, the alkynyl group employed in the disclosure contains 2-6 carbon atoms. In particular embodiments, the alkynyl group employed in the disclosure contains 2-5 carbon atoms. In some embodiments, the alkynyl group employed in the disclosure contains 2-4 carbon atoms. In another embodiment, the alkynyl group employed contains 2-3 carbon atoms. Representative alkynyl groups include, but are not limited to, ethynyl, 2-propynyl (propargyl), 1-propynyl, and the like.

As used herein, the term “aryl” used alone or as part of a larger moiety as in “aralkyl”, “aralkoxy”, or “aryloxyalkyl”, refers to an optionally substituted monocyclic and bicyclic ring systems having a total of five to 10 ring members, wherein at least one ring in the system is aromatic and wherein each ring in the system contains three to seven ring members. The term “aryl” may be used interchangeably with the term “aryl ring.” In particular embodiments, “aryl” refers to an aromatic ring system that includes, but not limited to, phenyl (“Ph”), biphenyl, naphthyl, anthracyl and the like, which may bear one or more substituents. Optionally, one or more heteroatoms, such as S, N, or O, may be incorporated into the aryl ring, providing a heteroaryl or heteroaromatic moiety, as defined below.

As used herein, the term “arylalkyl” refers to an alkyl group substituted with an aryl group (e.g., an aromatic or heteroaromatic group).

As used herein, the term “bivalent hydrocarbon chain” (also referred to as a “bivalent alkylene group”) is a polymethylene group, i.e., —(CH₂)_{z}—, wherein z is a positive integer from 1 to 30, from 1 to 20, from 1 to 12, from 1 to 8, from 1 to 6, from 1 to 4, from 1 to 3, from 1 to 2, from 2 to 30, from 2 to 20, from 2 to 10, from 2 to 8, from 2 to 6, from 2 to 4, or from 2 to 3.

As used herein, the term “carbonyl” refers to a monovalent or bivalent moiety containing a carbon-oxygen double bond. Non-limiting examples of carbonyl groups include aldehydes, ketones, carboxylic acids, ester, amide, enones, acyl halides, anhydrides, ureas, carbamates, carbonates, thioesters, lactones, lactams, hydroxamates, isocyanates, and chloroformates.

As used herein, the terms “cycloalkyl”, “cycloaliphatic”, “carbocycle”, or “carbocyclic”, used alone or as part of a larger moiety, refer to an optionally substituted saturated or partially unsaturated cyclic aliphatic monocyclic or bicyclic ring systems, as described herein, having from 3 to 10 members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons.

As used herein, the term “fucose” refers to the D or L form of fucose and may refer to an oxygen or carbon linked glycoside.

As used herein, the terms “halo” and “halogen” refer to an atom selected from fluorine (fluoro, —F), chlorine (chloro, —Cl), bromine (bromo, —Br), and iodine (iodo, —I).

As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but that is not aromatic. Unless otherwise specified, heteroaliphatic groups contain 1-6 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups.

As used herein, the term “heteroaralkyl” refers to an alkyl group substituted by a heteroaryl, wherein the alkyl and heteroaryl portions independently are optionally substituted.

As used herein, the term “heteroaryl” used alone or as part of a larger moiety, e.g., “heteroaralkyl”, or “heteroaralkoxy”, refers to an optionally substituted group having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, carbocyclic, or heterocyclic rings, where the radical or point of attachment is on the heteroaromatic ring. Non limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

As used herein, the term “heteroatom” refers to nitrogen, oxygen, or sulfur, and includes any oxidized form of nitrogen or sulfur, and any quaternized form of a basic nitrogen. The term “nitrogen” also includes a substituted nitrogen.

As used herein, the terms “heterocycle”, “heterocyclyl”, “heterocyclic radical”, and “heterocyclic ring” are used interchangeably and refer to a stable optionally substituted 5- to 7-membered monocyclic or 7- to 10-membered bicyclic heterocyclic moiety that is either saturated or partially unsaturated, and having, in addition to carbon atoms, one or more heteroatoms, as defined above. A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or carbocyclic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “unsaturated”, means that a moiety has one or more double or triple bonds.

As used herein, the term “partially unsaturated” refers to a ring moiety that includes at least one double or triple bond. The term “partially unsaturated” is intended to encompass rings having multiple sites of unsaturation, but it is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in particular embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph, which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{∘}₃, —OSiR^{∘}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur. Suitable divalent substituents on a saturated carbon atom of R^{∘} include=O and =S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic that may be substituted as defined below, or an unsubstituted 5- or 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic, which may be substituted as defined below, or an unsubstituted 5- to 6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R, —NR†₂, —C(O)R†, —C(O)OR†, —C(O)C(O)R†, —C(O)CH₂C(O)R†, —S(O)₂R†, —S(O)₂NR₂, —C(S)NR†₂, —C(NH)NR†₂, or —N(R†)S(O)₂R†; wherein each R is independently hydrogen, C₁₋₆ aliphatic that may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R† are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

As used herein, the term “suitable protecting group,” refers to amino protecting groups or hydroxyl protecting groups depending on its location within the compound and includes those described in detail in PROTECTING GROUPS IN ORGANIC SYNTHESIS, T. W. Greene and P. G. M. Wuts, 3^{rd} edition, John Wiley & Sons, 1999.

In any case where a chemical variable (e.g., an R group) is shown attached to a bond that crosses a bond of the ring, this means that one or more such variables are optionally attached to the ring having the crossed bond. Each R group on such a ring can be attached at any suitable position on the ring, this is generally understood to mean that the group is attached in place of a hydrogen atom on the parent ring. This includes the possibility that two R groups can be attached to the same ring atom. Furthermore, when more than one R group is present on a ring, each may be the same or different than other R groups attached thereto, and each group is defined independently of other groups that may be attached elsewhere on the same molecule, even though they may be represented by the same identifier.

As used herein, an “exogenous” molecule is one that is not present at significant levels in a patient unless administered to the patient. In particular embodiments, the patient is a mammal, e.g., a human, a dog, a cat, a rat, a minipig, etc. As used herein, a molecule is not present at significant levels in a patient if normal serum for that type of patient includes less than 0.1 mM of the molecule. In particular embodiments, normal serum for the patient may include less than 0.08 mM, less than 0.06 mM, or less than 0.04 mM of the molecule.

As used herein, the term “treat” (or “treating”, “treated”, “treatment”, etc.) refers to the administration of a conjugate of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition. For example, as used herein the term “treating diabetes” will refer in general to maintaining glucose blood levels near normal levels and may include increasing or decreasing blood glucose levels depending on a given situation.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the US Pharmacopeia for use in animals, including humans.

As used herein, the terms “effective amount” or “therapeutically effective amount” refer to a nontoxic but sufficient amount of an insulin analog to provide the desired effect. For example, one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective amount” in any individual case may be determined by one of ordinary skill.

As used herein, the term “patenteral” means not through the alimentary canal but by some other route such as intranasal, inhalation, subcutaneous, intramuscular, intraspinal, or intravenous.

As used herein, the term “insulin” means the active principle of the pancreas that affects the metabolism of carbohydrates in the animal body and is of value in the treatment of diabetes mellitus. The term includes synthetic and biotechnologically derived products that are the same as, or similar to, naturally occurring insulins in structure, use, and intended effect, and that are of value in the treatment of diabetes mellitus.

As used herein, the term “insulin or insulin molecule” is a generic term that includes the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond. As used herein, the terms “insulin” or “insulin molecule” encompasses all salt and non-salt forms of the insulin molecule. It will be appreciated that the salt form may be anionic or cationic depending on the insulin molecule. By “insulin” or “an insulin molecule”, it is intended that this disclosure encompasses both wild-type insulin and modified forms of insulin as long as they are bioactive (i.e., capable of causing a detectable reduction in glucose when administered in vivo).

The term “insulin analog” or “insulin analogue” as used herein includes any heterodimer analogue or single-chain analogue that comprises one or more modification(s) of the native A-chain peptide and/or B-chain peptide. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; adding any or all of terminal positions A1, B1, A21, and B30; or conjugating directly or by a polymeric or non-polymeric linker one or more acyl, polyethylglycine (“PEG”), or saccharide moiety (moieties); or any combination thereof. As exemplified by the N-linked glycosylated insulin analogues disclosed herein, the term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO2010/0080606, WO2009/099763, and WO2010/080609, the disclosures of which are incorporated herein by reference. Examples of single-chain insulin analogues also include but are not limited to those disclosed in published International Applications WO96/34882, WO95/516708, WO2005/054291, WO2006/097521, WO2007/104734, WO2007/104736, WO2007/104737, WO2007/104738, WO2007/096332, WO2009/132129; U.S. Pat. Nos. 5,304,473 and 6,630,348; and Kristensen et al., BIOCHEM. J. 305: 981-986 (1995), the disclosures of which are each incorporated herein by reference.

The term “insulin analog” or “insulin analogue” further includes single-chain and heterodimer polypeptide molecules that have little or no detectable activity at the insulin receptor but that have been modified to include one or more amino acid modifications or substitutions to have an activity at the insulin receptor that has at least 1%, 10%, 50%, 75%, or 90% of the activity at the insulin receptor as compared to native insulin and that further includes at least one N-linked glycosylation site. In particular aspects, the insulin analogue is a partial agonist that has from 2× to 100× less activity at the insulin receptor as does native insulin. In other aspects, the insulin analogue has enhanced activity at the insulin receptor, for example, the IGF^{B16B17} derivative peptides disclosed in published international application WO2010/080607 (which is incorporated herein by reference). These insulin analogues, which have reduced activity at the insulin growth hormone receptor and enhanced activity at the insulin receptor, include both heterodimers and single-chain analogues.

As used herein, the term “single-chain insulin or single-chain insulin analog” encompasses a group of structurally-related proteins wherein the A-chain peptide or functional analogue and the B-chain peptide or functional analogue are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analogue further includes three disulfide bonds: the first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

As used herein, the terms “connecting peptide” or C-peptide” refer to the connection moiety “C” of the B—C-A polypeptide sequence of a single chain prepro insulin-like molecule. Specifically, in the natural insulin chain, the C-peptide connects the amino acid at position 30 of the B-chain and the amino acid at position 1 of the A-chain. The term can refer to both the native insulin C-peptide, the monkey C-peptide, and any other peptide from 3 to 35 amino acids that connects the B-chain to the A-chain thus is meant to encompass any peptide linking the B-chain peptide to the A-chain peptide in a single-chain insulin analogue (see for example, U.S. Published Application Nos. US2009/0170750 and US2008/0057004 and WO96/34882) and in insulin precursor molecules such as disclosed in WO95/16708 and U.S. Pat. No. 7,105,314.

As used herein, the term “amino acid modification” refers to a substitution of an amino acid or the derivation of an amino acid by the addition and/or removal of chemical groups to/from the amino acid, and the term includes substitution with any of the 20 amino acids commonly found in human proteins, as well as atypical or non-naturally occurring amino acids. Commercial sources of atypical amino acids include Sigma-Aldrich (Milwaukee, WI), ChemPep Inc. (Miami, FL), and Genzyme Pharmaceuticals (Cambridge, MA). Atypical amino acids may be purchased from commercial suppliers, synthesized de novo, or chemically modified or derivatized from naturally occurring amino acids.

As used herein, the term “amino acid substitution” refers to the replacement of one amino acid residue by a different amino acid residue.

As used herein, the term “conservative amino acid substitution” is defined herein as exchanges within one of the following five groups:
I. Small aliphatic, nonpolar, or slightly polar residues: Ala, Ser, Thr, Pro, Gly;
II. Polar, negatively charged residues and their amides: Asp, Asn, Glu, Gln, cysteic acid, and homocysteic acid;
III. Polar, positively charged residues: His, Arg, Lys; Ornithine (Orn)
IV. Large, aliphatic, nonpolar residues: Met, Leu, Ile, Val, Cys, Norleucine (Nle), homocysteine
V. Large, aromatic residues: Phe, Tyr, Trp, acetyl phenylalanine

As used herein, the term “tetra-dentate linker” refers to a linker comprising a linker arm having a proximal end and a distal end wherein the proximal end is covalently linked to an amino acid on an insulin molecule and the distal end is covalently linked at or near the distal end to four ligand arms, each ligand arm having a distal end and a proximal end wherein the distal end is covalently linked to a ligand and the proximal end is covalently linked to the linker arm at or near the distal end of the linker arm.

As used herein, “plasma glucose” is usually 10% to 12% higher than “blood glucose” (considering blood glucose to be plasma+all blood cells).

The present disclosure provides methods for controlling the PK and/or PD profiles of insulin in a manner that is responsive to the systemic concentrations of a saccharide such as glucose. The methods are based in part on the discovery disclosed in U.S. Published Application No. US2011/0301083 that when particular insulin conjugates are modified to include high affinity saccharide ligands, such as branched trimannose, they could be made to exhibit PK/PD profiles that responded to saccharide concentration changes even in the absence of an exogenous multivalent saccharide-binding molecule such as the lectin Con A.

In general, the insulin conjugates of the present disclosure comprise an insulin or insulin analog molecule covalently attached to a tetra-valent sugar cluster at the A1, B1, or B29 amino acid of insulin or insulin analog. In particular embodiments, the tetra-valent sugar cluster is capable of competing with a saccharide (e.g., glucose or alpha-methylmannose) for binding to an endogenous saccharide-binding molecule, such as the Macrophage Mannose Receptor 1. In particular embodiments, the tetra-valent sugar cluster is capable of competing with glucose or alpha-methylmannose for binding to Con A. In particular embodiments, the linker is non-polymeric or highly branched. In particular embodiments, the conjugate may have a polydispersity index of one and a MW of less than about 20,000 Da. In particular embodiments, the conjugate is of formula (I) or of formula (II) or of formula (III) as defined and described herein. In particular embodiments, the conjugate is long acting (i.e., exhibits a PK profile that is more sustained than soluble RHI).

### Insulin Conjugates

In one aspect, the present disclosure provides an insulin or insulin analog molecule conjugated to at least one tetra-valent sugar cluster wherein the tetra-valent sugar cluster is provided by a branched linker having four arms (tetra-dentate linker, as discussed above) wherein each arm of the tetra-dentate linker is independently covalently linked to a ligand comprising or consisting of a monosaccharide, disaccharide, trisaccharide, tetrasaccharide, or branched trisaccharide. Thus, as used herein a tetra-valent sugar cluster comprises or consists of four ligands conjugated to a single amino acid on the insulin or insulin analog molecule. In particular embodiments, the amino acid is the Gly residue at the A1 position of the A-chain polypeptide, the Lys residue at the B29 position of the B-chain polypeptide, or the Phe residue at the B1 position of the B-chain polypeptide.

In particular aspects, the insulin or insulin analog molecule is conjugated to one, two, or three tetra-dentate linkers wherein each arm of each tetra-dentate linker is independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, all four ligands are fucose, branched mannose, dimannose, or mannose.

In particular aspects, the insulin or insulin analog molecule is conjugated to two tetra-dentate linkers wherein each arm of each tetra-dentate linker is independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, all four ligands are fucose, branched mannose, dimannose, or mannose.

In particular aspects, the insulin or insulin analog molecule is conjugated to three tetra-dentate linkers wherein each arm of each tetra-dentate linker is independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, all four ligands are fucose, branched mannose, dimannose, or mannose.

In particular aspects, the insulin or insulin analog molecule of the insulin conjugate disclosed herein is conjugated to a tetra-dentate linker wherein each arm of each tetra-dentate linker is independently covalently linked to a ligand comprising or consisting of a saccharide and is covalently attached to a linear linker linked to one ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, all four ligands are fucose, branched mannose, dimannose, or mannose.

In particular aspects, the insulin or insulin analog molecule of the insulin conjugate disclosed herein is conjugated to a tetra-dentate linker wherein each arm of each tetra-dentate linker is independently covalently linked to a ligand comprising or consisting of a saccharide and is covalently attached to a linker having two arms, each arm independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are selected from fucose, branched mannose, dimannose, or mannose. In particular aspects, at least three ligands are fucose, branched mannose, dimannose, or mannose. In particular aspects, all four ligands are fucose, branched mannose, dimannose, or mannose.

When the insulin conjugate is administered to a mammal at least one pharmacokinetic or pharmacodynamic property of the conjugate is sensitive to the serum concentration of a saccharide. In particular embodiments, the PK and/or PD properties of the conjugate are sensitive to the serum concentration of an endogenous saccharide such as glucose. In particular embodiments, the PK and/or PD properties of the conjugate are sensitive to the serum concentration of an exogenous saccharide, e.g., without limitation, mannose, fucose, N-acetyl glucosamine and/or alpha-methylmannose.

### Pharmacokinetic (“Pk”) and Pharmacodynamic (“Pd”) Properties

In various embodiments, the PK and/or PD behavior of the insulin conjugate may be modified by variations in the serum concentration of a saccharide. For example, from a PK perspective, the serum concentration curve may shift upward when the serum concentration of the saccharide (e.g., glucose) increases or when the serum concentration of the saccharide crosses a threshold (e.g., is higher than normal glucose levels).

In particular embodiments, the serum concentration curve of a conjugate disclosed herein is substantially different when administered to the mammal under fasted and hyperglycemic conditions. As used herein, the term “substantially different” means that the two curves are statistically different as determined by a student t-test (p<0.05). As used herein, the term “fasted conditions” means that the serum concentration curve was obtained by combining data from five or more fasted non-diabetic individuals. In particular embodiments, a fasted non-diabetic individual is a randomly selected 18- to 30-year old human who presents with no diabetic symptoms at the time blood is drawn and who has not eaten within 12 hours of the time blood is drawn. As used herein, the term “hyperglycemic conditions” means that the serum concentration curve was obtained by combining data from five or more fasted non-diabetic individuals in which hyperglycemic conditions (glucose Cₘₐₓ at least 100 mg/dL above the mean glucose concentration observed under fasted conditions) were induced by concurrent administration of conjugate and glucose. Concurrent administration of conjugate and glucose simply requires that the glucose Cₘₐₓ occur during the period when the conjugate is present at a detectable level in the serum. For example, a glucose injection (or ingestion) could be timed to occur shortly before, at the same time or shortly after the conjugate is administered. In particular embodiments, the conjugate and glucose are administered by different routes or at different locations. For example, in particular embodiments, the conjugate is administered subcutaneously while glucose is administered orally or intravenously.

In particular embodiments, the serum Cₘₐₓ of the conjugate is higher under hyperglycemic conditions as compared to fasted conditions. Additionally or alternatively, in particular embodiments, the serum area under the curve (“AUC”) of the conjugate is higher under hyperglycemic conditions as compared to fasted conditions. In various embodiments, the serum elimination rate of the conjugate is slower under hyperglycemic conditions as compared to fasted conditions. In particular embodiments, the serum concentration curve of the conjugates can be fit using a two-compartment bi-exponential model with one short and one long half-life. The long half-life appears to be particularly sensitive to glucose concentration. Thus, in particular embodiments, the long half-life is longer under hyperglycemic conditions as compared to fasted conditions. In particular embodiments, the fasted conditions involve a glucose Cₘₐₓ of less than 100 mg/dL (e.g., 80 mg/dL, 70 mg/dL, 60 mg/dL, 50 mg/dL, etc.). In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.). It will be appreciated that other PK parameters such as mean serum residence time (“MRT”), mean serum absorption time (“MAT”), etc. could be used instead of or in conjunction with any of the aforementioned parameters.

The normal range of glucose concentrations in humans, dogs, cats, and rats is 60 to 200 mg/dL. One skilled in the art will be able to extrapolate the following values for species with different normal ranges (e.g., the normal range of glucose concentrations in miniature pigs is 40 to 150 mg/dl). Glucose concentrations below 60 mg/dL are considered hypoglycemic. Glucose concentrations above 200 mg/dL are considered hyperglycemic. In particular embodiments, the PK properties of the conjugate may be tested using a glucose clamp method, and the serum concentration curve of the conjugate may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Additionally or alternatively, the serum Tₘₐₓ, serum Cₘₐₓ, MRT, MAT, and/or serum half-life may be substantially different at the two glucose concentrations. As discussed below, in particular embodiments, 100 mg/dL and 300 mg/dL may be used as comparative glucose concentrations. It is to be understood, however, that the present disclosure encompasses each of these embodiments with an alternative pair of comparative glucose concentrations including, without limitation, any one of the following pairs: 50 and 200 mg/dL, 50 and 300 mg/dL, 50 and 400 mg/dL, 50 and 500 mg/dL, 50 and 600 mg/dL, 100 and 200 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc.

Thus, in particular embodiments, the Cₘₐₓ of the conjugate is higher when administered to the mammal at the higher of the two glucose concentrations (e.g., 300 vs. 100 mg/dL glucose). In particular embodiments, the Cₘₐₓ of the conjugate is at least 50% (e.g., at least 100%, at least 200% or at least 400%) higher when administered to the mammal at the higher of the two glucose concentrations (e.g., 300 vs. 100 mg/dL glucose).

In particular embodiments, the AUC of the conjugate is higher when administered to the mammal at the higher of the two glucose concentrations (e.g., 300 vs. 100 mg/dL glucose). In particular embodiments, the AUC of the conjugate is at least 50% (e.g., at least 100%, at least 200% or at least 400%) higher when administered to the mammal at the higher of the two glucose concentrations (e.g., 300 vs. 100 mg/dL glucose).

In particular embodiments, the serum elimination rate of the conjugate is slower when administered to the mammal at the higher of the two glucose concentrations (e.g., 300 vs. 100 mg/dL glucose). In particular embodiments, the serum elimination rate of the conjugate is at least 25% (e.g., at least 50%, at least 100%, at least 200%, or at least 400%) faster when administered to the mammal at the lower of the two glucose concentrations (e.g., 100 vs. 300 mg/dL glucose).

In particular embodiments, the serum concentration curve of conjugates may be fit using a two-compartment bi-exponential model with one short and one long half-life. The long half-life appears to be particularly sensitive to glucose concentration. Thus, in particular embodiments, the long half-life is longer when administered to the mammal at the higher of the two glucose concentrations (e.g., 300 vs. 100 mg/dL glucose). In particular embodiments, the long half-life is at least 50% (e.g., at least 100%, at least 200% or at least 400%) longer when administered to the mammal at the higher of the two glucose concentrations (e.g., 300 vs. 100 mg/dL glucose).

In particular embodiments, the present disclosure provides a method in which the serum concentration curve of a conjugate is obtained at two different glucose concentrations (e.g., 300 vs. 100 mg/dL glucose); the two curves are fit using a two-compartment bi-exponential model with one short and one long half-life; and the long half-lives obtained under the two glucose concentrations are compared. In particular embodiments, this method may be used as an assay for testing or comparing the glucose sensitivity of one or more conjugates.

In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.). In particular embodiments, the fasted conditions involve a glucose Cₘₐₓ of less than 100 mg/dL (e.g., 80 mg/dL, 70 mg/dL, 60 mg/dL, 50 mg/dL, etc.). It will be appreciated that any of the aforementioned PK parameters such as serum Tₘₐₓ, serum Cₘₐₓ, AUC, MRT, MAT, and/or serum half-life could be compared.

From a PD perspective, the bioactivity of the conjugate may increase when the glucose concentration increases or when the glucose concentration crosses a threshold, e.g., is higher than normal glucose levels. In particular embodiments, the bioactivity of a conjugate is lower when administered under fasted conditions as compared to hyperglycemic conditions. In particular embodiments, the fasted conditions involve a glucose Cₘₐₓ of less than 100 mg/dL (e.g., 80 mg/dL, 70 mg/dL, 60 mg/dL, 50 mg/dL, etc.). In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.).

In particular embodiments, the PD properties of the conjugate may be tested by measuring the glucose infusion rate (“GIR”) required to maintain a steady glucose concentration. According to such embodiments, the bioactivity of the conjugate may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Thus, in particular embodiments, the bioactivity of the conjugate is higher when administered to the mammal at the higher of the two glucose concentrations (e.g., 300 vs. 100 mg/dL glucose). In particular embodiments, the bioactivity of the conjugate is at least 25% (e.g., at least 50% or at least 100%) higher when administered to the mammal at the higher of the two glucose concentrations (e.g., 300 vs. 100 mg/dL glucose).

In general, it will be appreciated that any of the PK and PD characteristics discussed in this section can be determined according to any of a variety of published pharmacokinetic and pharmacodynamic methods (see e.g., Baudys et al., BIOCONJUGATE CHEM. 9:176-183, 1998, for methods suitable for subcutaneous delivery). It is also to be understood that the PK and/or PD properties may be measured in any mammal (e.g., a human, a rat, a cat, a minipig, a dog, etc.). In particular embodiments, PK and/or PD properties are measured in a human. In particular embodiments, PK and/or PD properties are measured in a rat. In particular embodiments, PK and/or PD properties are measured in a minipig. In particular embodiments, PK and/or PD properties are measured in a dog.

### Ligand(s)

In general, a ligand comprises or consists of a monosaccharide, disaccharide, trisaccharide, tetrasaccharide, or branched trisaccharide. In particular aspects, the ligand comprises or consists of a monomannose, dimannose, trimannose, tetramannose, or branched trimannose. In particular aspects, the ligand comprises or consists of fucose, glucose, or N-glucosamine.

In particular embodiments, each ligand comprises a tetra-valent sugar cluster are capable of competing with a saccharide (e.g., glucose, alpha-methylmannose, or mannose) for binding to an endogenous saccharide-binding molecule (e.g., without limitation surfactant proteins A and D or members of the selectin family). In particular embodiments, the ligands are capable of competing with glucose or alpha-methylmannose for binding to the human macrophage mannose receptor 1 (“MRC1”). In particular embodiments, the ligands are capable of competing with a saccharide for binding to a non-human lectin (e.g., Con A). In particular embodiments, the ligands are capable of competing with glucose, alpha-methylmannose, or mannose for binding to a non-human lectin (e.g., Con A). Exemplary glucose-binding lectins include calnexin, calreticulin, N-acetylglucosamine receptor, selectin, asialoglycoprotein receptor, collectin (mannose-binding lectin), mannose receptor, aggrecan, versican, Pisum sativum agglutinin (“PSA”), Vicia faba lectin, lens culinaris lectin, soybean lectin, peanut lectin, lathyrus ochrus lectin, sainfoin lectin, Sophora japonica lectin, bowringia milbraedii lectin, Con A, and pokeweed mitogen.

In particular embodiments, one or more of the ligands may have the same chemical structure as glucose or may be a chemically related species of glucose, e.g., glucosamine. In various embodiments, it may be advantageous for one or more of the ligands to have a different chemical structure from glucose, e.g., in order to fine tune the glucose response of the conjugate. For example, in particular embodiments, one might use a ligand that includes glucose, mannose, fucose or derivatives of these (e.g., alpha-L-fucopyranoside, mannosamine, beta-linked N-acetyl mannosamine, methylglucose, methylmannose, ethylglucose, ethylmannose, propylglucose, propylmannose, etc.) and/or higher order combinations of these (e.g., a dimannose, linear and/or branched trimannose, etc.).

In particular embodiments, a ligand includes a monosaccharide. In particular embodiments, a ligand includes a disaccharide. In particular embodiments, a ligand includes a trisaccharide. In some embodiments, the ligand comprises or consists of a saccharide and one or more amine groups. In some embodiments, the ligand comprises or consists of a saccharide and ethyl group. In particular embodiments, the saccharide and amine group are separated by a C₁-C₆ alkyl group, e.g., a C₁-C₃ alkyl group. In some embodiments, the ligand is aminoethylglucose (“AEG”). In some embodiments, the ligand is aminoethylmannose (“AEM”). In some embodiments, the ligand is aminoethylbimannose (“AEBM”). In some embodiments, the ligand is aminoethyltrimannose (“AETM”). In some embodiments, the ligand is β-aminoethyl-N-acetylglucosamine (“AEGA”). In some embodiments, the ligand is aminoethylfucose (“AEF”). In particular embodiments, the saccharide is of the “D” configuration and in other embodiments, the saccharide is of the “L” configuration. Below are the structures of exemplary saccharides having an amine group separated from the saccharide by a C₂ ethyl group wherein R may be hydrogen or a carbonyl group of the linker. Other exemplary ligands will be recognized by those skilled in the art.

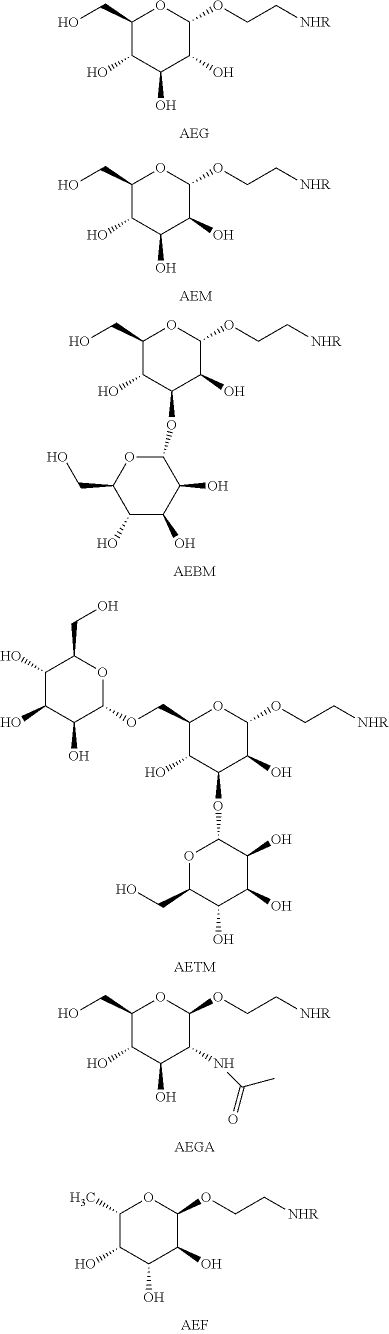

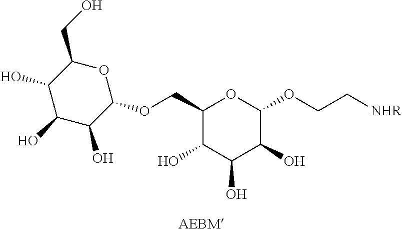

### Insulin

As used herein and as defined above, the term “insulin” or “insulin molecule” encompasses all salt and non-salt forms of the insulin molecule. It will be appreciated that the salt form may be anionic or cationic depending on the insulin molecule. By “insulin” or “an insulin molecule”, it is intended that this disclosure encompasses both wild-type insulin and modified forms of insulin as long as they are bioactive (i.e., capable of causing a detectable reduction in glucose when administered in vivo). Wild-type insulin includes insulin from any species whether in purified, synthetic, or recombinant form (e.g., human insulin, porcine insulin, bovine insulin, rabbit insulin, sheep insulin, etc.). A number of these are available commercially, e.g., from Sigma-Aldrich (St. Louis, MO). A variety of modified forms of insulin are known in the art (e.g., see Crotty and Reynolds, PEDIATR. EMERG. CARE. 23:903-905, 2007 and Gerich, AM. J. MED. 113:308-16, 2002). Modified forms of insulin (insulin analogs) may be chemically modified (e.g., by addition of a chemical moiety such as a PEG group or a fatty acyl chain as described below) and/or mutated (i.e., by addition, deletion, or substitution of one or more amino acids).

In particular embodiments, an insulin molecule of the present disclosure will differ from a wild-type insulin by 1-10 (e.g., 1-9, 1-8, 1-7, 1-6, 1-5, 1-4, 1-3, 1-2, 2-9, 2-8, 2-7, 2-6, 2-5, 2-4, 2-3, 3-9, 3-8, 3-7, 3-6, 3-5, 3-4, 4-9, 4-8, 4-7, 4-6, 4-5, 5-9, 5-8, 5-7, 5-6, 6-9, 6-8, 6-7, 7-9, 7-8, 8-9, 9, 8, 7, 6, 5, 4, 3, 2, or 1) amino acid substitutions, additions and/or deletions. In particular embodiments, an insulin molecule of the present disclosure will differ from a wild-type insulin by amino acid substitutions only. In particular embodiments, an insulin molecule of the present disclosure will differ from a wild-type insulin by amino acid additions only. In particular embodiments, an insulin molecule of the present disclosure will differ from wild-type insulin by both amino acid substitutions and additions. In particular embodiments, an insulin molecule of the present disclosure will differ from a wild-type insulin by both amino acid substitutions and deletions.

In particular embodiments, amino acid substitutions may be made on the basis of similarity in polarity, charge, solubility, hydrophobicity, hydrophilicity, and/or the amphipathic nature of the residues involved. In particular embodiments, a substitution may be conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine. In particular embodiments, the hydrophobic index of amino acids may be considered in choosing suitable mutations. The importance of the hydrophobic amino acid index in conferring interactive biological function on a polypeptide is generally understood in the art. Alternatively, the substitution of like amino acids can be made effectively on the basis of hydrophilicity. The importance of hydrophilicity in conferring interactive biological function of a polypeptide is generally understood in the art. The use of the hydrophobic index or hydrophilicity in designing polypeptides is further discussed in U.S. Pat. No. 5,691,198.

The wild-type sequence of human insulin (A-chain and B-chain) is shown below.

A-Chain (SEQ ID NO: 1):
GIVEQCCTSICSLYQLENYCN
B-Chain (SEQ ID NO: 2):
FVNQHLCGSHLVEALYLVCGERGFFYTPKT

In various embodiments, an insulin molecule of the present disclosure may be mutated at the B28 and/or B29 positions of the B-peptide sequence. For example, insulin lispro (HUMALOG™) is a rapid acting insulin mutant having the A-Chain of wild-type human insulin and in which the penultimate lysine and proline residues on the C-terminal end of the B-peptide have been reversed (Lys^{B28}Pro^{B29}-human insulin) (SEQ ID NO: 3).

B-Chain (SEQ ID NO: 3):
FVNQHLCGSHLVEALYLVCGERGFFYTKPT

This modification blocks the formation of insulin multimers. Insulin aspart (NOVOLOG™) is another rapid acting insulin mutant having the A-Chain of wild-type human insulin and in which proline at position B28 has been substituted with aspartic acid (Asp^{B28}-human insulin) (SEQ ID NO: 4).

B-Chain (SEQ ID NO: 4):
FVNQHLCGSHLVEALYLVCGERGFFYTDKT

This mutant also prevents the formation of multimers. In some embodiments, mutation at positions B28 and/or B29 is accompanied by one or more mutations elsewhere in the insulin polypeptide. For example, insulin glulisine (APIDRA™) is yet another rapid acting insulin mutant having the A-Chain of wild-type human insulin and in which aspartic acid at position B3 has been replaced by a lysine residue and lysine at position B29 has been replaced with a glutamic acid residue (Lys^{B3}Glu^{B29}-human insulin) (SEQ ID NO: 5).

B-Chain (SEQ ID NO: 5):
FVKQHLCGSHLVEALYLVCGERGFFYTPDT

In various embodiments, an insulin molecule of the present disclosure has an isoelectric point that is shifted relative to human insulin. In some embodiments, the shift in isoelectric point is achieved by adding one or more arginine residues to the N-terminus of the insulin A-peptide and/or the C-terminus of the insulin B-peptide. Examples of such insulin polypeptides include Arg^{A0}-human insulin, Arg^{B31}Arg^{B32}-human insulin, Gly^{A21}Arg^{B31}Arg^{B32}-human insulin, Arg^{A0}Arg^{B31}Arg^{B32}-human insulin, and Arg^{A0}Gly^{A1}Arg^{B31}Arg^{B32}-human insulin. By way of further example, insulin glargine (LANTUS™) is an exemplary long acting insulin mutant in which Asp^{A21} has been replaced by glycine (SEQ ID NO: 6), and two arginine residues have been added to the C-terminus of the B-peptide (SEQ ID NO: 7).

A-Chain (SEQ ID NO: 6):
GIVEQCCTSICSLYQLENYCG
B-Chain (SEQ ID NO: 7):
FVNQHLCGSHLVEALYLVCGERGFFYTPKTRR

The effect of these changes is to shift the isoelectric point, producing a solution that is completely soluble at pH 4. Thus, in some embodiments, an insulin molecule of the present disclosure comprises an A-peptide sequence wherein A21 is Gly and B-peptide sequence wherein B31 and B32 are Arg-Arg. It is to be understood that the present disclosure encompasses all single and multiple combinations of these mutations and any other mutations that are described herein (e.g., Gly^{A21}-human insulin, Gly^{A2}Arg^{B31}-human insulin, Arg^{B31}Arg^{B32}-human insulin, Arg^{B31}-human insulin).

In various embodiments, an insulin molecule of the present disclosure is truncated. For example, in particular embodiments, a B-peptide sequence of an insulin polypeptide of the present disclosure is missing B1, B2, B3, B26, B27, B28, B29, and/or B30. In particular embodiments, combinations of residues are missing from the B-peptide sequence of an insulin polypeptide of the present disclosure. For example, the B-peptide sequence may be missing residues B(1-2), B(1-3), B(29-30), B(28-30), B(27-30), and/or B(26-30). In some embodiments, these deletions and/or truncations apply to any of the aforementioned insulin molecules (e.g., without limitation to produce des(B30)-insulin lispro, des(B30)-insulin aspart, des(B30)-insulin glulisine, des(B30)-insulin glargine, etc.).

In some embodiments, an insulin molecule contains additional amino acid residues on the N- or C-terminus of the A or B-peptide sequences. In some embodiments, one or more amino acid residues are located at positions A0, A21, B0, and/or B31. In some embodiments, one or more amino acid residues are located at position A0. In some embodiments, one or more amino acid residues are located at position A21. In some embodiments, one or more amino acid residues are located at position B0. In some embodiments, one or more amino acid residues are located at position B31. In particular embodiments, an insulin molecule does not include any additional amino acid residues at positions A0, A21, B0, or B31.

In particular embodiments, an insulin molecule of the present disclosure is mutated such that one or more amidated amino acids are replaced with acidic forms. For example, asparagine may be replaced with aspartic acid or glutamic acid. Likewise, glutamine may be replaced with aspartic acid or glutamic acid. In particular, Asn^{A18}, Asn^{A21}, or Asn^{B3}, or any combination of those residues, may be replaced by aspartic acid or glutamic acid. Gln^{A15} or Gln^{B4}, or both, may be replaced by aspartic acid or glutamic acid. In particular embodiments, an insulin molecule has aspartic acid at position A21 or aspartic acid at position B3, or both.

One skilled in the art will recognize that it is possible to mutate yet other amino acids in the insulin molecule while retaining biological activity. For example, without limitation, the following modifications are also widely accepted in the art: replacement of the histidine residue of position B10 with aspartic acid (His^{B10}→Asp^{B10}); replacement of the phenylalanine residue at position B1 with aspartic acid (Phe^{B1}→Asp^{B1}); replacement of the threonine residue at position B30 with alanine (Thr^{B30}→Ala^{B30}); replacement of the tyrosine residue at position B26 with alanine (Tyr^{B26}→Ala^{B26}); and replacement of the serine residue at position B9 with aspartic acid (Ser^{B9}→Asp^{B9}).

In various embodiments, an insulin molecule of the present disclosure has a protracted profile of action. Thus, in particular embodiments, an insulin molecule of the present disclosure may be acylated with a fatty acid. That is, an amide bond is formed between an amino group on the insulin molecule and the carboxylic acid group of the fatty acid. The amino group may be the alpha-amino group of an N-terminal amino acid of the insulin molecule, or the amino group may be the epsilon-amino group of a lysine residue of the insulin molecule. An insulin molecule of the present disclosure may be acylated at one or more of the three amino groups that are present in wild-type human insulin or may be acylated on lysine residue that has been introduced into the wild-type human insulin sequence. In particular embodiments, an insulin molecule may be acylated at position B1. In particular embodiments, an insulin molecule may be acylated at position B29. In particular embodiments, the insulin molecule is acylated with a fatty acid molecule. In particular embodiments, the fatty acid is selected from myristic acid (C14), pentadecylic acid (C15), palmitic acid (C16), heptadecylic acid (C17) and stearic acid (C18). For example, insulin detemir (LEVEMIR™) is a long acting insulin mutant in which Thr^{B30} has been deleted, and a C14 fatty acid chain (myristic acid) has been attached to Lys^{B29}.

In some embodiments, the N-terminus of the A-peptide, the N-terminus of the B-peptide, the epsilon-amino group of Lys at position B29 or any other available amino group in an insulin molecule of the present disclosure is covalently linked to a fatty acid moiety of general formula:

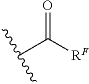

wherein R^{F} is hydrogen or a C₁₋₃₀ alkyl group. In some embodiments, R^{F} is a C₁₋₂₀ alkyl group, a C₃₋₁₉ alkyl group, a C₅₋₁₈ alkyl group, a C₆₋₁₇ alkyl group, a C₈₋₁₆ alkyl group, a C₁₀₋₁₅ alkyl group, or a C₁₂₋₁₄ alkyl group. In particular embodiments, the insulin polypeptide is conjugated to the moiety at the A1 position. In particular embodiments, the insulin polypeptide is conjugated to the moiety at the B1 position. In particular embodiments, the insulin polypeptide is conjugated to the moiety at the epsilon-amino group of Lys at position B29. In particular embodiments, position B28 of the insulin molecule is Lys and the epsilon-amino group of Lys^{B28} is conjugated to the fatty acid moiety. In particular embodiments, position B3 of the insulin molecule is Lys and the epsilon-amino group of Lys^{B3} is conjugated to the fatty acid moiety. In some embodiments, the fatty acid chain is 8-20 carbons long. In some embodiments, the fatty acid is octanoic acid (C8), nonanoic acid (C9), decanoic acid (C10), undecanoic acid (C11), dodecanoic acid (C12), or tridecanoic acid (C13). In particular embodiments, the fatty acid is myristic acid (C14), pentadecanoic acid (C15), palmitic acid (C16), heptadecanoic acid (C17), stearic acid (C18), nonadecanoic acid (C19), or arachidic acid (C20).

In various embodiments, an insulin molecule of the present disclosure includes the three wild-type disulfide bridges (i.e., one between position 7 of the A-chain and position 7 of the B-chain, a second between position 20 of the A-chain and position 19 of the B-chain, and a third between positions 6 and 11 of the A-chain). In particular embodiments, an insulin molecule is mutated such that the site of mutation is used as a conjugation point, and conjugation at the mutated site reduces binding to the insulin receptor (e.g., Lys^{A3}). In particular other embodiments, conjugation at an existing wild-type amino acid or terminus reduces binding to the insulin receptor (e.g., Gly^{A1}). In some embodiments, an insulin molecule is conjugated at position A4, A5, A8, A9, or B30. In particular embodiments, the conjugation at position A4, A5, A8, A9, or B30 takes place via a wild-type amino acid side chain (e.g., Glu^{A4}). In particular other embodiments, an insulin molecule is mutated at position A4, A5, A8, A9, or B30 to provide a site for conjugation (e.g., Lys^{A4}, Lys^{A5}, Lys^{A8}, Lys^{A9}, or Lys^{B30})

Methods for conjugating insulin molecules are described below. In particular embodiments, an insulin molecule is conjugated to a tetra-valent sugar cluster via the A1 amino acid residue. In particular embodiments, the A1 amino acid residue is glycine. It is to be understood however, that the present disclosure is not limited to N-terminal conjugation and that in particular embodiments an insulin molecule may be conjugated via a non-terminal A-chain amino acid residue. In particular, the present disclosure encompasses conjugation via the epsilon-amine group of a lysine residue present at any position in the A-chain (wild-type or introduced by site-directed mutagenesis). It will be appreciated that different conjugation positions on the A-chain may lead to different reductions in insulin activity. In particular embodiments, an insulin molecule is conjugated to the tetra-valent sugar cluster via the B1 amino acid residue. In particular embodiments, the B1 amino acid residue is phenylalanine. It is to be understood however, that the present disclosure is not limited to N-terminal conjugation and that in particular embodiments an insulin molecule may be conjugated via a non-terminal B-chain amino acid residue. In particular, the present disclosure encompasses conjugation via the epsilon-amine group of a lysine residue present at any position in the B-chain (wild-type or introduced by site-directed mutagenesis). For example, in particular embodiments an insulin molecule may be conjugated via the B29 lysine residue. In the case of insulin glulisine, conjugation to the at least one tetra-valent sugar cluster via the B3 lysine residue may be employed. It will be appreciated that different conjugation positions on the B-chain may lead to different reductions in insulin activity.

In particular embodiments, the tetra-valent sugar cluster is conjugated to more than one conjugation point on the insulin molecule. For example, an insulin molecule can be conjugated at both the A1 N-terminus and the B29 lysine. In some embodiments, amide conjugation takes place in carbonate buffer to conjugate at the B29 and A1 positions, but not at the B1 position. In other embodiments, an insulin molecule can be conjugated at the A1 N-terminus, the B1 N-terminus, and the B29 lysine. In yet other embodiments, protecting groups are used such that conjugation takes place at the B1 and B29 or at the B1 and A1 positions. It will be appreciated that any combination of conjugation points on an insulin molecule may be employed. In some embodiments, at least one of the conjugation points is a mutated lysine residue, e.g., Lys^{A3}

### Exemplary Insulin Conjugates

In various embodiments, the insulin conjugate of the present disclosure comprises an insulin or insulin analog molecule conjugated one tetra-valent sugar cluster, wherein the tetra-valent sugar cluster is provided by a branched linker having four arms (tetra-dentate linker), wherein each arm of the tetra-dentate linker is independently covalently linked to a ligand comprising or consisting of a monosaccharide, disaccharide, trisaccharide, tetrasaccharide, or branched trisaccharide. In particular embodiments, the ligands are independently selected from the group consisting of AEG, AEM, AEBM, AETM, AEGA, and AEF. In particular embodiments, the insulin molecule is conjugated via the A1 amino acid residue. In particular embodiments, the insulin molecule is conjugated via the B1 amino acid residue. In particular embodiments, the insulin molecule is conjugated via the epsilon-amino group of Lys^{B29}.

In particular embodiments, the insulin or insulin molecule of the above insulin conjugate may be conjugated to one or more additional linkers attached to one or more ligands, each ligand independently selected from AEG, AEM, AEBM, AETM, AEGA, and AEF. The additional linkers may be linear, bi-dentate, tri-dentate, quadra-dentate, etc., wherein each arm of the linker comprises a ligand, which may independently be selected from AEG, AEM, AEBM, AETM, AEGA, and AEF.

Thus, in particular embodiments, the insulin conjugate may comprise or consist of a tetra-valent sugar cluster conjugated to the amino group at position A1 of the insulin or insulin analog; or the amino group at position B1 of the insulin or insulin analog; or the amino group at position B29 of the insulin or insulin analog.

In particular embodiments, the insulin conjugate may comprise or consist of two tetra-valent sugar clusters (a first sugar cluster and a second sugar cluster) wherein each ligand comprising the first tetra-valent sugar cluster is independently a ligand selected from AEG, AEM, AEBM, AETM, AEGA, and AEF is conjugated to the amino group at position A1 and wherein each ligand comprising the second tetra-valent sugar cluster is independently a ligand selected from AEG, AEM, AEBM, AETM, AEGA, and AEF is conjugated to the amino group at position B1 or B29.

In particular embodiments, the insulin conjugate may comprise or consist of two tetra-valent sugar clusters (a first sugar cluster and a second sugar cluster) wherein each ligand comprising the first tetra-valent sugar cluster is independently a ligand selected from AEG, AEM, AEBM, AETM, AEGA, and AEF is conjugated to the amino group at position B1 and wherein each ligand comprising the second tetra-valent sugar cluster is independently a ligand selected from AEG, AEM, AEBM, AETM, AEGA, and AEF is conjugated to the amino group at position A1 or B29.

In particular embodiments, the insulin conjugate may comprise or consist of two tetra-valent sugar clusters (a first sugar cluster and a second sugar cluster) wherein each ligand comprising the first tetra-valent sugar cluster is independently a ligand selected from AEG, AEM, AEBM, AETM, AEGA, and AEF is conjugated to the amino group at position B29 and wherein each ligand comprising the second tetra-valent sugar cluster is independently a ligand selected from AEG, AEM, AEBM, AETM, AEGA, and AEF is conjugated to the amino group at position B1 or A1.

In particular embodiments, the insulin conjugate may comprise or consist of three tetra-valent sugar clusters (a first sugar cluster, a second sugar cluster, and a third sugar cluster) wherein each ligand comprising the first tetra-valent sugar cluster is independently a ligand selected from AEG, AEM, AEBM, AETM, AEGA, and AEF is conjugated to the amino group at position B29; wherein each ligand comprising the second tetra-valent sugar cluster is independently a ligand selected from AEG, AEM, AEBM, AETM, AEGA, and AEF is conjugated to the amino group at position B1 and wherein each ligand comprising the third tetra-valent sugar cluster is independently a ligand selected from AEG, AEM, AEBM, AETM, AEGA, and AEF is conjugated to the amino group at position A1.

In particular embodiments, the insulin or insulin analog molecule further includes an acyl group covalently linked to the A1 or both A1 and B1 N-terminal amino groups. In particular embodiments, the insulin or insulin analog molecule further includes a urea group covalently linked to the A1 and B1 N-terminal amino groups.

### Insulin Conjugates

This section describes some exemplary insulin or insulin analog conjugates.

In various embodiments, the conjugates may have the general formula (I):

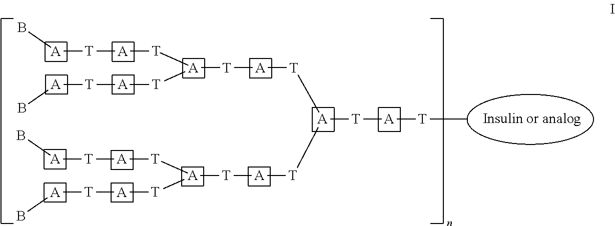

wherein:
(i) each occurrence of

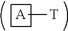

represents a repeat within a branch of the conjugate;
(ii) each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;
(iii) each occurrence of T is independently a covalent bond or a bivalent, straight or branched, saturated or unsaturated, optionally substituted C₁₋₃₀ hydrocarbon chain wherein one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group; (iv) each occurrence of R is independently hydrogen, a suitable protecting group, an acyl moiety, arylalkyl moiety, aliphatic moiety, aryl moiety, heteroaryl moiety, or heteroaliphatic moiety;
(v) —B is -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(vi) n is 1, 2, or 3.

In various embodiments, the conjugates may have the general formula (II):

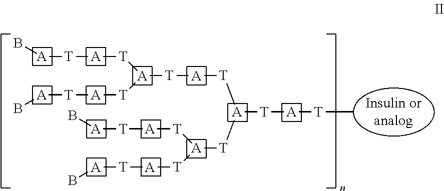

wherein:
(i) each occurrence of

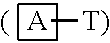

represents a repeat within a branch of the conjugate;
(ii) each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;
(iii) each occurrence of T is independently a covalent bond or a bivalent, straight or branched, saturated or unsaturated, optionally substituted C₁₋₃₀ hydrocarbon chain wherein one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;
(iv) each occurrence of R is independently hydrogen, a suitable protecting group, an acyl moiety, arylalkyl moiety, aliphatic moiety, aryl moiety, heteroaryl moiety, or heteroaliphatic moiety;
(v) —B is -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(vi) n is 1, 2, or 3.

In various embodiments, the conjugates may have the general formula (III):

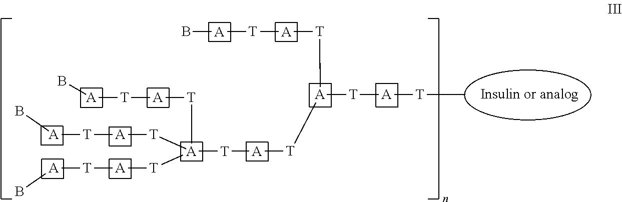

wherein:
(i) each occurrence of

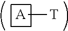

represents a repeat within a branch of the conjugate;
(ii) each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;
(iii) each occurrence of T is independently a covalent bond or a bivalent, straight or branched, saturated or unsaturated, optionally substituted C₁₋₃₀ hydrocarbon chain wherein one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;
(iv) each occurrence of R is independently hydrogen, a suitable protecting group, an acyl moiety, arylalkyl moiety, aliphatic moiety, aryl moiety, heteroaryl moiety, or heteroaliphatic moiety;
(v) —B is -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(vi) n is 1, 2, or 3.

### Description of Exemplary Groups

[symbol]

(Node)

In particular embodiments, each occurrence of [symbol] is independently an optionally substituted group selected from the group consisting of acyl, aliphatic, heteroaliphatic, aryl, heteroaryl, and heterocyclic. In some embodiments, each occurrence of [symbol] is the same. In some embodiments, the central [symbol] is different from all other occurrences of [symbol] . In particular embodiments, all occurrences of [symbol] are the same except for the central [symbol] .

In some embodiments, [symbol] is an optionally substituted aryl or heteroaryl group.

In some embodiments, [symbol] is a 2-, 3, 4, 6, or 8-membered aryl or heteroaryl group. In some embodiments, [symbol] is a 5- or 6-membered heterocyclic group. In particular embodiments, [symbol] is a heteroatom selected from N, O, or S. In some embodiments, [symbol] is nitrogen atom. In some embodiments, [symbol] is an oxygen atom. In some embodiments, [symbol] is sulfur atom. In some embodiments, [symbol] is a carbon atom. In some embodiments, [symbol] is the structure

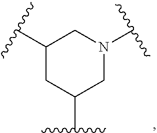 [SMILES: *C1CC(*)CN(*)C1]

### T (Spacer)

In particular embodiments, each occurrence of T is independently a bivalent, straight or branched, saturated or unsaturated, optionally substituted C₁₋₂₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group. In particular embodiments, one, two, three, four, or five methylene units of T are optionally and independently replaced. In particular embodiments, T is constructed from a C₁₋₁₀, C₁₋₈, C₁₋₆, C₁₋₄, C₂₋₁₂, C₄₋₁₂, C₆₋₁₂, C₈₋₁₂, or C₁₀₋₁₂ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group. In some embodiments, one or more methylene units of T is replaced by a heterocyclic group. In some embodiments, one or more methylene units of T is replaced by a triazole moiety. In particular embodiments, one or more methylene units of T is replaced by —C(O)—. In particular embodiments, one or more methylene units of T is replaced by —C(O)N(R)—. In particular embodiments, one or more methylene units of T is replaced by —O—.

In particular embodiments of the conjugate, the conjugate comprises or consists of the structure of conjugate I, wherein the insulin or insulin analog is conjugated to a tetra-valent linker selected from the group consisting of:

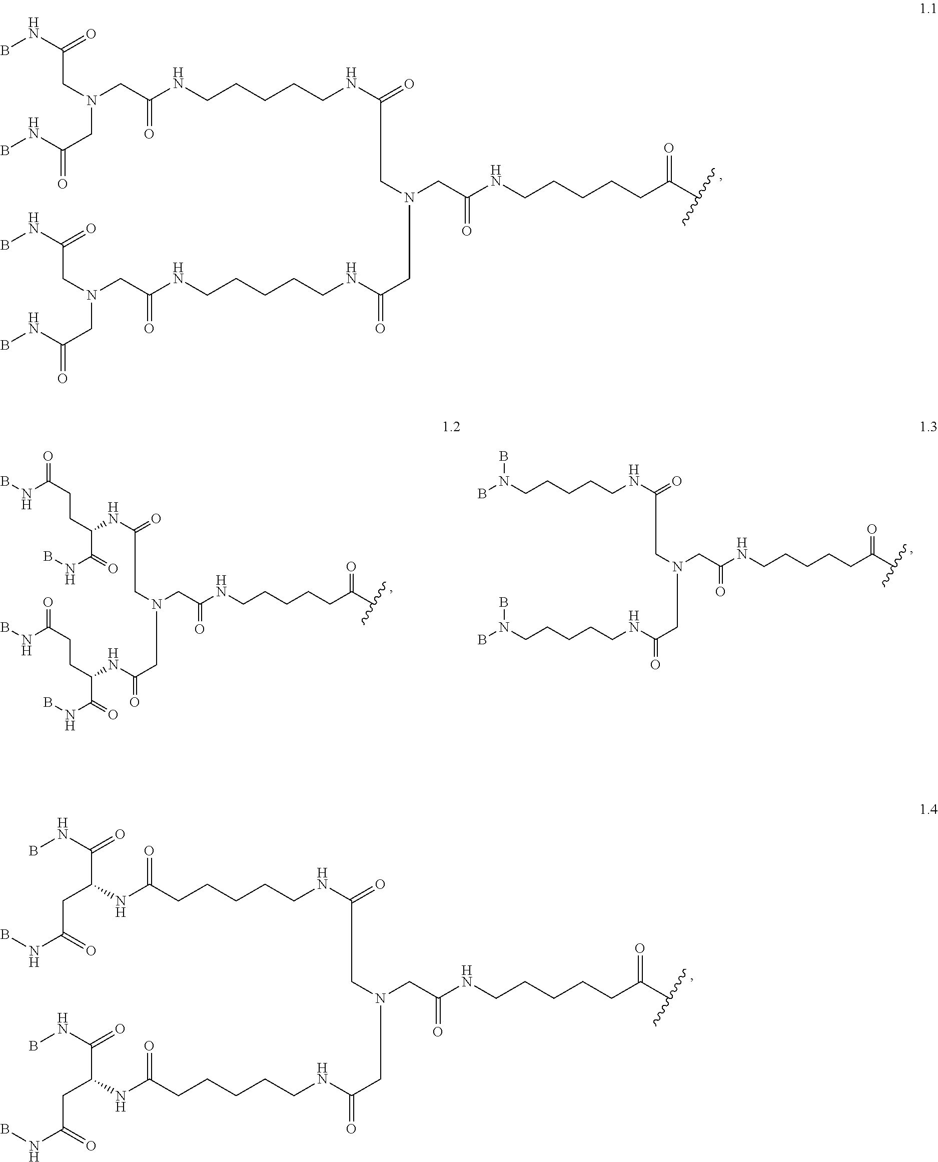

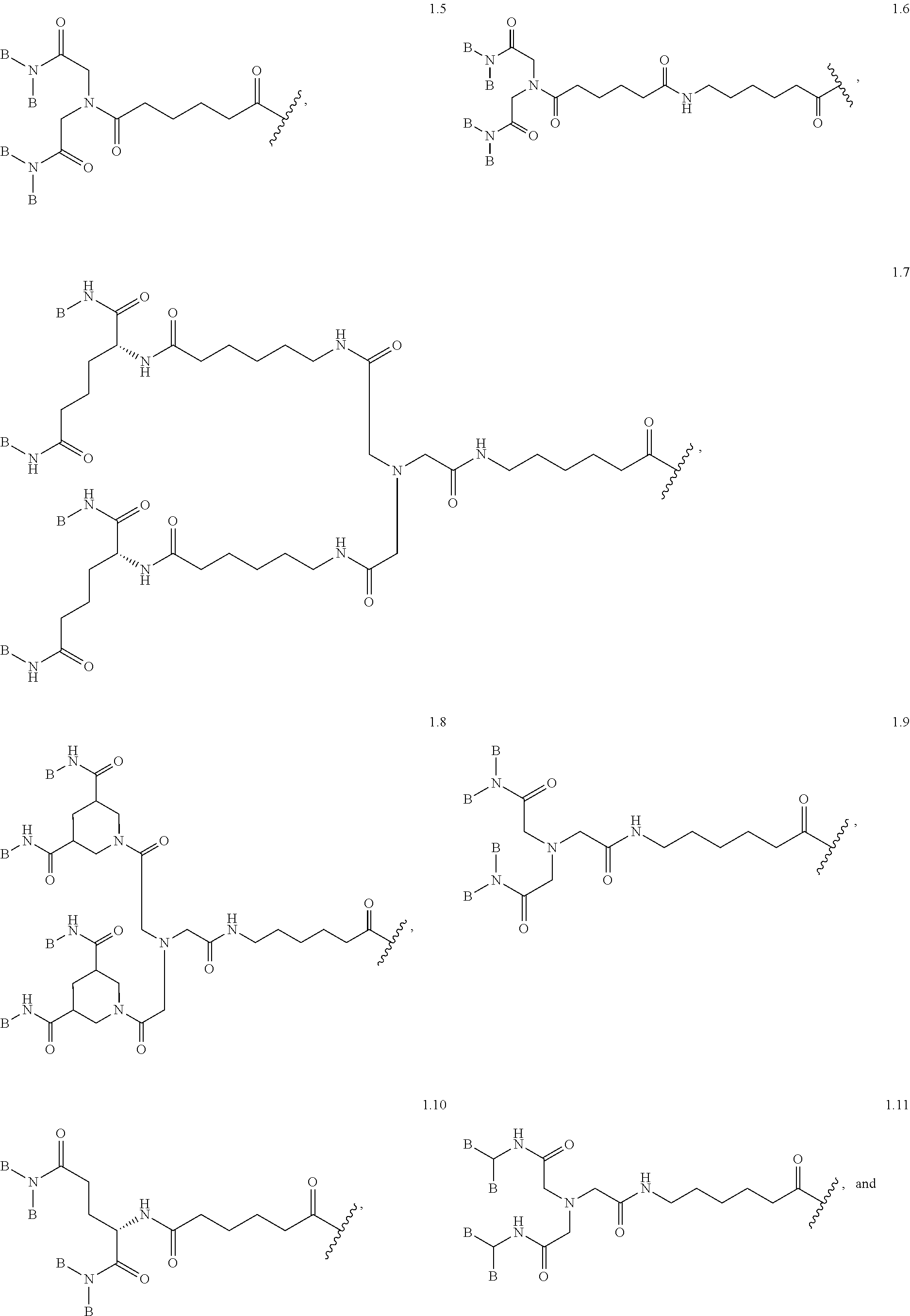

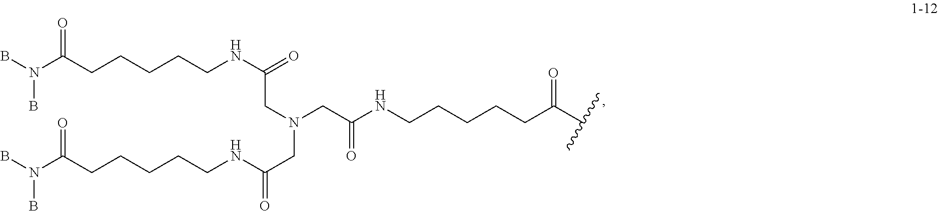

or the conjugate comprises the structure of conjugate II, wherein the insulin or insulin analog is conjugated to a tetra-valent linker selected from the group consisting of:

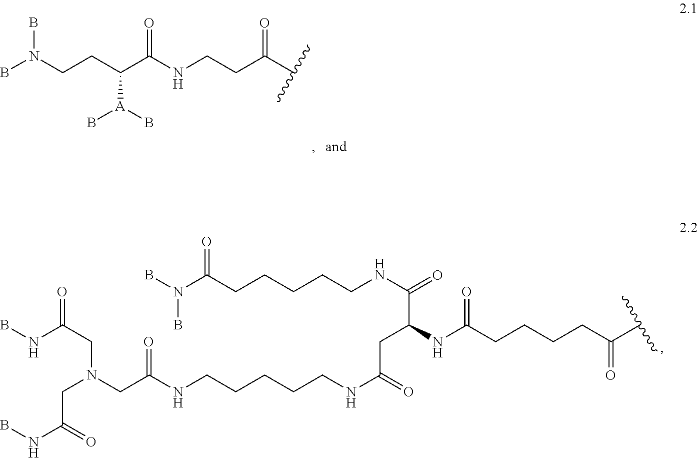

or the conjugate comprises the structure of conjugate III, wherein the insulin or insulin analog is conjugated to a tetra-valent linker selected from the group consisting of:

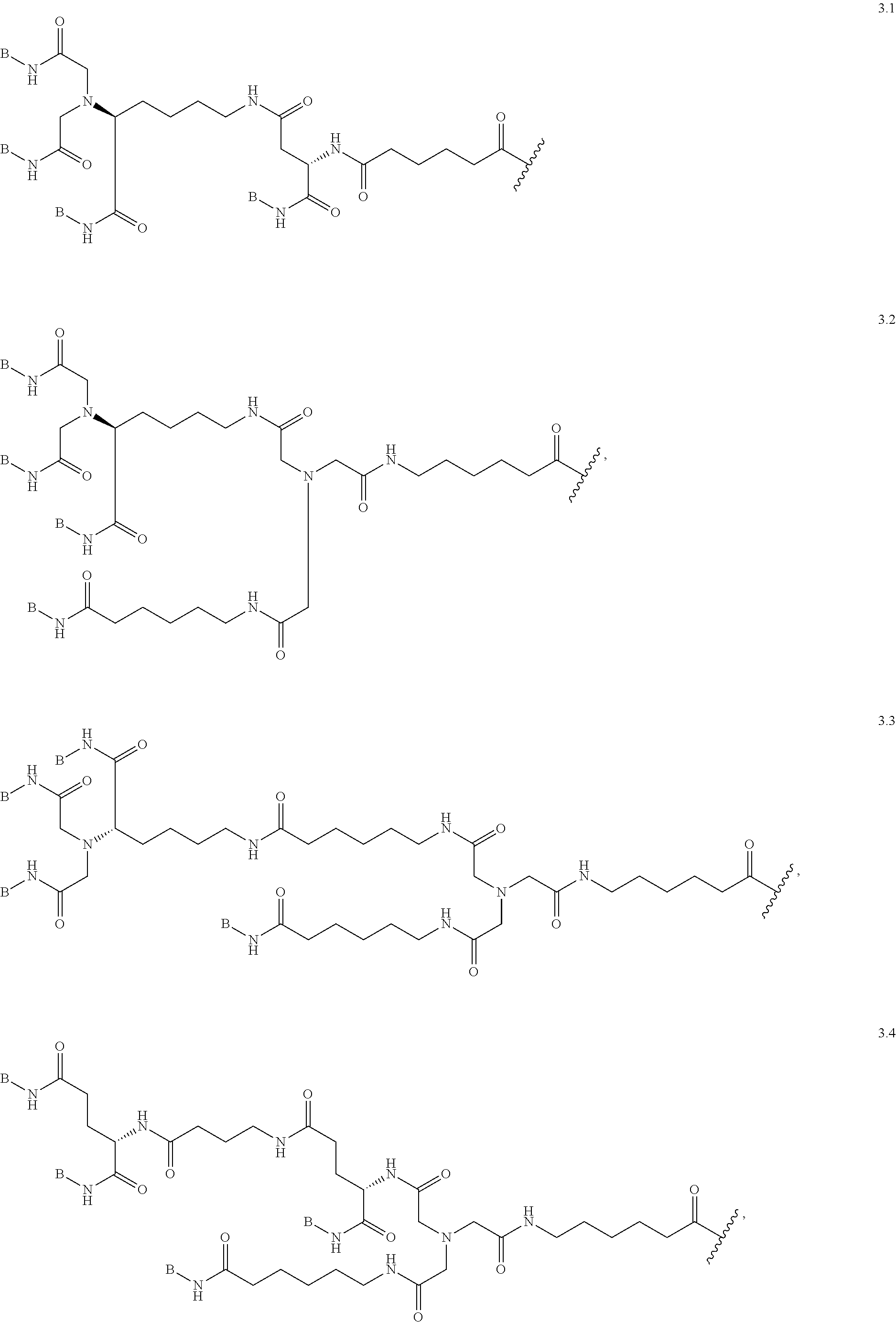

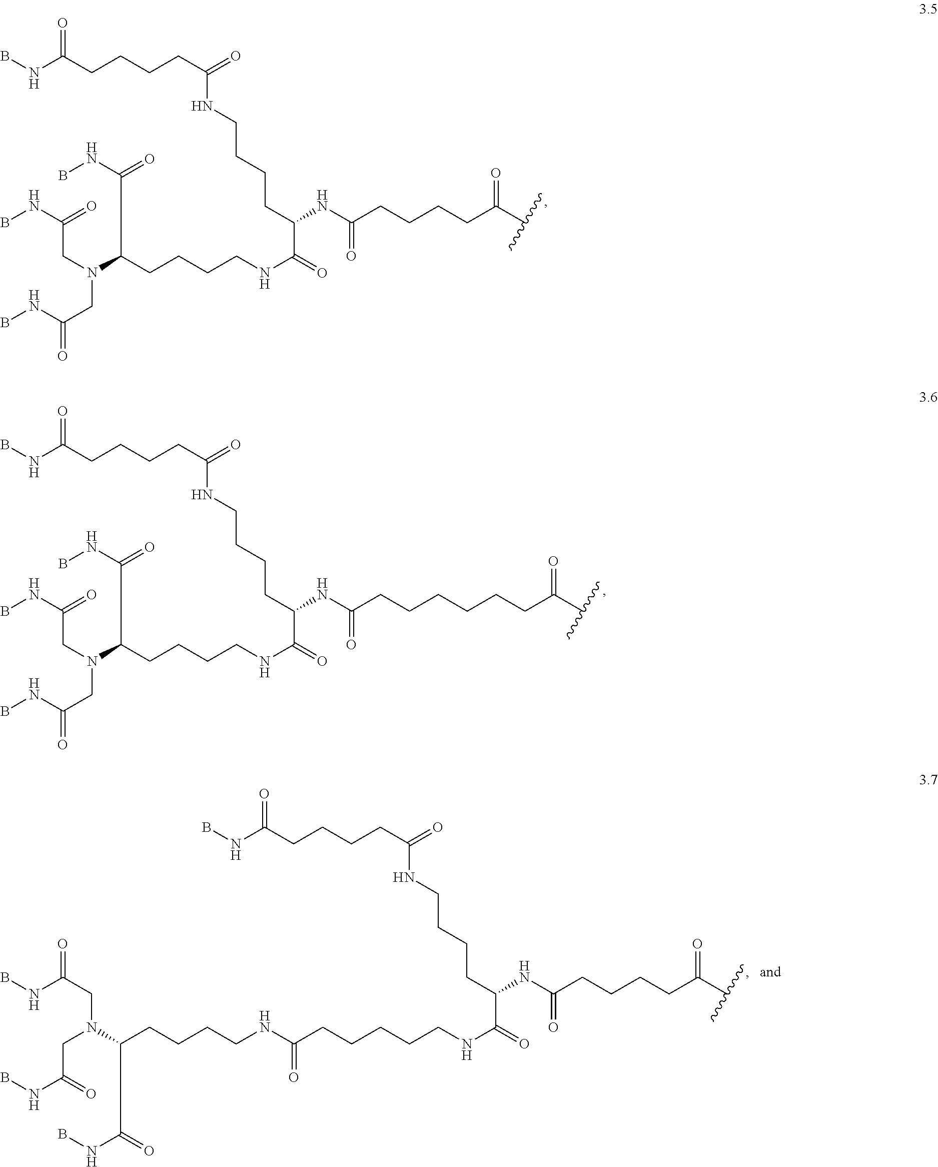

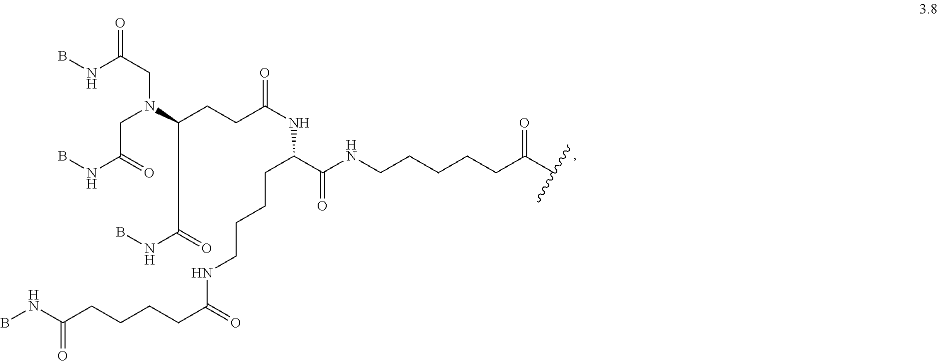

wherein a wavy line indicates the bond between the proximal end of the linker arm and amino acid on the insulin or insulin analog and wherein each B is independently -T-L^{B}-X, wherein each occurrence of X is independently the ligand and each occurrence of L^{B} is independently a covalent bond or a group derived from the covalent conjugation of a T with an X.

In particular embodiments, the insulin analog may comprise an A chain sequence comprising a sequence of GIVEQCCX₁SICSLYQLENYCX₂ (SEQ ID NO: 8); and a B chain sequence comprising a sequence of X₃LCGX₄X₅LVEALYLVCG ERGFF (SEQ ID NO: 9), or X₈VNQX₃LCGX₄X₅LVEALYLVCGERGFFYTX₆ X₇(SEQ ID NO: 10), wherein
X₁ is selected from the group consisting of threonine and histidine;
X₂ is selected from the group consisting of asparagine and glycine;
X₃ is selected from the group consisting of histidine and threonine;
X₄ is selected from the group consisting of alanine, glycine, and serine;
X₅ is selected from the group consisting of histidine, aspartic acid, glutamic acid, homocysteic acid, and cysteic acid;
X₆ is selected from the group consisting of aspartate-lysine dipeptide, a lysine-proline dipeptide, and a proline-lysine dipeptide;
X₇ is selected from the group consisting of threonine, alanine, and a threonine-arginine-arginine tripeptide; and
X₈ is selected from the group consisting of phenylalanine and desamino-phenylalanine.

In particular embodiments, the A-chain may have the amino acid sequence set forth in SEQ ID NO: 1 or SEQ ID NO: 6 and the B-chain may have the amino acid sequence set forth in SEQ ID NO: 2, SEQ ID NO: 3, SEQ ID NO: 4, or SEQ ID NO: 5. In particular embodiments, the insulin analog is a des B30 insulin analog, a des B29-B30 insulin analog, a des B28-B30 insulin analog, a des B27-B30 insulin analog, or a des B26-B30 insulin analog.

In particular embodiments, the insulin or insulin analog is conjugated to one, two, or three tetra-valent sugar clusters selected from the group consisting of ML-1, ML-2, ML-3, ML-4, ML-5, ML-6, ML-7, ML-8, ML-9, ML-10, ML-11, ML-12, ML-13, ML-14, ML-15, ML-16, ML-17, ML-18, ML-19, ML-20, ML-21, ML-22, ML-23, ML-24, ML-25, ML-26, ML-27, ML-28, ML-29, ML-30, ML-31, ML-32, ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, ML-52, and ML-53.

Exemplary human insulin oligosaccharide conjugates (IOCs) of the present disclosure include the IOCs having the following structures:

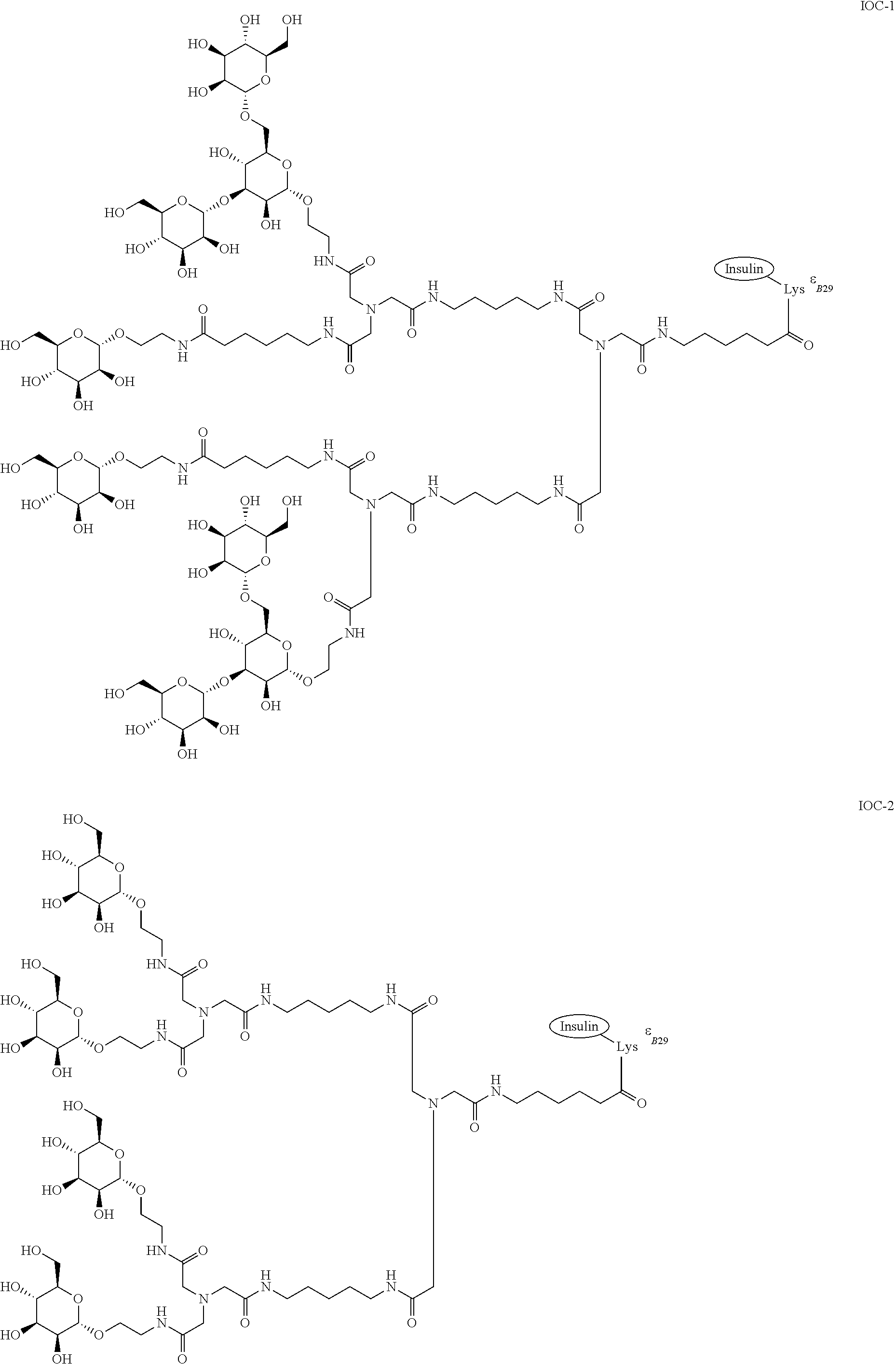

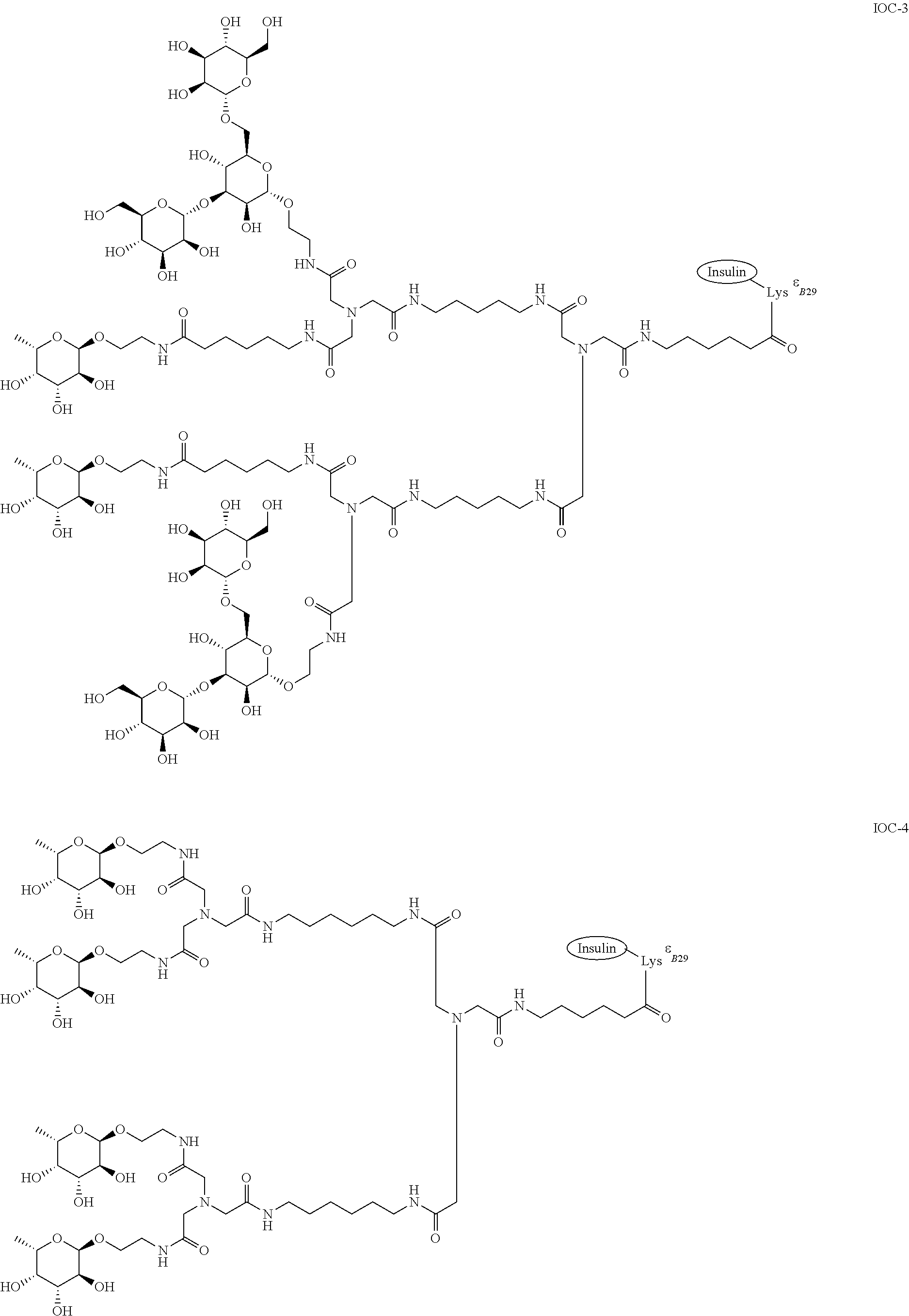

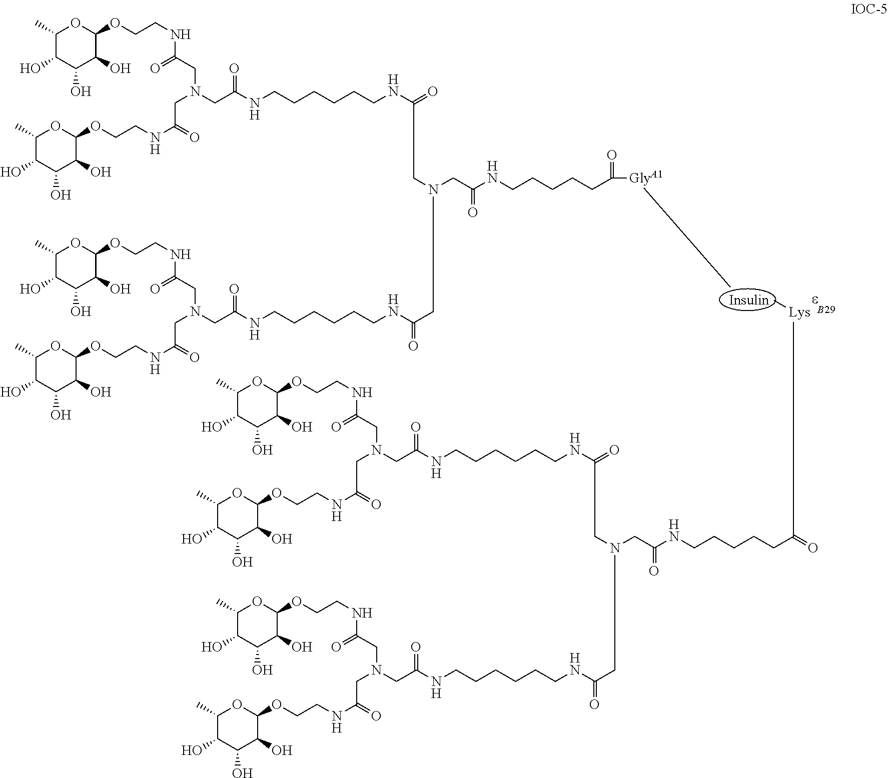

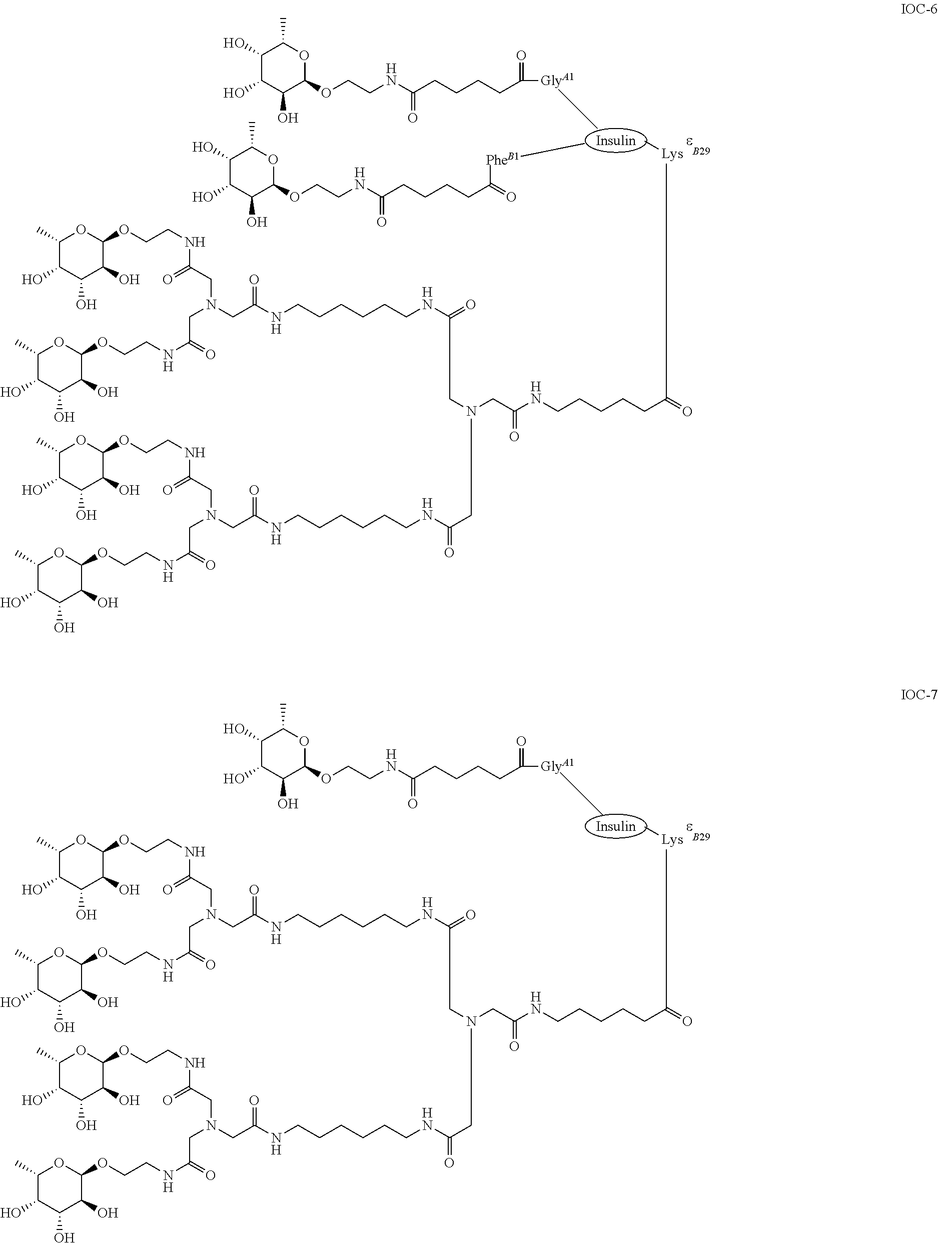

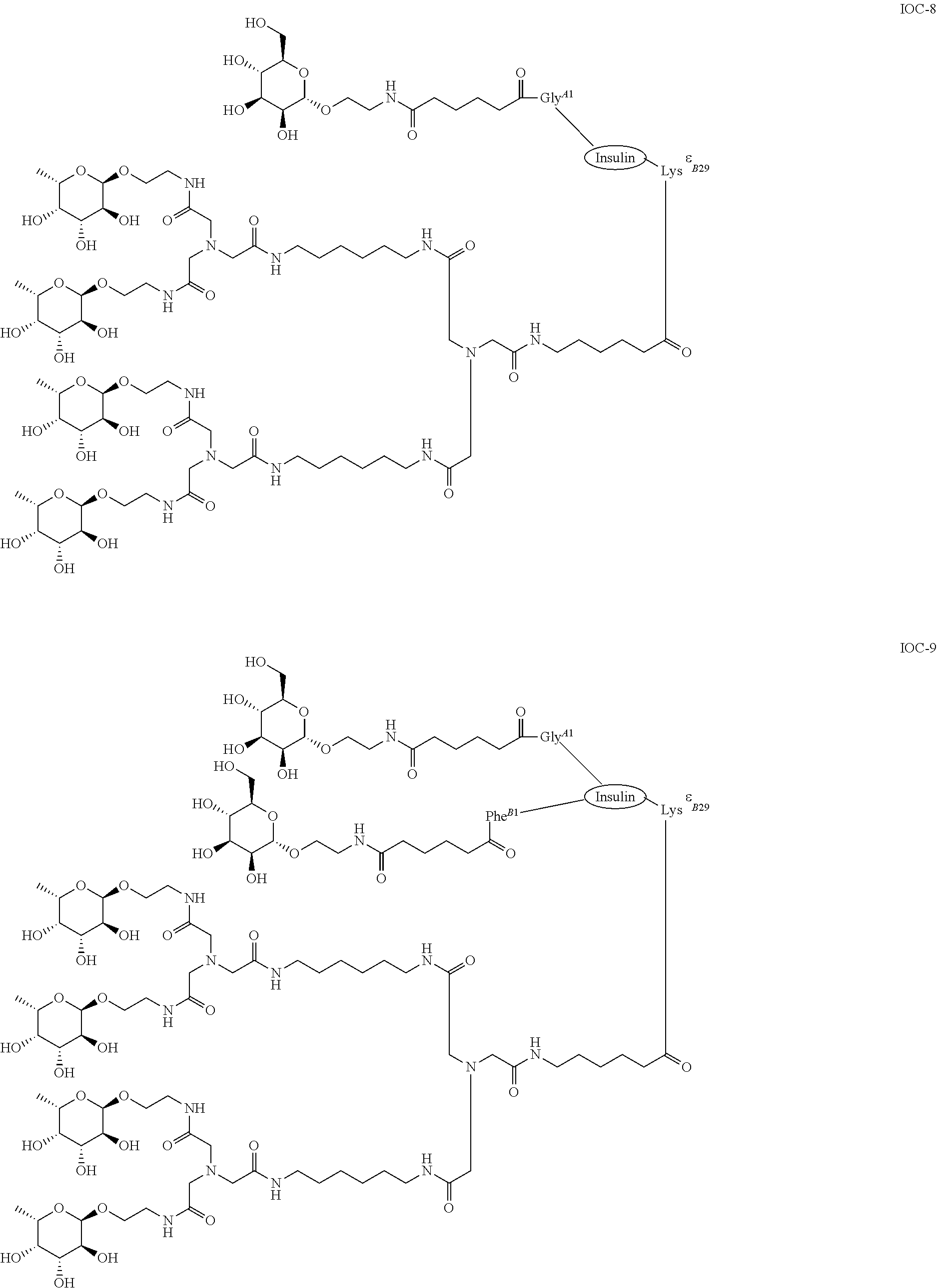

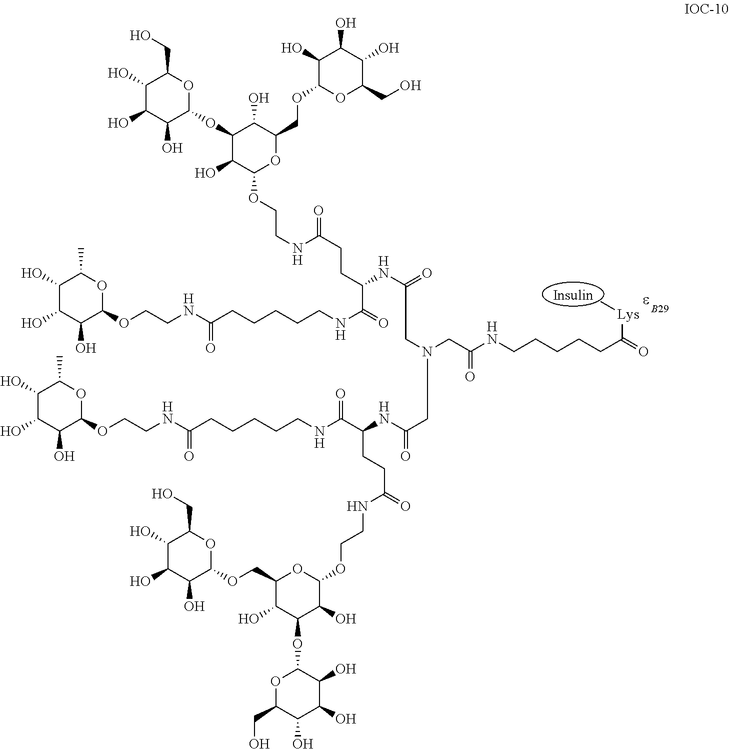

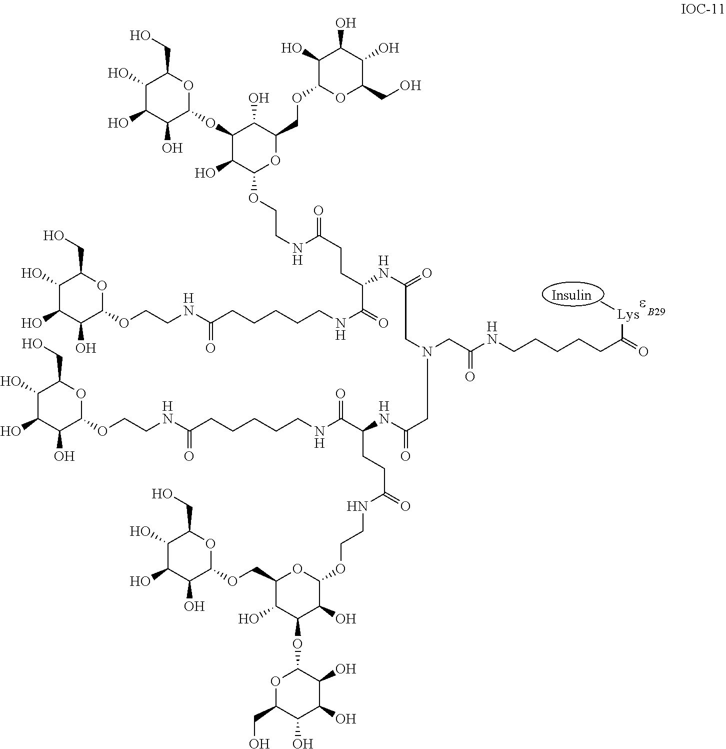

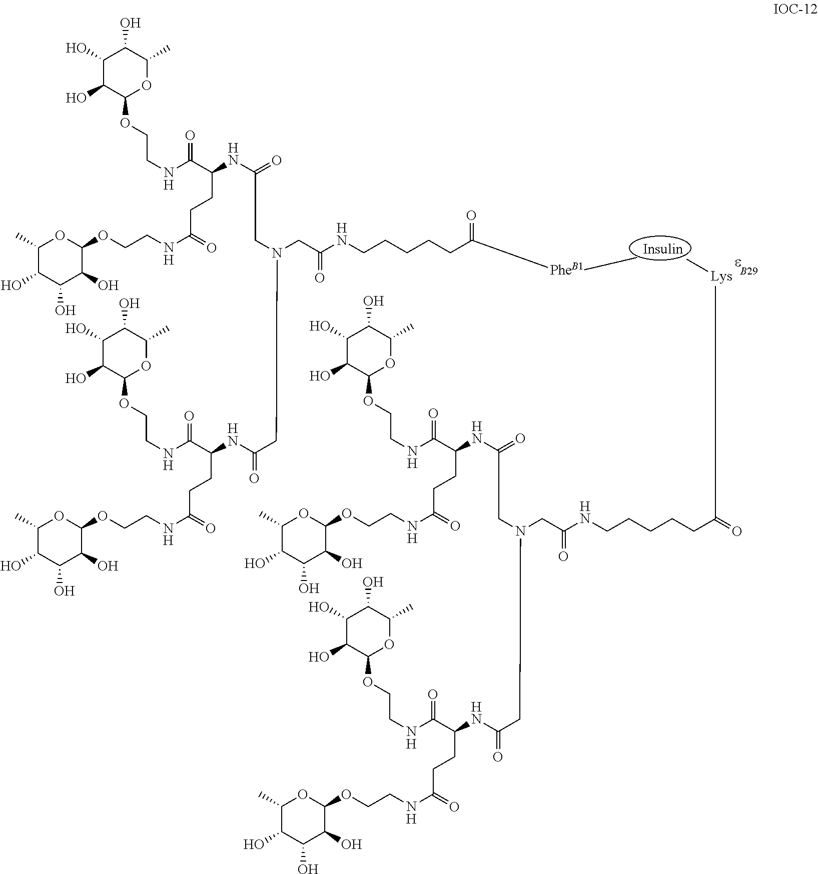

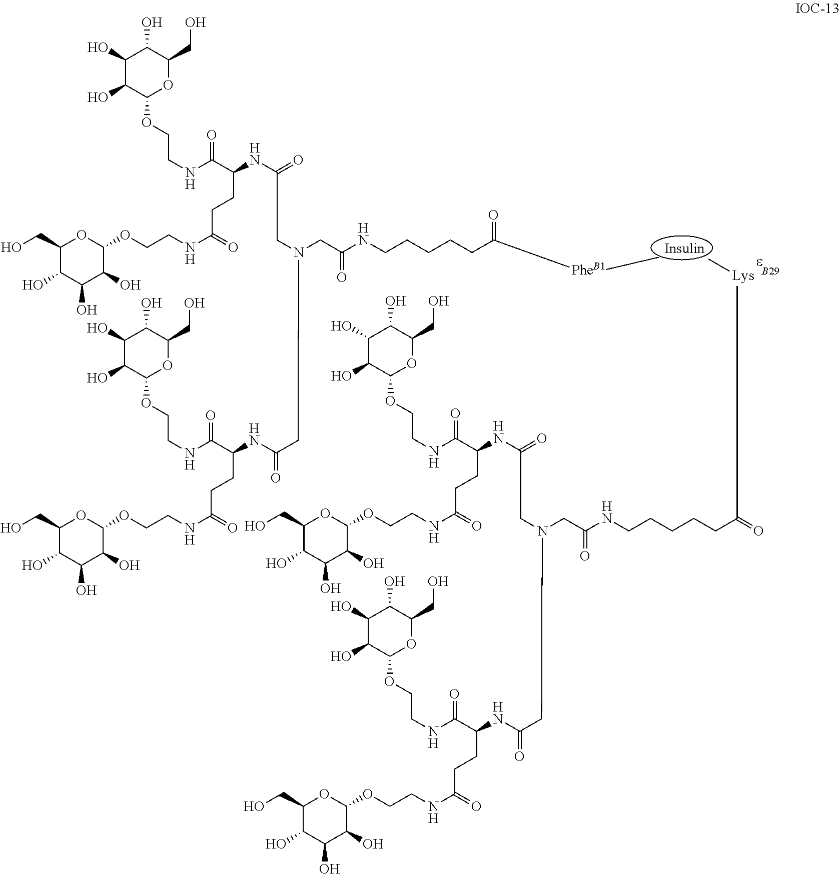

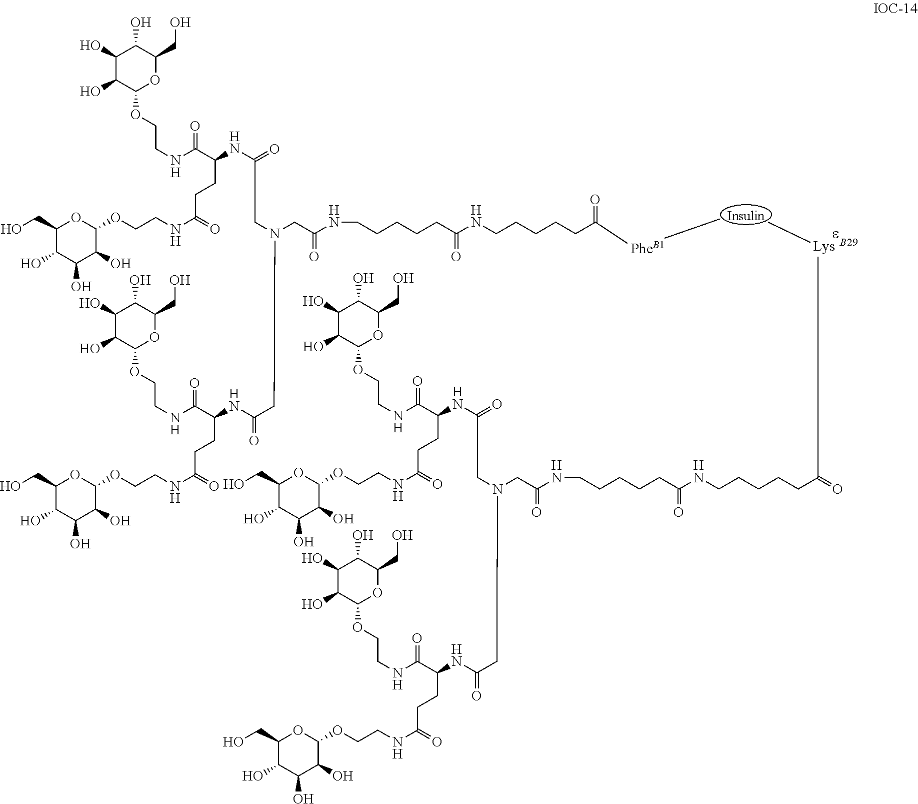

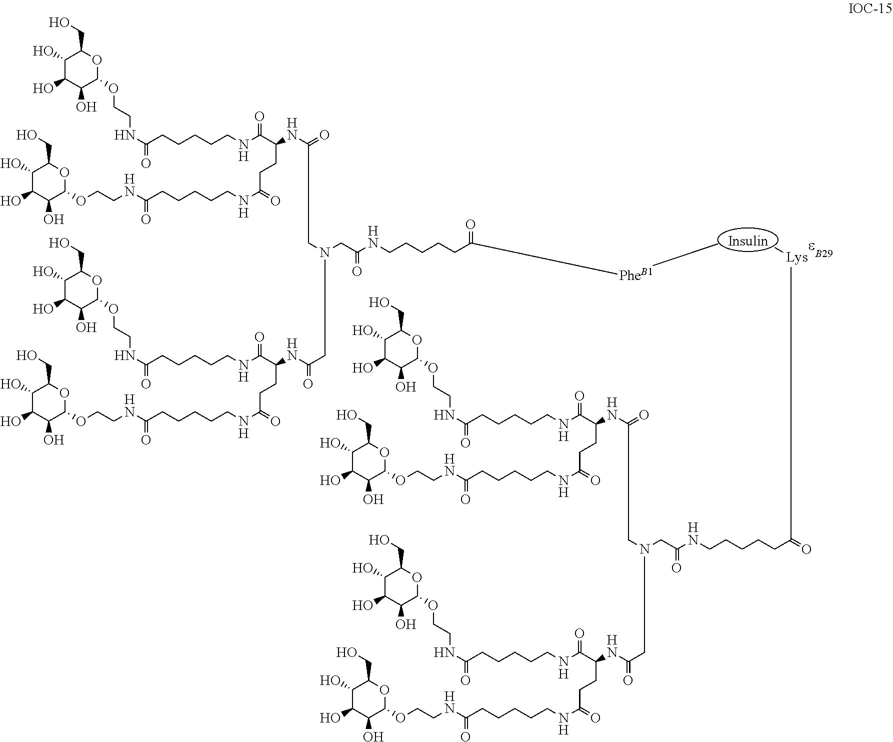

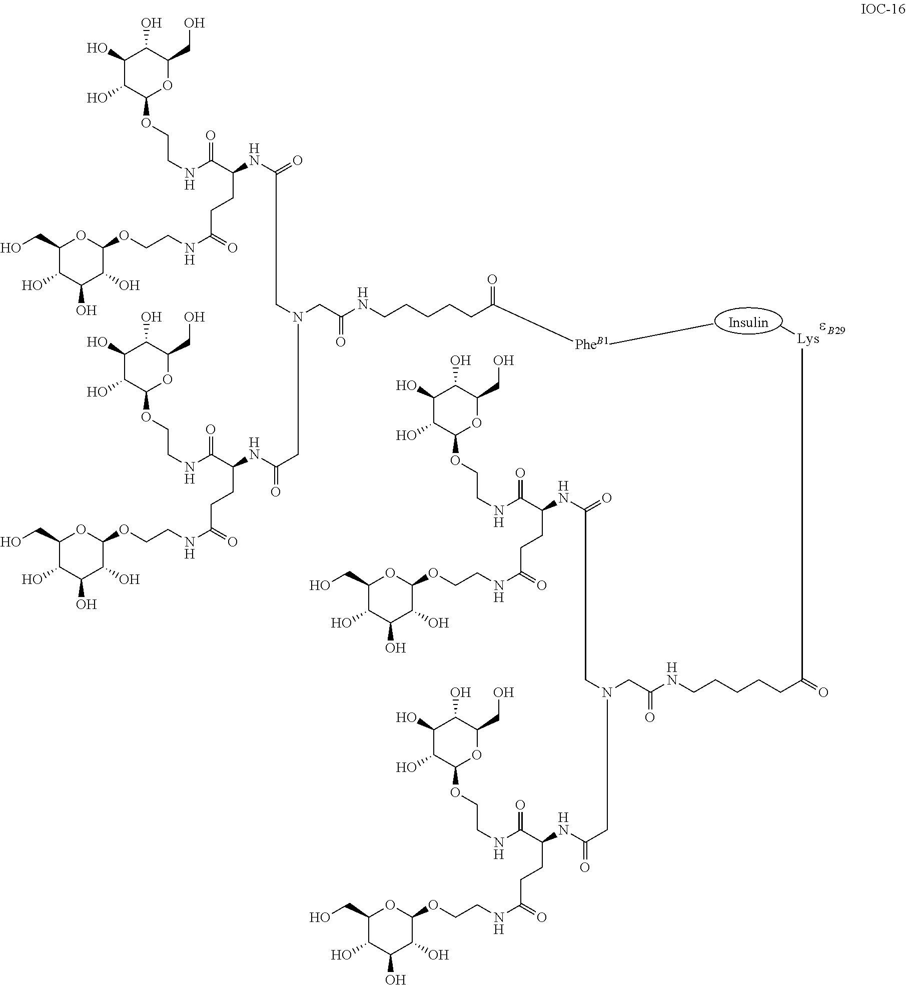

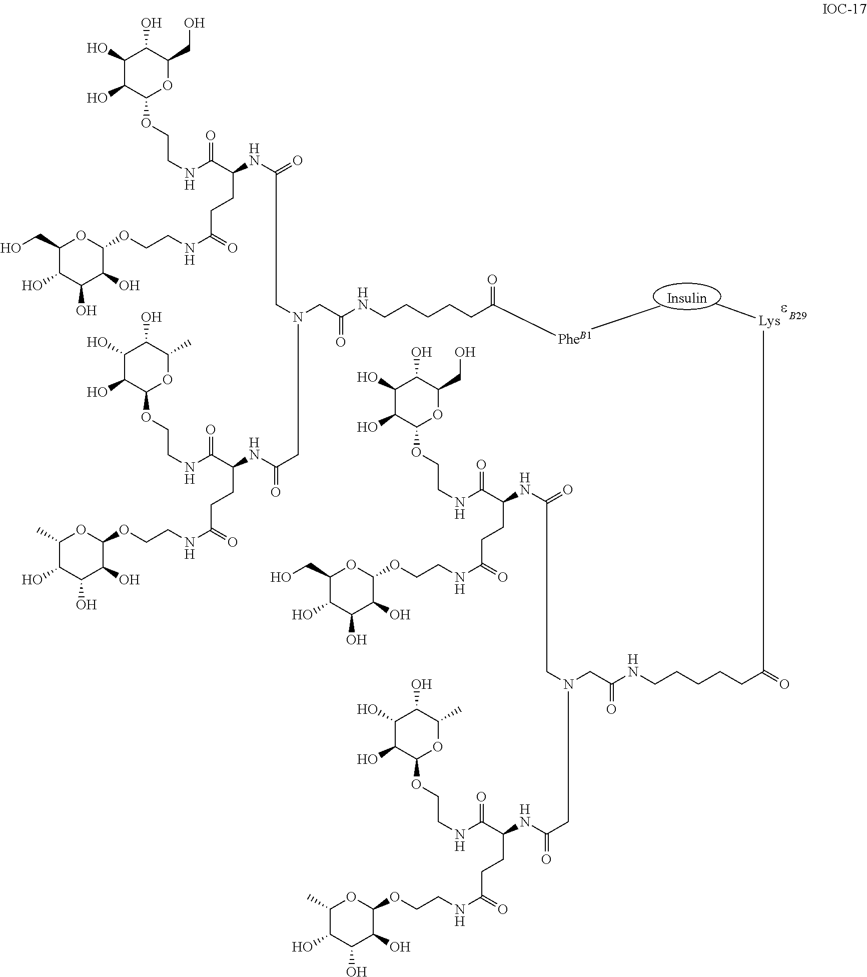

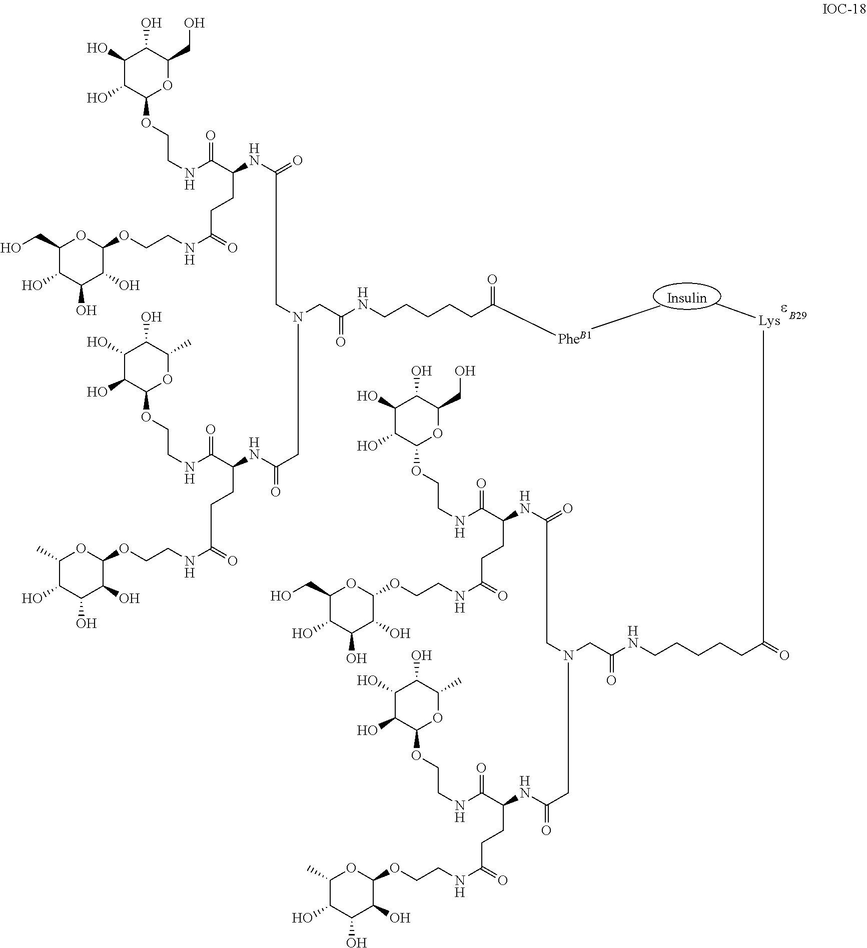

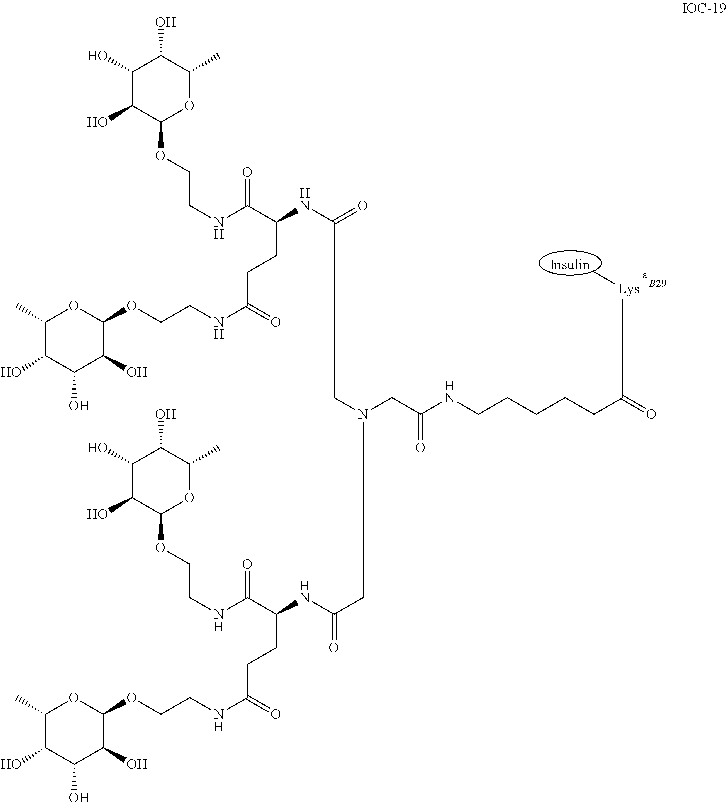

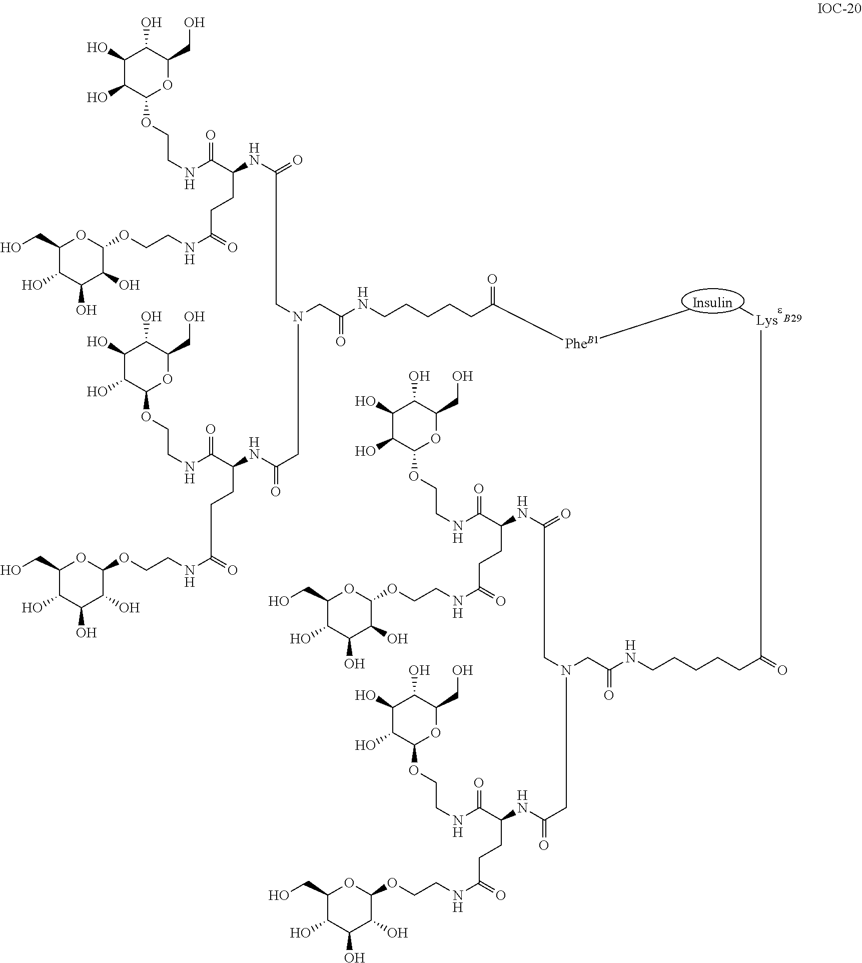

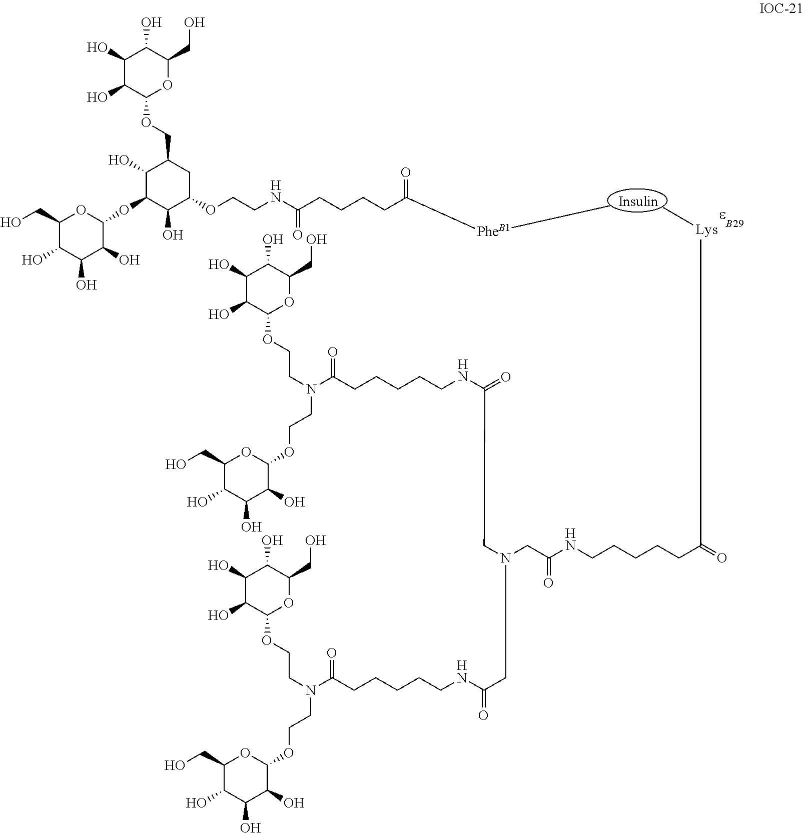

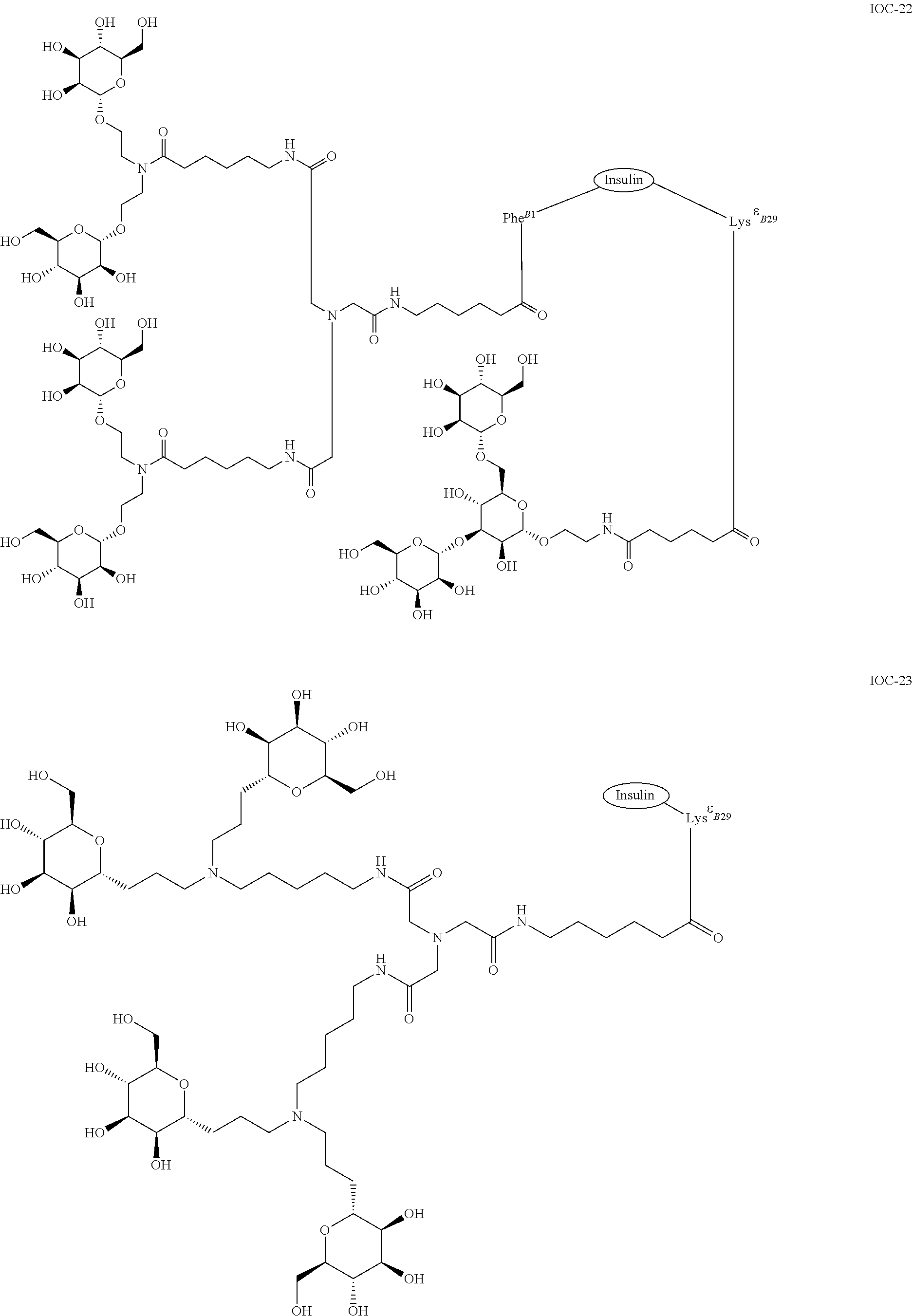

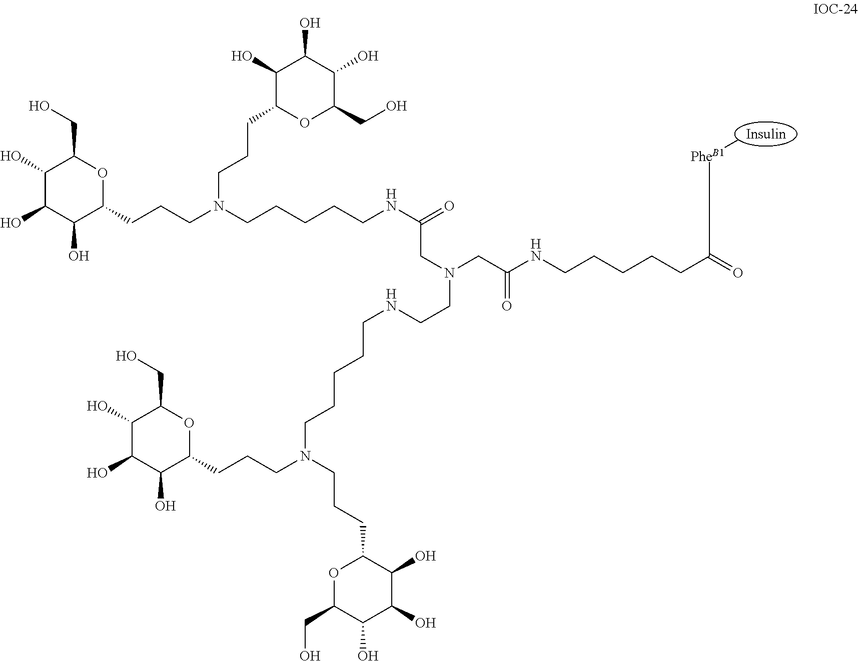

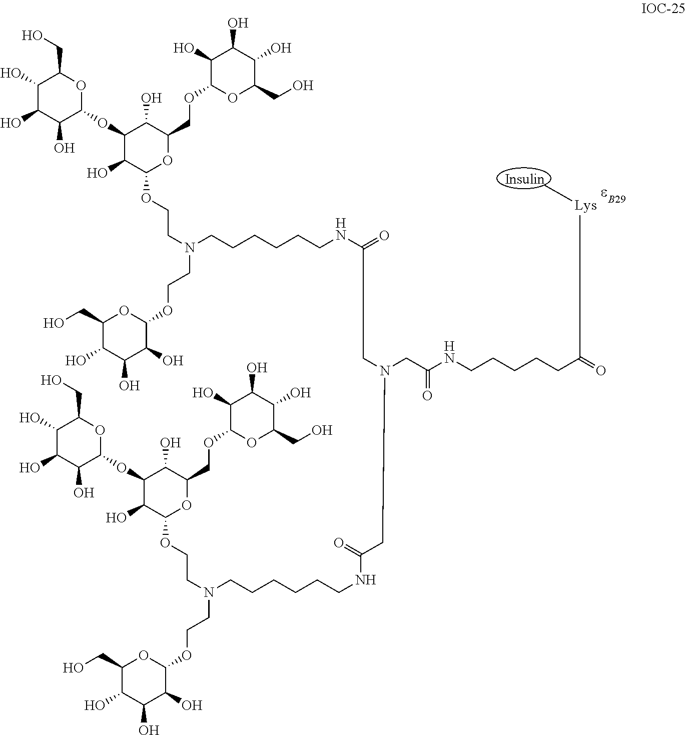

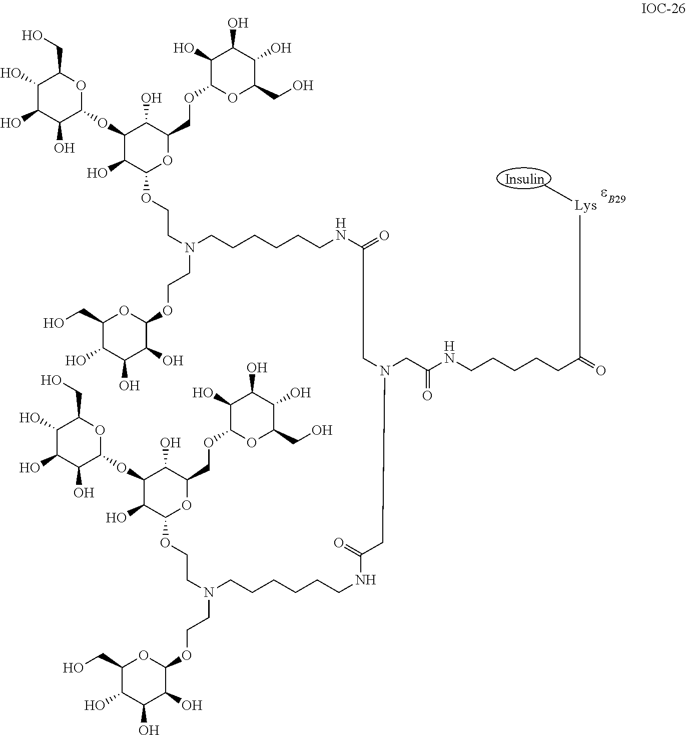

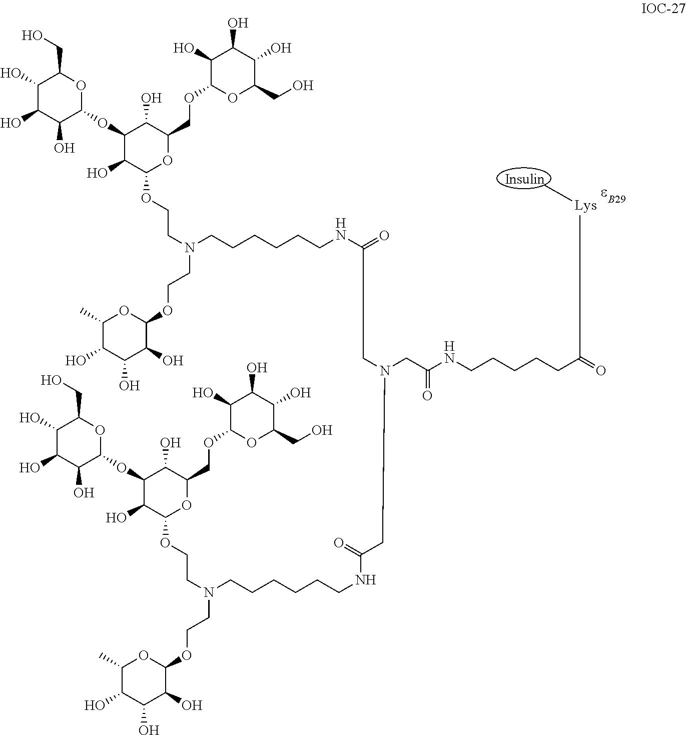

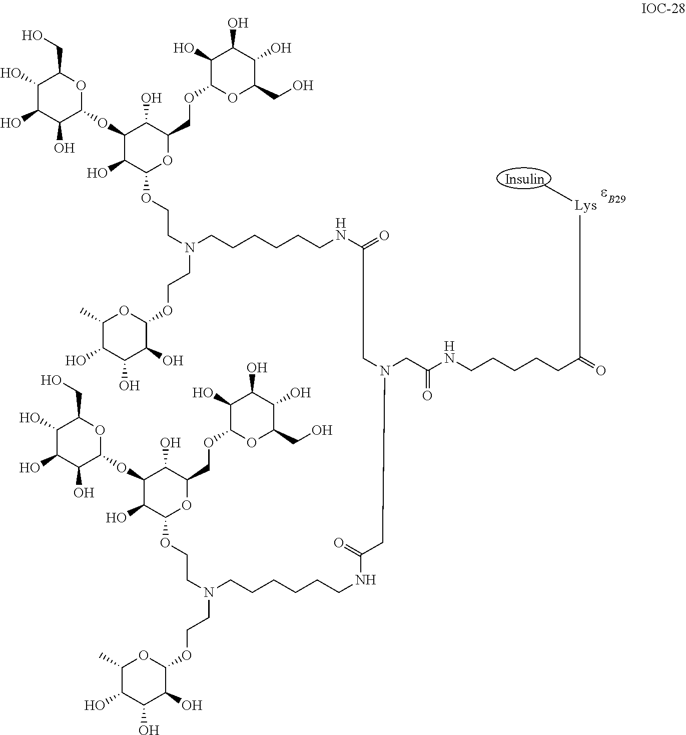

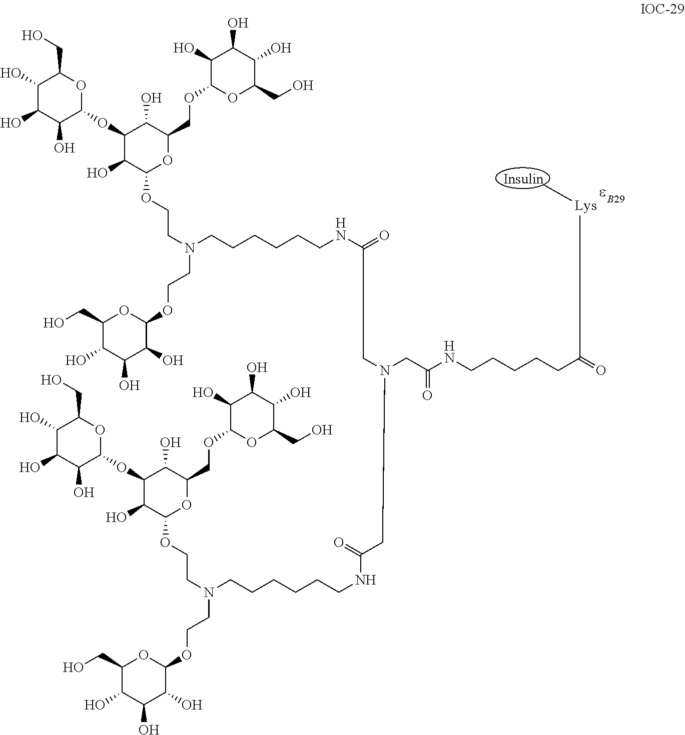

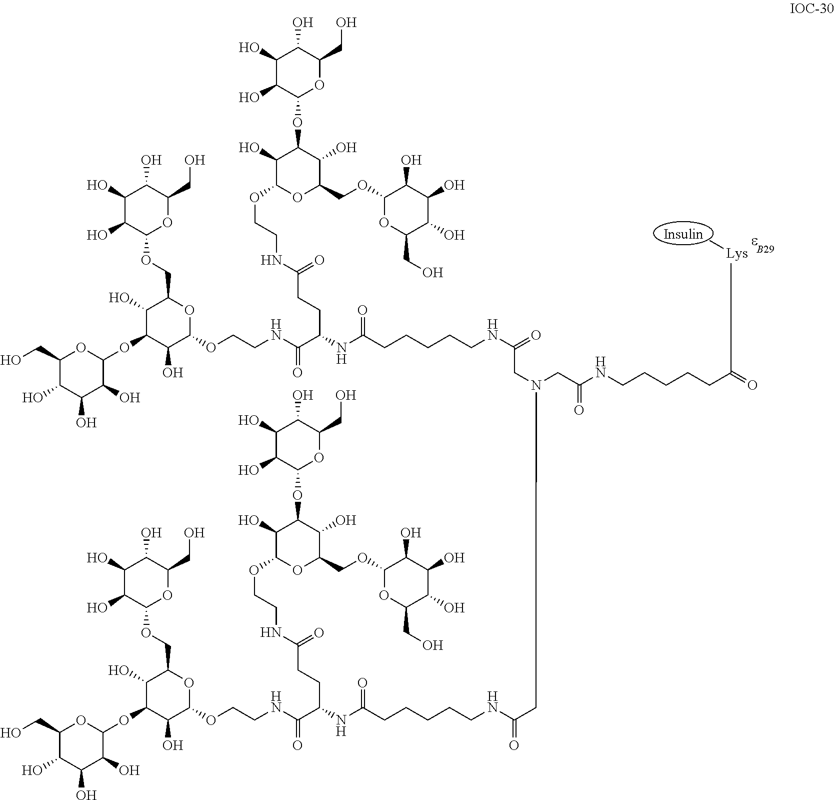

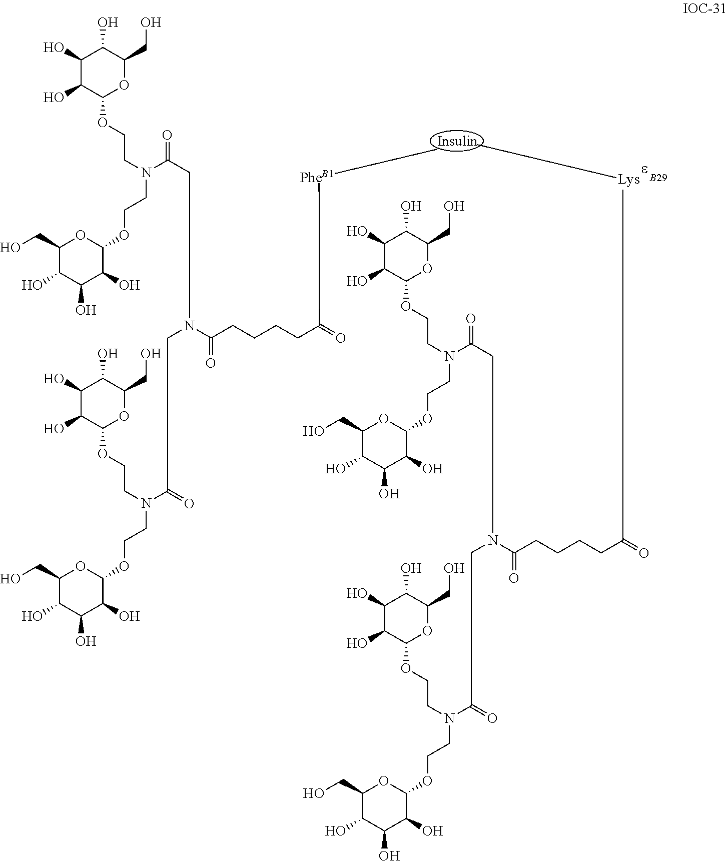

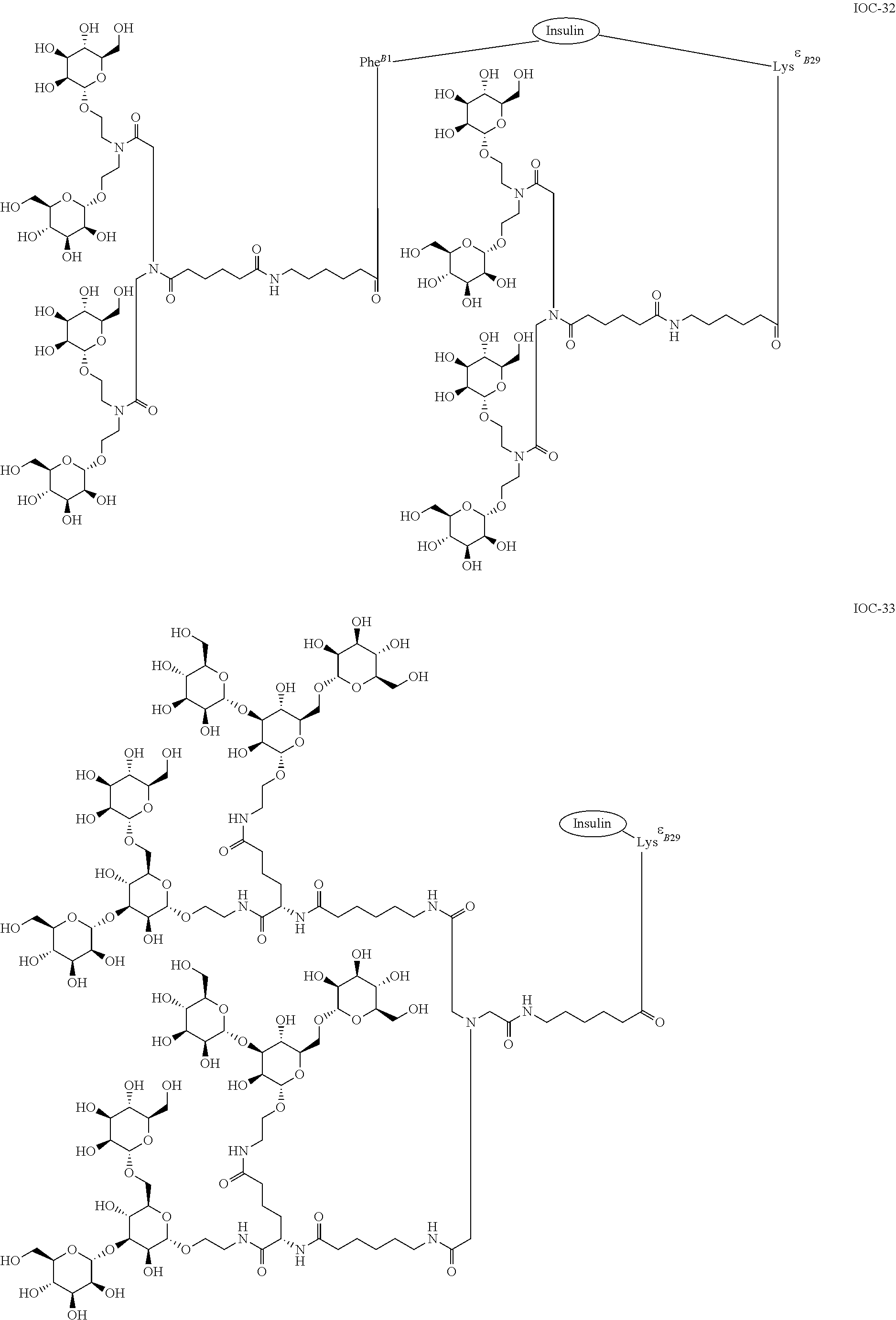

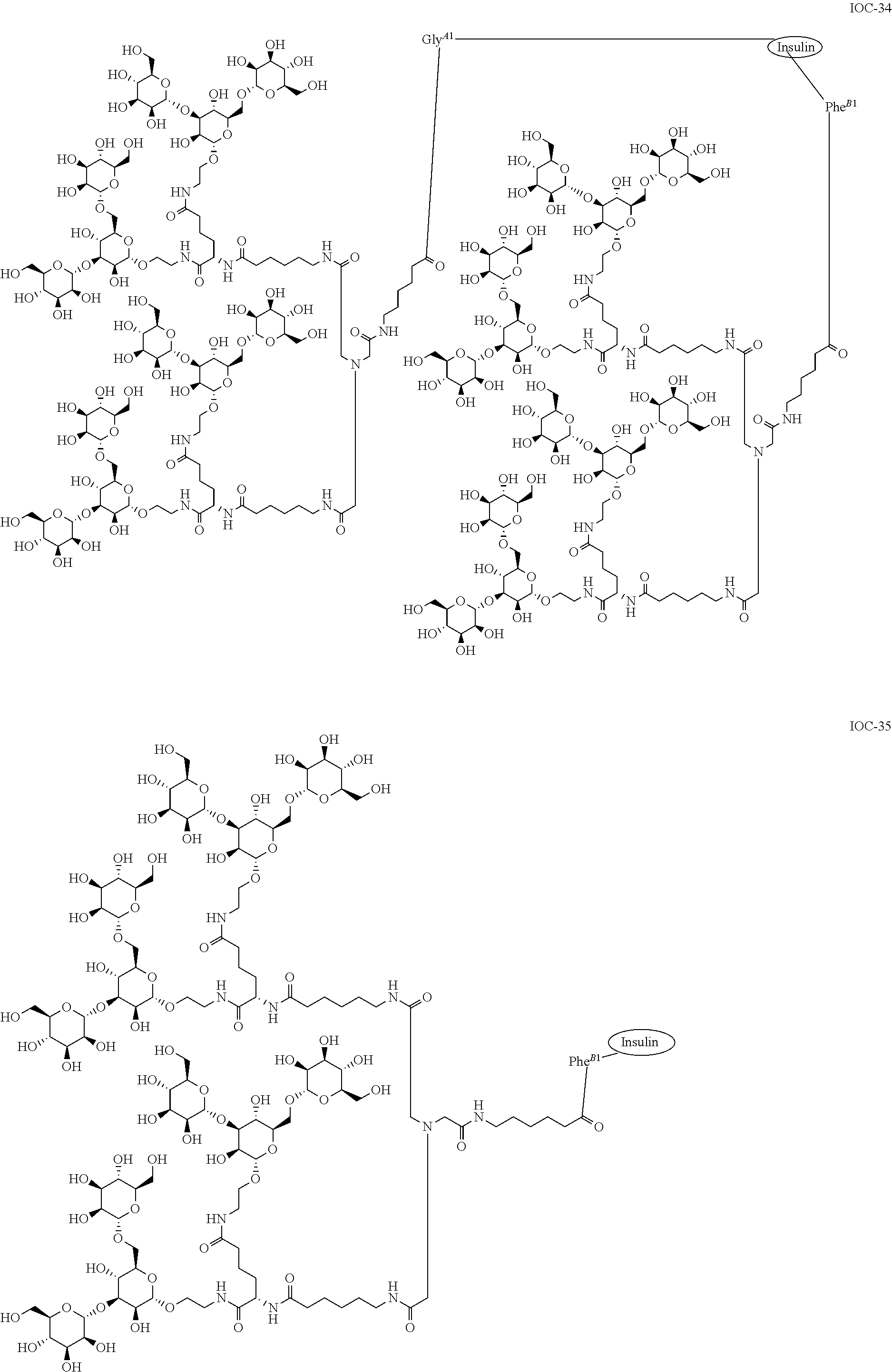

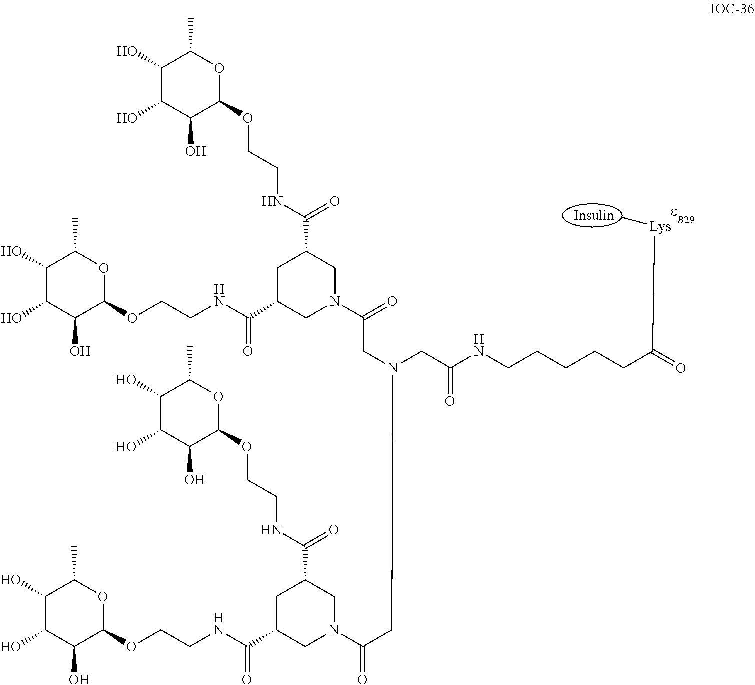

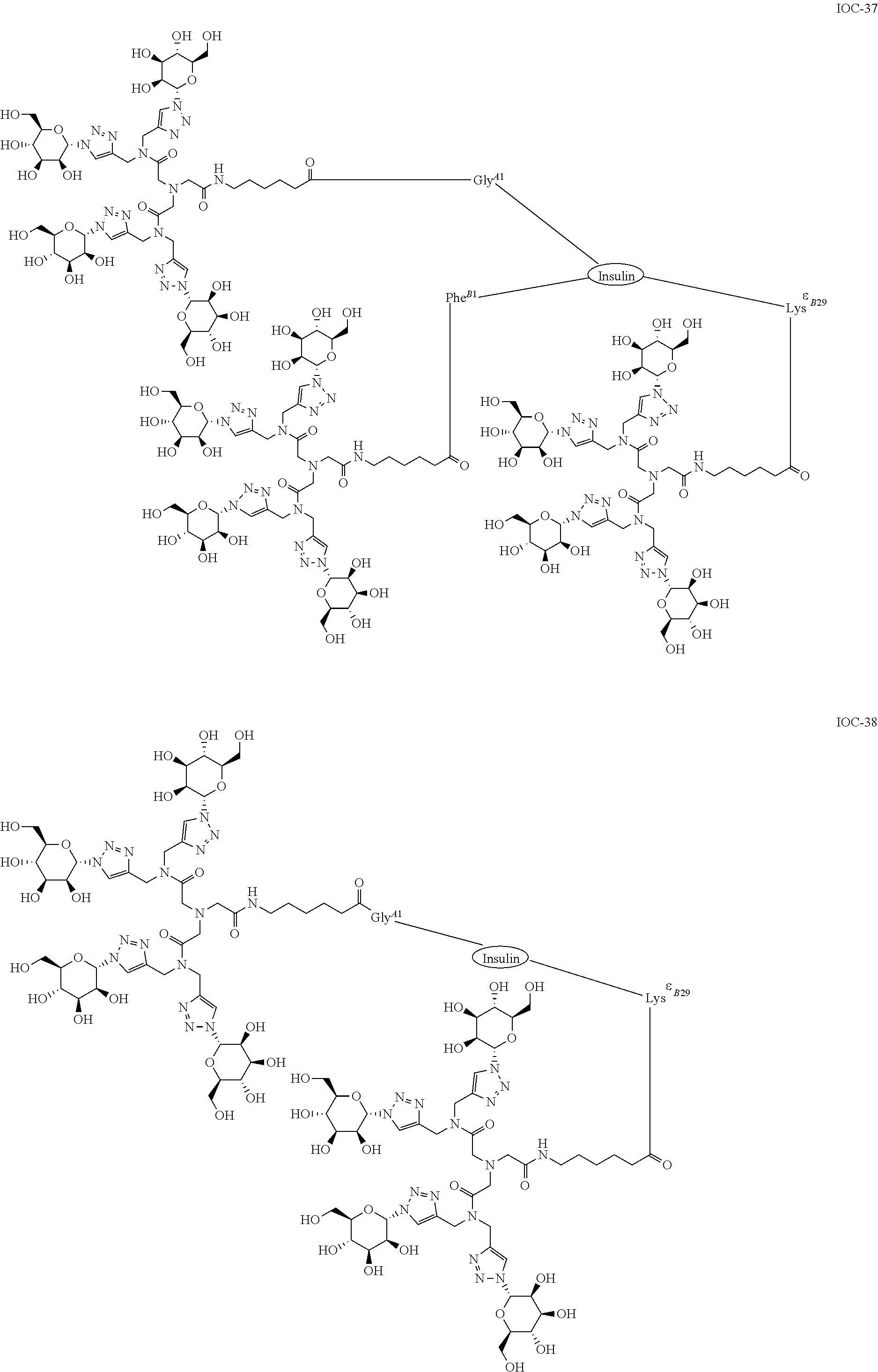

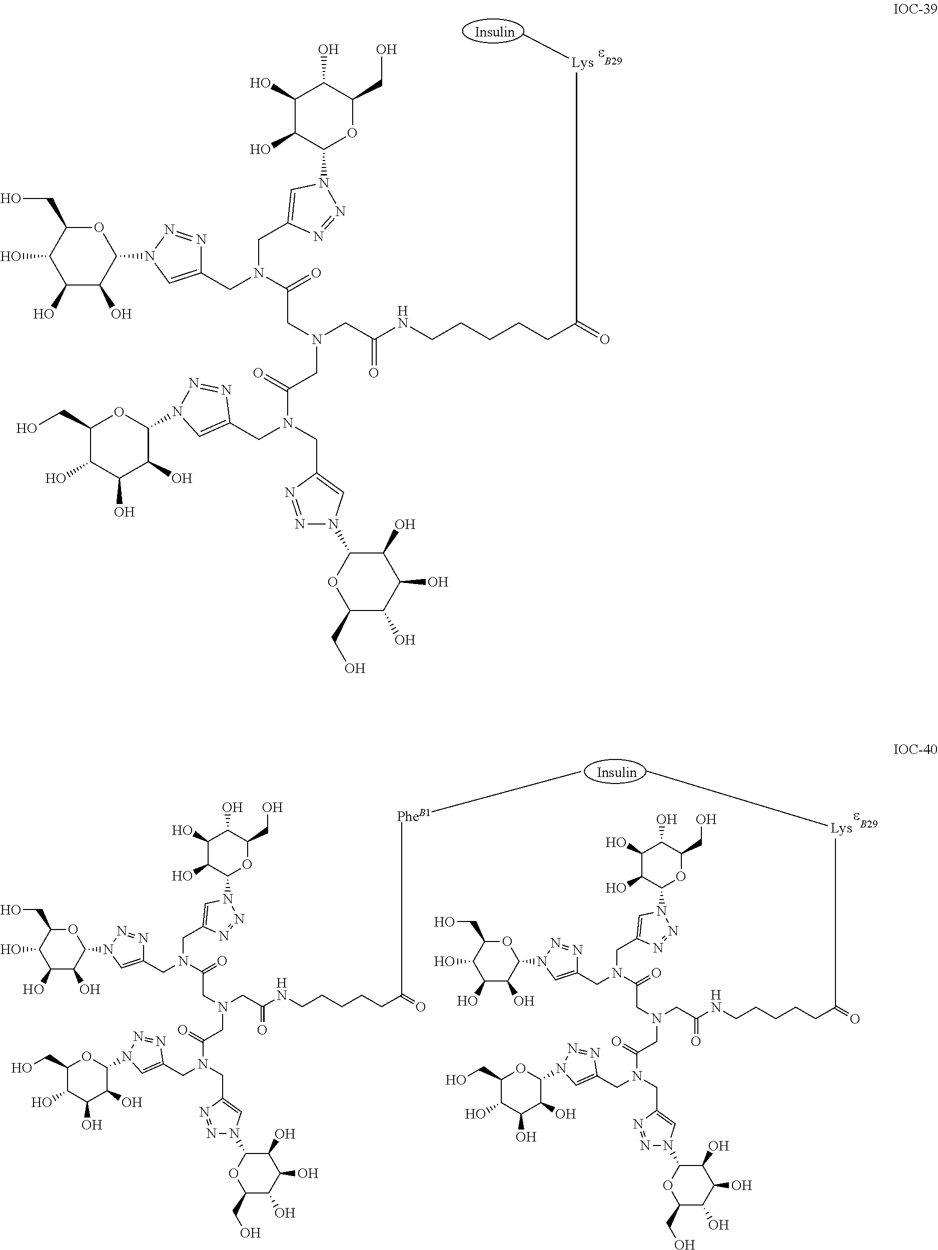

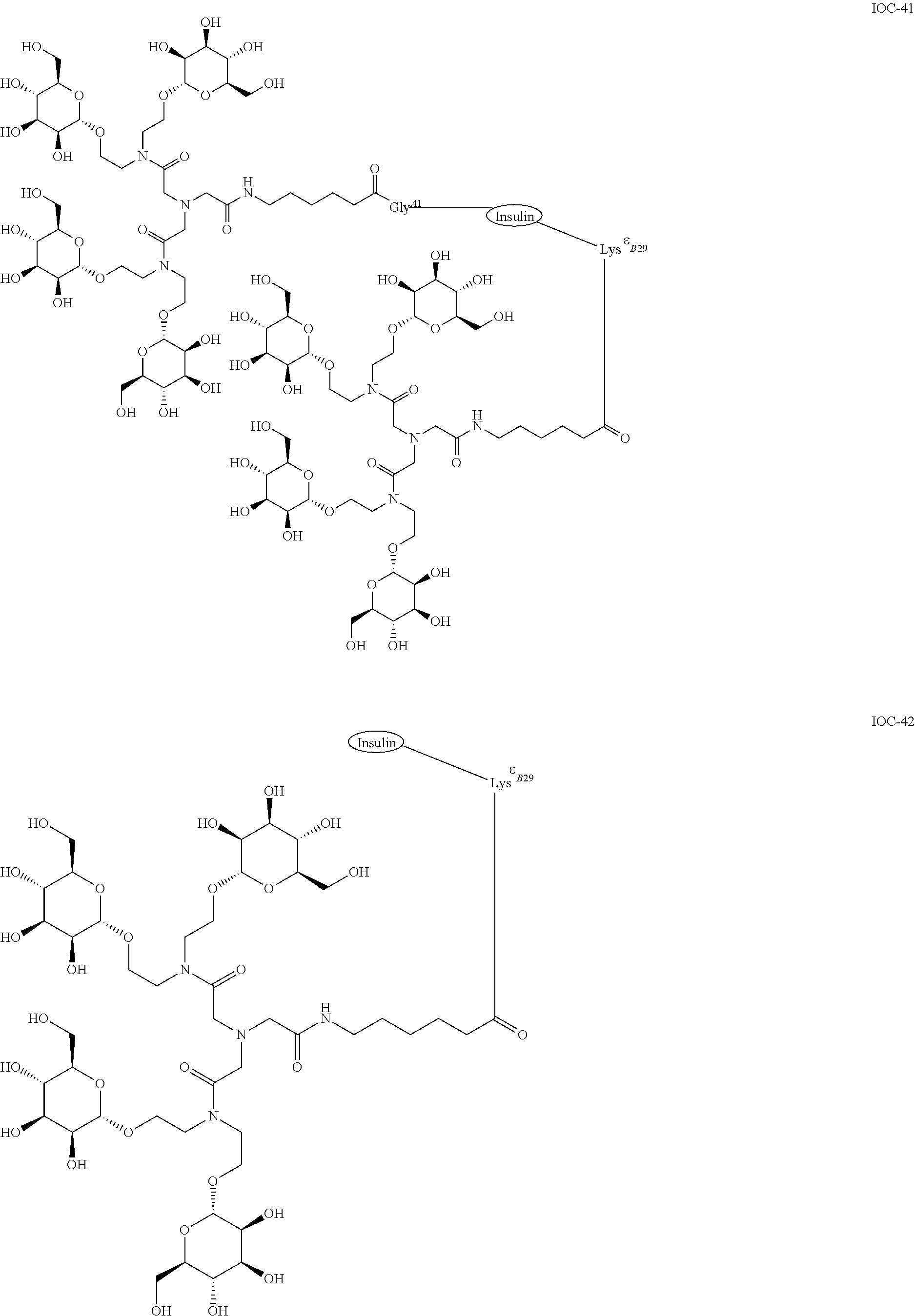

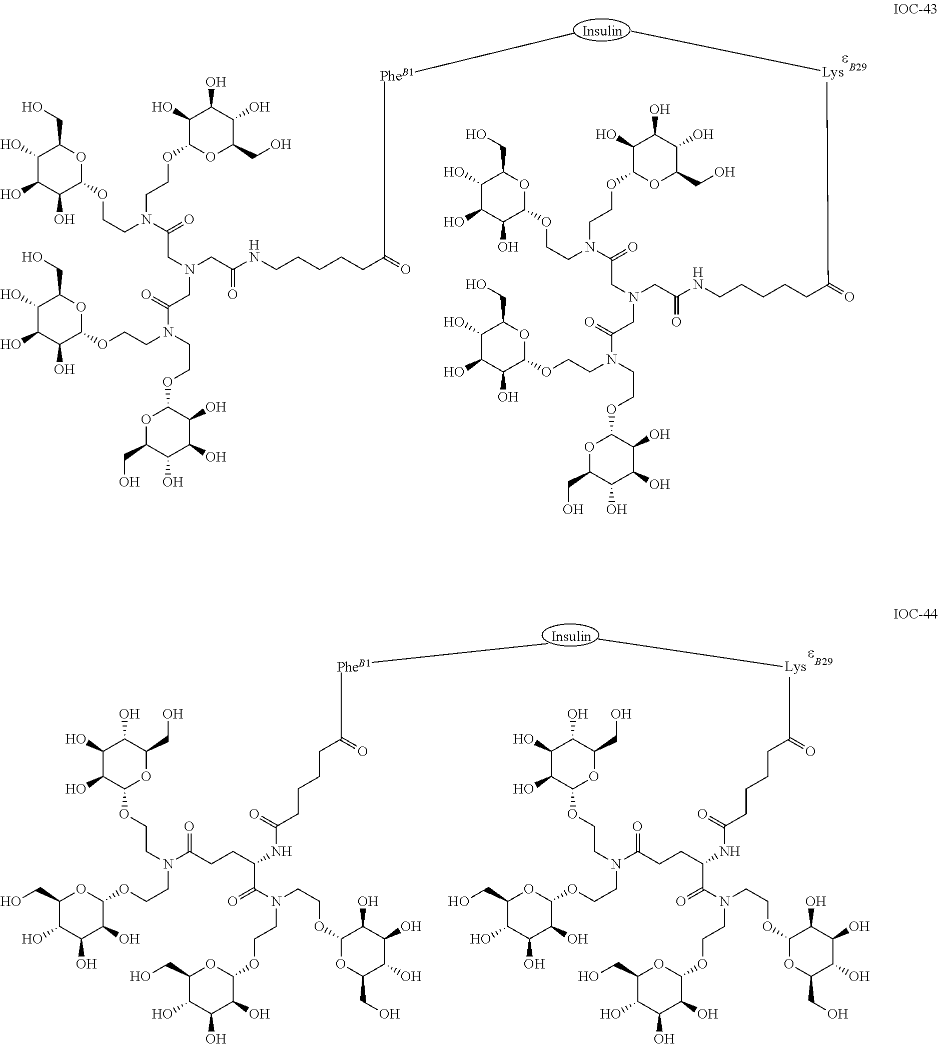

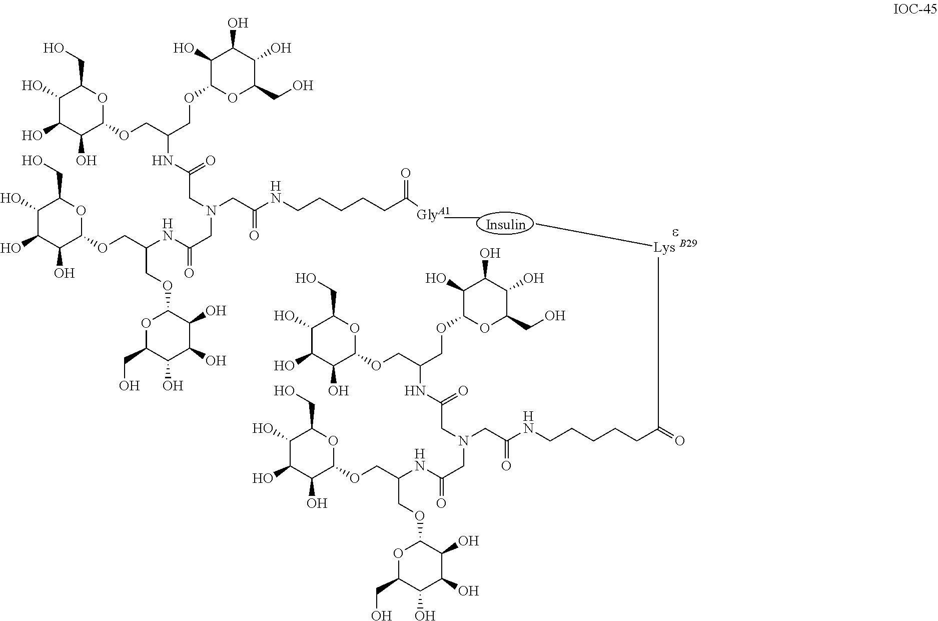

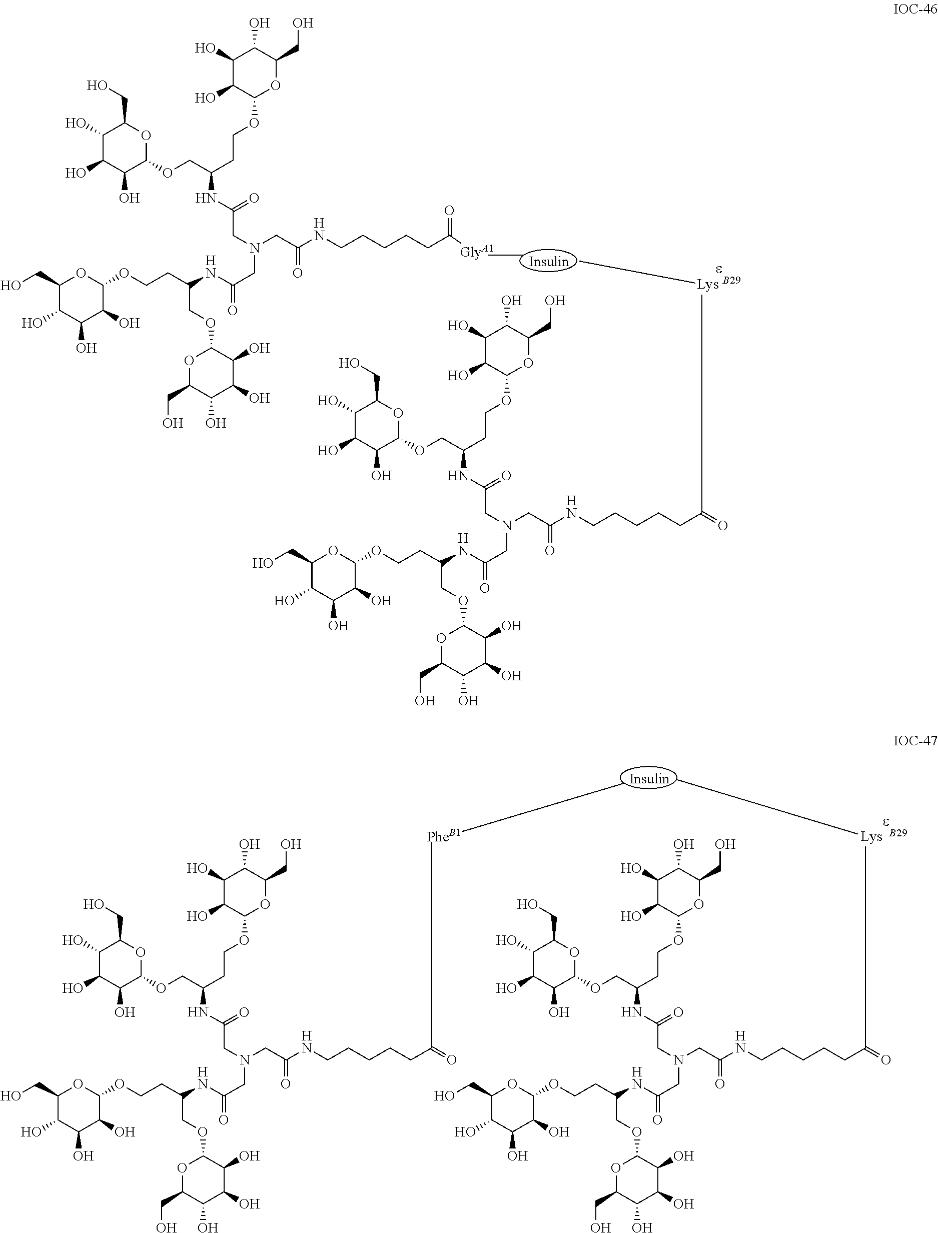

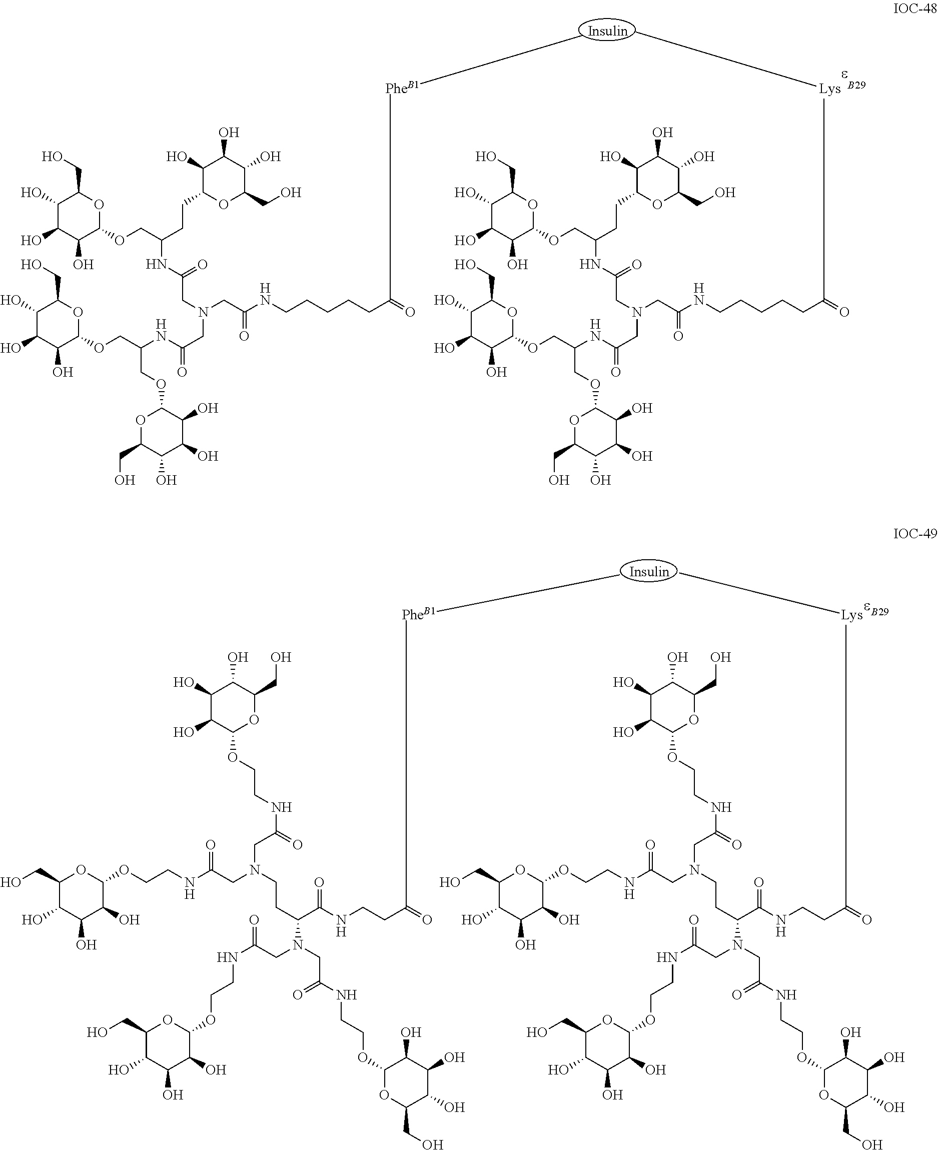

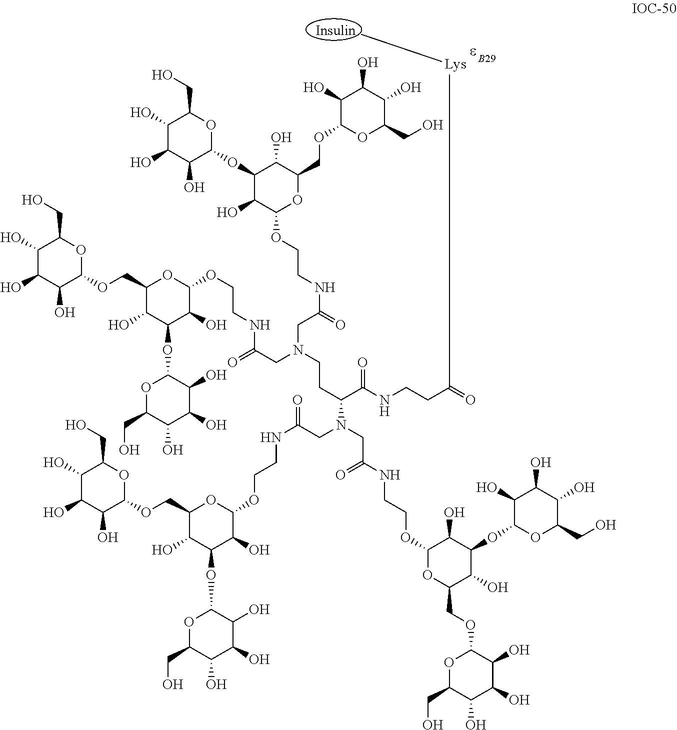

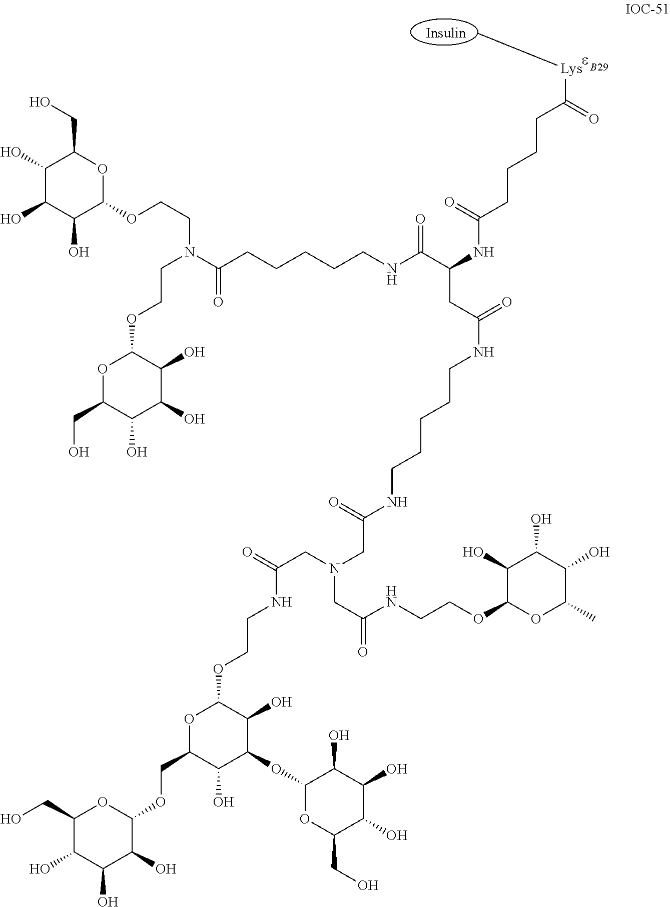

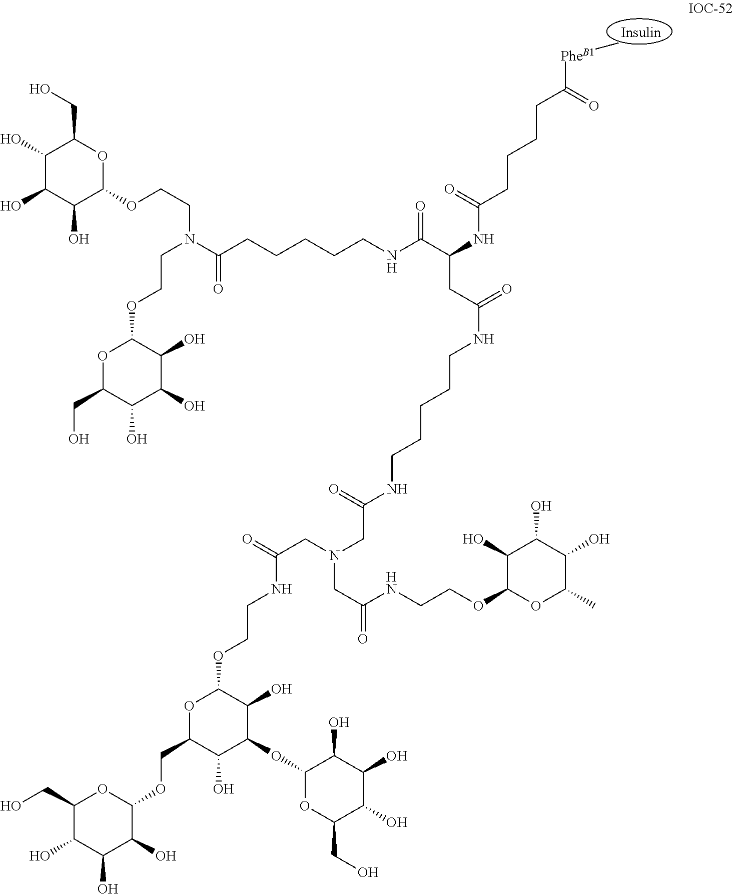

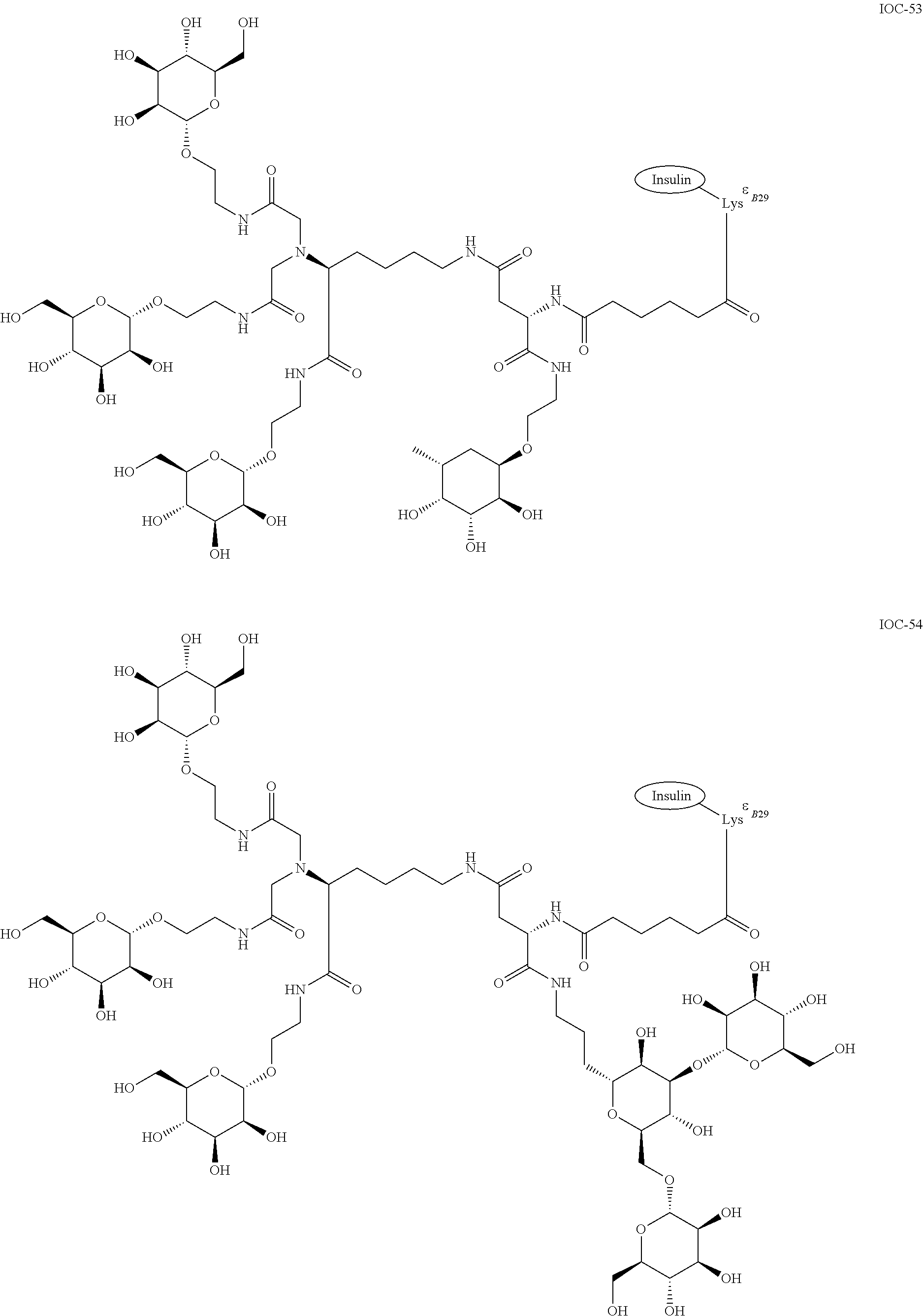

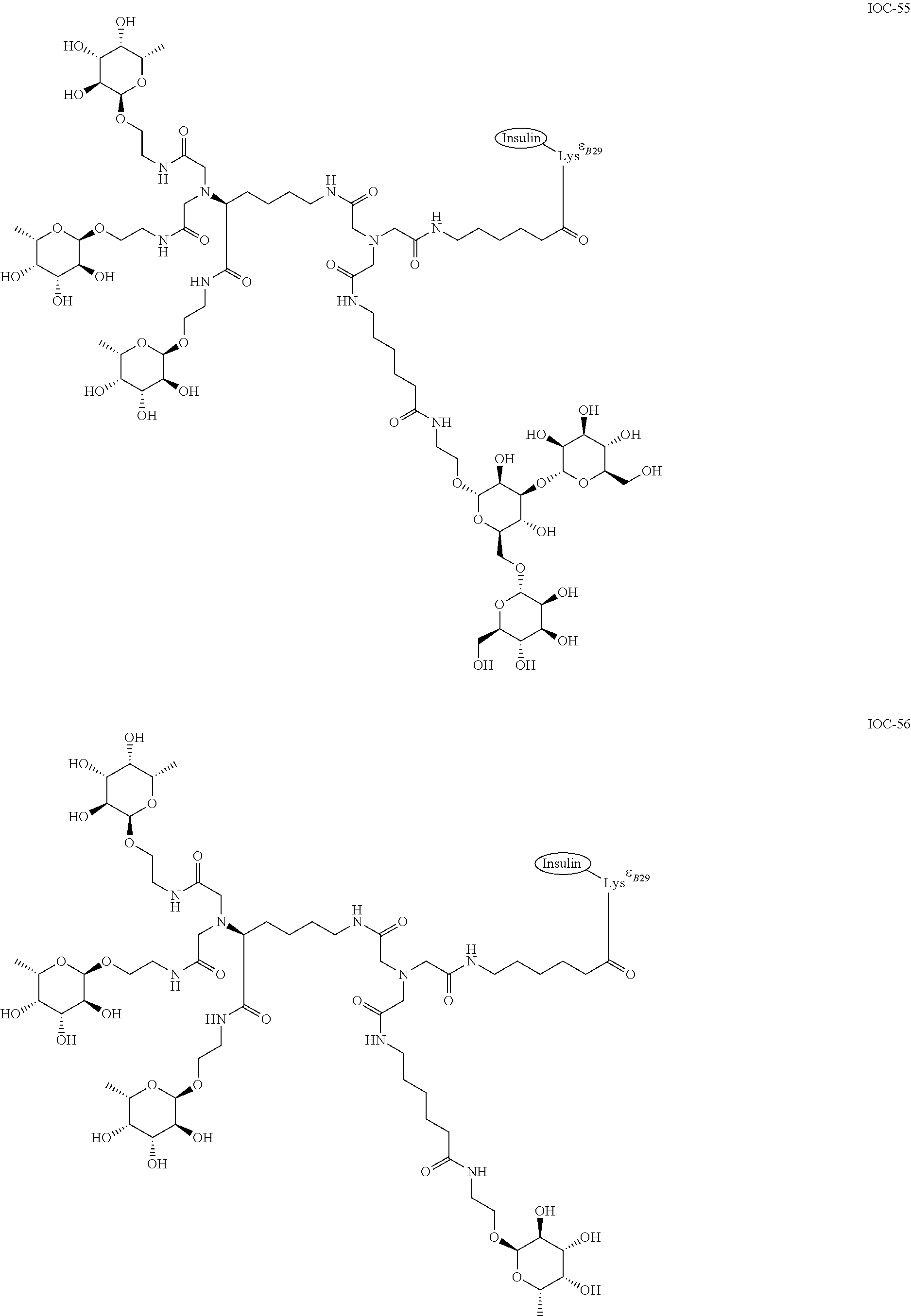

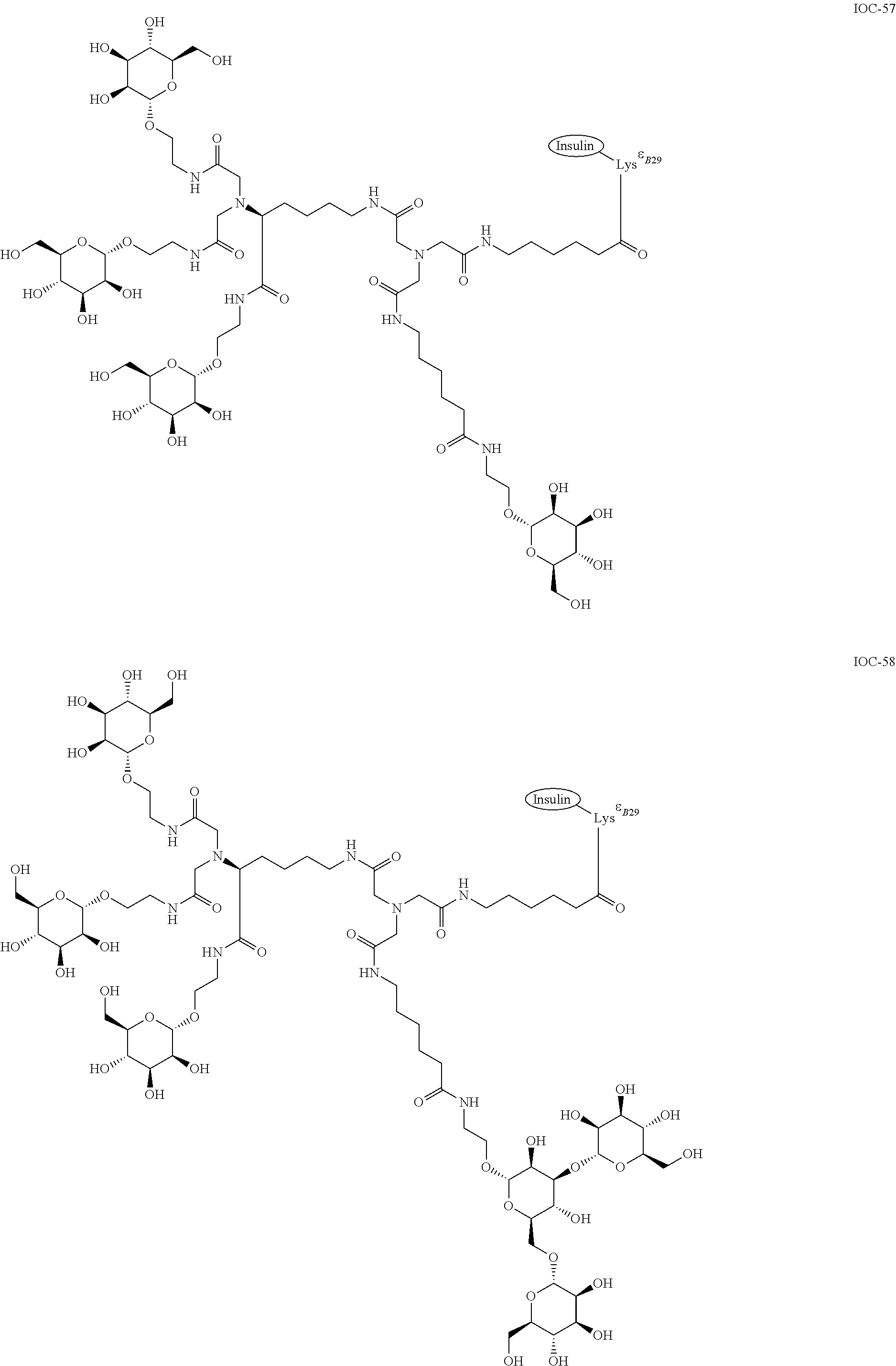

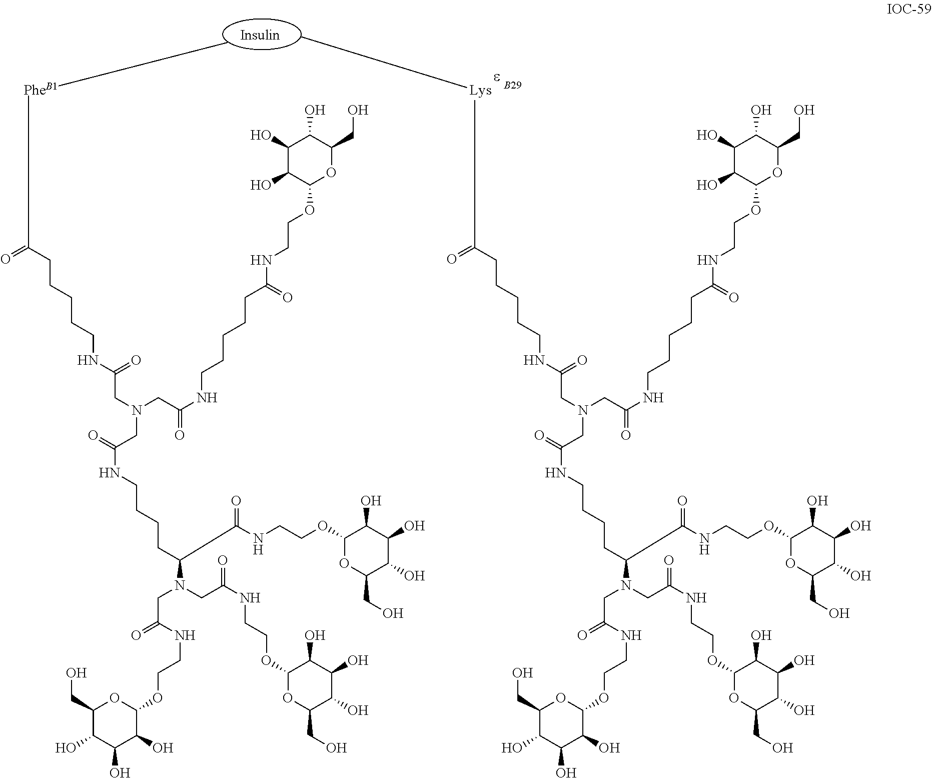

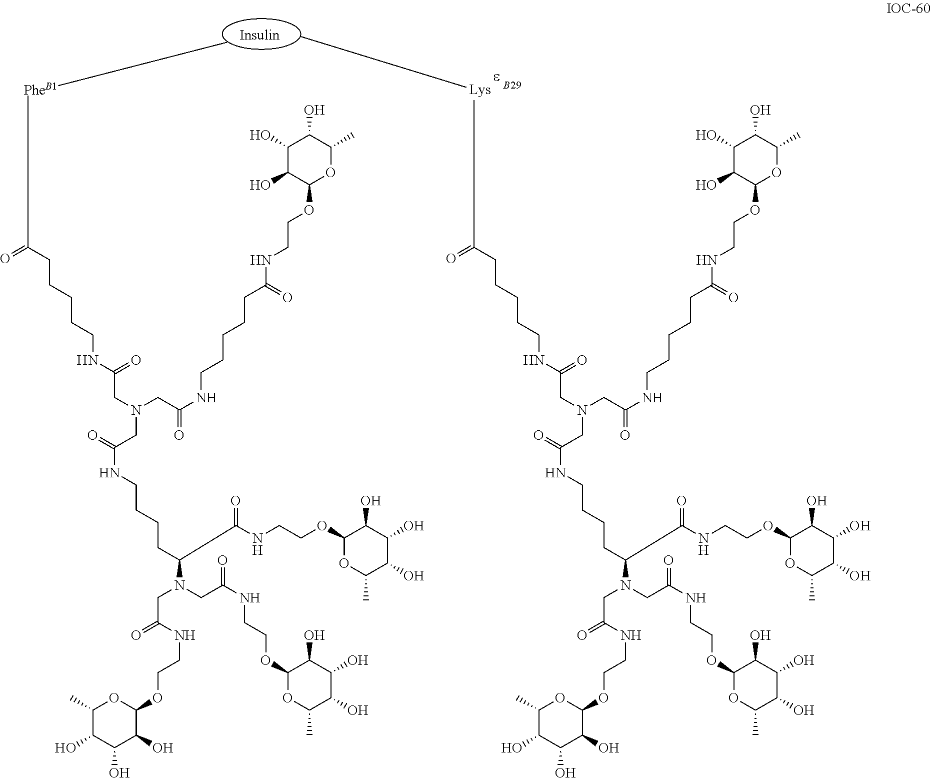

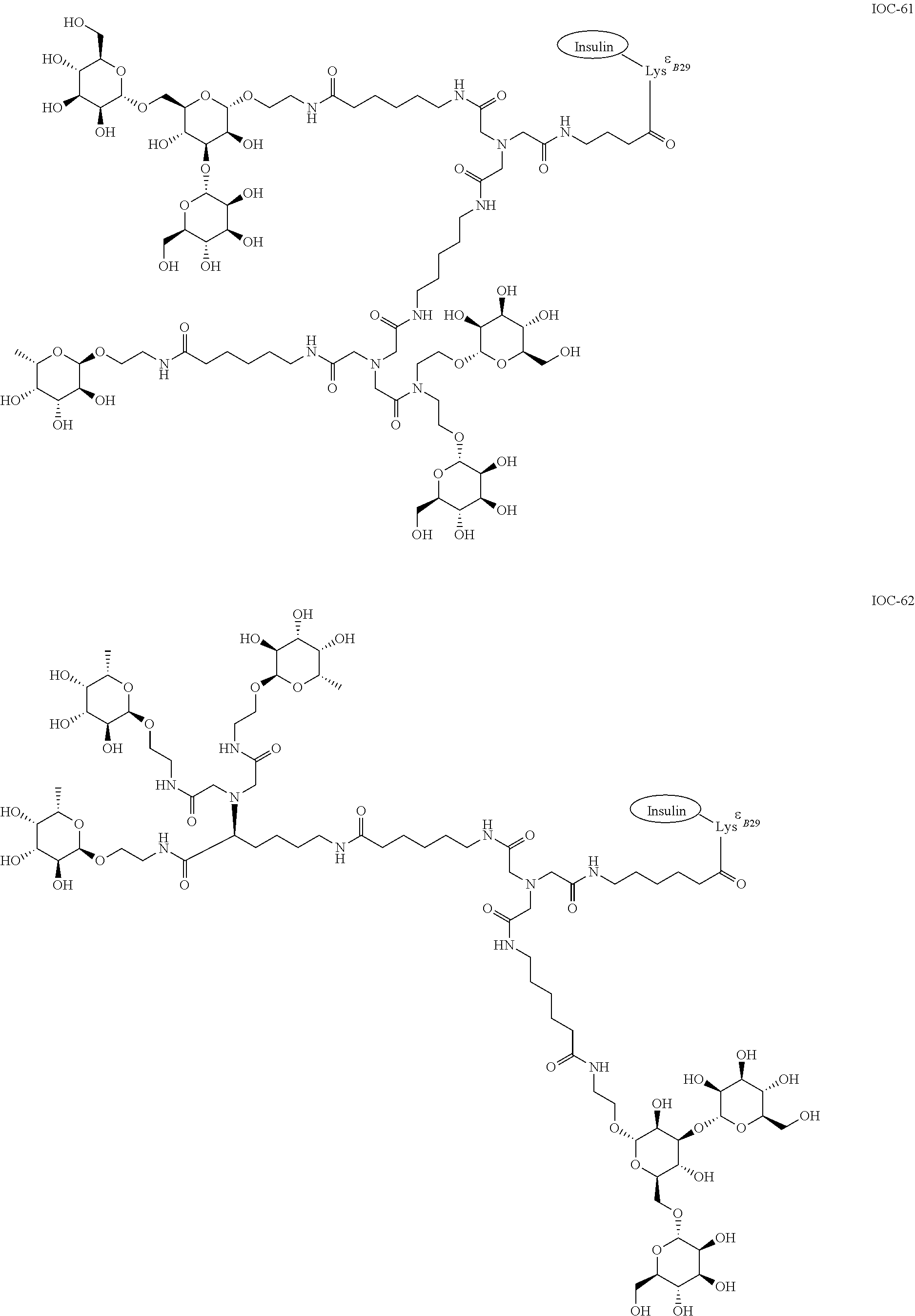

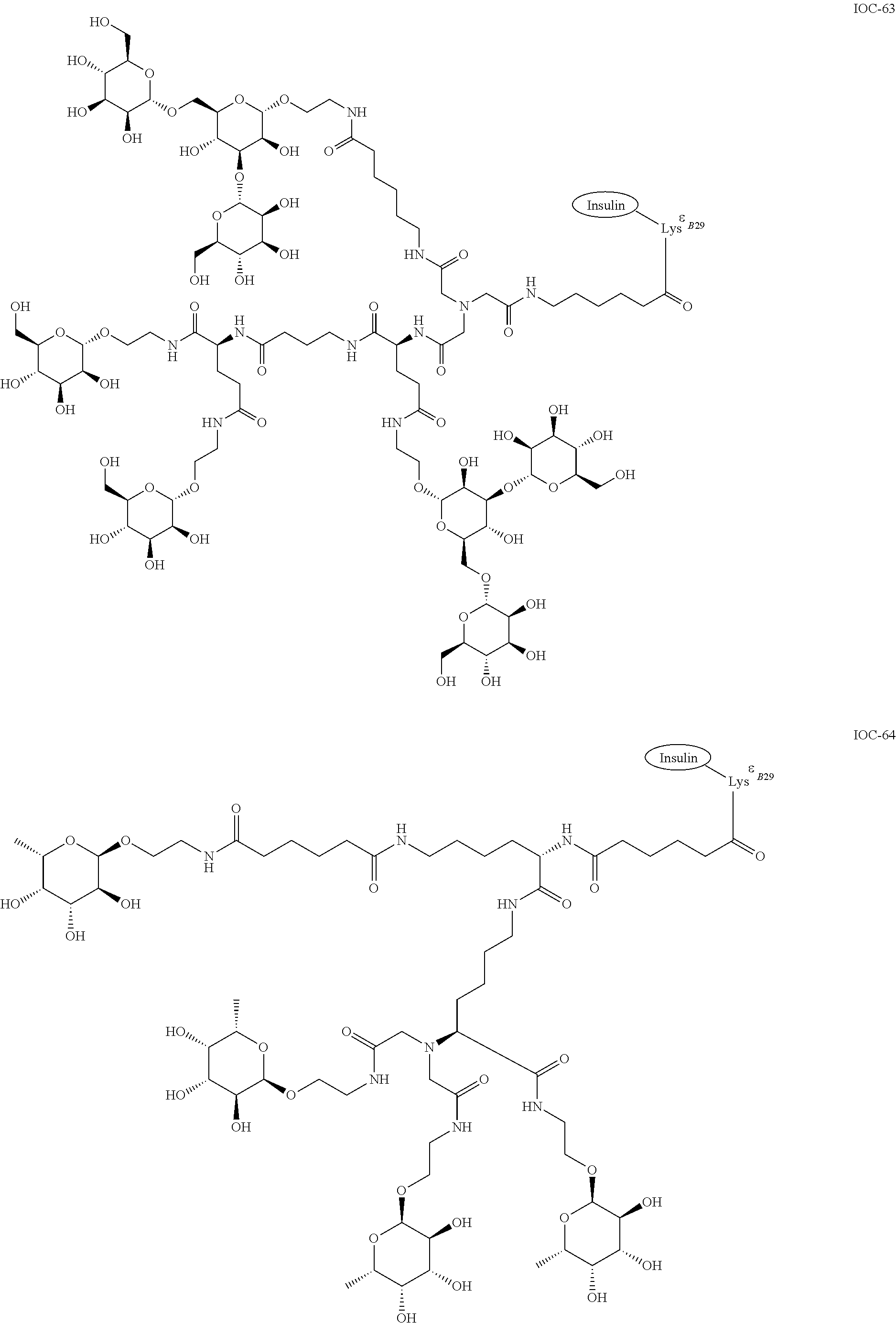

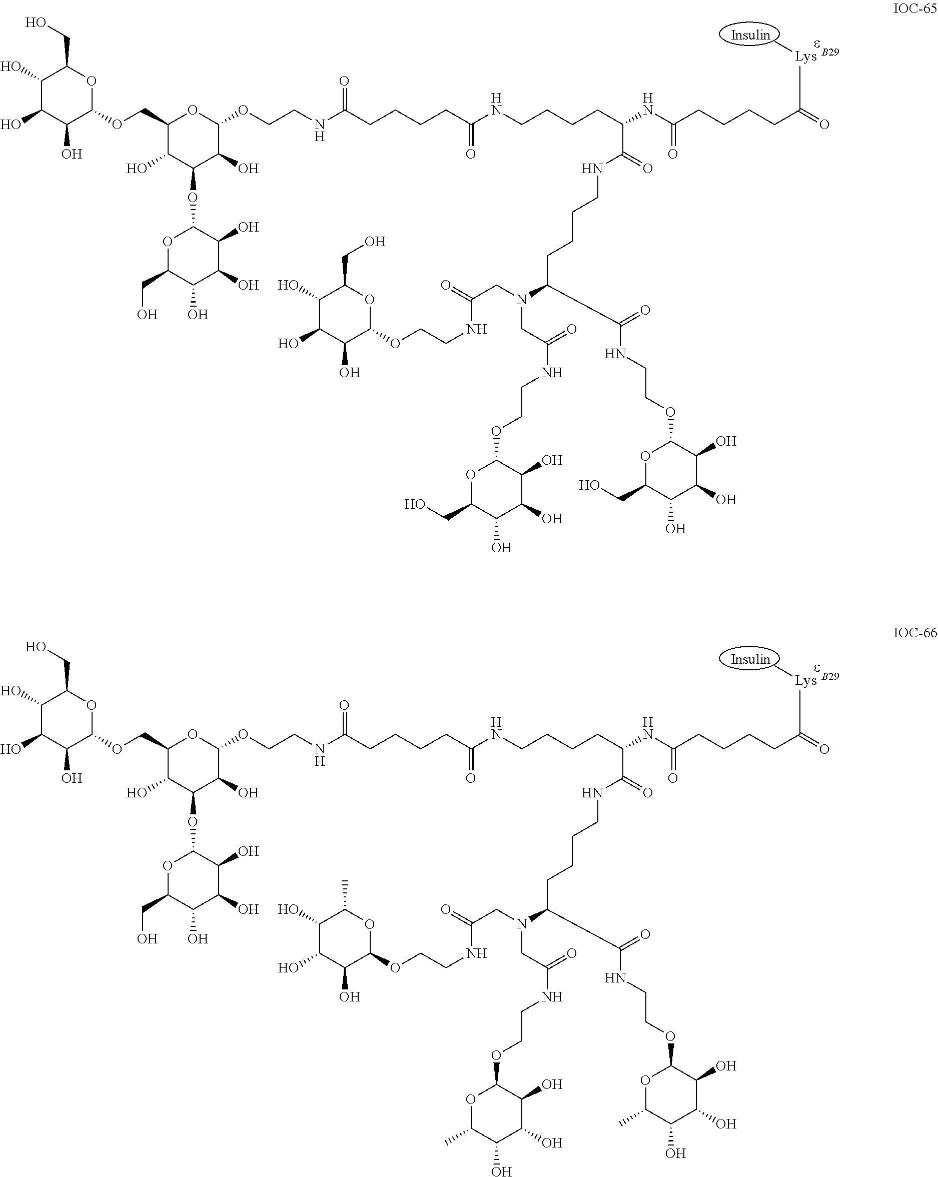

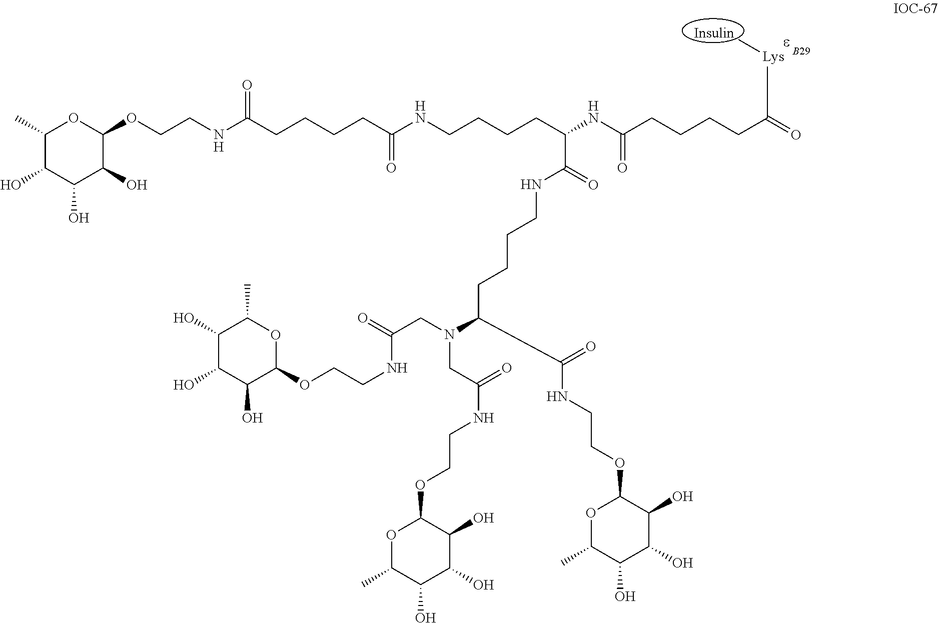

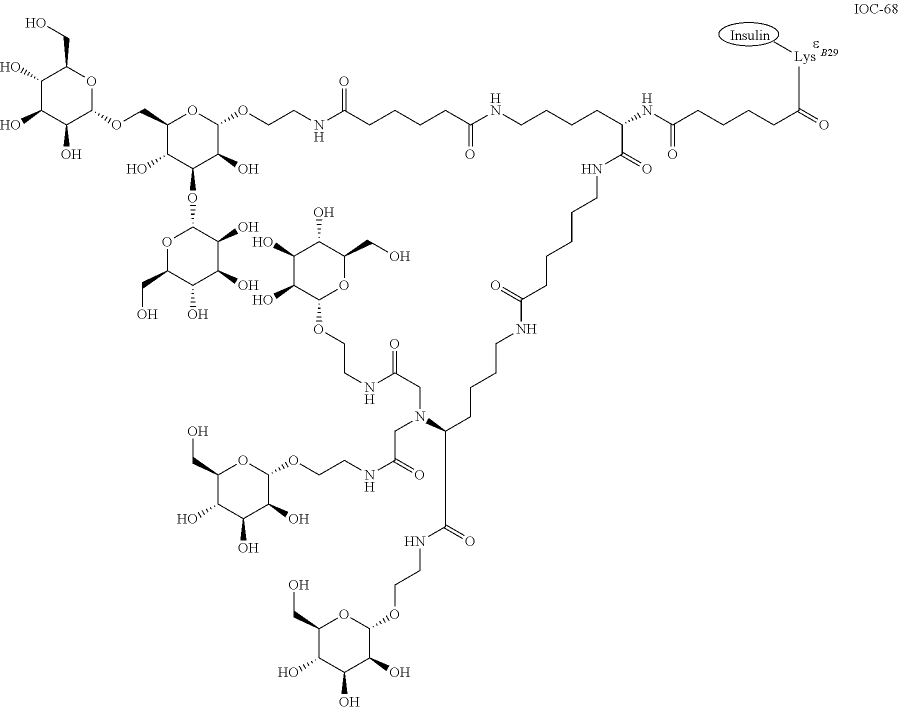

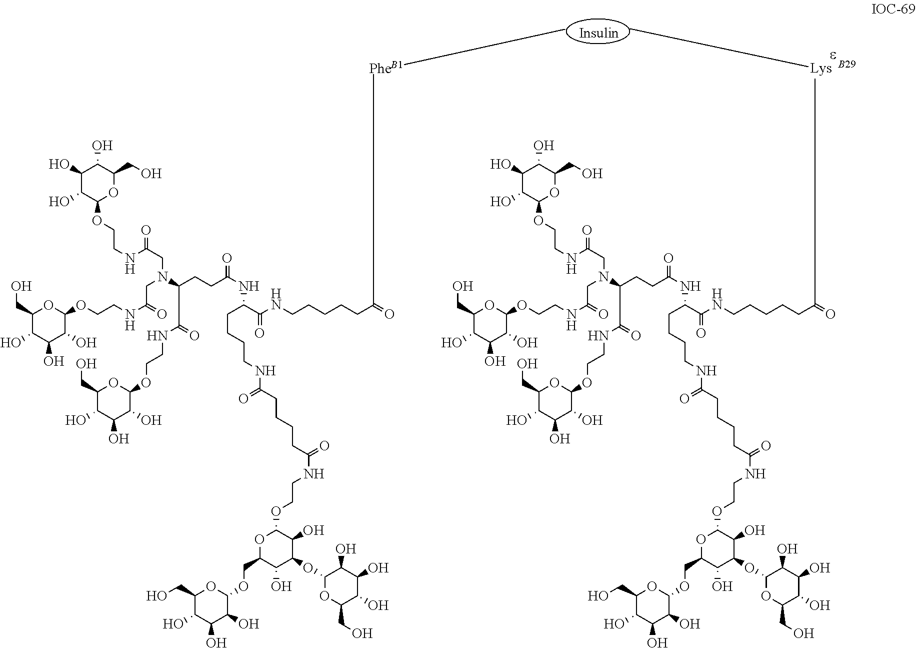

### Sustained Release Formulations

In particular embodiments, it may be advantageous to administer an insulin conjugate in a sustained fashion (i.e., in a form that exhibits an absorption profile that is more sustained than soluble recombinant human insulin). This will provide a sustained level of conjugate that can respond to fluctuations in glucose on a timescale that it more closely related to the typical glucose fluctuation timescale (i.e., hours rather than minutes). In particular embodiments, the sustained release formulation may exhibit a zero-order release of the conjugate when administered to a mammal under non-hyperglycemic conditions (i.e., fasted conditions).

It will be appreciated that any formulation that provides a sustained absorption profile may be used. In particular embodiments this may be achieved by combining the conjugate with other ingredients that slow its release properties into systemic circulation. For example, protamine zinc insulin (“PZI”) formulations may be used for this purpose. The present disclosure encompasses amorphous and crystalline forms of these PZI formulations.

Thus, in particular embodiments, a formulation of the present disclosure includes from about 0.05 to about 10 mg protamine/mg conjugate. For example, from about 0.2 to about 10 mg protamine/mg conjugate, e.g., about 1 to about 5 mg protamine/mg conjugate.

In particular embodiments, a formulation of the present disclosure includes from about 0.006 to about 0.5 mg zinc/mg conjugate. For example, from about 0.05 to about 0.5 mg zinc/mg conjugate, e.g., about 0.1 to about 0.25 mg zinc/mg conjugate.

In particular embodiments, a formulation of the present disclosure includes protamine and zinc in a ratio (w/w) in the range of about 100:1 to about 5:1, for example, from about 50:1 to about 5:1, e.g., about 40:1 to about 10:1. In particular embodiments, a PZI formulation of the present disclosure includes protamine and zinc in a ratio (w/w) in the range of about 20:1 to about 5:1, for example, about 20:1 to about 10:1, about 20:1 to about 15:1, about 15:1 to about 5:1, about 10:1 to about 5:1, about 10:1 to about 15:1.

One or more of the following components may be included in the PZI formulation: an antimicrobial preservative, an isotonic agent, and/or an unconjugated insulin molecule.

In particular embodiments, a formulation of the present disclosure includes an antimicrobial preservative (e.g., m-cresol, phenol, methylparaben, or propylparaben). In particular embodiments, the antimicrobial preservative is m-cresol. For example, in particular embodiments, a formulation may include from about 0.1 to about 1.0% v/v m-cresol. For example, from about 0.1 to about 0.5% v/v m-cresol, e.g., about 0.15 to about 0.35% v/v m-cresol.

In particular embodiments, a formulation of the present disclosure includes a polyol as isotonic agent (e.g., mannitol, propylene glycol, or glycerol). In particular embodiments, the isotonic agent is glycerol. In particular embodiments, the isotonic agent is a salt, e.g., NaCl. For example, a formulation may comprise from about 0.05 to about 0.5 M NaCl, e.g., from about 0.05 to about 0.25 M NaCl or from about 0.1 to about 0.2 M NaCl.

In particular embodiments, a formulation of the present disclosure includes an amount of unconjugated insulin molecule. In particular embodiments, a formulation includes a molar ratio of conjugated insulin molecule to unconjugated insulin molecule in the range of about 100:1 to 1:1, e.g., about 50:1 to 2:1 or about 25:1 to 2:1.

The present disclosure also encompasses the use of standard sustained (also called extended) release formulations that are well known in the art of small molecule formulation (e.g., see REMINGTON'S PHARMACEUTICAL SCIENCES, 19^{th} ed., Mack Publishing Co., Easton, PA, 1995). The present disclosure also encompasses the use of devices that rely on pumps or hindered diffusion to deliver a conjugate on a gradual basis. In particular embodiments, a long acting formulation may (additionally or alternatively) be provided by using a modified insulin molecule. For example, one could use insulin glargine (LANTUS™) or insulin detemir (LEVEMIR™) instead of wild-type human insulin in preparing the conjugate. Insulin glargine is an exemplary long acting insulin analog in which Asn at position A21 of the A-chain has been replaced by glycine and two arginine residues are at the C-terminus of the B-chain. The effect of these changes is to shift the isoelectric point, producing an insulin that is insoluble at physiological pH but is soluble at pH 4. Insulin detemir is another long acting insulin analog in which Thr at position B30 of the B-chain has been deleted and a C14 fatty acid chain has been attached to the Lys at position B29.

### Uses of Conjugates

In another aspect, the present disclosure provides methods of using the insulin conjugates. In general, the insulin conjugates can be used to controllably provide insulin to an individual in need in response to a saccharide (e.g., glucose or an exogenous saccharide such as mannose, alpha-methylmannose, L-fucose, etc.). The disclosure encompasses treating diabetes by administering an insulin conjugate of the present disclosure. Although the insulin conjugates can be used to treat any patient (e.g., dogs, cats, cows, horses, sheep, pigs, mice, etc.), they are most preferably used in the treatment of humans. An insulin conjugate may be administered to a patient by any route. In general, the present disclosure encompasses administration by oral, intravenous, intramuscular, intra-arterial, subcutaneous, intraventricular, transdermal, rectal, intravaginal, intraperitoneal, topical (as by powders, ointments, or drops), buccal, or as an oral or nasal spray or aerosol. General considerations in the formulation and manufacture of pharmaceutical compositions for these different routes may be found, for example, in REMINGTON'S PHARMACEUTICAL SCIENCES, 19^{th} ed., Mack Publishing Co., Easton, PA, 1995. In various embodiments, the conjugate may be administered subcutaneously, e.g., by injection. The insulin conjugate may be dissolved in a carrier for ease of delivery. For example, the carrier can be an aqueous solution including, but not limited to, sterile water, saline, or buffered saline.

In general, a therapeutically effective amount of the insulin conjugate will be administered. The term “therapeutically effective amount” means a sufficient amount of the insulin conjugate to treat diabetes at a reasonable benefit/risk ratio, which involves a balancing of the efficacy and toxicity of the insulin conjugate. In various embodiments, the average daily dose of insulin is in the range of 10 to 200 U, e.g., 25 to 100 U (where 1 Unit of insulin (“U”) is ˜0.04 mg). In particular embodiments, an amount of conjugate with these insulin doses is administered on a daily basis. In particular embodiments, an amount of conjugate with 5 to 10 times these insulin doses is administered on a weekly basis. In particular embodiments, an amount of conjugate with 10 to 20 times these insulin doses is administered on a bi-weekly basis. In particular embodiments, an amount of conjugate with 20 to 40 times these insulin doses is administered on a monthly basis.

In particular embodiments, a conjugate of the present disclosure may be used to treat hyperglycemia in a patient (e.g., a mammalian or human patient). In particular embodiments, the patient is diabetic. However, the present methods are not limited to treating diabetic patients. For example, in particular embodiments, a conjugate may be used to treat hyperglycemia in a patient with an infection associated with impaired glycemic control. In particular embodiments, a conjugate may be used to treat diabetes.

In particular embodiments, when an insulin conjugate or formulation of the present disclosure is administered to a patient (e.g., a mammalian patient), it induces less hypoglycemia than an unconjugated version of the insulin molecule. In particular embodiments, a formulation of the present disclosure induces a lower HbA1c value in a patient (e.g., a mammalian or human patient) than a formulation comprising an unconjugated version of the insulin molecule. In particular embodiments, the formulation leads to an HbA1c value that is at least 10% lower (e.g., at least 20% lower, at least 30% lower, at least 40% lower, or at least 50% lower) than a formulation comprising an unconjugated version of the insulin molecule. In particular embodiments, the formulation leads to an HbA1c value of less than 7%, e.g., in the range of about 4 to about 6%. In particular embodiments, a formulation comprising an unconjugated version of the insulin molecule leads to an HbA1c value in excess of 7%, e.g., about 8 to about 12%.

### Exogenous Trigger

As mentioned previously, the methods, conjugates and compositions that are described herein are not limited to glucose responsive conjugates. As demonstrated in the Examples, several exemplary insulin conjugates were also responsive to exogenous saccharides such as alpha-methylmannose. It will therefore be appreciated that, in particular embodiments, an insulin conjugate may be triggered by exogenous administration of a saccharide other than glucose, such as alpha-methylmannose or any other saccharide that can alter the PK or PD properties of the conjugate.

Once a conjugate has been administered as described above (e.g., as a sustained release formulation), it can be triggered by administration of a suitable exogenous saccharide. In a particular embodiment, a triggering amount of the exogenous saccharide is administered. As used herein, a “triggering amount” of exogenous saccharide is an amount sufficient to cause a change in at least one PK and/or PD property of the conjugate (e.g., Cₘₐₓ, AUC, half-life, etc. as discussed previously). It is to be understood that any of the aforementioned methods of administration for the conjugate apply equally to the exogenous saccharide. It is also to be understood that the methods of administration for the conjugate and exogenous saccharide may be the same or different. In various embodiments, the methods of administration are different (e.g., for purposes of illustration the conjugate may be administered by subcutaneous injection on a weekly basis while the exogenous saccharide is administered orally on a daily basis). The oral administration of an exogenous saccharide is of particular value because it facilitates patient compliance. In general, it will be appreciated that the PK and PD properties of the conjugate will be related to the PK profile of the exogenous saccharide. Thus, the conjugate PK and PD properties can be tailored by controlling the PK profile of the exogenous saccharide. As is well known in the art, the PK profile of the exogenous saccharide can be tailored based on the dose, route, frequency, and formulation used. For example, if a short and intense activation of the conjugate is desired then an oral immediate release formulation might be used. In contrast, if a longer less intense activation of conjugate is desired then an oral extended release formulation might be used instead. General considerations in the formulation and manufacture of immediate and extended release formulation may be found, for example, in REMINGTON'S PHARMACEUTICAL SCIENCES, 19^{th} ed., Mack Publishing Co., Easton, PA, 1995.

It will also be appreciated that the relative frequency of administration of a conjugate of the present disclosure and of an exogenous saccharide may be the same or different. In particular embodiments, the exogenous saccharide is administered more frequently than the conjugate. For example, in particular embodiment, the conjugate may be administered daily while the exogenous saccharide is administered more than once a day. In particular embodiment, the conjugate may be administered twice weekly, weekly, biweekly, or monthly, while the exogenous saccharide is administered daily. In particular embodiments, the conjugate is administered monthly, and the exogenous saccharide is administered twice weekly, weekly, or biweekly. Other variations on these schemes will be recognized by those skilled in the art and will vary depending on the nature of the conjugate and formulation used.

The following examples are intended to promote a further understanding of the present disclosure.

### Examples

### General Procedures

All chemicals were purchased from commercial sources, unless otherwise noted. Reactions sensitive to moisture or air were performed under nitrogen or argon using anhydrous solvents and reagents. The progress of reactions was monitored by analytical thin layer chromatography (“TLC”), high performance liquid chromatography-mass spectrometry (“HPLC-MS”), or ultra-performance liquid chromatography-mass spectrometry (“UPLC-MS”). TLC was performed on E. Merck TLC plates precoated with silica gel 60F-254, layer thickness 0.25 mm. The plates were visualized using 254 nm ultraviolet radiation (“UV”) and/or by exposure to cerium ammonium molybdate (“CAM”) or β-anisaldehyde staining solutions followed by charring. High performance liquid chromatography (“HPLC”) was conducted on an Agilent 1100 series HPLC using SUPELCO™ Ascentis Express C18 2.7 μm 3.0×100 mm column with gradient 10:90-99:1 v/v CH₃CN/H₂O+v 0.05% TFA over 4.0 min then hold at 98:2 v/v CH₃CN/H₂O+v 0.05% TFA for 0.75 min; flow rate 1.0 mL/min, UV range 200-400 nm (LC-MS Method A). Mass analysis was performed on a Waters MICROMASS™ ZQ™ with electrospray ionization in positive ion detection mode and the scan range of the mass-to-charge ratio was either 170-900 or 500-1500. Ultra-performance liquid chromatography (UPLC) was performed on a Waters ACQUITY™ UPLC™ system using the following methods:

UPLC-MS Method A: Waters ACQUITY™ UPLC™ BEH C18 1.7 μm 2.1×100 mm column with gradient 10:90-70:30 v/v CH₃CN/H₂O+v 0.1% TFA over 4.0 min and 70:30-95:5 v/v CH₃CN/H₂O+v 0.1% TFA over 40 sec; flow rate 0.3 mL/min, UV wavelength 200-300 nm.

UPLC-MS Method B: Waters ACQUITY™ UPLC™ BEH C18 1.7 μm 2.1×100 mm column with gradient 60:40-100:0 v/v CH₃CN/H₂O+v 0.1% TFA over 4.0 min and 100:0-95:5 v/v CH₃CN/H₂O+v 0.1% TFA over 40 sec; flow rate 0.3 mL/min, UV wavelength 200-300 nm.

UPLC-MS Method C: Waters ACQUITY™ UPLC™ HSS T3 1.7 μm 2.1×100 mm column with gradient 0:100-40:60 v/v CH₃CN/H₂O+v 0.05% TFA over 8.0 min and 40:60-10:90 v/v CH₃CN/H₂O+v 0.05% TFA over 2.0 min; flow rate 0.3 mL/min, UV wavelength 200-300 nm.

UPLC-MS Method D: Waters ACQUITY™ UPLC™ BEH C18 1.7 μm 2.1×100 mm column with gradient 0:100-60:40 v/v CH₃CN/H₂O+v 0.1% TFA over 8.0 min and 60:40-90:10 v/v CH₃CN/H₂O+v 0.1% TFA over 3.0 min and hold at 100:0 v/v CH₃CN/H₂O+v 0.1% TFA for 2 min; flow rate 0.3 mL/min, UV wavelength 200-300 nm.

UPLC-MS Method E: Waters ACQUITY™ UPLC™ BEH C8 1.7 μm 2.1×100 mm column with gradient 10:90-55:45 v/v CH₃CN/H₂O+v 0.1% TFA over 4.2 min and 100: 0-95:5 v/v CH₃CN/H₂O+v 0.1% TFA over 0.4 min; flow rate 0.3 mL/min, UV wavelength 200-300 nm.

UPLC-MS Method F: Waters ACQUITY™ UPLC™ BEH C8 1.7 μm 2.1×100 mm column with gradient 10:90-90:10 v/v CH₃CN/H₂O+v 0.1% TFA over 4.2 min and 90:10-95:5 v/v CH₃CN/H₂O+v 0.1% TFA over 0.4 min; flow rate 0.3 mL/min, UV wavelength 200-300 nm.

UPLC-MS Method G: Waters ACQUITY™ UPLC™ BEH300 C4 1.7 μm 2.1×100 mm column with gradient 10:90-90:10 v/v CH₃CN/H₂O+v 0.1% TFA over 4.0 min and 90:10-95:5 v/v CH₃CN/H₂O+v 0.1% TFA over 0.5 min; flow rate 0.3 mL/min, UV wavelength 200-300 nm.

Mass analysis was performed on a Waters MICROMASS™ LCT PREMIER™ XE with electrospray ionization in positive ion detection mode, and the scan range of the mass-to-charge ratio was 300-2000. The identification of the produced insulin conjugates was confirmed by comparing the theoretical molecular weight to the experimental value that was measured using UPLC-MS. For the determination of the position of sugar modification(s), specifically, insulin conjugates were subjected to DTT treatment (for a/b chain) or Glu-C digestion (with reduction and alkylation), and then the resulting peptides were analyzed by LC-MS. Based on the measured masses, the sugar positions were deduced.

Flash chromatography was performed using either a Biotage Flash Chromatography apparatus (Dyax Corp.) or a CoMBIFLASH™ Rf instrument (Teledyne Isco). Normal-phase chromatography was carried out on silica gel (20-70 μm, 60 Å pore size) in pre-packed cartridges of the size noted. Reverse-phase chromatography was carried out on C18-bonded silica gel (20-60 μm, 60-100 Å pore size) in pre-packed cartridges of the size noted. Preparative scale HPLC was performed on Gilson 333-334 binary system using Waters DELTA-PAK™ C4 15 μm, 300 Å, 50×250 mm column or KROMASIL™ C8 10 μm, 100 Å, 50×250 mm column, flow rate 85 mL/min, with gradient noted. Concentration of solutions was carried out on a rotary evaporator under reduced pressure or freeze-dried on a VirTis Freezemobile Freeze Dryer (SP Scientific).

¹H-NMR spectra were acquired at 500 MHz (or otherwise specified) spectrometers in deuterated solvents noted. Chemical shifts were reported in parts per million (“ppm”). Tetramethylsilane (“TMS”) or residual proton peak of deuterated solvents was used as an internal reference. Coupling constant (“J”) were reported in hertz (“Hz”).

### Abbreviations

Å Angstrom
ACN, MeCN Acetonitrile, CH₃CN
AcOH Acetic acid
aq Aqueous
BEH Ethylene bridged hybrid technology
BOC, Boc tert-butoxycarbonyl protecting group
Brine Saturated aqueous sodium chloride solution
BSA Bovine serum albumin
C18 Carbon 18
CAM Cerium ammonium molybdate
Cbz Carboxybenzyl
CHO Chinese hamster ovary
Cₘₐₓ The highest concentration of a drug in the blood
conc. Concentrated
CuI Copper iodide
CV Column volume
Da Daltons
DCC Dicyclohexylcarbodiimide
DCM Dichloromethane
DDT Dichlorodiphenyltrichloroethane
DELFH Eu-N1-ITC DELFIA™ Europium N1-isocyanate
DIPEA N,N-diisopropylethylamine or Hünig's Base
dL Deciliter
DMA N,N-dimethylacetamide
DMAP (4-dimethylamino)pyridine
DMF N,N-dimethylformamide
DMSO Dimethylsulfoxide
DTT Dithiothreitol
EDC N-(3-dimethylaminopropyl)-N′-ethylcarbodiimide hydrochloride
Et₂O, ether Diethyl ether
EtOAc Ethyl acetate
FBS Fasting blood sugar
FR Flow rate
g Grams
G418 GENETICIN™, aminoglycoside antibiotic
H, hr Hours
HATU O-(7-azabenzotriazol-1-yl)-N,N,N′,N′-tetramethyluronium hexafluorophosphate)
HbA1c Hemoglobin A1c
Hex Hexanes
HOBt 1-hydroxybenzotriazole hydrate
HPLC High performance liquid chromatography
HPLC-MS High performance liquid chromatography-mass spectroscopy
Hz Hertz
IGF Insulin-like growth factor
IPA Isopropyl alcohol
IR Insulin receptor
IR(B) Insulin receptor (type B)
J Coupling constant
kg Kilogram
LC-MS Liquid chromatography -mass spectroscopy
M Molar, moles per liter
m/e Mass/electron
m/z Mass-to-charge ratio
Me Methyl, CH₃—
mg Milligram
MHz Megahertz
min Minute
mL, ml Milliliter
mm Millimeter
mmol Millimole
MRC1 Mannose receptor C type 1
MS, ms Mass spectrum
MSD Meso scale discovery
MW Molecular weight
N Normality, number of mole equivalents per liter of solution
nm Nanometer
nM Nanomolar
NMM N-methylmorpholine
nmol Nanomole
OBD Optimum bed density
OtBu Tert-butyl ester
PBS Phosphate buffered saline
Pd/C Palladium on carbon
PE Petroleum ether
PFTU Pentafluorphenol-tetramethyluronium hexafluorophosphate
pIR Phosphorus insulin receptor
ppm Parts per million
psi Pounds per square inch
PZI Protamine zinc insulin
RPM, rpm Revolutions per minute
RT, rt Room temperature (˜25° C.)
sat., sat'd Saturated
sec Second(s)
SiO₂ Silicon dioxide
TEA Triethylamine
TFA Trifluoroacetic acid
TFAA Trifluoroacetic anhydride
THF Tetrahydrofuran
TLC Analytical thin layer chromatography
Tₘₐₓ The amount of time that a drug is present at the maximum concentration in serum
TMS Tetramethylsilane
t_{R} Retention time
TSTU N,N,N′,N′-tetramethyl-O—(N-succinimidyl)uronium tetrafluoroborate
UPLC Ultra-performance liquid chromatography
UPLC-MS Ultra-performance liquid chromatography -mass spectroscopy
UV Ultraviolet
V, v volume
v/v Volume per volume
w/w Weight ratio
WGA PVT PEI SPA Wheat germ agglutinin polyvinyltoluene polyethyleneimine scintillation proximity assay
Wt, wt, w Weight
z charge
μL, μl, uL, ul, Microliter
μm, um Micrometer
μmol, umol Micromole

### Example 1: 2,5-Dioxopyrrolidin-1-yl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oate (ML-1)

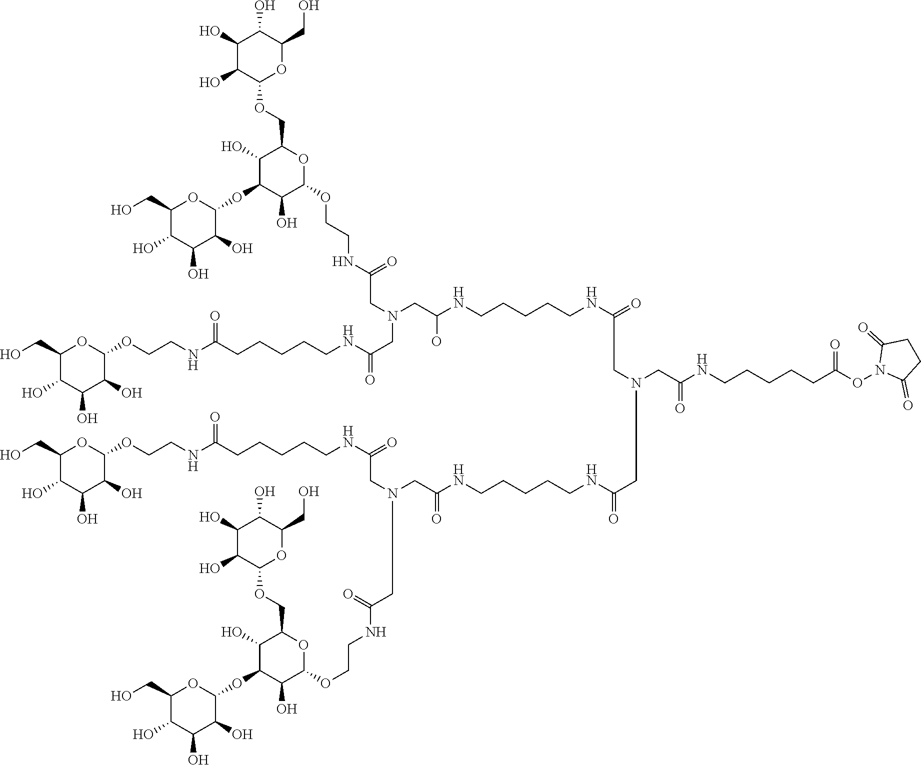 [SMILES: O=C(CCCCCNC(=O)CN(CC(=O)NCCCCCNC(=O)CN(CC(=O)NCCCCCNC(O)CN(CC(=O)NCCCCCC(=O)NCCO[C@H]1O[C@H](CO)[C@@H](O)[C@H](O)[C@@H]1O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CC(=O)NCCCCCC(=O)ON1C(=O)CCC1=O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCO[C@H]1O[C@H](CO)[C@@H](O)[C@H](O)[C@@H]1O]

### Step 1: 13-(Carboxymethyl)-3,11-dioxo-1-phenyl-2-oxa-4,10,13-triazapentadecan-15-oic acid

To a solution of benzyl (5-aminopentyl)carbamate hydrochloride (5.0 g, 18.33 mmol) in DMF (22 mL) at 0° C. was added K₂CO₃ (2.53 g, 18.33 mmol). After stirring at 0° C. for 2 h, the resulting suspension was filtered through a cake of CELITE™ diatomaceous earth, and the filtrate was added to a solution of 2-(2,6-dioxomorpholino)acetic acid (3.17 g, 18.33 mmol) in DMF (22 mL) at 0° C. After stirring at 0° C. for 30 min, the resulting mixture was warmed to rt and stirred overnight. The mixture was concentrated, and the residue was suspended in water (20 mL) and stirred at rt for 30 min. The product was collected by filtration, washed with a small amount of ACN to give the title compound. UPLC-MS Method A: m/z=410.1 (z=1); t_{R}=3.95 min.

### Step 2: Benzyl [6-({2-[(α-D-mannopyranosyl)oxy]ethyl}amino)-6-oxohexyl]carbamate

To a solution of 2-aminoethyl α-D-mannopyranoside (1.0 g, 4.48 mmol) in DMF (20 mL) at 0° C. was added a solution 2,5-dioxopyrrolidin-1-yl 6-{[(benzyloxy)carbonyl]amino} hexanoate (2.2 g, 5.38 mmol) in DMF (10 mL) over 30 min followed by slow addition of TEA (937 μL, 6.72 mmol). After stirring at rt for 16 h, the reaction mixture was concentrated and purified by C18 reverse phase chromatography to isolate the title compound. UPLC-MS Method A: m/z=471.1 (z=1); t_{R}=3.63 min.

### Step 3: 6-Amino-N-{2-[(α-D-mannopyranosyl)oxy]ethyl}hexanamide

Benzyl [6-({2-[(α-D-mannopyranosyl)oxy]ethyl}amino)-6-oxohexyl]carbamate was dissolved in water (20 mL) and placed under N₂. Pd/C (20.0 mg, 0.184 mmol) was added, and the resulting mixture was degassed three times with H₂. After stirring at rt for 1 h, the resulting mixture was filtered through a cake of CELITE™ diatomaceous earth and washed with water. The filtrate was freeze-dried to give the title compound. UPLC-MS Method A: m/z=337.1 (z=1); t_{R}=0.86 min.

### Step 4: 13-(2-{[6-({2-[(α-D-Mannopyranosyl)oxy]ethyl}amino)-6-oxohexyl]amino}-2-oxoethyl)-3,11-dioxo-1-phenyl-2-oxa-4,10,13-triazapentadecan-15-oic acid

To a suspension of 13-(carboxymethyl)-3,11-dioxo-1-phenyl-2-oxa-4,10,13-triazapentadecan-15-oic acid in DCM (20 mL) at 0° C. was added TFAA (647 μL, 4.58 mmol). After stirring at 0° C. for 3 h, the mixture was cooled to −30° C. To the resulting mixture was added dropwise a solution of TEA (1.226 mL, 8.79 mmol) in DMF (20 mL) over 30 min. After stirring at −30° C. for an additional 30 min, to the resulting mixture was added a solution of 6-amino-N-{2-[(α-D-mannopyranosyl)oxy]ethyl}hexanamide in DMF (20 mL). After stirring at rt for 16 h, the mixture was concentrated, and the residue was purified by C18 reverse phase chromatography to give the title compound. UPLC-MS Method A: m/z=728.5 (z=1); t_{R}=4.84 min.

### Step 5: 2,5-Dioxopyrrolidin-1-yl 13-(2-{[6-({2-[(α-D-mannopyranosyl)oxy]ethyl}amino)-6-oxohexyl]amino}-2-oxoethyl)-3,11-dioxo-1-phenyl-2-oxa-4,10,13-triazapentadecan-15-oate

To a solution of 13-(2-{[6-({2-[(α-D-mannopyranosyl)oxy]ethyl}amino)-6-oxohexyl]amino}-2-oxoethyl)-3,11-dioxo-1-phenyl-2-oxa-4,10,13-triazapentadecan-15-oic acid in DMF (6 mL) at rt was added DIPEA (58 μL, 0.330 mmol) followed by addition of TSTU (136 mg, 0.330 mmol). After stirring for 5 h, the reaction was quenched with TFA (47 μL, 0.605 mmol) and stirred for an additional 15 min. The mixture was concentrated, and the residue was taken into water and lyophilized to give the title compound. UPLC-MS Method A: m/z=825.4 (z=1); t_{R}=2.99 min.

### Step 6: Benzyl {1-[(α-D-mannopyranosyl)oxy]-4,11,15-trioxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16-tetraazahenicosan-21-yl}carbamate

To a solution of 2,5-dioxopyrrolidin-1-yl 13-(2-{[6-({2-[(α-D-mannopyranosyl)oxy]ethyl}amino)-6-oxohexyl]amino}-2-oxoethyl)-3,11-dioxo-1-phenyl-2-oxa-4,10,13-triazapentadecan-15-oate (227 mg, 0.275 mmol) in DMF at rt was added 2-({α-D-mannopyranosyl-(1→3)-[α-D-mannopyranosyl-(1→6)]-α-D-mannopyranosyl}oxy)ethan-1-amine (226 mg, 0.413 mmol, WO2015/051052A2). After stirring for 16 h, the reaction mixture was concentrated, and the residue was purified by C18 reverse phase chromatography to give the title compound. UPLC-MS Method A: m/z=1256.6 (z=1); t_{R}=4.49 min.

### Step 7: 6-{2-[{2-[(5-Aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino}ethyl)amino]acetamido}-N-{2-[(α-D-mannopyranosyl)oxy]ethyl}hexanamide

To a solution of benzyl {1-[(α-D-mannopyranosyl)oxy]-4,11,15-trioxo-13-(2-oxo-2-{[2-({α-D-mannopyranosyl-(1→3)-[α-D-mannopyranosyl-(1→6)]-α-D-mannopyranosyl}oxy) ethyl]amino}ethyl)-3,10,13,16-tetraazahenicosan-21-yl}carbamate (238.1 mg, 0.189 mmol) in water (10 mL) was added Pd/C (0.6 mg, 5.68 μmol). The resulting mixture was degassed three times with H₂, and then stirred under a balloon of H₂ for 1 h. The catalyst was filtered off through a cake of CELITE™ diatomaceous earth and washed with water. The filtrate was freeze-dried to give the title compound. UPLC-MS Method A: m/z=1123.6 (z=1); t_{R}=4.03 min.

### Step 8: Benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oate

To a solution of 2,2′-[(2-{[6-(benzyloxy)-6-oxohexyl]amino}-2-oxoethyl)azanediyl]diacetic acid (20 mg, 0.051 mmol) in DMF (2 mL) at rt was added EDC (21.4 mg, 0.112 mmol), HOBt (0.8 mg, 5.07 μmol) and TEA (25 μL, 0.177 mmol). After stirring for 30 min, to the resulting mixture was added a solution of 6-{2-[{2-[(5-aminopentyl)amino]-2-oxoethyl}(2-oxo-2-{[2-({α-D-mannopyranosyl-(1-3)-[α-D-mannopyranosyl-(1→6)]-α-D-mannopyranosyl}oxy) ethyl]amino}ethyl)amino]acetamido}-N-{2-[(α-D-mannopyranosyl)oxy]ethyl}hexanamide (125 mg, 0.112 mmol) in DMF (2 mL) dropwise. After stirring for 16 h, the mixture was concentrated, and the residue was purified by C18 reverse phase chromatography to provide the title compound. UPLC-MS Method A: m/z=1302.3 (z=2); t_{R}=2.38 min.

### Step 9: 1-[(α-D-Mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oic acid

A mixture of benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oate (60 mg, 0.023 mmol) and Pd/C (2.5 mg, 0.023 mmol) was degassed with H₂ and stirred under a balloon of H₂ for 1 h. The catalyst was filtered off through a cake of CELITE™ diatomaceous earth and washed with water. The filtrate was freeze-dried to give the title compound. UPLC-MS Method A: m/z=1258.2 (z=2); t_{R}=1.64 min.

### Step 10: 2,5-Dioxopyrrolidin-1-yl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oate

To a solution of 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15, 23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oic acid (50.8 mg, 0.020 mmol) in DMF (2 mL) was added DIPEA (16 μL, 0.093 mmol) and TSTU (9.97 mg, 0.024 mmol). After stirring for 1 h, to the reaction mixture was added TFA (10.3 μL, 0.133 mmol) and stirred for an additional 15 min. The mixture was titrated in ACN and centrifuged at 3500 rpm for 25 min, and the product was isolated by decantation. UPLC-MS Method A: m/z=1306.7 (z=2); t_{R}=1.78 min.

### Example 2: 2,5-Dioxopyrrolidin-1-yl 1-[(α-L-fucopyranosyl)oxy)-6-[2-({2-[(α-L-fucopyranosyl)oxy]ethyl}amino)-2-oxoethyl]-4,8,16,20-tetraoxo-18-{4,8,16-trioxo-6-[2-oxo-2-({2-[(α-D-mannopyranosyl)oxy]ethyl}amino)ethyl]-1-[(α-D-mannopyranosyl)oxy]-3,6,9,15-tetraazaheptadecan-17-yl}-3,6,9,15,18,21-hexaazaheptacosan-27-oate (ML-2)

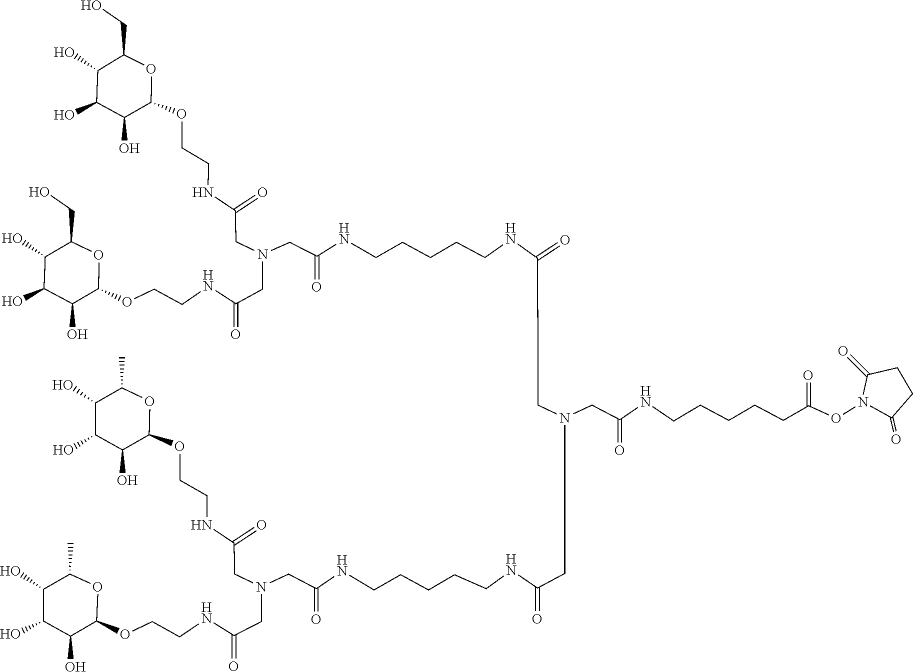 [SMILES: C[C@@H]1O[C@@H](OCCNC(=O)CN(CC(=O)NCCCCCNC(=O)CN(CC(=O)NCCCCCNC(=O)CN(CC(=O)NCCO[C@H]2O[C@H](CO)[C@@H](O)[C@H](O)[C@@H]2O)CC(=O)NCCO[C@H]2O[C@H](CO)[C@@H](O)[C@H](O)[C@@H]2O)CC(=O)NCCCCCC(=O)ON2C(=O)CCC2=O)CC(=O)NCCO[C@@H]2O[C@@H](C)[C@@H](O)[C@@H](O)[C@@H]2O)[C@@H](O)[C@H](O)[C@@H]1O]

### Step 1: Benzyl [5-(2-{bis[2-({2-[(α-D-mannopyranosyl)oxy]ethyl}amino)-2-oxoethyl]amino}acetamido)pentyl]carbamate

To a solution of 13-(carboxymethyl)-3,11-dioxo-1-phenyl-2-oxa-4,10,13-triazapentadecan-15-oic acid (400 mg, 0.98 mmol) and 2-aminoethyl α-D-mannopyranoside (545 mg, 2.44 mmol) in DMF (8 mL) was added EDC (749 mg, 3.91 mmol) and DMAP (358 mg, 2.93 mmol). After stirring at rt for 96 h, the mixture was concentrated, and the residue was purified by C18 reverse phase chromatography to give the title compound. UPLC-MS Method D: m/z=819.0 (z=1); t_{R}=4.58 min.

### Step 2: 2,2′-({2-[(5-Aminopentyl)amino]-2-oxoethyl}azanediyl)bis(N-{2-[(α-D-mannopyranosyl)oxy]ethyl}acetamide)

The title compound was prepared using the procedure analogous to that described for Example 1, Step 3, substituting benzyl [5-(2-{bis[2-({2-[(α-D-mannopyranosyl)oxy]ethyl}amino)-2-oxoethyl]amino}acetamido)pentyl]carbamate for benzyl [6-({2-[(α-D-mannopyranosyl)oxy]ethyl}amino)-6-oxohexyl]carbamate. UPLC-MS Method D: m/z=685.0 (z=1); t_{R}=2.09 min.

### Step 3: 18-(2-{[6-(Benzyloxy)-6-oxohexyl]amino}-2-oxoethyl)-1-[(α-D-mannopyranosyl)oxy]-6-[2-({2-[(α-D-mannopyranosyl)oxy]ethyl}amino)-2-oxoethyl]-4,8,16-trioxo-3,6,9,15,18-pentaazaicosan-20-oic acid

The title compound was prepared using the procedure analogous to that described for Example 1, Step 4, substituting 2,2′-((2-((6-(benzyloxy)-6-oxohexyl)amino)-2-oxoethyl) azanediyl)diacetic acid for 13-(carboxymethyl)-3,11-dioxo-1-phenyl-2-oxa-4,10,13-triazapentadecan-15-oic acid and 2,2′-({2-[(5-aminopentyl)amino]-2-oxoethyl}azanediyl)bis(N-{2-[(α-D-mannopyranosyl)oxy]ethyl}acetamide) for 6-amino-N-{2-[(α-D-mannopyranosyl)oxy]ethyl}hexanamide, respectively. UPLC-MS Method D: m/z=1061.0 (z=1); t_{R}=5.14 min.

### Step 4: Benzyl [5-(2-{bis[2-({2-[(α-L-fucopyranosyl)oxy]ethyl}amino)-2-oxoethyl]amino}acetamido)pentyl]carbamate

To a solution of 13-(carboxymethyl)-3,11-dioxo-1-phenyl-2-oxa-4,10,13-triazapentadecan-15-oic acid (380 mg, 0.93 mmol) and 2-aminoethyl α-L-fucopyranoside (480 mg, 2.32 mmol) in DMF (4 mL) was added EDC (712 mg, 3.71 mmol) and DMAP (340 mg, 2.78 mmol). After stirring at rt for 96 h, the mixture was concentrated, and the residue was purified by reverse phase C18 chromatography to isolate the title compound. Method D: m/z=788.0 (z=1); t_{R}=4.90 min.

### Step 5: 2,2′-({2-[(5-Aminopentyl)amino]-2-oxoethyl}azanediyl)bis(N-{2-[(α-L-fucopyranosyl) oxy]ethyl}acetamide)

The title compound was prepared using the procedure analogous to that described for Example 1, Step 3, substituting benzyl [5-(2-{bis[2-({2-[(α-L-fucopyranosyl)oxy]ethyl} amino)-2-oxoethyl]amino}acetamido)pentyl]carbamate for benzyl [6-({2-[(α-D-mannopyranosyl)oxy]ethyl}amino)-6-oxohexyl]carbamate. UPLC-MS Method D: m/z=654.0 (z=1); t_{R}=2.45 min.

### Step 6: Benzyl 1-[(α-L-fucopyranosyl)oxy]-6-[2-({2-[(α-L-fucopyranosyl)oxy]ethyl}amino)-2-oxoethyl]-4,8,16,20-tetraoxo-18-{4,8,16-trioxo-6-[2-oxo-2-({2-[(α-D-mannopyranosyl)oxy]ethyl}amino)ethyl]-1-[(α-D-mannopyranosyl)oxy]-3,6,9,15-tetraazaheptadecan-17-yl}-3,6,9,15,18,21-hexaazaheptacosan-27-oate

To a mixture of 18-(2-{[6-(benzyloxy)-6-oxohexyl]amino}-2-oxoethyl)-1-[(α-D-mannopyranosyl)oxy]-6-[2-({2-[(α-D-mannopyranosyl)oxy]ethyl}amino)-2-oxoethyl]-4,8,16-trioxo-3,6,9,15,18-pentaazaicosan-20-oic acid (200 mg, 0.188 mmol) and 2,2′-({2-[(5-aminopentyl)amino]-2-oxoethyl}azanediyl)bis(N-{2-[(α-L-fucopyranosyl)oxy]ethyl}acetamide) (185 mg, 0.282 mmol) in DMF (3 mL) was added EDC (72 mg, 0.377 mmol) and DMAP (46 mg, 0.377 mmol). After stirring at rt overnight, the reaction mixture was concentrated, and the residue was purified by reverse phase C18 chromatography to give the title compound. UPLC-MS Method D: m/z=1697.0 (z=1); t_{R}=4.76 min.

### Step 7: 1-[(α-L-Fucopyranosyl)oxy]-6-[2-({2-[(α-L-fucopyranosyl)oxy]ethyl}amino)-2-oxoethyl]-4,8,16,20-tetraoxo-18-{4,8,16-trioxo-6-[2-oxo-2-({2-[(α-D-mannopyranosyl) oxy]ethyl}amino) ethyl]-1-[(α-D-mannopyranosyl)oxy]-3,6,9,15-tetraazaheptadecan-17-yl}-3,6,9,15,18,21-hexaazaheptacosan-27-oic acid

The title compound was prepared using the procedures analogous to that described for Example 1, Step 3, substituting benzyl 1-[(α-L-fucopyranosyl)oxy]-6-[2-({2-[(α-L-fucopyranosyl)oxy]ethyl}amino)-2-oxoethyl]-4,8,16,20-tetraoxo-18-{4,8,16-trioxo-6-[2-oxo-2-({2-[(α-D-mannopyranosyl)oxy]ethyl}amino)ethyl]-1-[(α-D-mannopyranosyl)oxy]-3,6,9,15-tetraazaheptadecan-17-yl}-3,6,9,15,18,21-hexaazaheptacosan-27-oate for benzyl [6-({2-[(α-D-mannopyranosyl)oxy]ethyl}amino)-6-oxohexyl]carbamate. UPLC-MS Method D: m/z=1607.0 (z=1); t_{R}=3.33 min.

### Step 8: 2,5-Dioxopyrrolidin-1-yl 1-[(α-L-fucopyranosyl)oxy]-6-[2-({2-[(α-L-fucopyranosyl) oxy]ethyl}amino)-2-oxoethyl]-4,8,16,20-tetraoxo-18-{4,8,16-trioxo-6-[2-oxo-2-({2-[(α-D-mannopyranosyl)oxy]ethyl}amino)ethyl]-1-[(α-D-mannopyranosyl)oxy-3,6,9,15-tetraazaheptadecan-17-yl}-3,6,9,15,18,21-hexaazaheptacosan-27-oate

The title compound was prepared using the procedure analogous to that described for Example 1, Step 10, substituting 1-[(α-L-fucopyranosyl)oxy]-6-[2-({2-[(α-L-fucopyranosyl) oxy]ethyl}amino)-2-oxoethyl]-4,8,16,20-tetraoxo-18-{4,8,16-trioxo-6-[2-oxo-2-({2-[(α-D-mannopyranosyl)oxy]ethyl}amino)ethyl]-1-[(α-D-mannopyranosyl)oxy]-3,6,9,15-tetraazaheptadecan-17-yl}-3,6,9,15,18,21-hexaazaheptacosan-27-oic acid for 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oic acid. UPLC-MS Method D: m/z=1704.0 (z=1); t_{R}=3.64 min.

### Example 3: 2,5-Dioxopyrrolidin-1-yl 1-[(α-L-fucopyranosyl)oxy]-25-{24-[(α-L-fucoopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oate (ML-3)

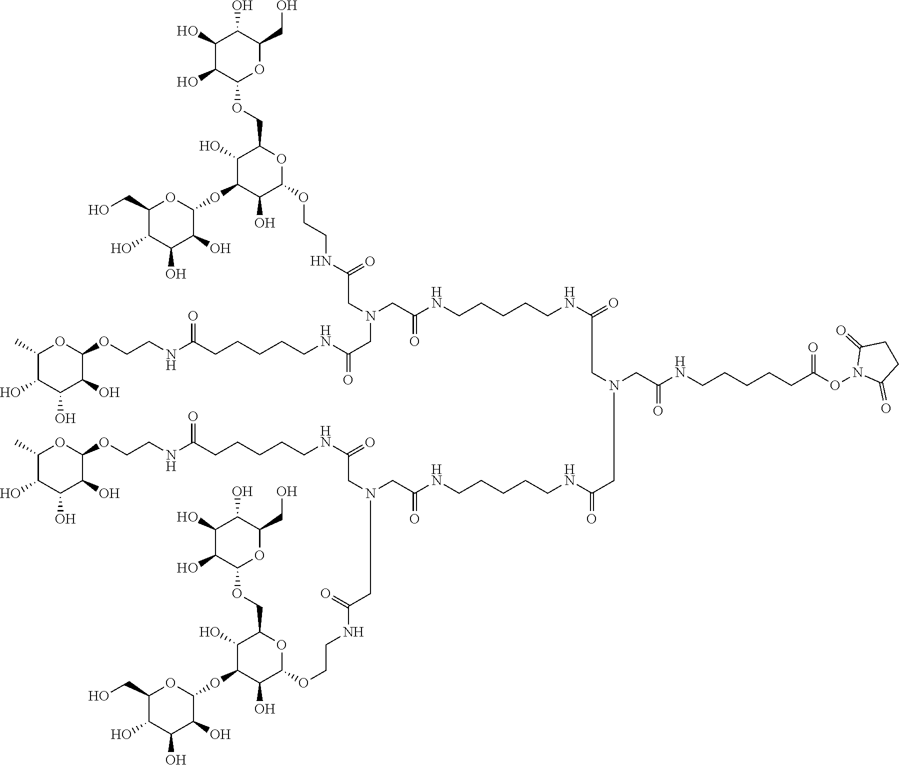 [SMILES: C[C@@H]1O[C@@H](OCCNC(=O)CCCCCNC(=O)CN(CC(=O)NCCCCCNC(=O)CN(CC(=O)NCCCCCNC(=O)CN(CC(=O)NCCCCCC(=O)NCCO[C@@H]2O[C@@H](C)[C@@H](O)[C@@H](O)[C@@H]2O)CC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)[C@@H](O)[C@H](O)[C@@H]1O]

The title compound was prepared using procedures analogous to those described for Example 1, Steps 1 to 10, substituting 2-aminoethyl α-L-fucopyranoside for 2-aminoethyl α-D-mannopyranoside in Step 2. UPLC-MS Method A: m/z=1290.7 (z=2); t_{R}=1.88 min.

### Example 4: 2,5-Dioxopyrrolidin-1-yl 1-[(α-L-fucopyranosyl)oxy]-19-{1-[(α-L-fucopyranosyl)oxy]-6-[2-({2-[(α-L-fucopyranosyl)oxy]ethyl}amino)-2-oxoethyl]-4,8,17-trioxo-3,6,9,16-tetraazaoctadecan-18-yl}-6-[2-({2-[(α-L-fucopyranosyl)oxy]ethyl}amino)-2-oxoethyl]-4,8,17,21-tetraoxo-3,6,9,16,19,22-hexaazaoctacosan-28-oate (ML-4)

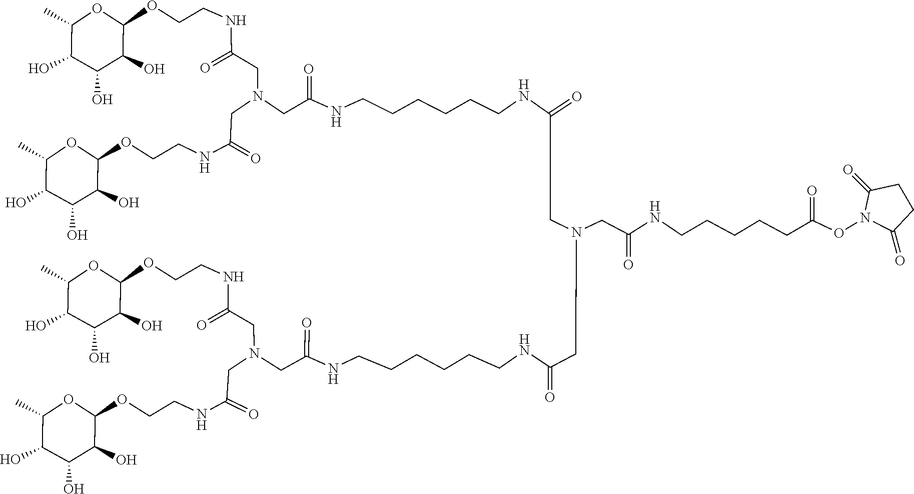 [SMILES: C[C@@H]1O[C@@H](OCCNC(=O)CN(CC(=O)NCCCCCCNC(=O)CN(CC(=O)NCCCCCCNC(=O)CN(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)CC(=O)NCCCCCC(=O)ON2C(=O)CCC2=O)CC(=O)NCCO[C@@H]2O[C@@H](C)[C@@H](O)[C@@H](O)[C@@H]2O)[C@@H](O)[C@H](O)[C@@H]1O]

### Step 1: 14-(Carboxymethyl)-3,12-dioxo-1-phenyl-2-oxa-4,11,14-triazahexadecan-16-oic acid

The title compound was prepared using the procedure analogous to that described for Example 1, Step 1, substituting benzyl (6-aminohexyl)carbamate for benzyl (5-aminopentyl) carbamate. UPLC-MS Method A: m/z=424.21 (z=1); t_{R}=3.58 min.

### Step 2: Benzyl [6-(2-{bis[2-({2-[(α-L-fucopyranosyl)oxy]ethyl}amino)-2-oxoethyl]amino}acetamido)hexyl]carbamate

The title compound was prepared using the procedure analogous to that described for Example 2, Step 4, substituting 14-(carboxymethyl)-3,12-dioxo-1-phenyl-2-oxa-4,11,14-triazahexadecan-16-oic acid for 13-(carboxymethyl)-3,11-dioxo-1-phenyl-2-oxa-4,10,13-triazapentadecan-15-oic acid. UPLC-MS Method A: m/z=802.42 (z=1); t_{R}=3.22 min.

### Step 3: 2,2′-({2-[(6-Aminohexyl)amino]-2-oxoethyl}azanediyl)bis(N-{2-[(α-L-fucopyranosyl)oxy]ethyl}acetamide)

The title compound was prepared using the procedure analogous to that described for Example 1, Step 3, substituting benzyl [6-(2-{bis[2-({2-[(α-L-fucopyranosyl)oxy]ethyl} amino)-2-oxoethyl]amino}acetamido)hexyl]carbamate for benzyl [6-({2-[(α-D-mannopyranosyl)oxy]ethyl}amino)-6-oxohexyl]carbamate in Step 3. UPLC-MS Method A: m/z=668.40 (z=1); t_{R}=1.38 min.

### Step 4: 2,5-Dioxopyrrolidin-1-yl 1-[(α-L-fucopyranosyl)oxy]-19-{1-[(α-L-fucopyranosyl)oxy]-6-[2-({2-[(α-L-fucopyranosyl)oxy]ethyl}amino)-2-oxoethyl]-4,8,17-trioxo-3,6,9,16-tetraazaoctadecan-18-yl}-6-[2-({2-[(α-L-fucopyranosyl)oxy]ethyl}amino)-2-oxoethyl]-4,8,17,21-tetraoxo-3,6,9,16,19,22-hexaazaoctacosan-28-oate

The title compound was prepared using procedures analogous to those described for Example 1, Steps 8 to 10, substituting 2,2′-({2-[(6-aminohexyl)amino]-2-oxoethyl}azanediyl) bis(N-{2-[(α-L-fucopyranosyl)oxy]ethyl}acetamide) for 6-{2-[{2-[(5-aminopentyl)amino]-2-oxoethyl}(2-oxo-2-{[2-({α-D-mannopyranosyl-(1-3)-[α-D-mannopyranosyl-(1→6)]-α-D-mannopyranosyl}oxy)ethyl]amino}ethyl)amino]acetamido}-N-{2-[(α-D-mannopyranosyl)oxy]ethyl}hexanamide in Step 8. UPLC-MS Method A: m/z=1700.98 (z=1); t_{R}=2.47 min.

### Example 5: 2,5-Dioxopyrrolidin-1-yl (S)-1-[(α-L-fucopyranosyl)oxy]-16-(2-{[(2S)-1-{[6-({2-[(α-L-fucopyranosyl)oxy]ethyl}amino)-6-oxohexyl]amino}-1,5-dioxo-5-{[2-({α-D-mannopyranosyl-(1→3)-[α-D-mannopyranosyl-(1→6)]-α-D-mannopyranosyl}oxy)ethyl]amino}pentan-2-yl]amino}-2-oxoethyl)-4,11,14,18-tetraoxo-12-(3-oxo-3-{[2-({α-D-mannopyranosyl-(1→3)-[α-D-mannopyranosyl-(1→6)]-α-D-mannopyranosyl}oxy)ethyl]amino}propyl)-3,10,13,16,19-pentaazapentacosan-25-oate (ML-5)

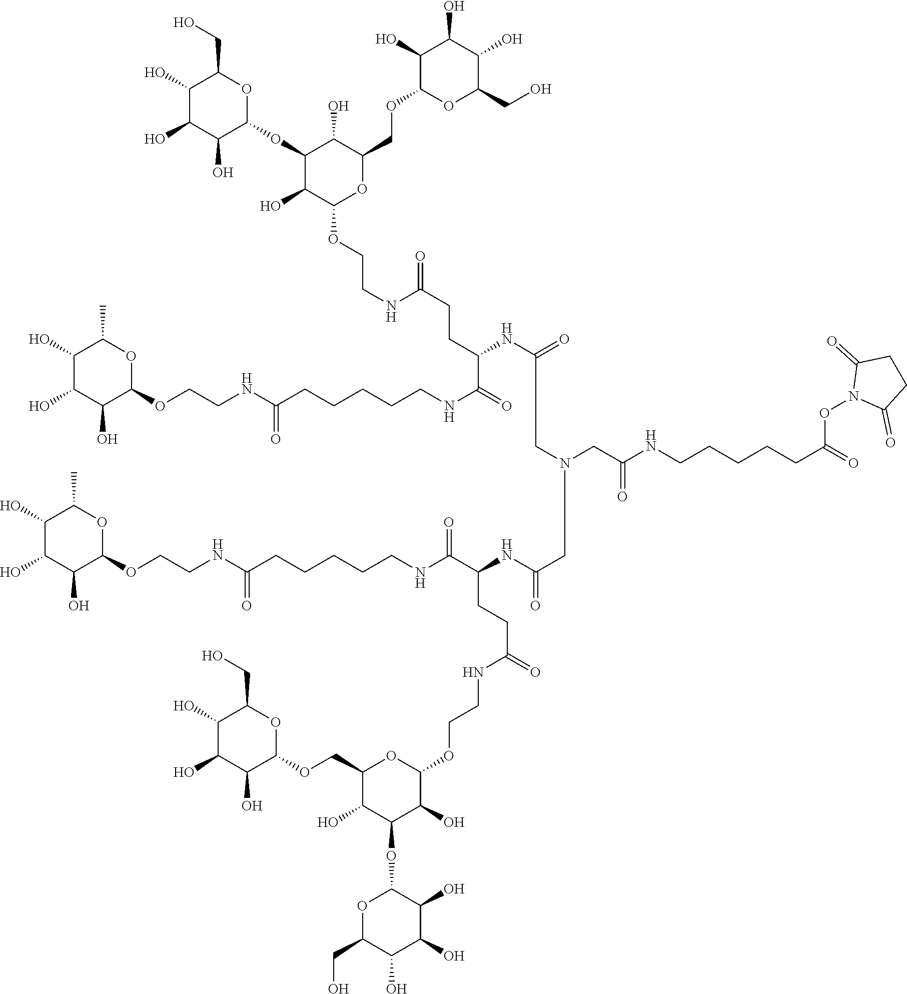 [SMILES: C[C@@H]1O[C@@H](OCCNC(=O)CCCCCNC(=O)[C@H](CCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)NC(=O)CN(CC(=O)NCCCCCC(=O)ON2C(=O)CCC2=O)CC(=O)N[C@@H](CCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)C(=O)NCCCCCC(=O)NCCO[C@@H]2O[C@@H](C)[C@@H](O)[C@@H](O)[C@@H]2O)[C@@H](O)[C@H](O)[C@@H]1O]

### Step 1: Benzyl (S)-4-{[(benzyloxy)carbonyl]amino}-5-{[6-({2-[(α-L-fucopyranosyl)oxy]ethyl}amino)-6-oxohexyl]amino}-5-oxopentanoate

To a mixture of 6-amino-N-{2-[(α-L-fucopyranosyl)oxy]ethyl}hexanamide (518 mg, 1.62 mmol) and Z-L-glutamic acid γ-benzyl ester (500 mg, 1.35 mmol) in DMF (8 mL) at rt was added EDC (645 mg, 3.37 mmol), HOBt (20 mg, 0.135 mmol) and TEA (19 μL, 0.135 mmol). After stirring at rt for 16 h, the mixture was concentrated, and the residue was purified by reverse phase C18 chromatography to give the title compound. UPLC-MS Method A: m/z=674.0 (z=1); t_{R}=5.11 min.

### Step 2: (S)-4-{[(Benzyloxy)carbonyl]amino}-5-{[6-({2-[(α-L-fucopyranosyl)oxy]ethyl}amino)-6-oxohexyl]amino}-5-oxopentanoic acid

To a solution of benzyl (S)-4-{[(benzyloxy)carbonyl]amino}-5-{[6-({2-[(α-L-fucopyranosyl)oxy]ethyl}amino)-6-oxohexyl]amino}-5-oxopentanoate (669 mg, 0.993 mmol) in a mixture of THE (5 mL), MeOH (2 mL) and water (2 mL) at rt was added 1N aq. NaOH (1.192 mL, 1.19 mmol). The resulting mixture was stirred at rt for 3 h. The reaction was quenched by addition of AcOH (80 μL, 1.39 mmol). The resulting mixture was concentrated, and the residue was purified by reverse phase C18 chromatography to provide the title compound. UPLC-MS Method A: m/z=584.0 (z=1); t_{R}=3.35 min.

### Step 3: Benzyl (2S)-(1-{[6-({2-[(α-L-fucopyranosyl)oxy]ethyl}amino)-6-oxohexyl]amino}-1,5-dioxo-5-{[2-({α-D-mannopyranosyl-(1→3)-[α-D-mannopyranosyl-(1→6)]-α-D-mannopyranosyl}oxy)ethyl]amino}pentan-2-yl)carbamate

To a solution of (S)-4-{[(benzyloxy)carbonyl]amino}-5-{[6-({2-[(α-L-fucopyranosyl) ngoxy]ethyl}amino)-6-oxohexyl]amino}-5-oxopentanoic acid (415 mg, 0.71 mmol) and 2-({α-D-mannopyranosyl-(1→3)-[α-D-mannopyranosyl-(1→6)]-α-D-mannopyranosyl}oxy) ethan-1-amine (506 mg, 0.924 mmol) in DMF (5 mL) at rt was added EDC (341 mg, 1.78 mmol) and DMAP (87 mg, 0.71 mmol). After stirring at rt for 16 h, the mixture was concentrated, and the residue was purified by reverse phase C18 chromatography to give the title compound. UPLC-MS Method A: m/z=1114.0 (z=1); t_{R}=2.61 min.

### Step 4: (2S)-2-Amino-N¹-[6-({2-[(α-L-fucopyranosyl)oxy]ethyl}amino)-6-oxohexyl]-N⁵-[2-({α-D-mannopyranosyl-(1→3)-[α-D-mannopyranosyl-(1→6)]-α-D-mannopyranosyl}oxy)ethyl]pentanediamide

The title compound was prepared using the procedure analogous to that described for Example 1, Step 3, substituting benzyl (S)-(1-{[6-({2-[(α-L-fucopyranosyl)oxy]ethyl} amino)-6-oxohexyl]amino}-1,5-dioxo-5-{[2-({α-D-mannopyranosyl-(1-3)-[α-D-mannopyranosyl-(1→6)]-α-D-mannopyranosyl}oxy)ethyl]amino}pentan-2-yl)carbamate for benzyl [6-({2-[(α-D-mannopyranosyl)oxy]ethyl}amino)-6-oxohexyl]carbamate. UPLC-MS Method A: m/z=978.0 (z=1); t_{R}=1.27 min.

### Step 5: 2,5-Dioxopyrrolidin-1-yl (S)-1-[(α-L-fucopyranosyl)oxy]-16-(2-{[(2)-1-{[6-({2-[(α-L-fucopyranosyl)oxy]ethyl}amino)-6-oxohexyl]amino}-1,5-dioxo-5-{[2-({α-D-mannopyranosyl-(1→3)-[α-D-mannopyranosyl-(1-+6)]-α-D-mannopyranosyl}oxy)ethyl]amino}pentan-2-yl]amino}-2-oxoethyl)-4,11,14,18-tetraoxo-12-(3-oxo-3-{[2-({α-D-mannopyranosyl-(1→3)-[α-D-mannopyranosyl-(1→6)]-α-D-mannopyranosyl}oxy)ethyl]amino}propyl)-3,10,13,16,19-pentaazapentacosan-25-oate

The title compound was prepared using procedures analogous to those described for Example 1, Steps 8 to 10, substituting (S)-2-amino-N¹-[6-(2-[(α-L-fucopyranosyl)oxy]ethyl amino)-6-oxohexyl]-N5-[2-({α-D-mannopyranosyl-(1-3)-[α-D-mannopyranosyl-(1→6)]-α-D-mannopyranosyl} oxy)ethyl]pentanediamide for 6-]2-[{2-[(5-aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino}ethyl)amino]acetamido-N-{2-[(α-D-mannopyranosyl)oxy]ethyl}hexanamide in Step 8. UPLC-MS Method A: m/z=1162.4 (z=2); t_{R}=2.94 min.

### Example 6: 2,5-Dioxopyrrolidin-1-yl (S)-1-[(α-D-mannopyranosyl)oxy]-16-(2-{[(2S)-1-{[6-({2-[(α-D-mannopyranosyl)oxy]ethyl}amino)-6-oxohexyl]amino}-1,5-dioxo-5-{[2-({α-D-mannopyranosyl-(1→3)-[α-D-mannopyranosyl-(1→6)]-α-D-mannopyranosyl}oxy)ethyl]amino}pentan-2-yl]amino}-2-oxoethyl)-4,11,14,18-tetraoxo-12-(3-oxo-3-{[2-({α-D-mannopyranosyl-(1→3)-[α-D-mannopyranosyl-(1→6)]-α-D-mannopyranosyl}oxy)ethyl]amino}propyl)-3,10,13,16,19-pentaazapentacosan-25-oate (ML-6)

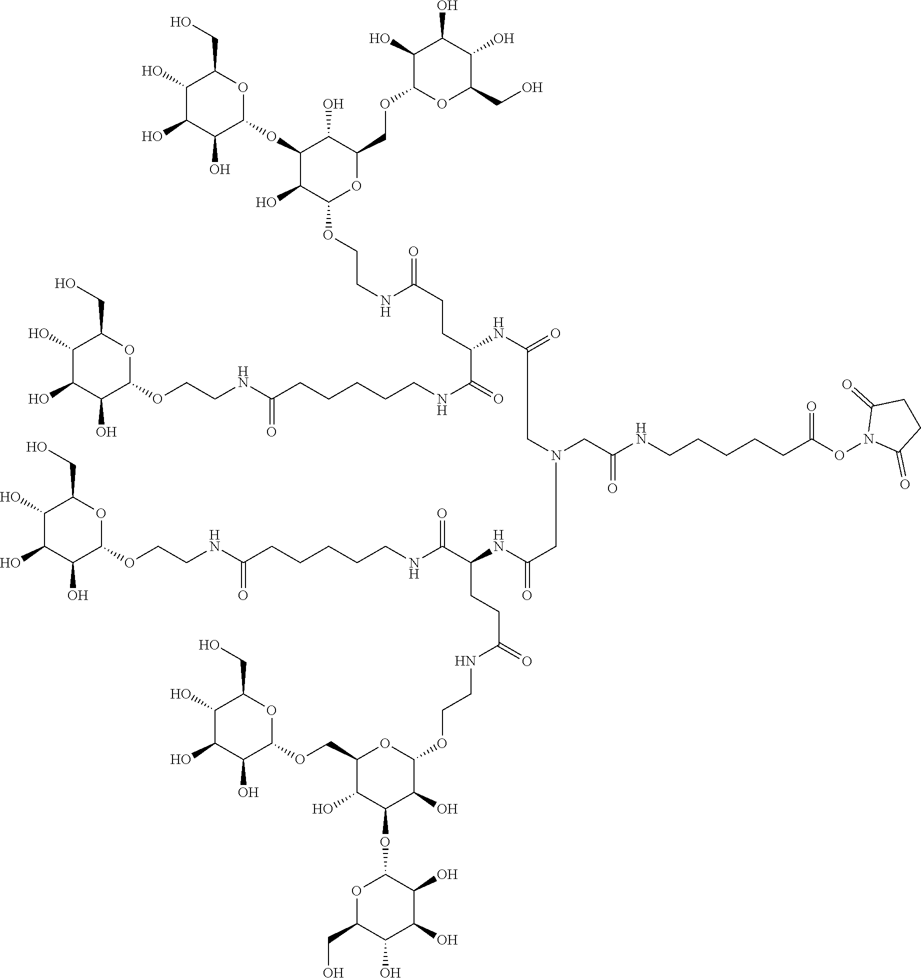 [SMILES: O=C(CCCCCNC(=O)[C@H](CCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NC(=O)CN(CC(=O)NCCCCCC(=O)ON1C(=O)CCC1=O)CC(=O)N[C@@H](CCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)C(=O)NCCCCCC(=O)NCCO[C@H]1O[C@H](CO)[C@@H](O)[C@H](O)[C@@H]1O)NCCO[C@H]1O[C@H](CO)[C@@H](O)[C@H](O)[C@@H]1O]

The title compound was prepared using procedures analogous to those described for Example 5, Steps 1 to 5, substituting 6-amino-N-{2-[(α-D-mannopyranosyl)oxy]ethyl}hexanamide for 6-amino-N-{2-[(α-L-fucopyranosyl)oxy]ethyl}hexanamide in Step 1. UPLC-MS Method A: m/z=1178.4 (z=2); t_{R}=3.46 min.

### Example 7: 2,5-Dioxopyrrolidin-1-yl (S)-11-(2-{[(2S)-1,5-bis({2-[(α-L-fucopyranosyl)oxy]ethyl}amino)-1,5-dioxopentan-2-yl]amino}-2-oxoethyl)-1-[(α-L-fucopyranosyl)oxy]-7-({2-[(α-L-fucopyranosyl)oxy]ethyl}carbamoyl)-4,9,13-trioxo-3,8,11,14-tetraazaicosan-20-oate (ML-7)

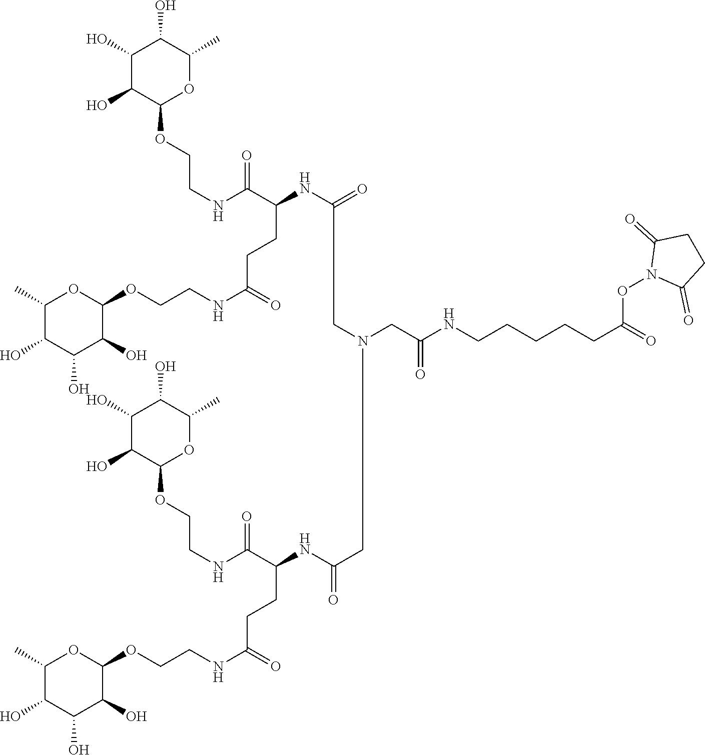 [SMILES: C[C@@H]1O[C@@H](OCCNC(=O)CC[C@H](NC(=O)CN(CC(=O)NCCCCCC(=O)ON2C(=O)CCC2=O)CC(=O)N[C@@H](CCC(=O)NCCO[C@@H]2O[C@@H](C)[C@@H](O)[C@@H](O)[C@@H]2O)C(=O)NCCO[C@@H]2O[C@@H](C)[C@@H](O)[C@@H](O)[C@@H]2O)C(=O)NCCO[C@@H]2O[C@@H](C)[C@@H](O)[C@@H](O)[C@@H]2O)[C@@H](O)[C@H](O)[C@@H]1O]

### Step 1: Benzyl (2S)-[1,5-bis({2-[(α-L-fucopyranosyl)oxy]ethyl}amino)-1,5-dioxopentan-2-yl]carbamate

To a solution of Z-L-glutamic acid (1.0 g, 3.56 mmol) in DMF (60 mL) at rt was added EDC hydrochloride (2.05 g, 10.67 mmol), HOBt (54.4 mg, 0.356 mmol), TEA (1.487 mL, 10.67 mmol) and 2-aminoethyl α-L-fucopyranoside (1.62 g, 7.82 mmol). After stirring at rt for 16 h, the reaction mixture was concentrated, and the residue was purified by reverse phase C18 chromatography to give the title compound. UPLC-MS Method A: m/z=660.3 (z=1); t_{R}=2.31 min.

### Step 2: (2S)-2-Amino-N¹,N⁵-bis{2-[(α-L-fucopyranosyl)oxy]ethyl}pentanediamide

The title compound was prepared using the procedure analogous to that described for Example 1, Step 3, substituting benzyl (2S)-[1,5-bis({2-[(α-L-fucopyranosyl)oxy]ethyl} amino)-1,5-dioxopentan-2-yl]carbamate for benzyl [6-({2-[(α-D-mannopyranosyl)oxy]ethyl} amino)-6-oxohexyl]carbamate. UPLC-MS Method A: m/z=526.3 (z=1); t_{R}=0.91 min.

### Step 3: 2,5-Dioxopyrrolidin-1-yl (S)-11-(2-{[(2S)-1,5-bis({2-[(α-L-fucopyranosyl)oxy]ethyl}amino)-1,5-dioxopentan-2-yl]amino}-2-oxoethyl)-1-[(α-L-fucopyranosyl)oxy]-7-({2-[(α-L-fucopyranosyl)oxy]ethyl}carbamoyl)-4,9,13-trioxo-3, 8,11,14-tetraazaicosan-20-oate

The title compound was prepared using procedures analogous to those described for Example 1, Steps 8 to 10, substituting (2S)-2-amino-N¹,N⁵-bis{2-[(α-L-fucopyranosyl)oxy]ethyl}pentanediamide for 6-{2-[{2-[(5-aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino}ethyl)amino]acetamido}-N-{2-[(α-D-mannopyranosyl)oxy]ethyl}hexanamide in Step 8. UPLC-MS Method A: m/z=1416.8 (z=1); t_{R}=1.87 min.

### Example 8: 2,5-Dioxopyrrolidin-1-yl (S)-11-(2-{[(2S)-1,5-bis({2-[(α-D-mannopyranosyl) oxy]ethyl}amino)-1,5-dioxopentan-2-yl]amino}-2-oxoethyl)-1-[(α-D-mannopyranosyl)oxy]-7-({2-[(α-D-mannopyranosyl)oxy]ethyl}carbamoyl)-4,9,13-trioxo-3,8,11,14-tetraazaicosan-20-oate (ML-8)

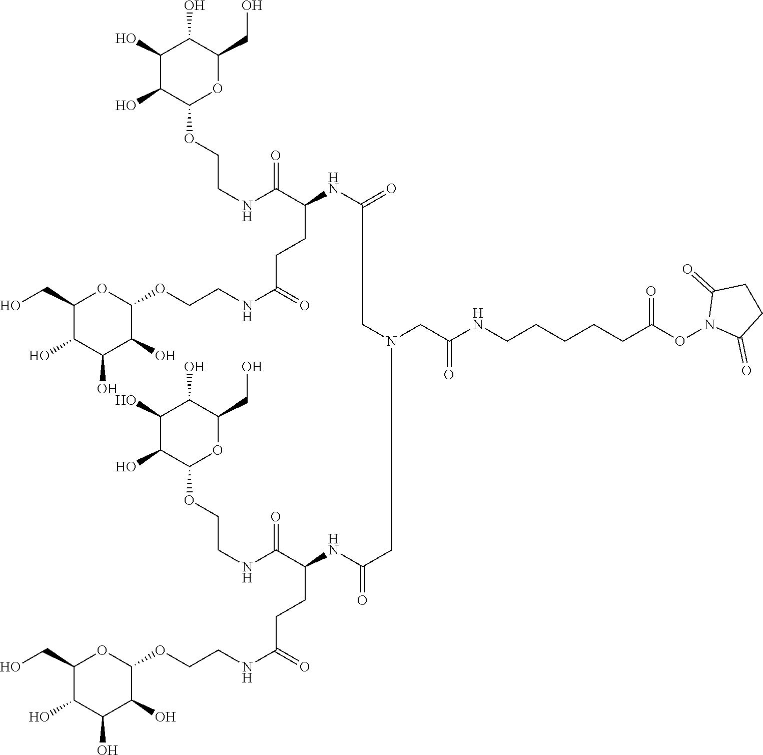 [SMILES: O=C(CC[C@H](NC(=O)CN(CC(=O)NCCCCCC(=O)ON1C(=O)CCC1=O)CC(=O)N[C@@H](CCC(=O)NCCO[C@H]1O[C@H](CO)[C@@H](O)[C@H](O)[C@@H]1O)C(=O)NCCO[C@H]1O[C@H](CO)[C@@H](O)[C@H](O)[C@@H]1O)C(=O)NCCO[C@H]1O[C@H](CO)[C@@H](O)[C@H](O)[C@@H]1O)NCCO[C@H]1O[C@H](CO)[C@@H](O)[C@H](O)[C@@H]1O]

The title compound was prepared using procedures analogous to those described for Example 7, Steps 1 to 3, substituting 2-aminoethyl α-D-mannopyranoside for 2-aminoethyl α-L-fucopyranoside in Step 1. UPLC-MS Method A: m/z=1480.50 (z=1); t_{R}=4.12 min.

### Example 9: 2,5-Dioxopyrrolidin-1-yl (S)-11-(2-{[(2S)-1,5-bis({2-[(α-D-mannopyranosyl) oxy]ethyl}amino)-1,5-dioxopentan-2-yl]amino}-2-oxoethyl)-1-[(α-D-mannopyranosyl)oxy]-7-({2-[(α-D-mannopyranosyl)oxy]ethyl}carbamoyl)-4,9,13,20-tetraoxo-3,8,11,14,21-pentaazaheptacosan-27-oate (ML-9)

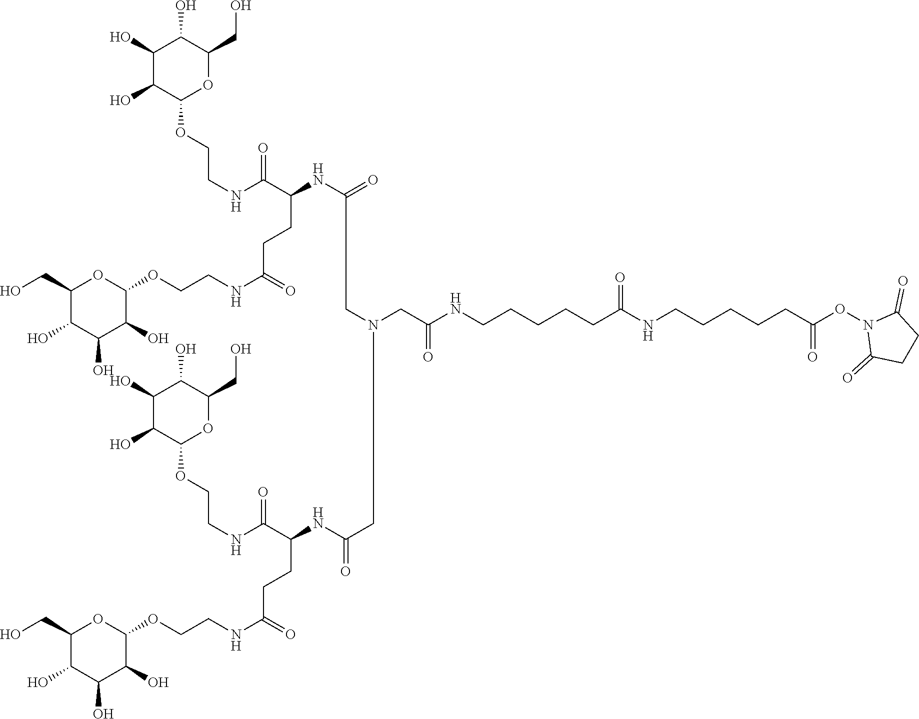 [SMILES: O=C(CCCCCNC(=O)CN(CC(=O)N[C@@H](CCC(=O)NCCO[C@H]1O[C@H](CO)[C@@H](O)[C@H](O)[C@@H]1O)C(=O)NCCO[C@H]1O[C@H](CO)[C@@H](O)[C@H](O)[C@@H]1O)CC(=O)N[C@@H](CCC(=O)NCCO[C@H]1O[C@H](CO)[C@@H](O)[C@H](O)[C@@H]1O)C(=O)NCCO[C@H]1O[C@H](CO)[C@@H](O)[C@H](O)[C@@H]1O)NCCCCCC(=O)ON1C(=O)CCC1=O]

### Step 1: Benzyl (S)-11-(2-{[(2S)-1,5-bis({2-[(α-D-mannopyranosyl)oxy]ethyl}amino)-1,5-dioxopentan-2-yl]amino}-2-oxoethyl)-1-[(α-D-mannopyranosyl)oxy]-7-({2-[(α-D-mannopyranosyl)oxy]ethyl}carbamoyl)-4,9,13,20-tetraoxo-3,8,11,14,21-pentaazaheptacosan-27-oate

To a solution of benzyl 6-aminohexanoate 4-methylbenzenesulfonate (63.8 mg, 0.162 mmol) in DMF (1.0 mL) at 0° C. was added DIPEA (31 μL, 0.175 mmol) dropwise. After stirring 15 min at 0° C., to the resulting mixture was added 2,5-dioxopyrrolidin-1-yl (S)-11-(2-{[(2S)-1,5-bis({2-[(α-D-mannopyranosyl)oxy]ethyl}amino)-1,5-dioxopentan-2-yl]amino}-2-oxoethyl)-1-[(α-D-mannopyranosyl)oxy]-7-({2-[(α-D-mannopyranosyl)oxy]ethyl}carbamoyl)-4,9,13-trioxo-3,8,11,14-tetraazaicosan-20-oate (Example 8, 96 mg, 0.065 mmol) in DMF (1 mL) dropwise. After stirring for 2 h, the reaction mixture was concentrated, and the residue was re-dissolved in water and purified by reverse phase C4 HPLC, eluting with 2-30% ACN (0.1% TFA) in water (0.1% TFA) over 25 min. The desired fractions were collected, concentrated, and freeze-dried to give the desired product. LC-MS Method A: m/z=794.22 (z=2); t_{R}=1.07 min.

### Step 2: 2,5-Dioxopyrrolidin-1-yl (S)-11-(2-{[(2S)-1,5-bis({2-[(α-D-mannopyranosyl)oxy]ethyl}amino)-1,5-dioxopentan-2-yl]amino}-2-oxoethyl)-1-[(α-D-mannopyranosyl)oxy]-7-({2-[(α-D-mannopyranosyl)oxy]ethyl}carbamoyl)-4,9,13,20-tetraoxo-3,8,11,14,21-pentaazaheptacosan-27-oate

The title compound was prepared using procedures analogous to those described for Example 1, Steps 9 to 10, substituting benzyl (S)-11-(2-{[(2S)-1,5-bis({2-[(α-D-mannopyranosyl)oxy]ethyl}amino)-1,5-dioxopentan-2-yl]amino}-2-oxoethyl)-1-[(α-D-mannopyranosyl)oxy]-7-({2-[(α-D-mannopyranosyl)oxy]ethyl}carbamoyl)-4,9,13,20-tetraoxo-3,8,11,14,21-pentaazaheptacosan-27-oate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oate in Step 9. UPLC-MS Method A: m/z=1593.77 (z=1); t_{R}=2.98 min.

### Example 10: 2,5-Dioxopyrrolidin-1-yl (S)-18-[2-({(12S)-1,25-bis[(α-D-mannopyranosyl) oxy]-4,11,15,22-tetraoxo-3,10,16,23-tetraazapentacosan-12-yl}amino)-2-oxoethyl]-1-[(α-D-mannopyranosyl)oxy]-14-{[6-({2-[(α-D-mannopyranosyl)oxy]ethyl}amino)-6-oxohexyl]carbamoyl}-4,11,16,20-tetraoxo-3,10,15,18,21-pentaazaheptacosan-27-oate (ML-10)

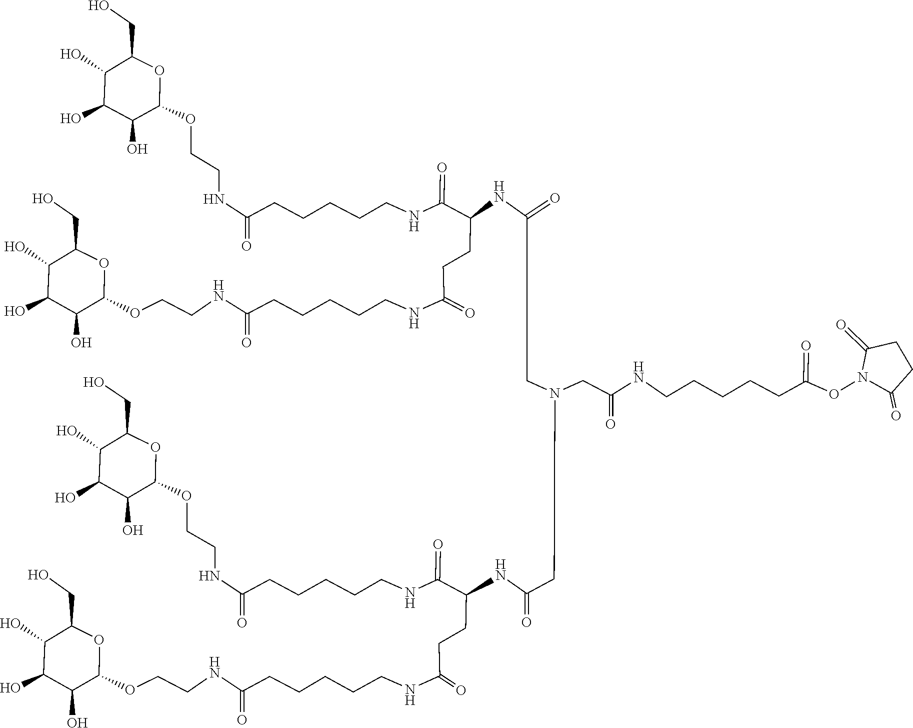 [SMILES: O=C(CCCCCNC(=O)CC[C@H](NC(=O)CN(CC(=O)NCCCCCC(=O)ON1C(=O)CCC1=O)CC(=O)N[C@@H](CCC(=O)NCCCCCC(=O)NCCO[C@H]1O[C@H](CO)[C@@H](O)[C@H](O)[C@@H]1O)C(=O)NCCCCCC(=O)NCCO[C@H]1O[C@H](CO)[C@@H](O)[C@H](O)[C@@H]1O)C(=O)NCCCCCC(=O)NCCO[C@H]1O[C@H](CO)[C@@H](O)[C@H](O)[C@@H]1O)NCCO[C@H]1O[C@H](CO)[C@@H](O)[C@H](O)[C@@H]1O]

The title compound was prepared using procedures analogous to those described for Example 7, Steps 1 to 3, substituting 6-amino-N-{2-[(α-D-mannopyranosyl)oxy]ethyl}hexanamide for 2-aminoethyl α-L-fucopyranoside in Step 1. UPLC-MS Method A: m/z=976.56 (z=2); t_{R}=3.90 min.

### Example 11: 2,5-Dioxopyrrolidin-1-yl (S)-11-(2-{[(2S)-1,5-bis({2-[(α-D-glucopyranosyl) oxy]ethyl}amino)-1,5-dioxopentan-2-yl]amino}-2-oxoethyl)-1-[(α-D-glucopyranosyl)oxy]-7-({2-[(α-D-glucopyranosyl)oxy]ethyl}carbamoyl)-4,9,13-trioxo-3,8,11,14-tetraazaicosan-20-oate (ML-11)

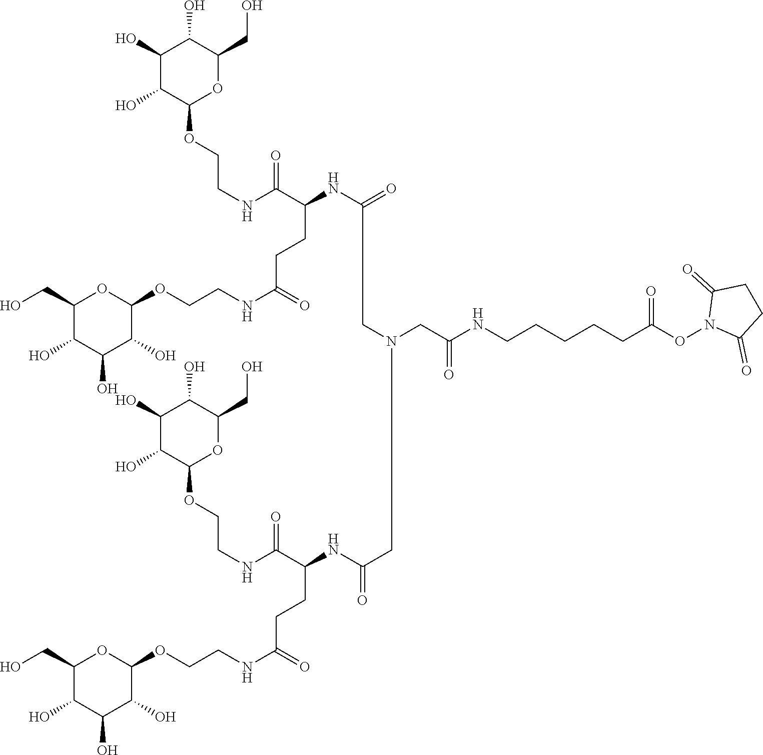 [SMILES: O=C(CC[C@H](NC(=O)CN(CC(=O)NCCCCCC(=O)ON1C(=O)CCC1=O)CC(=O)N[C@@H](CCC(=O)NCCO[C@@H]1O[C@H](CO)[C@@H](O)[C@H](O)[C@H]1O)C(=O)NCCO[C@@H]1O[C@H](CO)[C@@H](O)[C@H](O)[C@H]1O)C(=O)NCCO[C@@H]1O[C@H](CO)[C@@H](O)[C@H](O)[C@H]1O)NCCO[C@@H]1O[C@H](CO)[C@@H](O)[C@H](O)[C@H]1O]

The title compound was prepared using procedures analogous to those described for Example 7, Steps 1 to 3, substituting 2-aminoethyl α-D-glucopyranoside for 2-aminoethyl α-L-fucopyranoside in Step 1. UPLC-MS Method A: m/z=1480.80 (z=1); t_{R}=1.50 min.

### Example 12: 2,5-Dioxopyrrolidin-1-yl (S)-11-(2-{[(2S)-1,5-bis({2-[(α-L-fucopyranosyl)oxy]ethyl}amino)-1,5-dioxopentan-2-yl]amino}-2-oxoethyl)-4,9,13-trioxo-1-[(α-D-mannopyranosyl)oxy]-7-({2-[(α-D-mannopyranosyl)oxy]ethyl}carbamoyl)-3,8,11,14-tetraazaicosan-20-oate (ML-12)

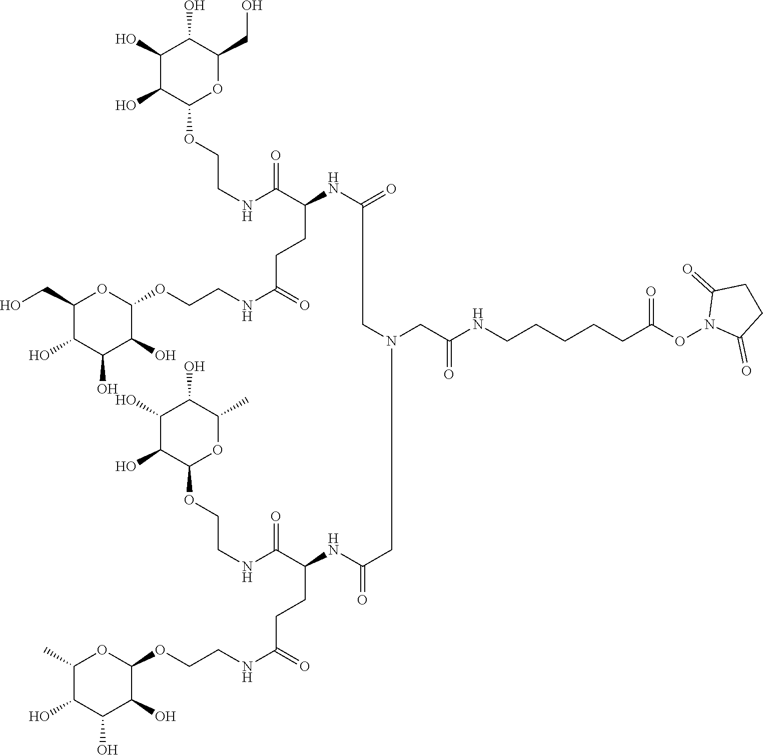 [SMILES: C[C@@H]1O[C@@H](OCCNC(=O)CC[C@H](NC(=O)CN(CC(=O)NCCCCCC(=O)ON2C(=O)CCC2=O)CC(=O)N[C@@H](CCC(=O)NCCO[C@H]2O[C@H](CO)[C@@H](O)[C@H](O)[C@@H]2O)C(=O)NCCO[C@H]2O[C@H](CO)[C@@H](O)[C@H](O)[C@@H]2O)C(=O)NCCO[C@@H]2O[C@@H](C)[C@@H](O)[C@@H](O)[C@@H]2O)[C@@H](O)[C@H](O)[C@@H]1O]

The title compound was prepared using procedures analogous to those described for Example 2 (Step 3 and Steps 6 to 8) substituting (S)-2-amino-N¹,N⁵-bis{2-[(α-D-mannopyranosyl)oxy]ethyl}pentanediamide for 2,2′-({2-[(5-aminopentyl)amino]-2-oxoethyl}azanediyl)bis(N-{2-[(α-D-mannopyranosyl)oxy]ethyl}acetamide) in Step 3, and (S)-2-amino-N¹,N⁵-bis{2-[(α-L-fucopyranosyl)oxy]ethyl}pentanediamide for 2,2′-({2-[(5-aminopentyl) amino]-2-oxoethyl}azanediyl)bis(N-{2-[(α-L-fucopyranosyl)oxy]ethyl}acetamide) in Step 6, respectively. UPLC-MS Method A: m/z=1448.66 (z=1); t_{R}=1.67 min.

### Example 13: 2,5-Dioxopyrrolidin-1-yl (S)-11-(2-{[(2S)-1,5-bis({2-[(α-L-fucopyranosyl) oxy]ethyl}amino)-1,5-dioxopentan-2-yl]amino}-2-oxoethyl)-4,9,13-trioxo-1-[(α-D-glucopyranosyl)oxy]-7-({2-[(α-D-glucopyranosyl)oxy]ethyl}carbamoyl)-3,8,11,14-tetraazaicosan-20-oate (ML-13)

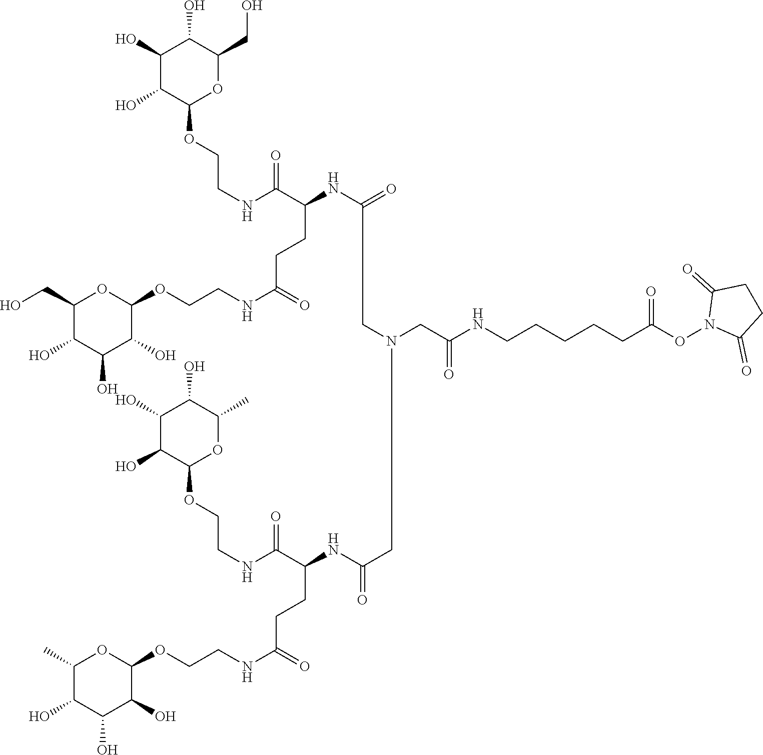 [SMILES: C[C@@H]1O[C@@H](OCCNC(=O)CC[C@H](NC(=O)CN(CC(=O)NCCCCCC(=O)ON2C(=O)CCC2=O)CC(=O)N[C@@H](CCC(=O)NCCO[C@@H]2O[C@H](CO)[C@@H](O)[C@H](O)[C@H]2O)C(=O)NCCO[C@@H]2O[C@H](CO)[C@@H](O)[C@H](O)[C@H]2O)C(=O)NCCO[C@@H]2O[C@@H](C)[C@@H](O)[C@@H](O)[C@@H]2O)[C@@H](O)[C@H](O)[C@@H]1O]

The title compound was prepared using procedures analogous to those described for Example 2 (Step 3 and Steps 6 to 8) substituting (S)-2-amino-N¹,N⁵-bis{2-[(α-L-fucopyranosyl) oxy]ethyl}pentanediamide for 2,2′-({2-[(5-aminopentyl)amino]-2-oxoethyl} azanediyl)bis(N-{2-[(α-D-mannopyranosyl)oxy]ethyl}acetamide) in Step 3, and (S)-2-amino-N¹,N⁵-bis{2-[(α-D-glucopyranosyl)oxy]ethyl}pentanediamide for 2,2′-({2-[(5-aminopentyl) amino]-2-oxoethyl}azanediyl)bis(N-{2-[(α-L-fucopyranosyl)oxy]ethyl}acetamide) in Step 6, respectively. UPLC-MS Method A: m/z=1448.66 (z=1); t_{R}=1.69 min.

### Example 14: 2,5-Dioxopyrrolidin-1-yl (S)-11-(2-{[(2S)-1,5-bis({2-[(α-D-glucopyranosyl) oxy]ethyl}amino)-1,5-dioxopentan-2-yl]amino}-2-oxoethyl)-4,9,13-trioxo-1-[(α-D-mannopyranosyl)oxy]-7-({2-[(α-D-mannopyranosyl)oxy]ethyl}carbamoyl)-3,8,11,14-tetraazaicosan-20-oate (ML-14)

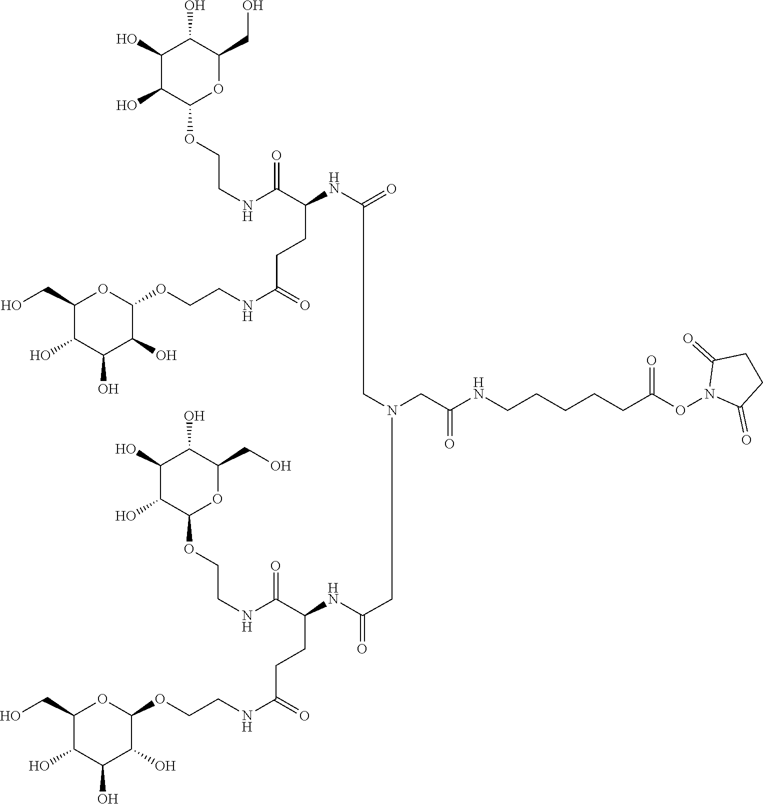 [SMILES: O=C(CC[C@H](NC(=O)CN(CC(=O)NCCCCCC(=O)ON1C(=O)CCC1=O)CC(=O)N[C@@H](CCC(=O)NCCO[C@@H]1O[C@H](CO)[C@@H](O)[C@H](O)[C@H]1O)C(=O)NCCO[C@@H]1O[C@H](CO)[C@@H](O)[C@H](O)[C@H]1O)C(=O)NCCO[C@H]1O[C@H](CO)[C@@H](O)[C@H](O)[C@@H]1O)NCCO[C@H]1O[C@H](CO)[C@@H](O)[C@H](O)[C@@H]1O]

The title compound was prepared using procedures analogous to those described for Example 2, Step 3 and Steps 6 to 8, substituting (S)-2-amino-N¹,N⁵-bis{2-[(α-D-glucopyranosyl) oxy]ethyl}pentanediamide for 2,2′-({2-[(5-aminopentyl)amino]-2-oxoethyl} azanediyl)bis(N-{2-[(α-D-mannopyranosyl)oxy]ethyl}acetamide) in Step 3, and (S)-2-amino-N¹,N⁵-bis{2-[(α-D-mannopyranosyl)oxy]ethyl}pentanediamide for 2,2′-({2-[(5-aminopentyl) amino]-2-oxoethyl}azanediyl)bis(N-{2-[(α-L-fucopyranosyl)oxy]ethyl}acetamide) in Step 6, respectively. UPLC-MS Method A: m/z=1480.65 (z=1); t_{R}=0.22 min.

### Example 15: 2,5-Dioxopyrrolidin-1-yl 13-(2-{[6-(bis{2-[(α-D-mannopyranosyl)oxy]ethyl}amino)-6-oxohexyl]amino}-2-oxoethyl)-1-[(α-D-mannopyranosyl)oxy]-3-{2-[(α-D-mannopyranosyl)oxy]ethyl}-4,11,15-trioxo-3,10,13,16-tetraazadocosan-22-oate (ML-15)

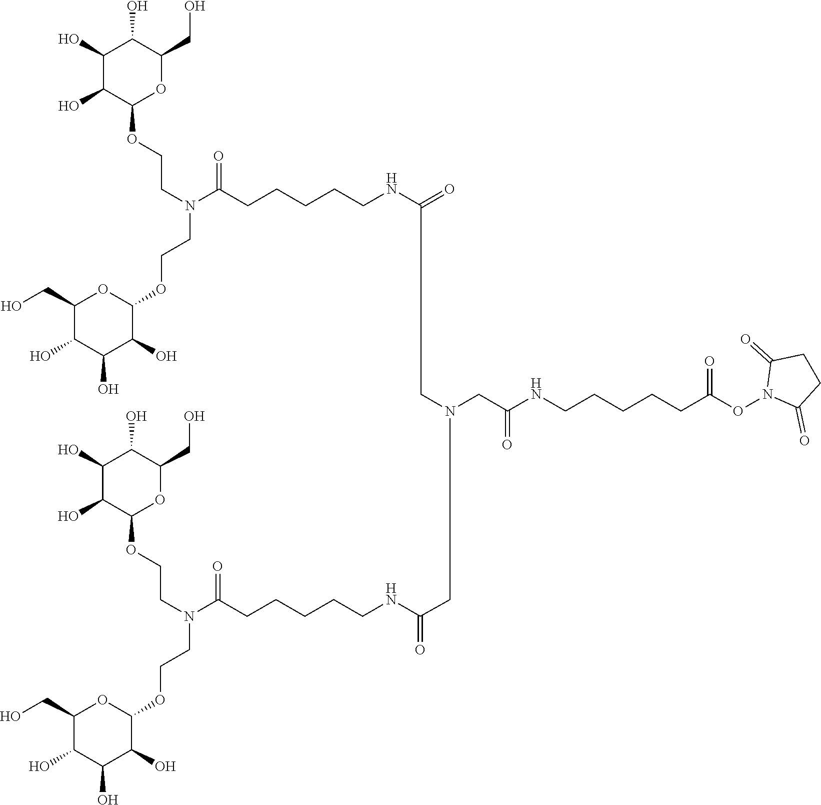 [SMILES: O=C(CN(CC(=O)NCCCCCC(=O)N(CCO[C@@H]1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCO[C@@H]1O[C@H](CO)[C@@H](O)[C@H](O)[C@@H]1O)CCO[C@H]1O[C@H](CO)[C@@H](O)[C@H](O)[C@@H]1O)NCCCCCC(=O)ON1C(=O)CCC1=O]

### Step 1: Benzyl 13-(2-{[6-(bis{2-[(α-D-mannopyranosyl)oxy]ethyl}amino)-6-oxohexyl]amino}-2-oxoethyl)-1-[(α-D-mannopyranosyl)oxy]-3-{2-[(α-D-mannopyranosyl)oxy]ethyl}-4,11,15-trioxo-3,10,13,16-tetraazadocosan-22-oate

To a solution of 2,2′-[(2-{[6-(benzyloxy)-6-oxohexyl]amino}-2-oxoethyl)azanediyl]diacetic acid (100 mg, 0.254 mmol) and 6-amino-N,N-bis{2-[(α-D-mannopyranosyl)oxy]ethyl}hexanamide (290 mg, 0.534 mmol, WO 2019/125878 A1) in DMF (4 mL) at 0° C. was added EDC (130 mg, 0.678 mmol) and HOBt (52.5 mg, 0.343 mmol). After stirring at 0° C. for 30 min and then at rt overnight, the reaction mixture was concentrated down, and the residue was purified by C18 reverse phase chromatography, eluting with 5 to 60% ACN in water, to give the title compound. UPLC-MS Method A: calculated mass for C₆₃H₁₀₆N₄O₃₁ 1442.69, observed m/z=1443.79 (z=1); t_{R}=2.77 min.

### Step 2: 2,5-Dioxopyrrolidin-1-yl 13-(2-{[6-(bis{2-[(α-D-mannopyranosyl)oxy]ethyl}amino)-6-oxohexyl]amino}-2-oxoethyl)-1-[(α-D-mannopyranosyl)oxy]-3-{2-[(α-D-mannopyranosyl) oxy]ethyl}-4,11,15-trioxo-3,10,13,16-tetraazadocosan-22-oate

The title compound was prepared using procedures analogous to those described for Example 1, Steps 9 to 10, substituting benzyl 13-(2-{[6-(bis{2-[(α-D-mannopyranosyl)oxy]ethyl}amino)-6-oxohexyl]amino}-2-oxoethyl)-1-[(α-D-mannopyranosyl)oxy]-3-{2-[(α-D-mannopyranosyl)oxy]ethyl}-4,11,15-trioxo-3,10,13,16-tetraazadocosan-22-oate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy) ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oate in Step 9. UPLC-MS Method A: calculated mass for C₆₀H₁₀₃N₇O₃₃ 1449.66, m/z=1451.2 (z=1); t_{R}=0.47 min.

### Example 16: 2,5-Dioxopyrrolidin-1-yl 13-{2-[(5-{bis[3-(α-D-mannopyranosyl)propyl]amino}pentyl)amino]-2-oxoethyl}-1-(α-D-mannopyranosyl)-4-[3-(α-D-mannopyranosyl) propyl]-11,15-dioxo-4,10,13,16-tetraazadocosan-22-oate (ML-16)

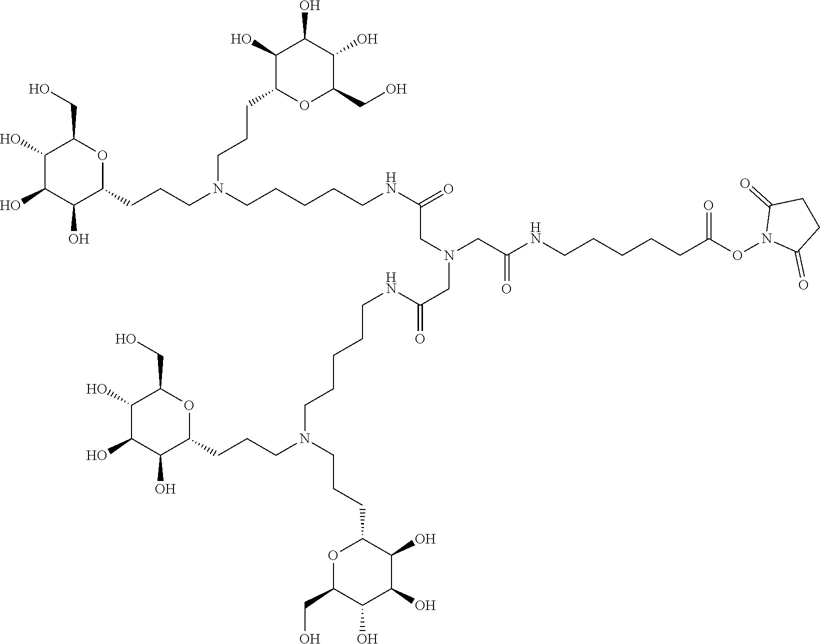 [SMILES: O=C(CN(CC(=O)NCCCCCN(CCC[C@H]1O[C@H](CO)[C@@H](O)[C@H](O)[C@@H]1O)CCC[C@H]1O[C@H](CO)[C@@H](O)[C@H](O)[C@@H]1O)CC(=O)NCCCCCN(CCC[C@H]1O[C@H](CO)[C@@H](O)[C@H](O)[C@@H]1O)CCC[C@H]1O[C@H](CO)[C@@H](O)[C@H](O)[C@@H]1O)NCCCCCC(=O)ON1C(=O)CCC1=O]

### Step 1: 3-(2,3,4,6-Tetra-O-benzyl-α-D-mannopyranosyl)propanal

To a solution of 3-(2,3,4,6-tetra-O-benzyl-α-D-mannopyranosyl)propanal (10 g, 17.16 mmol, J. Am. Chem. Soc. 2002, 124, 8637-8643) in DCM (150 mL) at 0° C. was added Dess-Martin Periodinane (3-oxo-1,3-dihydro-1λ5,2-benziodoxole-1,1,1-triyl triacetate, 10.92 g, 25.7 mmol). The resulting mixture was stirred at 0° C. for 3 h, and then washed with sat. NaHCO₃ (3×150 mL), sat. NaCl (125 mL) and dried over Na₂SO₄. The mixture was filtered, and the filtrate was concentrated. The residue was purified by silica gel column chromatography (220 g), eluting with 0-100% EtOAc in hexanes to give the title compound, which was used without further purification.

### Step 2: Benzyl (5-{bis[3-(2,3,4,6-tetra-O-benzyl-α-D-mannopyranosyl)propyl]amino}pentyl)carbamate

To a mixture of 3-(2,3,4,6-tetra-O-benzyl-α-D-mannopyranosyl)propanal (1.597 g, 2.75 mmol) and benzyl (5-aminopentyl)carbamate hydrochloride (300 mg, 1.1 mmol) in DCM (12 mL) at rt was added sodium triacetoxyborohydride (699 mg, 3.3 mmol). After stirring at rt for 16 h, the resulting mixture was diluted with DCM (30 mL). The mixture was washed with sat. NaHCO₃ (2×50 mL), dried over MgSO₄ and filtered. The filtrate was concentrated, and the residue was purified by reverse phase chromatography to isolate the title compound. UPLC-MS Method A: m/z=1365.72 (z=1); t_{R}=3.97 min.

### Step 3: N¹,N¹-bis[3-(α-D-Mannopyranosyl)propyl]pentane-1,5-diamine

A mixture of benzyl (5-{bis[3-(2,3,4,6-tetra-O-benzyl-α-D-mannopyranosyl)propyl]amino}pentyl)carbamate (1.1 g, 0.8 mmol) and conc. HCl (267 μL, 3.25 mmol) in CH₃OH (10 mL) at rt was added 10% Pd/C (86 mg). The resulting mixture was hydrogenated on a Parr shaker at 40 psi for 5 h. The mixture was filtered through a cake of CELITE™ diatomaceous earth, and the filtrate was evaporated to give the title compound. UPLC-MS Method A: m/z=511.3 (z=1); t_{R}=1.08 min.

### Step 4: 2,5-Dioxopyrrolidin-1-yl 13-{2-[(5-{bis[3-(α-D-mannopyranosyl)propyl]amino]pentyl) amino]-2-oxoethyl}-1-(α-D-mannopyranosyl)-4-[3-(α-D-mannopyranosyl)propyl-11,15-dioxo-4,10,13,16-tetraazadocosan-22-oate

The title compound was prepared using procedures analogous to those described for Example 1, Steps 8 to 10, substituting N¹,N¹-bis[3-(α-D-mannopyranosyl)propyl]pentane-1,5-diamine for 6-{2-[{2-[(5-aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino} ethyl)amino]acetamido}-N-{2-[(α-D-mannopyranosyl)oxy]ethyl}hexanamide in Step 8. UPLC-MS Method A: m/z=1386.8 (z=1); t_{R}=1.08 min.

### Example 17: 2,5-Dioxopyrrolidin-1-yl 1-[(α-D-mannopyranosyl)oxy]-13-(2-{[6-({2-[(α-D-mannopyranosyl)oxy]ethyl}[2-({α-D-mannopyranosyl-(1→3)-[α-D-mannopyranosyl-(1→6)]-α-D-mannopyranosyl}oxy)ethyl]amino)hexyl]amino}-2-oxoethyl)-11,15-dioxo-3-[2-({α-D-mannopyranosyl-(1→3)-[α-D-mannopyranosyl-(1→6)]-α-D-mannopyranosyl}oxy) ethyl]-3,10,13,16-tetraazadocosan-22-oate (ML-17)

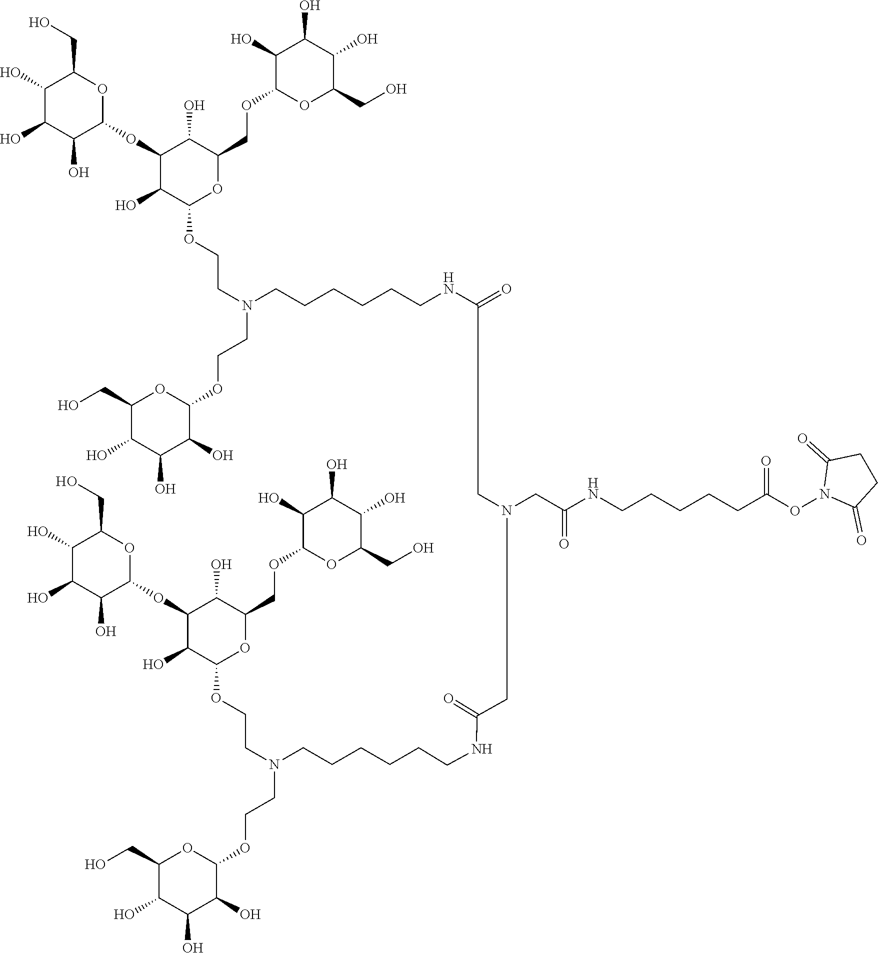 [SMILES: O=C(CN(CC(=O)NCCCCCCN(CCO[C@H]1O[C@H](CO)[C@@H](O)[C@H](O)[C@@H]1O)CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CC(=O)NCCCCCC(=O)ON1C(=O)CCC1=O)NCCCCCCN(CCO[C@H]1O[C@H](CO)[C@@H](O)[C@H](O)[C@@H]1O)CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl (6-oxohexyl)carbamate

To a solution of benzyl (6-hydroxyhexyl)carbamate (3.0 g, 11.94 mmol) in DCM (100 mL) was added Dess-Martin Periodinane (6.58 g, 15.52 mmol). After stirring at rt for 2 h, the reaction mixture was washed with sat. NaHCO₃ (2×100 mL), sat. NaCl (75 mL) and dried over Na₂SO₄. The reaction mixture was filtered, and the filtrate was concentrated and purified by silica gel chromatography to isolate the title compound. ¹H-NMR δ (ppm)(CHCl₃-d): 1.41-1.35 (2H, m), 1.58-1.52 (2H, m), 1.68-1.63 (2H, m), 2.46 (2H, t, J=7.51 Hz), 3.22 (2H, q, J=6.76 Hz), 4.81 (1H, s), 5.12 (2H, s), 7.38 (3H, d, J=4.29 Hz), 9.78 (1H, s).

### Step 2: 2-Aminoethyl (2,3,4,6-tetra-O-benzoyl-α-D-mannopyranosyl)-(1→3)-[2,3,4,6-tetra-O-benzoyl-α-D-mannopyranosyl-(1→6)]-2,4-di-O-benzoyl-α-D-mannopyranoside

To a mixture of 2-azidoethyl (2,3,4,6-tetra-O-benzoyl-α-D-mannopyranosyl)-(1-3)-[2,3,4,6-tetra-O-benzoyl-α-D-mannopyranosyl-(1→6)]-2,4-di-O-benzoyl-α-D-mannopyranoside (25 g, 15.5 mmol, WO2010/088294 A1) in EtOAc (300 mL) and 10% Pd/C (1.65 g) was stirred at rt under H₂ for 16 h. The catalyst was filtered through CELITE™ diatomaceous earth, and the filtrate was concentrated. The residue was purified by silica gel chromatography to give the title compound. UPLC-MS Method A: m/z=1588.6 (z=1); t_{R}=4.03 min.

### Step 3: Benzyl (6-{[2-({(2,3,4,6-tetra-O-benzoyl-α-D-mannopyranosyl)-(1→3)-[2,3,4,6-tetra-O-benzoyl-α-D-mannopyranosyl-(1→6)]-2,4-di-O-benzoyl-α-D-mannopyranosyl}oxy)ethyl]amino}hexyl)carbamate

To a solution of 2-aminoethyl (2,3,4,6-tetra-O-benzoyl-α-D-mannopyranosyl)-(1-3)-[2,3,4,6-tetra-O-benzoyl-α-D-mannopyranosyl-(1→6)]-2,4-di-O-benzoyl-α-D-mannopyranoside (12.7 g, 8.02 mmol) and benzyl (6-oxohexyl)carbamate (1.0 g, 4.01 mmol) in DCM (100 mL) was added AcOH (241 μL, 4.01 mmol) and sodium triacetoxyborohydride (1.28 g, 6.02 mmol). After stirring at rt for 16 h, the mixture was concentrated, and the residue was re-dissolved in EtOAc (300 mL) and washed with sat. NaHCO₃ (2×300 mL), sat. NaCl (200 mL) and dried over Na₂SO₄. The mixture was filtered, and the filtrate was concentrated. The residue was purified by silica gel chromatography to isolate the title compound.

¹H-NMR δ(ppm)(CHCl₃-d): 1.37-1.30 (4H, m), 1.61 (2H, br. s), 3.13 (2H, t, J=7.65 Hz), 3.81 (2H, d, J=11.05 Hz), 4.13 (1H, br. s), 4.20 (1H, dd, J=10.80, 6.43 Hz), 4.33 (1H, dd, J=12.64, 3.15 Hz), 4.40-4.37 (2H, m), 4.50-4.47 (1H, m), 4.55-4.52 (1H, m), 4.61 (1H, dd, J=12.47, 2.65 Hz), 4.67 (1H, d, J=12.48 Hz), 4.74 (1H, br. s), 4.89 (1H, s), 5.09 (1H, s), 5.18 (1H, d, J=1.80 Hz), 5.21 (1H, s), 5.38 (1H, t, J=2.54 Hz), 5.44-5.42 (1H, m), 5.73 (1H, dd, J=10.07, 3.29 Hz), 5.80-5.78 (2H, m), 5.94 (1H, t, J=9.89 Hz), 6.02-5.98 (1H, m), 6.08 (1H, t, J=10.08 Hz), 6.16 (1H, t, J=10.11 Hz), 7.37-7.38 (11H, m), 7.52 (2H, dd, J=13.91, 7.51 Hz), 7.62-7.55 (7H, m), 7.74 (2H, dd, J=7.93, 1.42 Hz), 7.77 (2H, dd, J=7.91, 1.39 Hz), 7.88-7.85 (4H, m), 8.06-8.03 (4H, m), 8.11-8.08 (4H, m), 8.17-8.15 (2H, m), 8.32-8.30 (2H, m).

### Step 4: Benzyl[6-({2-[(2,3,4,6-tetra-O-acetyl-α-D-mannopyranosyl)oxy]ethyl}[2-({(2,3,4,6-tetra-O-benzoyl-α-D-mannopyranosyl)-(1→3)-[2,3,4,6-tetra-O-benzoyl-α-D-mannopyranosyl-(1→6)]-2,4-di-O-benzoyl-α-D-mannopyranosyl}oxy)ethyl]amino)hexyl]carbamate

To a stirring solution of benzyl (6-{[2-({(2,3,4,6-tetra-O-benzoyl-α-D-mannopyranosyl)-(1→3)-[2,3,4,6-tetra-O-benzoyl-α-D-mannopyranosyl-(1→6)]-2,4-di-O-benzoyl-α-D-mannopyranosyl}oxy)ethyl]amino}hexyl)carbamate (2 g, 1.1 mmol) and 2-oxoethyl 2,3,4,6-tetra-O-acetyl-α-D-mannopyranoside (1.29 g, 3.3 mmol, WO2019/125878 A1) in DCM (15 mL) was added AcOH (63 μL, 1.1 mmol) and, 10 min later, sodium triacetoxyborohydride (465 mg, 2.2 mmol). The resulting mixture was stirred at rt overnight and then concentrated. The residue was taken up in EtOAc (100 mL) and washed with sat. NaHCO₃ (100 mL), sat. NaCl (50 mL) and dried over Na₂SO₄. The mixture was filtered, and the filtrate was concentrated. The residue was purified by normal phase chromatography to give the title compound.

### Step 5: Benzyl[6-({2-[(α-D-mannopyranosyl)oxy]ethyl}[2-({(α-D-mannopyranosyl)-(1→3)-[α-D-mannopyranosyl-(1→6)]-α-D-mannopyranosyl}oxy)ethyl]amino)hexyl]carbamate

To a solution of benzyl [6-({2-[(2,3,4,6-tetra-O-acetyl-α-D-mannopyranosyl)oxy]ethyl}[2-({(2,3,4,6-tetra-O-benzoyl-α-D-mannopyranosyl)-(1→3)-[2,3,4,6-tetra-O-benzoyl-α-D-mannopyranosyl-(1→6)]-2,4-di-O-benzoyl-α-D-mannopyranosyl}oxy)ethyl]amino)hexyl]carbamate (2.4 g, 1.09 mmol) in a mixture of DCM (15 mL), and anhydrous MeOH (15 mL) was added sodium methoxide (2.2 mL of a 0.5M solution in MeOH, 1.09 mmol). After stirring at rt for 16 h, to the reaction mixture was added MeOH (15 mL) and sodium methoxide (0.5 mL of a 0.5M solution in MeOH, 0.25 mmol). After stirring for 72 h, the reaction mixture was concentrated and added dropwise to stirred ACN (80 mL). The mixture was then centrifuged at 3500 rpm for 15 min; the solvent was decanted; and the pellet was re-suspended in ACN (80 mL) and centrifuged at 3500 rpm for a further 15 min. The solvent was decanted, and the solid was dried to isolate the title compound. UPLC-MS Method A: m/z=987.5 (z=1); t_{R}=3.72 min.

### Step 6: 6-({2-[(α-D-Mannopyranosyl)oxy]ethyl}[2-({(α-D-mannopyranosyl)-(1→3)-[α-D-mannopyranosyl-(1→6)]-α-D-mannopyranosyl}oxy)ethyl]amino)hexylamine

The title compound was prepared using the procedure analogous to that described for Example 1, substituting benzyl [6-({2-[(α-D-mannopyranosyl)oxy]ethyl}[2-({(α-D-mannopyranosyl)-(1→3)-[α-D-mannopyranosyl-(1→6)]-α-D-mannopyranosyl}oxy)ethyl]amino) hexyl]carbamate for benzyl [6-({2-[(α-D-mannopyranosyl)oxy]ethyl}amino)-6-oxohexyl]carbamate in Step 3. UPLC-MS Method A: m/z=853.4 (z=1); t_{R}=1.00 min.

### Step 7: 2,5-Dioxopyrrolidin-1-yl 1-[(α-D-mannopyranosyl)oxy]-13-(2-{[6-({2-[(α-D-mannopyranosyl)oxy]ethyl}[2-({α-D-mannopyranosyl-(1→3)-[α-D-mannopyranosyl-(1→6)]-α-D-mannopyranosyl}oxy)ethyl]amino)hexyl]amino}-2-oxoethyl)-11,15-dioxo-3-[2-({α-D-mannopyranosyl-(1→3)-[α-D-mannopyranosyl-(1→6)]-α-D-mannopyranosyl}oxy)ethyl-3,10,13,16-tetraazadocosan-22-oate

The title compound was prepared using procedures analogous to that described for Example 1, Steps 8 to 10, substituting 6-({2-[(α-D-mannopyranosyl)oxy]ethyl}[2-({(α-D-mannopyranosyl)-(1→3)-[α-D-mannopyranosyl-(1→6)]-α-D-mannopyranosyl}oxy)ethyl]amino) hexylamine for 6-{2-[{2-[(5-aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino}ethyl)amino]acetamido}-N-{2-[(α-D-mannopyranosyl)oxy]ethyl}hexanamide in Step 8. UPLC-MS Method A: m/z=1036.5 (z=2); t_{R}=4.01 min.

### Example 18: 2,5-Dioxopyrrolidin-1-yl 1-[(β-D-mannopyranosyl)oxy]-13-(2-{[6-({2-[(β-D-mannopyranosyl)oxy]ethyl}[2-({α-D-mannopyranosyl-(1→3)-[α-D-mannopyranosyl-(1→6)]-α-D-mannopyranosyl}oxy)ethyl]amino)hexyl]amino}-2-oxoethyl)-11,15-dioxo-3-[2-({α-D-mannopyranosyl-(1→3)-[α-D-mannopyranosyl-(1→6)]-α-D-mannopyranosyl}oxy) ethyl]-3,10,13,16-tetraazadocosan-22-oate (ML-18)

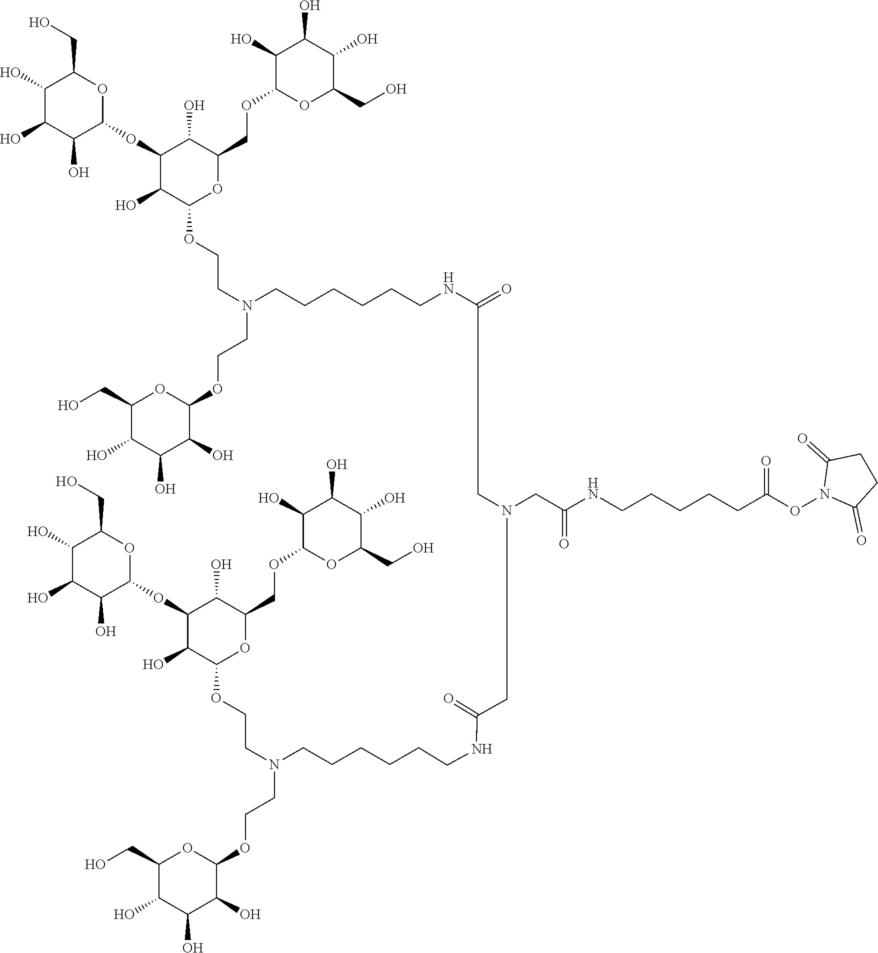 [SMILES: O=C(CN(CC(=O)NCCCCCCN(CCO[C@@H]1O[C@H](CO)[C@@H](O)[C@H](O)[C@@H]1O)CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CC(=O)NCCCCCC(=O)ON1C(=O)CCC1=O)NCCCCCCN(CCO[C@@H]1O[C@H](CO)[C@@H](O)[C@H](O)[C@@H]1O)CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for Example 17, Steps 4 to 7, substituting 2-oxoethyl 2,3,4,6-tetra-O-acetyl-β-D-mannopyranoside for 2-oxoethyl 2,3,4,6-tetra-O-acetyl-α-D-mannopyranoside in Step 4. UPLC-MS Method A: m/z=1036.5 (z=2); t_{R}=3.84 min.

### Example 19: 2,5-Dioxopyrrolidin-1-yl 1-[(α-L-fucopyranosyl)oxy]-13-(2-{[6-({2-[(α-L-fucopyranosyl)oxy]ethyl}[2-({α-D-mannopyranosyl-(1→3)-[α-D-mannopyranosyl-(1→6)]-α-D-mannopyranosyl}oxy)ethyl]amino)hexyl]amino}-2-oxoethyl)-11,15-dioxo-3-[2-({α-D-mannopyranosyl-(1→3)-[α-D-mannopyranosyl-(1→6)]-α-D-mannopyranosyl}oxy)ethyl]-3,10,13,16-tetraazadocosan-22-oate (ML-19)

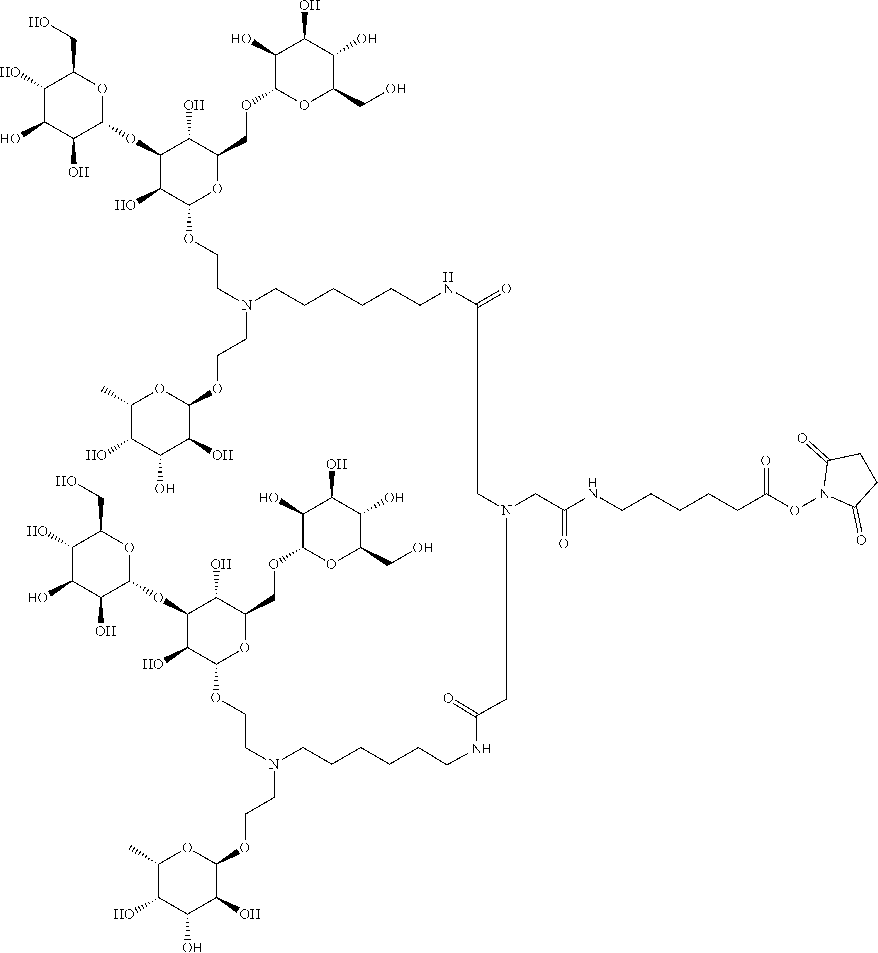 [SMILES: C[C@@H]1O[C@@H](OCCN(CCCCCCNC(=O)CN(CC(=O)NCCCCCCN(CCO[C@@H]2O[C@@H](C)[C@@H](O)[C@@H](O)[C@@H]2O)CCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)[C@@H](O)[C@H](O)[C@@H]1O]

The title compound was prepared using procedures analogous to those described for Example 17, Steps 4 to 7, substituting 2-oxoethyl 2,3,4-tri-O-acetyl-α-L-fucopyranoside for 2-oxoethyl 2,3,4,6-tetra-O-acetyl-α-D-mannopyranoside in Step 4. UPLC-MS Method A: m/z=1020.3 (z=2); t_{R}=4.23 min.

### Example 20: 2,5-Dioxopyrrolidin-1-yl 1-[(β-L-fucopyranosyl)oxy]-13-(2-{[6-({2-[(β-L-fucopyranosyl)oxy]ethyl}[2-({α-D-mannopyranosyl-(1→3)-[α-D-mannopyranosyl-(1→6)]-α-D-mannopyranosyl}oxy)ethyl]amino)hexyl]amino}-2-oxoethyl)-11,15-dioxo-3-[2-({α-D-mannopyranosyl-(1→3)-[α-D-mannopyranosyl-(1→6)]-α-D-mannopyranosyl}oxy)ethyl]-3,10,13,16-tetraazadocosan-22-oate (ML-20)

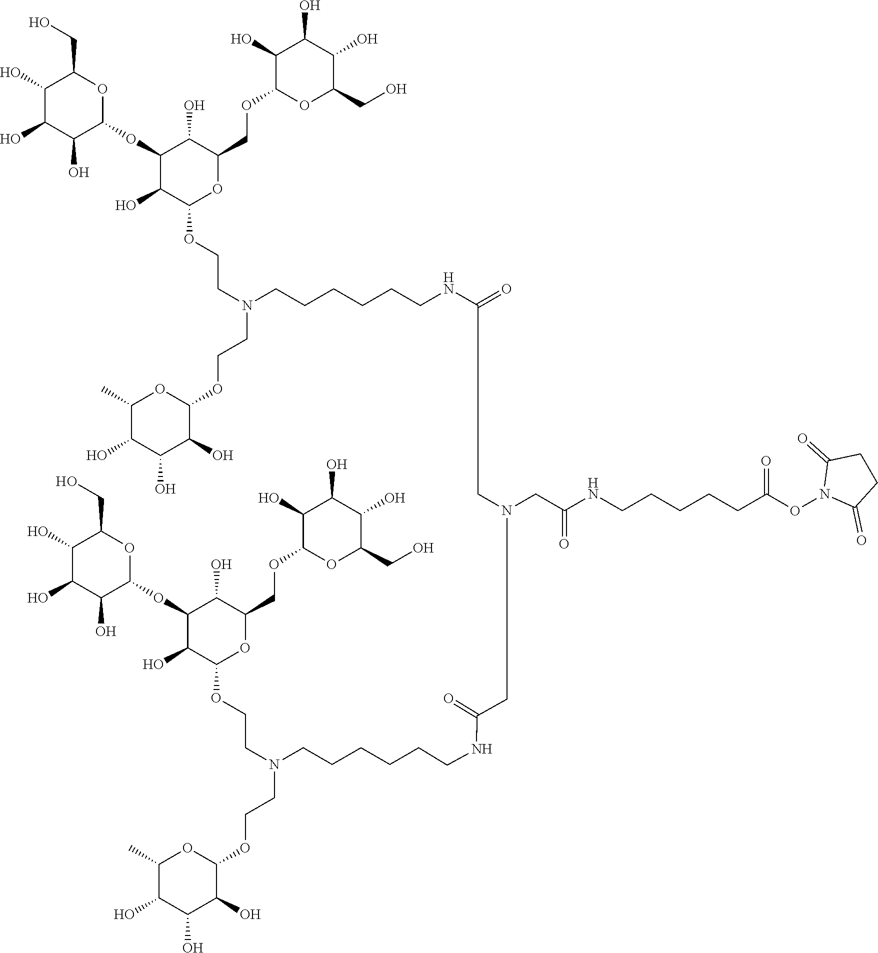 [SMILES: C[C@@H]1O[C@H](OCCN(CCCCCCNC(=O)CN(CC(=O)NCCCCCCN(CCO[C@H]2O[C@@H](C)[C@@H](O)[C@@H](O)[C@@H]2O)CCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)[C@@H](O)[C@H](O)[C@@H]1O]

The title compound was prepared using procedures analogous to those described for Example 17, Steps 4 to 7, substituting 2-oxoethyl 2,3,4-tri-O-acetyl-β-L-fucopyranoside for 2-oxoethyl 2,3,4,6-tetra-O-acetyl-α-D-mannopyranoside in Step 4. UPLC-MS Method A: m/z=1020.5 (z=2); t_{R}=3.94 min.

### Example 21: 2,5-Dioxopyrrolidin-1-yl 1-[(β-D-glucopyranosyl)oxy]-13-(2-{[6-({2-[(β-D-glucopyranosyl)oxy]ethyl}[2-({α-D-mannopyranosyl-(1→3)-[α-D-mannopyranosyl-(1→6)]-α-D-mannopyranosyl}oxy)ethyl]amino)hexyl]amino}-2-oxoethyl)-11,15-dioxo-3-[2-({α-D-mannopyranosyl-(1→3)-[α-D-mannopyranosyl-(1→6)]-α-D-mannopyranosyl}oxy)ethyl]-3,10,13,16-tetraazadocosan-22-oate (ML-21)

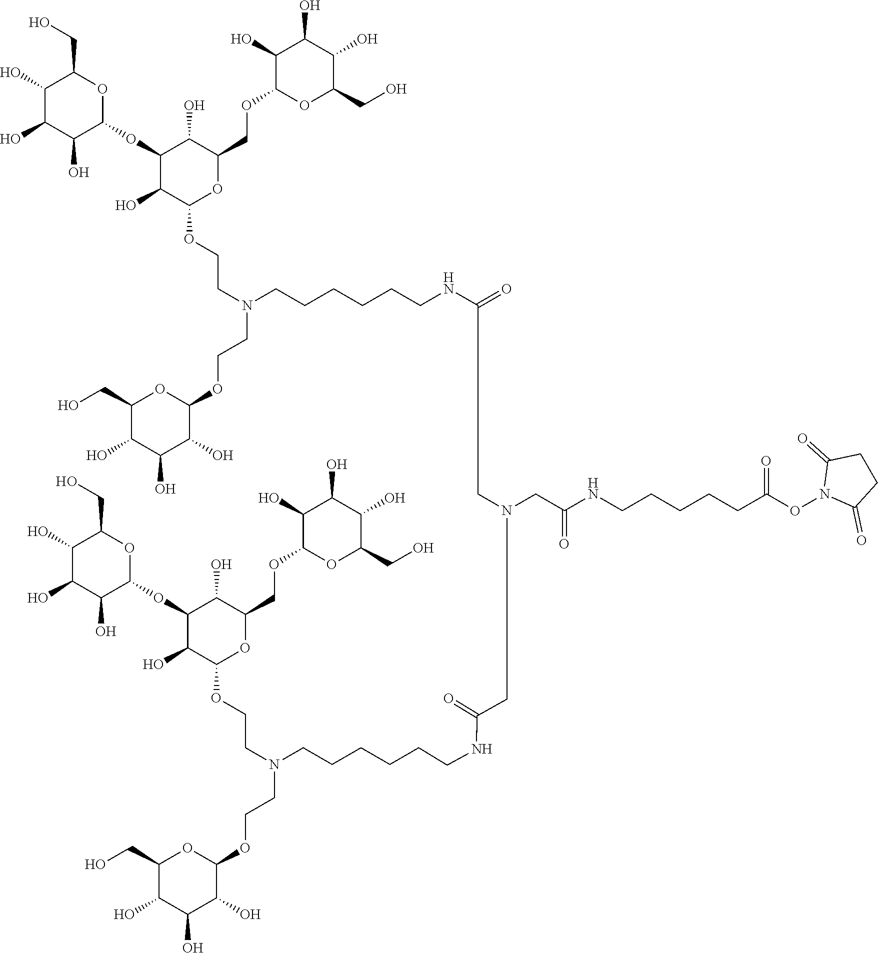 [SMILES: O=C(CN(CC(=O)NCCCCCCN(CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CCO[C@@H]1O[C@H](CO)[C@@H](O)[C@H](O)[C@H]1O)CC(=O)NCCCCCC(=O)ON1C(=O)CCC1=O)NCCCCCCN(CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CCO[C@@H]1O[C@H](CO)[C@@H](O)[C@H](O)[C@H]1O]

The title compound was prepared using procedures analogous to those described for Example 17, Step 4 to 7, substituting 2-oxoethyl 2,3,4,6-tri-O-acetyl-β-L-fucopyranoside for 2-oxoethyl 2,3,4,6-tetra-O-acetyl-α-D-mannopyranoside in Step 4. UPLC-MS Method A: m/z=1036.5 (z=2); t_{R}=4.23 min.

### Example 22: 2,5-Dioxopyrrolidin-1-yl (S)-18-[2-({6-[((S)-1,5-bis{[2-({α-D-mannopyranosyl-(1→3)-[α-D-mannopyranosyl-(1→6)]-α-D-mannopyranosyl}oxy)ethyl]amino}-1,5-dioxopentan-2-yl)amino]-6-oxohexyl}amino)-2-oxoethyl]-1-({α-D-mannopyranosyl-(1→3)-[α-D-mannopyranosyl-(1→6)]-α-D-mannopyranosyl}oxy)-7-{[2-({α-D-mannopyranosyl-(1→3)-[α-D-mannopyranosyl-(1→6)]-α-D-mannopyranosyl}oxy)ethyl]carbamoyl}-4,9,16,20-tetraoxo-3,8,15,18,21-pentaazaheptacosan-27-oate (ML-22)

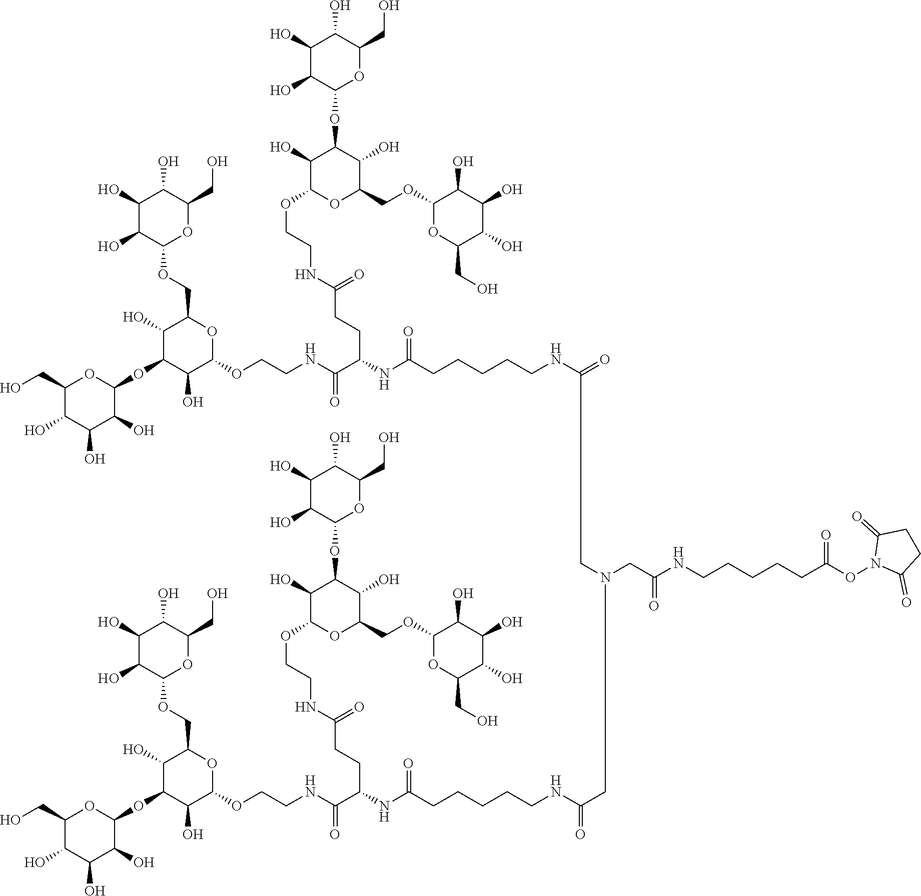

### Step 1: (6-{[(Benzyloxy)carbonyl]amino}hexanoyl)-L-glutamic acid

To a stirred solution of (S)-2-aminopentanedioic acid (5.0 g, 34.0 mmol) in DMF (100 mL) at 0° C. was added 2,5-dioxopyrrolidin-1-yl 6-{[(benzyloxy)carbonyl]amino}hexanoate (14.78 g, 40.8 mmol) in DMF (20 mL) and 5 min later Hunig's Base (17.81 mL, 102 mmol). After stirring at rt for overnight, the reaction mixture was concentrated, and residue was purified by reverse phase C18 column (128 g), eluting with 0-40% ACN in water, to afford the desired product.

### Step 2: bis(2,5-Dioxopyrrolidin-1-yl) (6-{[(benzyloxy)carbonyl)amino]hexanoyl}-L-glutamate

To a solution of (6-{[(benzyloxy)carbonyl]amino}hexanoyl)-L-glutamic acid (1.08 g, 2.74 mmol) in DMF (10 mL) at 0° C. was added TSTU (2.061 g, 6.85 mmol) in DMF (5 mL) and Hunig's Base (1.052 mL, 6.02 mmol). After stirring at 0° C. for 2 h, the crude product was used without further purification.

### Step 3: Benzyl (S)-{6-[(1,5-bis{[2-({α-D-mannopyranosyl-(1→3)-[α-D-mannopyranosyl-(1-+6)]-α-D-mannopyranosyl}oxy)ethyl]amino}-1,5-dioxopentan-2-yl)amino]-6-oxohexyl}carbamate

To a stirred solution of 2-({α-D-mannopyranosyl-(1-3)-[α-D-mannopyranosyl-(1→6)]-α-D-mannopyranosyl}oxy)ethan-1-amine (2.260 g, 4.13 mmol) in DMF (10 mL) at 0° C. was added bis(2,5-dioxopyrrolidin-1-yl) (6-{[(benzyloxy)carbonyl)amino]hexanoyl}-L-glutamate (0.81 g, 1.376 mmol) in DMF and Hunig's Base (481 μL, 2.75 mmol). After stirring at rt for 5 h, the reaction was incomplete. To the reaction mixture at 0° C. was added DMAP (50 mg, 0.413 mmol) in DMF (15 mL) and then EDC (264 mg, 1.376 mmol). After stirring at rt for overnight, the reaction mixture was concentrated, and the residue was re-dissolved in water and purified by reverse phase C18 column (128 g), eluting with 0-40% ACN in water to give the title compound.

### Step 4: (S)-2-(6-Aminohexanamido)-N¹,N⁵-bis[2-({α-D-mannopyranosyl-(1→3)-[α-D-mannopyranosyl-(1→6)]-α-D-mannopyranosyl}oxy)ethyl]pentanediamide

The title compound was prepared using the procedure analogous to that described for Example 1, Step 3, substituting benzyl (S)-{6-[(1,5-bis{[2-({α-D-mannopyranosyl-(1-3)-[α-D-mannopyranosyl-(1→6)]-α-D-mannopyranosyl}oxy)ethyl]amino}-1,5-dioxopentan-2-yl)amino]-6-oxohexyl}carbamate for benzyl [6-({2-[(α-D-mannopyranosyl)oxy]ethyl}amino)-6-oxohexyl]carbamate. UPLC-MS Method A: m/z=685.0 (z=1); t_{R}=2.09 min.

### Step 5: 2,5-Dioxopyrrolidin-1-yl (S)-18-[2-({6-[((S)-1,5-bis{[2-({α-D-mannopyranosyl-(1→3)-[α-D-mannopyranosyl-(1→6)]-α-D-mannopyranosyl}oxy)ethyl]amino}-1,5-dioxopentan-2-yl)amino]-6-oxohexyl}amino)-2-oxoethyl]-1-({α-D-mannopyranosyl-(1→3)-[α-D-mannopyranosyl-(1→6)]-α-D-mannopyranosyl}oxy)-7-{[2-({α-D-mannopyranosyl-(1→3)-[α-D-mannopyranosyl-(1→6)]-α-D-mannopyranosyl}oxy)ethylcarbamoyl}-4,9,16,20-tetraoxo-3,8,15,18,21-pentaazaheptacosan-27-oate

The title compound was prepared using procedures analogous to those described for Example 1, Steps 8 to 10, substituting (S)-2-(6-aminohexanamido)-N¹,N⁵-bis[2-({α-D-mannopyranosyl-(1→3)-[α-D-mannopyranosyl-(1→6)]-α-D-mannopyranosyl}oxy)ethyl]pentanediamide for 6-{2-[{2-[(5-aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino}ethyl)amino]acetamido}-N-{2-[(α-D-mannopyranosyl)oxy]ethyl}hexanamide in Step 8. UPLC-MS Method A: m/z=1502.63, t_{R}=4.04 min.

### Example 23: 2,5-Dioxopyrrolidin-1-yl 6-{bis[2-(bis{2-[(α-D-mannopyranosyl)oxy]ethyl}amino)-2-oxoethyl]amino}-6-oxohexanoate (ML-23)

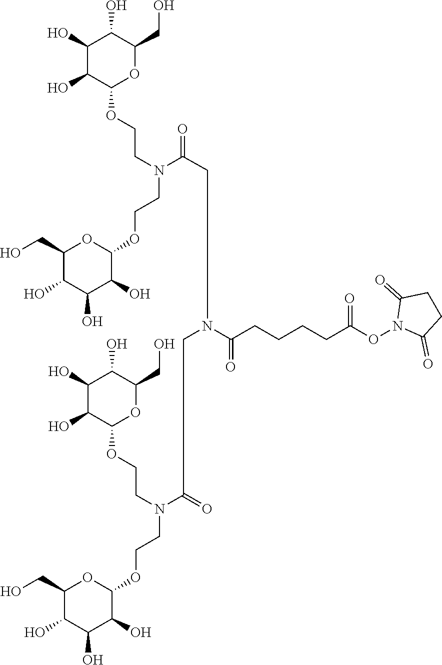 [SMILES: O=C(CCCCC(=O)N(CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)ON1C(=O)CCC1=O]

### Step 1: Benzyl 6-{bis[2-(bis{2-[(2,3,4,6-tetra-O-acetyl-α-D-mannopyranosyl)oxy]ethyl}amino)-2-oxoethyl]amino}-6-oxohexanoate

To a solution of 2,2′-((6-(benzyloxy)-6-oxohexanoyl)azanediyl)diacetic acid (600 mg, 1.708 mmol, WO 2015/051052 Å2) and bis{2-[(2,3,4,6-tetra-O-α-D-mannopyranosyl)oxy]ethyl}amine (3.27 g, 4.27 mmol, WO 2018/175272 A1) in DMF (15 mL) at rt was added DIPEA (1.491 mL, 8.54 mmol), HOBt (523 mg, 3.42 mmol) and EDC (1.309 g, 6.83 mmol). After stirring overnight, the reaction mixture was concentrated, and the residue was re-dissolved in EtOAc (100 mL). The mixture was washed with 1N HCl (100 mL), sat. NaHCO₃ (100 mL), and brine (100 mL). The mixture was filtered, and the filtrate was dried over Na₂SO₄ and concentrated. The residue was purified by silica gel chromatography (80 g), flow rate=80 mL/min, gradient 0-10000 of EtOAc in hexanes, to give the title compound. UPLC-MS Method A: m/z=1846.7 (z=1); t_{R}=4.69 min.

### Step 2: 6-{bis[2-(bis{2-[(α-D-Mannopyranosyl)oxy]ethyl}amino)-2-oxoethyl]amino}-6-oxohexanoic acid

To a stirred solution of benzyl 6-{bis[2-(bis{2-[(2,3,4,6-tetra-O-acetyl-α-D-mannopyranosyl)oxy]ethyl}amino)-2-oxoethyl]amino}-6-oxohexanoate (2.2 g, 1.191 mmol) in MeOH (10 mL) at rt was added NaOCH₃ (32 mg, 0.596 mmol). After stirring at rt for 30 min, the reaction mixture was concentrated. The residue was re-dissolved in water (12 mL), to which was added NaOH (2.382 mL, 2.382 mmol, 1N) at rt, and the resulting mixture was stirred at rt for overnight. The pH value of the reaction mixture was then adjusted to 5 using 1N HCl. The resulting mixture was concentrated and purified by reverse phase chromatography (130 g C18). B=0-40% CH₃CN/Water in 1 h, to give the title compound.

### Step 3: 2,5-Dioxopyrrolidin-1-yl 6-{bis[2-(bis{2-[(α-D-mannopyranosyl)oxy]ethyl}amino)-2-oxoethyl]amino}-6-oxohexanoate

The title compound was prepared using the procedure analogous to that described for Example 1, Step 10, substituting 6-{bis[2-(bis{2-[(α-D-mannopyranosyl)oxy]ethyl}amino)-2-oxoethyl]amino}-6-oxohexanoic acid for 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oic acid. UPLC-MS Method A: m/z=1181.6 (z=1); t_{R}=0.2 min.

### Example 24: 2,5-Dioxopyrrolidin-1-yl 6-(6-{bis[2-(bis{2-[(α-D-mannopyranosyl)oxy]ethyl}amino)-2-oxoethyl]amino}-6-oxohexanamido)hexanoate (ML-24)

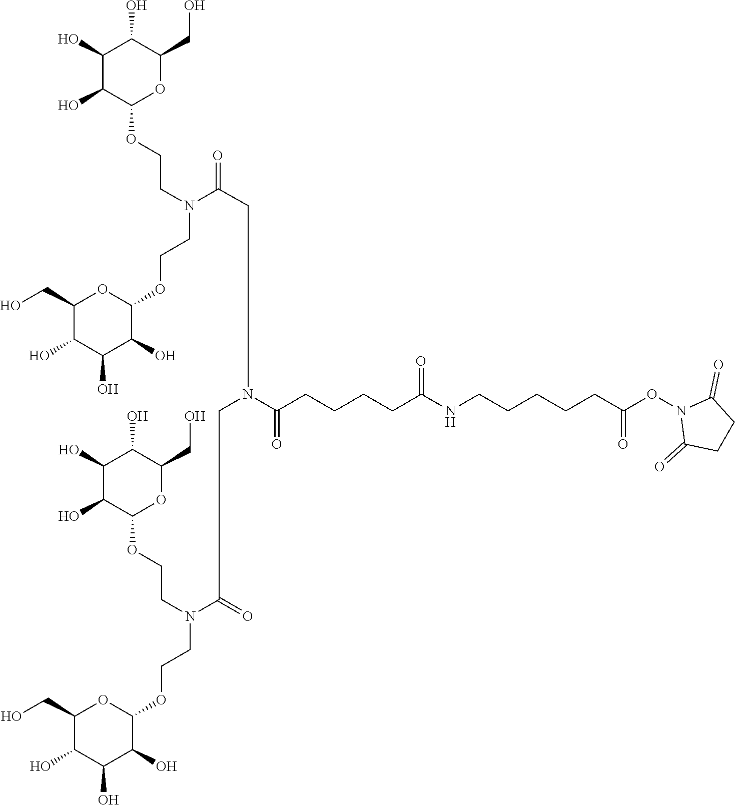 [SMILES: O=C(CCCCC(=O)N(CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NCCCCCC(=O)ON1C(=O)CCC1=O]

The title compound was prepared using procedures analogous to those described for Example 9, Steps 1 to 2, substituting 2,5-dioxopyrrolidin-1-yl 6-{bis[2-(bis{2-[(α-D-mannopyranosyl)oxy]ethyl}amino)-2-oxoethyl]amino}-6-oxohexanoate (ML-23) for 2,5-dioxopyrrolidin-1-yl (S)-11-(2-{[(2S)-1,5-bis({2-[(α-D-mannopyranosyl)oxy]ethyl}amino)-1,5-dioxopentan-2-yl]amino}-2-oxoethyl)-1-[(α-D-mannopyranosyl)oxy]-7-({2-[(α-D-mannopyranosyl)oxy]ethyl}carbamoyl)-4,9,13-trioxo-3,8,11,14-tetraazaicosan-20-oate (ML-8) in Step 1. UPLC-MS Method A: m/z=1294.6 (z=1); t_{R}=3.44 min.

### Example 25: 2,5-Dioxopyrrolidin-1-yl (S)-19-(2-{[6-({(2S)-1,6-bis[(2-{α-D-mannopyranosyl-(1→3)-[α-D-mannopyranosyl-(1→6)]-α-D-mannopyranosyl}ethyl)amino]-1,6-dioxohexan-2-yl}amino)-6-oxohexyl]amino}-2-oxoethyl)-1-{α-D-mannopyranosyl-(1→3)-[α-D-mannopyranosyl-(1→6)]-α-D-mannopyranosyl}-8-[(2-{α-D-mannopyranosyl-(1→3)-[α-D-mannopyranosyl-(1→6)]-α-D-mannopyranosyl}ethyl)carbamoyl]-4,10,17,21-tetraoxo-3,9,16,19,22-pentaazaoctacosan-28-oate (ML-25)

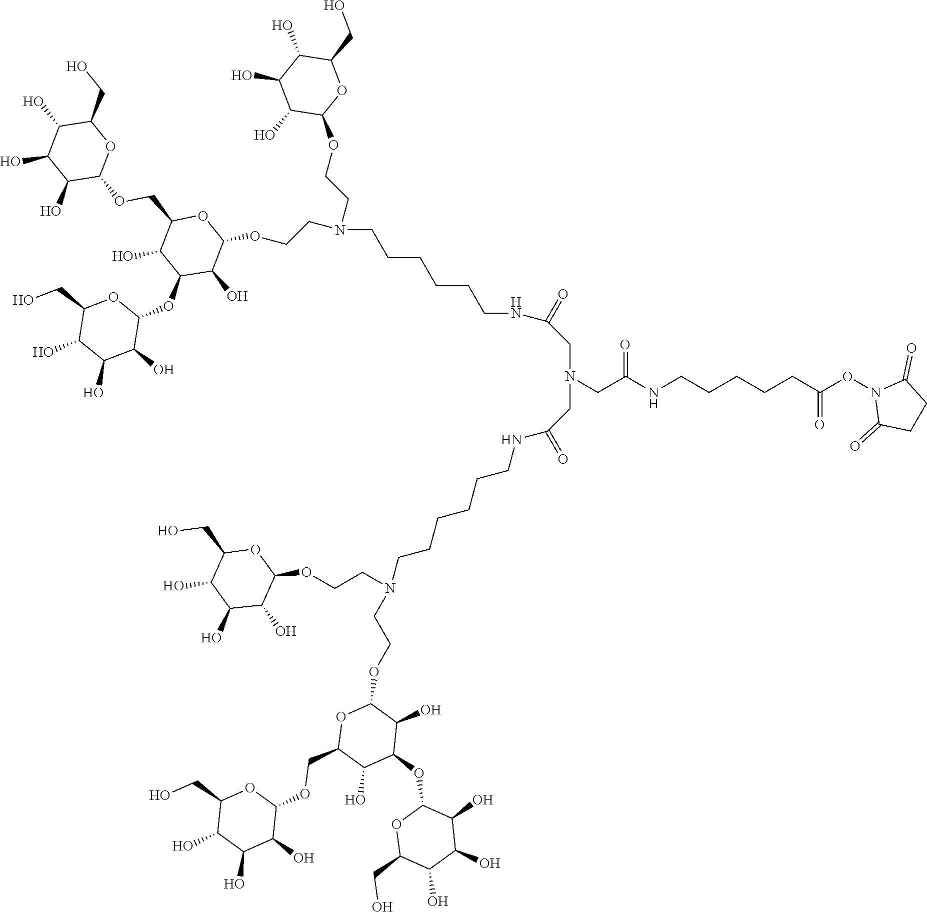 [SMILES: O=C(CN(CC(=O)NCCCCCCN(CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CCO[C@@H]1O[C@H](CO)[C@@H](O)[C@H](O)[C@H]1O)CC(=O)NCCCCCC(=O)ON1C(=O)CCC1=O)NCCCCCCN(CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CCO[C@@H]1O[C@H](CO)[C@@H](O)[C@H](O)[C@H]1O]

The title compound was prepared using procedures analogous to those described for Example 22, substituting (S)-2-aminohexanedioic acid for (S)-2-aminopentanedioic acid in Step 1. UPLC-MS Method A: m/z=1516.76, t_{R}=1.28 min.

### Example 26: 2,5-Dioxopyrrolidin-1-yl 6-[2-(bis{2-[(3S,5R)-3,5-bis({2-[(α-L-fucopyranosyl) oxy]ethyl}carbamoyl)piperidin-1-yl]-2-oxoethyl}amino)acetamido]hexanoate (ML-26)

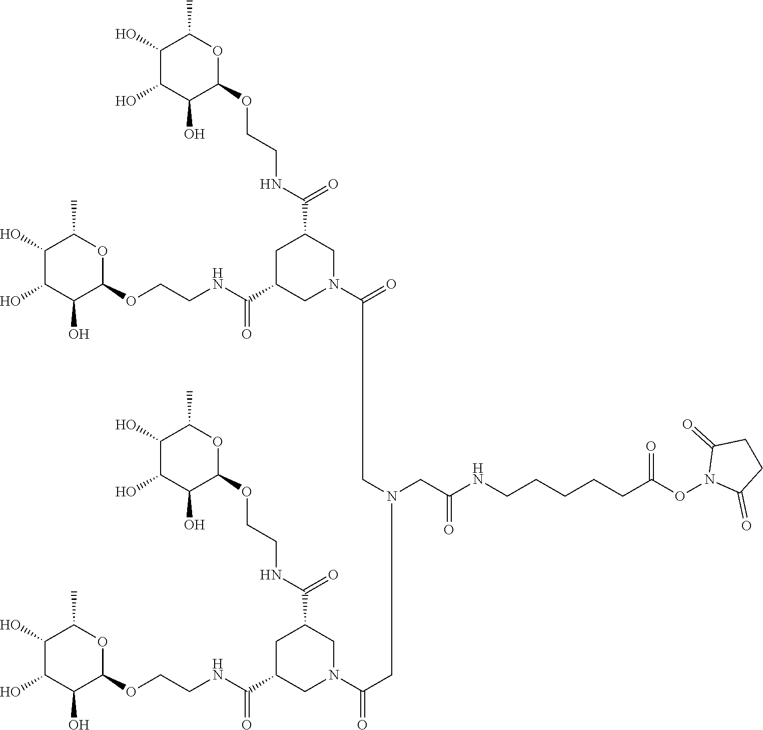 [SMILES: C[C@@H]1O[C@@H](OCCNC(=O)[C@@H]2C[C@H](C(=O)NCCO[C@@H]3O[C@@H](C)[C@@H](O)[C@@H](O)[C@@H]3O)CN(C(=O)CN(CC(=O)NCCCCCC(=O)ON3C(=O)CCC3=O)CC(=O)N3C[C@H](C(=O)NCCO[C@@H]4O[C@@H](C)[C@@H](O)[C@@H](O)[C@@H]4O)C[C@H](C(=O)NCCO[C@@H]4O[C@@H](C)[C@@H](O)[C@@H](O)[C@@H]4O)C3)C2)[C@@H](O)[C@H](O)[C@@H]1O]

### Step 1: N³,N⁵-bis{2-[(α-L-Fucopyranosyl)oxy]ethyl}pyridine-3,5-dicarboxamide

To a stirred solution of pyridine-3,5-dicarboxylic acid (400 mg, 2.394 mmol) in DMF (30 mL) at rt was added 2-aminoethyl α-L-fucopyranoside (1.488 g, 7.18 mmol), DMAP (731 mg, 5.98 mmol) and EDC (2.294 g, 11.97 mmol). After stirring for overnight, the reaction mixture was diluted with water (30 mL), and the mixture was extracted with EtOAc (3×30 mL). The aqueous layer was separated and concentrated. The residue was purified by reverse phase chromatography (C18, 300 g), eluting with B=0-30% water/ACN, to give the title compound.

### Step 2: (3R,5S)—N³,N⁵-bis{2-[(α-L-Fucopyranosyl)oxyethyl}piperidine-3,5-dicarboxamide

To a stirred solution of N³,N⁵-bis{2-[(α-L-fucopyranosyl)oxy]ethyl}pyridine-3,5-dicarboxamide (890 mg, 1.631 mmol) in water (20 mL) at rt was added PtO₂ (111 mg, 0.489 mmol). The mixture was degassed and allowed to stir under a balloon of H₂ at rt. After stirring for 2 h, the reaction mixture was filtered through a cake of CELITE™ diatomaceous earth and washed with water, and the filtrates were combined and concentrated. The residue was redissolved in MeOH and centrifuged. The supernatant was concentrated to give the desired product, which was used without further purification.

### Step 3: 2,5-Dioxopyrrolidin-1-yl 6-[2-(bis{2-[(3S,5R)-3,5-bis({2-[(α-L-fucopyranosyl)oxy]ethyl} carbamoyl)piperidin-1-yl]-2-oxoethyl}amino)acetamido]hexanoate

The title compound was prepared using procedures analogous to those described for Example 1, Steps 8 to 10, substituting (3R,5S)—N³,N⁵-bis{2-[(α-L-fucopyranosyl)oxy]ethyl}piperidine-3,5-dicarboxamide for 6-{2-[{2-[(5-aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino}ethyl)amino]acetamido}-N-{2-[(α-D-mannopyranosyl)oxy]ethyl}hexanamide in Step 8. UPLC-MS Method A: m/z=1468.72 (z=1); t_{R}=2.24 min.

### Example 27: 2,5-Dioxopyrrolidin-1-yl 6-(2-{bis[2-(bis{[1-(α-D-mannopyranosyl)-1H-1,2,3-triazol-4-yl]methyl}amino)-2-oxoethyl]amino}acetamido)hexanoate (ML-27)

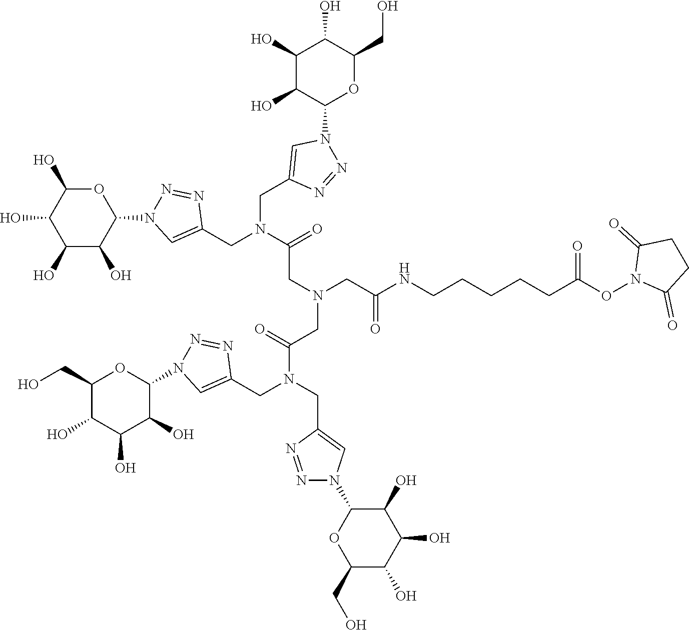 [SMILES: O=C(CN(CC(=O)N(Cc1cn([C@H]2O[C@H](O)[C@@H](O)[C@H](O)[C@@H]2O)nn1)Cc1cn([C@H]2O[C@H](CO)[C@@H](O)[C@H](O)[C@@H]2O)nn1)CC(=O)N(Cc1cn([C@H]2O[C@H](CO)[C@@H](O)[C@H](O)[C@@H]2O)nn1)Cc1cn([C@H]2O[C@H](CO)[C@@H](O)[C@H](O)[C@@H]2O)nn1)NCCCCCC(=O)ON1C(=O)CCC1=O]

### Step 1: Benzyl di(prop-2-yn-1-yl)carbamate

To a stirred solution benzyl prop-2-yn-1-ylcarbamate (3.0 g, 15.86 mmol) in DMF (3.96 mL) at 0° C. was added NaH (824 mg, 20.61 mmol, 60% dispersion in mineral oil) and, 10 min later, 3-bromoprop-1-yne (4.715 g, 31.7 mmol, 80% in toluene). After stirring at rt overnight, the reaction mixture was partitioned a mixture of (150 mL, hexanes/EtOAc v/v=2:1) and water (100 mL). The aqueous layer was separated and extracted with a mixture (50 mL, hexanes/EtOAc v/v=2:1). The organic layers were combined, dried over Na₂SO₄, and concentrated. The residue was purified by silica gel chromatography (80 g), eluting with 0-100% EtOAc/Hex, to give the title compound.

### Step 2: Benzyl bis{[1-(2,3,4,6-tetra-O-acetyl-α-D-mannopyranosyl)-1H-1,2,3-triazol-4-yl]methyl}carbamate

To a mixture of benzyl di(prop-2-yn-1-yl)carbamate (0.5 g, 2.200 mmol) and 2,3,4,6-tetra-O-acetyl-α-D-mannopyranosyl azide (2.464 g, 6.60 mmol) in DMF (11.00 mL) was added DIPEA (1.921 ml, 11.00 mmol) and CuI (419 mg, 2.200 mmol). After stirring at 60° C. for 30 min, the reaction mixture was stirred at rt overnight and then partitioned between EtOAc (150 mL) and water (100 mL). The inorganic solids were filtered off, and the aqueous layer was separated and further extracted with EtOAc (3×50 mL). The organic layers were combined, extracted with brine, dried over Na₂SO₄ and concentrated. The residue was purified on silica gel chromatography (120 g), eluting with 0-50% EtOAc/Hex, to give the title compound. UPLC-MS indicates formation of product at t_{R}=2.19 min, m/z=974.

### Step 3: Benzyl bis{[1-(α-D-mannopyranosyl)-1H-1,2,3-triazol-4-yl]methyl}carbamate

To a solution of benzyl bis{[1-(2,3,4,6-tetra-O-acetyl-α-D-mannopyranosyl)-1H-1,2,3-triazol-4-yl]methyl}carbamate (1.70 g, 1.746 mmol) in MeOH (14.55 mL) was added NaOCH₃ (94 mg, 1.746 mmol). After stirring at rt for 3 h, to the reaction mixture was added hydrochloric acid (2.62 mL, 2.62 mmol) with external cooling. The solvents were removed using a rotary evaporator with the bath temperature of 45° C., and traces of HCl were removed by co-evaporation with 20 mL of water. The reaction appeared complete with t_{R}=1.04 min and m/z=638.

### Step 4: bis{[1-(α-D-Mannopyranosyl)-1H-1,2,3-triazol-4-yl]methyl}amine

To a solution of benzyl bis{[1-(α-D-mannopyranosyl)-1H-1,2,3-triazol-4-yl]methyl}carbamate (1.2 g, 1.724 mmol) in water (34.5 mL) was added Pearlman's catalyst (20% palladium hydroxide on carbon, 242 mg, 0.345 mmol). The mixture was shaken on Parr shaker under 50 psi of H₂. After shaking for 2 h, the reaction mixture was filtered through a cake of CELITE™ diatomaceous earth. The filtrate was freeze-dried to give the title compound. LCMS indicated reaction complete with the product eluting at t_{R}=0.09 min m/z=504.

### Step 5: 2,5-Dioxopyrrolidin-1-yl 6-(2-{bis[2-(bis{[1-(α-D-mannopyranosyl)-1H-1,2,3-triazol-4-yl]methyl}amino)-2-oxoethyl]amino}acetamido)hexanoate

The title compound was prepared using procedures analogous to those described for Example 1, Steps 8 to 10, substituting bis{[1-(α-D-mannopyranosyl)-1H-1,2,3-triazol-4-yl]methyl}amine for 6-{2-[{2-[(5-aminopentyl)amino]-2-oxoethyl}(2-oxo-2-{[2-({α-D-mannopyranosyl-(1→3)-[α-D-mannopyranosyl-(1→6)]-α-D-mannopyranosyl}oxy)ethyl]amino}ethyl)amino]acetamido}-N-{2-[(α-D-mannopyranosyl)oxy]ethyl}hexanamide in Step 8. UPLC-MS Method A: m/z=1372.37 (z=1); t_{R}=1.91 min.

### Example 28: 2,5-Dioxopyrrolidin-1-yl 6-(2-{bis[2-(bis{2-[(α-D-mannopyranosyl)oxy]ethyl}amino)-2-oxoethyl]amino}acetamido)hexanoate (ML-28)

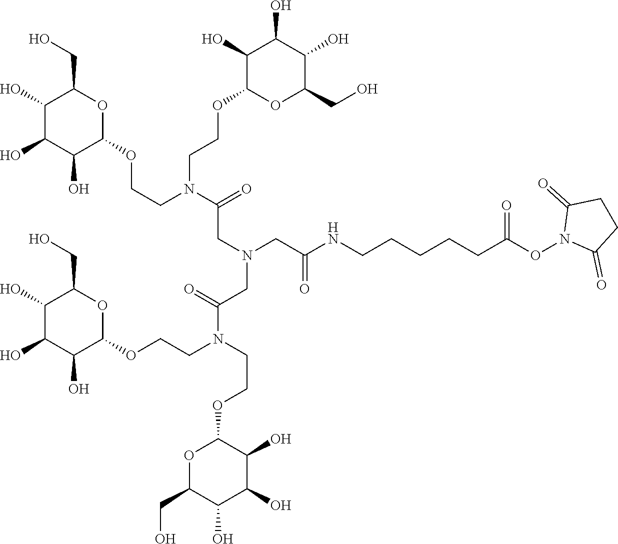 [SMILES: O=C(CN(CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NCCCCCC(=O)ON1C(=O)CCC1=O]

The title compound was prepared using procedures analogous to those described for Example 23, substituting 2,2′-((2-((6-(benzyloxy)-6-oxohexyl)amino)-2-oxoethyl)azanediyl) diacetic acid for 2,2′-((6-(benzyloxy)-6-oxohexanoyl)azanediyl)diacetic acid in Step 1. UPLC-MS Method A: m/z=1224.35 (z=1); t_{R}=1.91 min.

### Example 29: 2,5-Dioxopyrrolidin-1-yl (S)-6-{[1,5-bis(bis{2-[(α-D-mannopyranosyl)oxy]ethyl}amino)-1,5-dioxopentan-2-yl]amino}-6-oxohexanoate (ML-29)

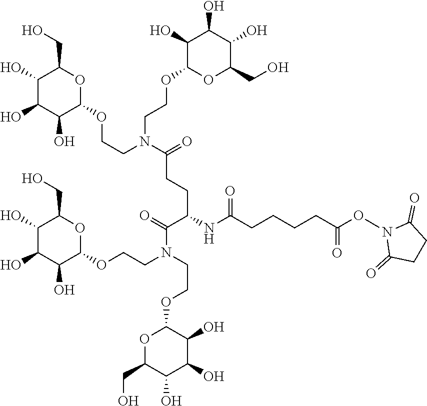 [SMILES: O=C(CCCCC(=O)ON1C(=O)CCC1=O)N[C@@H](CCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)C(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl (S)-[1,5-bis(bis{2-[(2,3,4,6-tetra-O-acetyl-α-D-mannopyranosyl)oxy]ethyl}amino)-1, 5-dioxopentan-2-yl]carbamate

To a stirred solution of N-Cbz-L-glutamic acid (500 mg, 1.778 mmol) and bis{2-[(2,3,4,6-tetra-O-α-D-mannopyranosyl)oxy]ethyl}amine (3.40 g, 4.44 mmol, WO 2018/175272 A1) in DMF (17.8 mL) was added DIPEA (1.863 mL, 10.67 mmol), HOBt (817 mg, 5.33 mmol), and 10 min later EDC (1.022 g, 5.33 mmol). After stirring at overnight, the reaction mixture was concentrated. The residue was redissolved in EtOAc (100 mL), washed with 1N HCl (100 mL), sat. NaHCO₃ (100 mL), and brine (100 mL), dried over Na₂SO₄ and concentrated. The residue was purified by silica gel chromatography (80 g), eluting with 0-100% of EtOAc in hexanes, to give the title compound.

### Step 2: (S)-2-Amino-N′,N¹,N⁵,N⁵-tetrakis{2-[(2,3,4,6-tetra-O-acetyl-α-D-mannopyranosyl)oxy]ethyl}pentanediamide

In a 500 mL round bottom flask filled with N₂ was charged Pearlman's catalyst (214 mg, 0.305 mmol) and a solution of benzyl (S)-[1,5-bis(bis{2-[(2,3,4,6-tetra-O-acetyl-α-D-mannopyranosyl)oxy]ethyl}amino)-1,5-dioxopentan-2-yl]carbamate (2.17 g, 1.221 mmol) in EtOAc (24.43 mL). The resulting mixture was hydrogenated under a balloon of H₂ overnight. The catalyst was filtered off, and the filtrate was concentrated to give the crude title compound for use without further purification.

### Step 3: Benzyl (S)-6-{[1,5-bis(bis{2-[(2,3,4,6-tetra-O-acetyl-α-D-mannopyranosyl)oxy]ethyl}amino)-1,5-dioxopentan-2-yl]amino}-6-oxohexanoate

To a solution of (S)-2-amino-N¹,N¹,N⁵,N⁵-tetrakis{2-[(2,3,4,6-tetra-O-acetyl-α-D-mannopyranosyl)oxy]ethyl}pentanediamide (2.07 g, 1.260 mmol) in DMF (13 μL) at rt was added 6-(benzyloxy)-6-oxohexanoic acid (357 mg, 1.512 mmol), HOBt (232 mg, 1.512 mmol), DIPEA (660 μL, 3.78 mmol), and EDC (290 mg, 1.512 mmol) 5 min later. After stirring at rt overnight, the reaction mixture was partitioned between a mixture of EtOAc (70 mL), hexanes (35 mL), and 1M HCl (50 mL). The organic layer was washed with sat. NaHCO₃ (50 mL), brine (50 mL), dried over Na₂SO₄ and concentrated. The residue was purified by silica gel chromatography (40 g), eluting with 0-100% of EtOAc in hexanes, to give the title compound.

### Step 4: 2,5-Dioxopyrrolidin-1-yl (S)-6-{[1,5-bis(bis{2-[(α-D-mannopyranosyl)oxy]ethyl}amino)-1,5-dioxopentan-2-yl]amino}-6-oxohexanoate

The title compound was prepared using procedures analogous to those described for Example 23, Steps 2 to 3, substituting benzyl (S)-6-{[1,5-bis(bis{2-[(2,3,4,6-tetra-O-acetyl-α-D-mannopyranosyl)oxy]ethyl}amino)-1,5-dioxopentan-2-yl]amino}-6-oxohexanoate for benzyl 6-{bis[2-(bis{2-[(2,3,4,6-tetra-O-acetyl-α-D-mannopyranosyl)oxy]ethyl}amino)-2-oxoethyl]amino}-6-oxohexanoate in Step 2. UPLC-MS Method A: m/z=1195.48 (z=1); t_{R}=3.78 min.

### Example 30: 2,5-Dioxopyrrolidin-1-yl 6-(2-{bis[2-({1,3-bis[(α-D-mannopyranosyl)oxy]propan-2-yl}amino)-2-oxoethyl]amino}acetamido)hexanoate (ML-30)

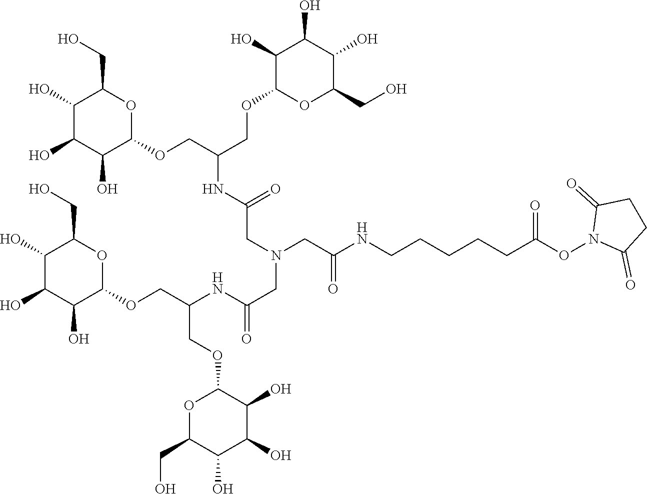 [SMILES: O=C(CN(CC(=O)NC(CO[C@H]1O[C@H](CO)[C@@H](O)[C@H](O)[C@@H]1O)CO[C@H]1O[C@H](CO)[C@@H](O)[C@H](O)[C@@H]1O)CC(=O)NC(CO[C@H]1O[C@H](CO)[C@@H](O)[C@H](O)[C@@H]1O)CO[C@H]1O[C@H](CO)[C@@H](O)[C@H](O)[C@@H]1O)NCCCCCC(=O)ON1C(=O)CCC1=O]

### Step 1: Benzyl {1,3-bis[(2,3,4,6-tetra-O-benzoyl-α-D-mannopyranosyl)oxy]propan-2-yl}carbamate

To a mixture of benzyl (1,3-dihydroxypropan-2-yl)carbamate (230 mg, 1.021 mmol), 2,3,4,6-tetra-O-benzoyl-D-mannopyranosyl trichloroacetimidate (1.6 g, 2.159 mmol, ORG. LETT. 2003, 5, 4041) and dried 4 Å molecular sieves in DCM (25 mL) at −30° C. was added trimethylsilyl trifluoromethanesulfonate (50 μL, 1.021 mmol). The mixture was allowed to gradually warm up to rt. After stirring for 6 h, the reaction was quenched with TEA (400 μL, 2.87 mmol). The reaction mixture was filtered, and the filtrate was concentrated. The residue was purified by flash chromatography on silica gel (80 g, eluting with 0-65% EtOAc in hexanes to give the title compound.

¹H-NMR (CDCl₃) δ 8.05-8.00 (4H, m), 7.95-7.80 (8H, m), 7.75-7.65 (4H, m), 7.55-7.45 (4H, m), 7.40-7.10 (25H, m), 6.15-6.05 (2H, m), 5.85-5.80 (2H, m), 5.75-5.70 (2H, m), 5.30-5.05 (4H, m), 4.70-4.60 (2H, m), 4.55-4.40 (4H, m), 4.25-4.20 (1H, m), 4.08-4.00 (3H, m), 3.90-3.85 (1H, m), 3.75-3.70 (1H, m).

### Step 2: Benzyl {1,3-bis[(α-D-mannopyranosyl)oxy]propan-2-yl}carbamate

To a solution of benzyl {1,3-bis[(2,3,4,6-tetra-O-benzoyl-α-D-mannopyranosyl)oxy]propan-2-yl}carbamate (1.05 g, 0.76 mmol) in CH₃OH (20 mL) was added NaOCH₃ (250 μL, 0.125 mmol, 0.5M in CH₃OH). After stirring at rt for 24 h, an ion exchange resin (AMBERLITE™ IR 120, CAS Registry No. 78922-04-0) ion exchange resin (pre-washed with CH₃OH 3×20 mL) was added to the reaction mixture. The resulting mixture was allowed to stir for additional 15 min. The resin was filtered off and washed with CH₃OH (3×5 mL). The filtrate was concentrated to give the title compound. UPLC Method A: calculated for C₂₃H₃₅NO₁₄ 549.21, observed m/e: 550.26 [M+1]; t_{R}=4.15 min.

### Step 3: 2,5-Dioxopyrrolidin-1-yl 6-(2-{bis[2-({1,3-bis[(α-D-mannopyranosyl)oxy]propan-2-yl}amino)-2-oxoethyl]amino}acetamido)hexanoate

The title compound was prepared using procedures analogous to those described for Example 1, Steps 7 to 10, substituting benzyl {1,3-bis[(α-D-mannopyranosyl)oxy]propan-2-yl}carbamate for benzyl {1-[(α-D-mannopyranosyl)oxy]-4,11,15-trioxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16-tetraazahenicosan-21-yl}carbamate acid in Step 7. UPLC-MS Method B: calculated exact mass for C₄₆H₇₇N₅O₃₁ 1195.46, observed m/z=1196.55 (z=1); t_{R}=1.18 min.

### Example 31: 2,5-Dioxopyrrolidin-1-yl 6-(2-{bis[2-({(R)-1,4-bis[(α-D-mannopyranosyl)oxy]butan-2-yl}amino)-2-oxoethyl]amino}acetamido)hexanoate (ML-31)

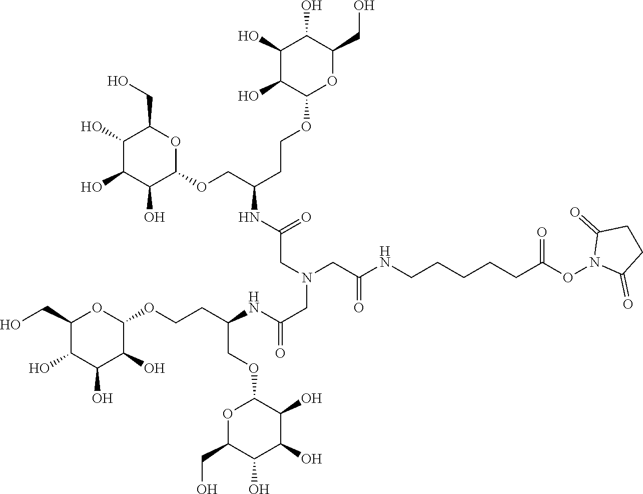 [SMILES: O=C(CN(CC(=O)N[C@H](CCO[C@H]1O[C@H](CO)[C@@H](O)[C@H](O)[C@@H]1O)CO[C@H]1O[C@H](CO)[C@@H](O)[C@H](O)[C@@H]1O)CC(=O)N[C@H](CCO[C@H]1O[C@H](CO)[C@@H](O)[C@H](O)[C@@H]1O)CO[C@H]1O[C@H](CO)[C@@H](O)[C@H](O)[C@@H]1O)NCCCCCC(=O)ON1C(=O)CCC1=O]

The title compound was prepared using procedures analogous to those described for Example 30, substituting benzyl (R)-(1,4-dihydroxybutan-2-yl)carbamate for benzyl (1,3-dihydroxypropan-2-yl)carbamate in Step 1. UPLC-MS Method B: calculated for exact mass C₄₈H₈₁N₅O₃₁ 1223.49, observed m/z=1224.50 (z=1); t_{R}=1.31 min.

### Example 32: 2,5-Dioxopyrrolidin-1-yl (2R)-3-(2,4-bis{bis[2-({2-[(α-D-mannopyranosyl)oxy]ethyl}amino)-2-oxoethyl]amino}butanamido)propanoate (ML-32)

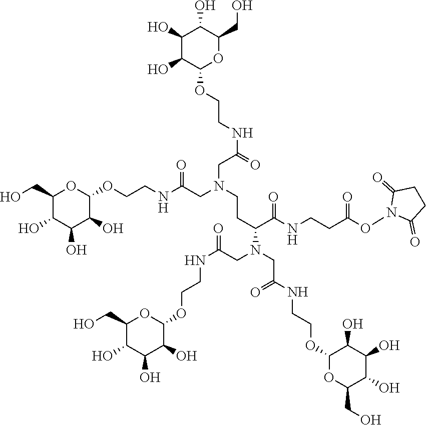 [SMILES: O=C(CN(CC[C@H](C(=O)NCCC(=O)ON1C(=O)CCC1=O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O)NCCO[C@H]1O[C@H](CO)[C@@H](O)[C@H](O)[C@@H]1O]

### Step 1: Benzyl (R)-3-{2,4-bis[(tert-butoxycarbonyl)amino]butanamido}propanoate

To a stirred solution of (R)-2,4-bis[(tert-butoxycarbonyl)amino]butanoic acid (7.99 g, 25.10 mmol) in DMF (85 mL) at was added DIPEA (17.53 mL, 100 mmol), HOBt (7.69 g, 50.2 mmol), EDC (9.62 g, 50.2 mmol) and β-alanine benzyl ester p-toluenesulfonate salt (13.23 g, 37.6 mmol). After stirring at rt for overnight, the reaction mixture was concentrated and partitioned between a mixture of EtOAc (100 mL), hexanes (50 mL), and sat. NaHCO₃ (100 mL). The organic phase was separated and washed with brine (100 mL), dried over Na₂SO₄, and concentrated. The residue was purified by silica gel chromatography (80 g), eluting with 0-60% of EtOAc in hexanes, to give the title compound. UPLC-MS Method A: m/z=480.28 (z=1); t_{R}=2.57 min.

### Step 2: Benzyl (R)-3-(2,4-diaminobutanamido)propanoate

A mixture of (R)-benzyl 3-{2,4-bis[(tert-butoxycarbonyl)amino]butanamido} propanoate (4.32 g, 9.01 mmol) in formic acid (60 mL, 1564 mmol) was stirred at rt for overnight. The reaction mixture was concentrated to give the title product. UPLC-MS Method A: m/z=280.12 (z=1); t_{R}=0.40 min.

### Step 3: Benzyl (R)-7-(2-{bis[2-(tert-butoxy)-2-oxoethyl]amino}ethyl)-6-[2-(tert-butoxy)-2-oxoethyl]-2,2-dimethyl-4,8-dioxo-3, 5-dioxa-6,9-diazadodecan-12-oate

To a stirred solution of (R)-benzyl 3-(2,4-diaminobutanamido)propanoate (2.52 g, 9.02 mmol) in DMF (65 mL) at 0° C. was added DIPEA (18.91 mL, 108 mmol) and tert-butyl bromoacetate (5.96 mL, 39.7 mmol). After stirring at rt for overnight, the reaction mixture was diluted with water (150 mL) and extracted with EtOAc (2×200 mL). The organic phase was separated, dried over MgSO₄ and concentrated. The residue was purified by silica gel chromatography (330 g), eluting with 0-60% EtOAc/isohexane, to give the title product. UPLC-MS Method A: m/z=736.55 (z=1); t_{R}=2.81 min.

### Step 4: (R)-2,2′-[(4-{[3-(Benzyloxy)-3-oxopropyl]amino}-3-[(carboxymethyl)(carboxyoxy) amino]-4-oxobutyl)azanediyl]diacetic acid

To a stirred solution of benzyl (R)-7-(2-{bis[2-(tert-butoxy)-2-oxoethyl]amino} ethyl)-6-[2-(tert-butoxy)-2-oxoethyl]-2,2-dimethyl-4,8-dioxo-3,5-dioxa-6,9-diazadodecan-12-oate (1.05 g, 1.427 mmol) in DCM (10 mL) at rt was added TFA (10 mL, 130 mmol). After stirring for overnight, the reaction mixture was concentrated. The residue was purified by reverse phase chromatography (C18 43 g), eluting with 0-50% of water/ACN, to give the title compound. UPLC-MS Method A: m/z=512.12 (z=1); t_{R}=1.42 min.

### Step 5: bis(2,5-Dioxopyrrolidin-1-yl) 2,2′-[(4-{[3-(benzyloxy)-3-oxopropyl]amino}-3-({2-[(2,5-dioxopyrrolidin-1-yl)oxy]-2-oxoethyl}({[(2,5-dioxopyrrolidin-1-yl)oxy]carbonyl}oxy)amino)-4-oxobutyl)azanediyl](R)-diacetate

To a stirred solution of (R)-2,2′-[(4-{[3-(benzyloxy)-3-oxopropyl]amino}-3-[(carboxymethyl)(carboxyoxy)amino]-4-oxobutyl)azanediyl]diacetic acid (150 mg, 0.293 mmol) in DMF (3 mL) at 0° C. was added TSTU (441 mg, 1.466 mmol) in DMF (2 mL) and 5 min later DIPEA (255 μL, 1.466 mmol). After stirring at rt for 2 h, the crude product without further purification was taken onto next step. UPLC-MS Method A: m/z=900.48 (z=1); t_{R}=2.18 min.

### Step 6: Benzyl (2R)-3-(2,4-bis{bis[2-({2-[(α-D-mannopyranosyl)oxy]ethyl}amino)-2-oxoethyl]amino}butanamido)propanoate

To a stirred solution of 2-aminoethyl α-D-mannopyranoside (655 mg, 2.93 mmol) in DMF (4 mL) at 0° C. was added and the mixture was stirred bis(2,5-dioxopyrrolidin-1-yl) 2,2′-[(4-{[3-(benzyloxy)-3-oxopropyl]amino}-3-({2-[(2,5-dioxopyrrolidin-1-yl)oxy]-2-oxoethyl} ({[(2,5-dioxopyrrolidin-1-yl)oxy]carbonyl}oxy)amino)-4-oxobutyl)azanediyl](R)-diacetate (264 mg, 0.293 mmol) in DMF (5 mL). After stirring at rt for overnight, the reaction mixture was concentrated. The residue was purified by reverse phase HPLC, eluting 5-30% water (0.1% TFA)/CH₃CN (0.1% TFA), to give the title product. UPLC-MS (C4, 2 min): 667.11 (M+2/2) at 1.22 min.

### Step 7: 2,5-Dioxopyrrolidin-1-yl (2R)-3-(2,4-bis{bis[2-({2-[(α-D-mannopyranosyl)oxy]ethyl}amino)-2-oxoethyl]amino}butanamido)propanoate

The title compound was prepared using procedures analogous to those described for Example 1, Steps 9 to 10, substituting benzyl (2R)-3-(2,4-bis{bis[2-({2-[(α-D-mannopyranosyl) oxy]ethyl}amino)-2-oxoethyl]amino}butanamido)propanoate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy) ethyl]amino}ethyl)-3,9,12,15,22-pentaazatetracosyl}-4,11,15,23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oate in Step 9. UPLC-MS Method (C18, 2 min): m/z=670.58 (z=2); t_{R}=0.30 min.

### Example 33: 2,5-Dioxopyrrolidin-1-yl (2R)-3-{2,4-bis[bis(2-{[2-({α-D-mannopyranosyl-(1→3)-[α-D-mannopyranosyl-(1→6)]-α-D-mannopyranosyl}oxy)ethyl]amino}-2-oxoethyl)amino]butanamido}propanoate (ML-33)

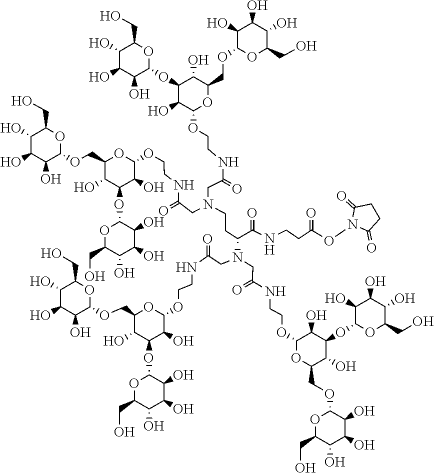

The title compound was prepared using procedures analogous to those described for Example 32, Steps 6 to 7, substituting 2-({α-D-mannopyranosyl-(1-3)-[α-D-mannopyranosyl-(1→6)]-α-D-mannopyranosyl}oxy)ethan-1-amine for 2-aminoethyl α-D-mannopyranoside in

### Step 6. UPLC-MS Method (C18, 2 min): m/z=670.58 (z=2); t_{R}=0.30 min. UPLC-MS Method G: m/z=1319.03 (z=2); t_{R}=2.89 min.

### Example 34: 2,5-Dioxopyrrolidin-1-yl (S)-18-{[6-(bis{2-[(α-D-mannopyranosyl)oxy]ethyl}amino)-6-oxohexyl]carbamoyl}-1-[(α-L-fucopyranosyl)oxy]-4,8,16,20-tetraoxo-6-(2-oxo-2-{[2-({α-D-mannopyranosyl-(1→3)-[α-D-mannopyranosyl-(1→6)]-α-D-mannopyranosyl}oxy)ethyl]amino}ethyl)-3,6,9,15,19-pentaazapentacosan-25-oate (ML-34)

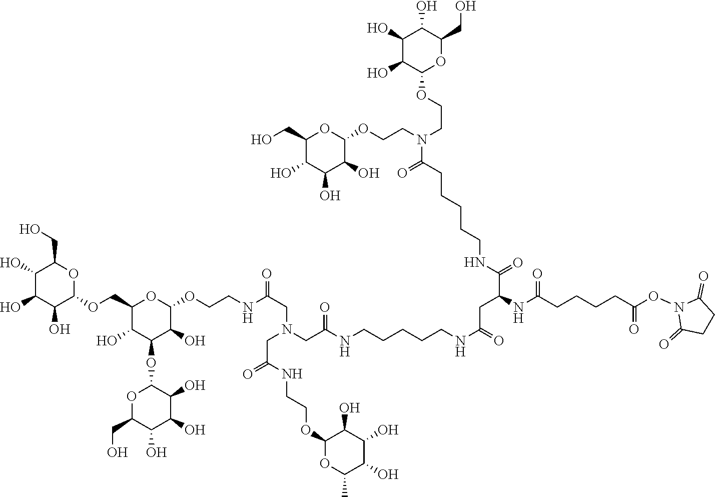 [SMILES: C[C@@H]1O[C@@H](OCCNC(=O)CN(CC(=O)NCCCCCNC(=O)C[C@H](NC(=O)CCCCC(=O)ON2C(=O)CCC2=O)C(=O)NCCCCCC(=O)N(CCO[C@H]2O[C@H](CO)[C@@H](O)[C@H](O)[C@@H]2O)CCO[C@H]2O[C@H](CO)[C@@H](O)[C@H](O)[C@@H]2O)CC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)[C@@H](O)[C@H](O)[C@@H]1O]

### Step 1: 13-[2-({2-[(α-L-Fucopyranosyl)oxy]ethyl}amino)-2-oxoethyl]-3,11-dioxo-1-phenyl-2-oxa-4,10,13-triazapentadecan-15-oic acid

To a suspension of 13-(carboxymethyl)-3,11-dioxo-1-phenyl-2-oxa-4,10,13-triazapentadecan-15-oic acid (1.5 g, 3.66 mmol) in DCM (4 mL) at 0° C. was added TFAA (637 μL, 4.58 mmol). After stirring at 0° C. for 3 h, the reaction mixture was cooled down to −30° C., to which was added a solution of TEA (1.226 mL, 8.79 mmol) in DMF (2 mL) dropwise via syringe and, 30 min later, 2-aminoethyl α-L-fucopyranoside (873 mg, 4.21 mmol) in DMF (2 mL). The reaction mixture allowed to gradually warm up to rt and stirred overnight. The reaction mixture was concentrated and purified by reverse phase chromatography (120 g C18), eluting with 0-30% ACN in water, to afford the title compound.

### Step 2: Benzyl [5-(2-{[2-({2-[(α-L-fucopyranosyl)oxy]ethyl}amino)-2-oxoethyl](2-oxo-2-{[2-({α-D-mannopyranosyl-(1→3)-[α-D-mannopyranosyl-(1-+6)]-α-D-mannopyranosyl}oxy)ethyl]amino}ethyl)amino}acetamido)pentyl]carbamate

To a solution of 13-[2-({2-[(α-L-fucopyranosyl)oxy]ethyl}amino)-2-oxoethyl]-3,11-dioxo-1-phenyl-2-oxa-4,10,13-triazapentadecan-15-oic acid (600 mg, 1.002 mmol) in mixed solvent (DMF/water v/v=2/1, 3 mL) was added 2-({α-D-mannopyranosyl-(1-3)-[α-D-mannopyranosyl-(1→6)]-α-D-mannopyranosyl}oxy)ethan-1-amine (823 mg, 1.503 mmol), EDC (240 mg, 1.253 mmol) and HOBt (192 mg, 1.253 mmol). After stirring at rt overnight, the reaction mixture was concentrated, and the residue was purified by reverse phase chromatography (120 g C18), eluting with 0-30% ACN in water, to give the title compound.

### Step 3: N-(5-Aminopentyl)-2-{[2-({2-[(α-L-fucopyranosyl)oxy]ethyl}amino)-2-oxoethyl](2-oxo-2-{[2-({α-D-mannopyranosyl-(1→3)-[α-D-mannopyranosyl-(1→6)]-α-D-mannopyranosyl}oxy) ethyl]amino}ethyl)amino}acetamide

A mixture of benzyl [5-(2-{[2-({2-[(α-L-fucopyranosyl)oxy]ethyl}amino)-2-oxoethyl](2-oxo-2-{[2-({α-D-mannopyranosyl-(1-3)-[α-D-mannopyranosyl-(1→6)]-α-D-mannopyranosyl}oxy)ethyl]amino}ethyl)amino}acetamido)pentyl]carbamate (400 mg, 0.355 mmol) and Pd/C (37.7 mg, 0.355 mmol) in water (10 mL) was stirred under a balloon of H₂ at rt overnight. The catalyst was filtered off, and the filtrate was freeze-dried to give the title compound.

### Step 4: Benzyl[6-(bis{2-[(2,3,4,6-tetra-O-acetyl-α-D-mannopyranosyl)oxy]ethyl}amino)-6-oxohexyl]carbamate

To a solution of bis{2-[(2,3,4,6-tetra-O-acetyl-α-D-mannopyranosyl)oxy]ethyl} amine (2.0 g, 2.61 mmol) in DMF (3 mL) at 0° C. was added 2,5-dioxopyrrolidin-1-yl 6-{[(benzyloxy) carbonyl]amino}hexanoate (2.366 g, 6.53 mmol) and DIPEA (547 μL, 3.13 mmol). After stirring at rt overnight, the reaction mixture was concentrated, and the residue was purified by reverse phase chromatography (120 g C18), eluting with 0-40% ACN in water, to give the title compound.

### Step 5: Benzyl [6-(bis{2-[(α-D-mannopyranosyl)oxy]ethyl}amino)-6-oxohexyl]carbamate

To a solution of benzyl [6-(bis{2-[(2,3,4,6-tetra-O-acetyl-α-D-mannopyranosyl)oxy]ethyl}amino)-6-oxohexyl]carbamate (2.0 g, 1.974 mmol) in MeOH (10 mL) at rt was added NaOCH₃ (395 μL, 0.197 mmol, 0.5M). After stirring at rt overnight, to the reaction mixture was added pre-washed ion exchange resin (DOWEX™) H⁺ form (5.7 g). The resin was filtered off, and the filtrate was concentrated. The residue was purified by reverse phase chromatography (120 g C18), eluting with 0-15% ACN in water, to give the title compound.

¹H-NMR δ(ppm)(CH₃OH-d4): 1.40 (3H, d, J=9.06 Hz), 1.58-1.52 (2H, m), 1.68-1.62 (2H, m), 2.06 (0H, s), 2.49 (2H, t, J=7.56 Hz), 3.15 (2H, t, J=6.94 Hz), 3.51-3.47 (2H, m), 3.74-3.59 (12H, m), 3.94-3.80 (6H, m), 5.09 (2H, s), 7.32 (1H, d, J=6.30 Hz), 7.37-7.36 (3H, m).

### Step 6: bis{2-[(α-D-Mannopyranosyl)oxy]ethyl}amine

A mixture of benzyl [6-(bis{2-[(α-D-mannopyranosyl)oxy]ethyl}amino)-6-oxohexyl]carbamate (1.0 g, 1.478 mmol) Pd/C (79 mg, 0.074 mmol) in water (10 mL) was stirred under a balloon of H₂ at rt for overnight. The catalyst was filtered off, and the filtrate was freeze-dried to give the title compound.

### Step 7: Benzyl (S)-3-{[(benzyloxy)carbonyl]amino}-4-{[6-(bis{2-[(α-D-mannopyranosyl)oxy]ethyl}amino)-6-oxohexyl]amino}-4-oxobutanoate

To a solution of bis{2-[(α-D-mannopyranosyl)oxy]ethyl}amine (0.8 g, 1.474 mmol) in DMF (2.0 mL) and water (1.0 mL) was added EDC (424 mg, 2.212 mmol) and HOBt (339 mg, 2.212 mmol). After stirring at rt overnight, the reaction mixture was concentrated. The residue was purified by reverse phase chromatography (C18 120 g), eluting with 0-50% ACN in water, to give the title compound.

### Step 8: (S)-3-Amino-4-{[6-(bis{2-[(α-D-mannopyranosyl)oxy]ethyl}amino)-6-oxohexyl]amino}-4-oxobutanoic acid

A mixture of benzyl (S)-3-{[(benzyloxy)carbonyl]amino}-4-{[6-(bis{2-[(α-D-mannopyranosyl)oxy]ethyl}amino)-6-oxohexyl]amino}-4-oxobutanoate (690 mg, 0.782 mmol) and Pd/C (167 mg, 0.156 mmol) in water (10 mL) was stirred under a balloon of H₂ at rt overnight. The catalyst was filtered off, and the filtrate was freeze-dried to give the title compound.

### Step 9: (S)-3-[6-(Benzyloxy)-6-oxohexanamido]-4-{[6-(bis{2-[(α-D-mannopyranosyl) oxy]ethyl}amino)-6-oxohexyl]amino}-4-oxobutanoic acid

To a solution of (S)-3-amino-4-{[6-(bis{2-[(α-D-mannopyranosyl)oxy]ethyl} amino)-6-oxohexyl]amino}-4-oxobutanoic acid (475.5 mg, 0.723 mmol) in DMF (3 mL) at rt was added benzyl (2,5-dioxopyrrolidin-1-yl) adipate (301 mg, 0.904 mmol) and DIPEA (158 μL, 0.904 mmol). After stirring at rt for 2 h, the reaction mixture was concentrated, and the residue was purified by reverse phase chromatography (120 g C18), eluting with 0-25% ACN in water, to give the title compound.

### Step 10: Benzyl (S)-18-{[6-(bis{2-[(α-D-mannopyranosyl)oxy]ethyl}amino)-6-oxohexyl]carbamoyl}-1-[(α-L-fucopyranosyl)oxy]-4,8,16,20-tetraoxo-6-(2-oxo-2-{[2-({α-D-mannopyranosyl-(1→3)-[α-D-mannopyranosyl-(1→6)]-α-D-mannopyranosyl}oxy)ethyl]amino}ethyl)-3,6,9,15,19-pentaazapentacosan-25-oate

To a solution of (S)-3-[6-(benzyloxy)-6-oxohexanamido]-4-{[6-(bis{2-[(α-D-mannopyranosyl)oxy]ethyl}amino)-6-oxohexyl]amino}-4-oxobutanoic acid (100 mg, 0.114 mmol) in DMF (3 mL) at rt was added N-(5-aminopentyl)-2-{[2-({2-[(α-L-fucopyranosyl) oxy] ethyl}amino)-2-oxoethyl](2-oxo-2-{[2-({α-D-mannopyranosyl-(1-3)-[α-D-mannopyranosyl-(1→6)]-α-D-mannopyranosyl}oxy)ethyl]amino}ethyl)amino}acetamide (113 mg, 0.114 mmol), HOBt (21.85 mg, 0.143 mmol), and EDC (27.4 mg, 0.143 mmol). After stirring at rt for 2 h, the reaction mixture was concentrated, and the residue was purified by reverse phase chromatography (120 g C18), eluting with 0-25% ACN in water, to give the title compound.

### Step 11: 2,5-Dioxopyrrolidin-1-yl (S)-18-{[6-(bis{2-[(α-D-mannopyranosyl)oxy]ethyl}amino)-6-oxohexyl]carbamoyl}-1-[(α-L-fucopyranosyl)oxy]-4,8,16,20-tetraoxo-6-(2-oxo-2-{[2-({α-D-mannopyranosyl-(1→3)-[α-D-mannopyranosyl-(1→6)]-α-D-mannopyranosyl}oxy)ethyl]15 amino}ethyl)-3,6,9,15,19-pentaazapentacosan-25-oate

The title compound was prepared using procedures analogous to those described for Example 1, Steps 9 to 10, substituting benzyl (S)-18-{[6-(bis{2-[(α-D-mannopyranosyl) oxy]ethyl}amino)-6-oxohexyl]carbamoyl}-1-[(α-L-fucopyranosyl)oxy]-4,8,16,20-tetraoxo-6-(2-oxo-2-{[2-({α-D-mannopyranosyl-(1-3)-[α-D-mannopyranosyl-(1→6)]-α-D-mannopyranosyl}oxy)ethyl]amino}ethyl)-3,6,9,15,19-pentaazapentacosan-25-oate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1→3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy)ethyl]amino}ethyl)-3,10,13,16,22,25,28-heptaazatetratriacontan-34-oate in Step 9. UPLC-MS Method A: (C18, 2 min): m/z=670.58 (z=2); t_{R}=0.30 min. UPLC-MS Method G: m/z=1319.03 (z=2); t_{R}=2.89 min.

### Example 35: 2,5-Dioxopyrrolidin-1-yl (7S,15S)-15-({2-[(α-L-fucopyranosyl)oxy]ethyl}carbamoyl)-4,13,17-trioxo-6-[2-oxo-2-({2-[(α-D-mannopyranosyl)oxy]ethyl}amino)ethyl]-1-phenoxy-7-({2-[(α-D-mannopyranosyl)oxy]ethyl}carbamoyl)-3,6,12,16-tetraazadocosan-22-oate (ML-35)

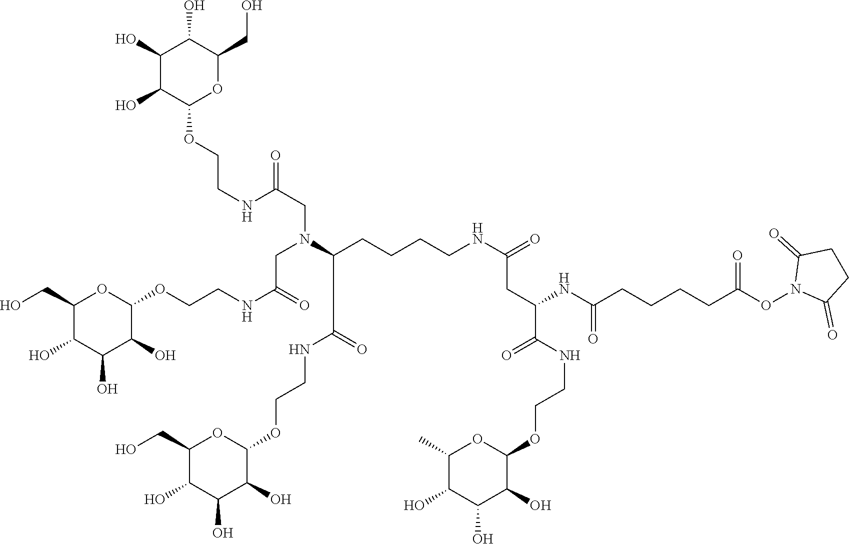 [SMILES: C[C@@H]1O[C@@H](OCCNC(=O)[C@H](CC(=O)NCCCC[C@@H](C(=O)NCCO[C@H]2O[C@H](CO)[C@@H](O)[C@H](O)[C@@H]2O)N(CC(=O)NCCO[C@H]2O[C@H](CO)[C@@H](O)[C@H](O)[C@@H]2O)CC(=O)NCCO[C@H]2O[C@H](CO)[C@@H](O)[C@H](O)[C@@H]2O)NC(=O)CCCCC(=O)ON2C(=O)CCC2=O)[C@@H](O)[C@H](O)[C@@H]1O]

### Step 1: Benzyl (S)-3-{[(benzyloxy)carbonyl]amino}-4-({2-[(α-L-fucopyranosyl)oxy]ethyl}amino)-4-oxobutanoate

To a suspension of N-Cbz-L-aspartic acid β-benzyl ester (2.0 g, 5.60 mmol) in DMF (4 mL) at rt was added a solution of 2-aminoethyl α-L-fucopyranoside (1.160 g, 5.60 mmol) in water (2 mL), HOBt (1.714 g, 11.19 mmol), and EDC (2.146 g, 11.19 mmol). After stirring at rt overnight, the reaction mixture was concentrated, and the residue was purified by reverse phase HPLC (C4 50×250 mm column), from 10% to 60% ACN in water with 0.1% TFA, and lyophilized.

### Step 2: (S)-3-Amino-4-({2-[(α-L-fucopyranosyl)oxy]ethyl}amino)-4-oxobutanoic acid

A mixture of benzyl (S)-3-{[(benzyloxy)carbonyl]amino}-4-({2-[(α-L-fucopyranosyl) oxy]ethyl}amino)-4-oxobutanoate (2.3 g, 4.21 mmol) and Pd/C (0.224 g, 0.210 mmol) in a mixed solvent of water (40 mL), EtOAc (10 mL), and ACN (10 mL) was stirred under a balloon of H₂ at rt overnight. The catalyst was filtered off, and the filtrate was freeze-dried to give the title compound.

### Step 3: (S)-3-[6-(Benzyloxy)-6-oxohexanamido]-4-({2-[(α-L-fucopyranosyl)oxy]ethyl}amino)-4-oxobutanoic acid

To the suspension of (S)-3-amino-4-({2-[(α-L-fucopyranosyl)oxy]ethyl}amino)-4-oxobutanoic acid (1.344 g, 4.03 mmol) in DMF (7 mL) at rt was added DIPEA (1.626 mL, 9.31 mmol) and benzyl (2,5-dioxopyrrolidin-1-yl) adipate (1.344 g, 4.03 mmol). After stirring at rt overnight, the reaction mixture was concentrated, and the residue was purified by reverse phase HPLC C4 50×250 mm, FR=90 ml/min, wavelength=210 nm, ramp 20 min, from 10% to 40% ACN in water (0.1% TFA), t_{R}=14.3 min, lyophilized to powder, and the second major product is the hydrolyzed acid.

### Step 4: Benzyl (7S,15S)-15-({2-[(α-L-fucopyranosyl)oxy]ethyl}carbamoyl)-4,13,17-trioxo-6-[2-oxo-2-({2-[(α-D-mannopyranosyl)oxy]ethyl}amino)ethyl]-1-phenoxy-7-({2-[(α-D-mannopyranosyl)oxy]ethyl}carbamoyl)-3,6,12,16-tetraazadocosan-22-oate

To the suspension of (S)-3-[6-(benzyloxy)-6-oxohexanamido]-4-({2-[(α-L-fucopyranosyl)oxy]ethyl}amino)-4-oxobutanoic acid (260 mg, 0.481 mmol) in DMF (3 mL) at rt was added N-{2-[(α-D-mannopyranosyl)oxy]ethyl]-N²,N²-bis[2-({2-[(α-D-mannopyranosyl)oxy]ethyl]amino)-2-oxoethyl]-L-lysinamide (422 mg, 0.481 mmol, WO 2018175272 A1), EDC (184 mg, 0.962 mmol), and HOBt (147 mg, 0.962 mmol). After stirring at rt over weekend, the reaction mixture was concentrated, and the residue was purified by reverse phase chromatography (C18 120 g), eluting with 0-30% ACN in water, to give the title compound.

### Step 5: 2,5-Dioxopyrrolidin-1-yl (7S,15S)-15-({2-[(α-L-fucopyranosyl)oxy]ethyl}carbamoyl)-4,13,17-trioxo-6-[2-oxo-2-({2-[(α-D-mannopyranosyl)oxy]ethyl}amino)ethyl]-1-phenoxy-7-({2-[(α-D-mannopyranosyl)oxy]ethyl}carbamoyl)-3,6,12,16-tetraazadocosan-22-oate

The title compound was prepared using procedures analogous to those described for Example 1, Steps 9 to 10, substituting benzyl (7S,15S)-15-({2-[(α-L-fucopyranosyl)oxy]ethyl}carbamoyl)-4,13,17-trioxo-6-[2-oxo-2-({2-[(α-D-mannopyranosyl)oxy]ethyl}amino) ethyl]-1-phenoxy-7-({2-[(α-D-mannopyranosyl)oxy]ethyl}carbamoyl)-3,6,12,16-tetraazadocosan-22-oate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oate in Step 9. UPLC-MS Method A: m/z=670.58 (z=2); t_{R}=0.30 min. UPLC-MS Method G: m/z=1319.03 (z=2); t_{R}=2.89 min.

### Example 36: 2,5-Dioxopyrrolidin-1-yl (7S,15S)-15-{[2-({α-D-mannopyranosyl-(1→3)-[α-D-mannopyranosyl-(1→6)]-α-D-mannopyranosyl}oxy)ethyl]carbamoyl}-4,13,17-trioxo-6-[2-oxo-2-({2-[(α-D-mannopyranosyl)oxy]ethyl}amino)ethyl]-1-phenoxy-7-({2-[(α-D-mannopyranosyl)oxy]ethyl}carbamoyl)-3,6,12,16-tetraazadocosan-22-oate (ML-36)

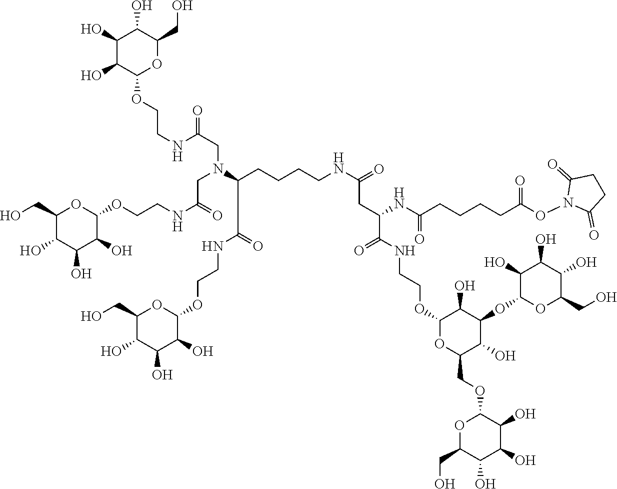 [SMILES: O=C(C[C@H](NC(=O)CCCCC(=O)ON1C(=O)CCC1=O)C(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O]

The title compound was prepared using procedures analogous to those described for Example 35, substituting 2-({α-D-mannopyranosyl-(1-3)-[α-D-mannopyranosyl-(1→6)]-α-D-mannopyranosyl}oxy)ethan-1-amine for 2-aminoethyl α-L-fucopyranoside in Step 1. UPLC-MS Method A: m/z=670.58 (z=2); t_{R}=0.30 min. UPLC-MS Method G: m/z=1319.03 (z=2); t_{R}=2.89 min.

### Example 37: 2,5-Dioxopyrrolidin-1-yl (S)-1-[(α-L-fucopyranosyl)oxy]-6-[2-({2-[(α-L-fucopyranosyl)oxy]ethyl}amino)-2-oxoethyl]-7-({2-[(α-L-fucopyranosyl)oxy]ethyl}carbamoyl)-4,13,17-trioxo-15-{2-oxo-2-[(6-oxo-6-{[2-({α-D-mannopyranosyl-(1→3)-[α-D-mannopyranosyl-(1→6)]-α-D-mannopyranosyl}oxy)ethyl]amino}hexyl)amino]ethyl}-3,6,12,15,18-pentaazatetracosan-24-oate (ML-37)

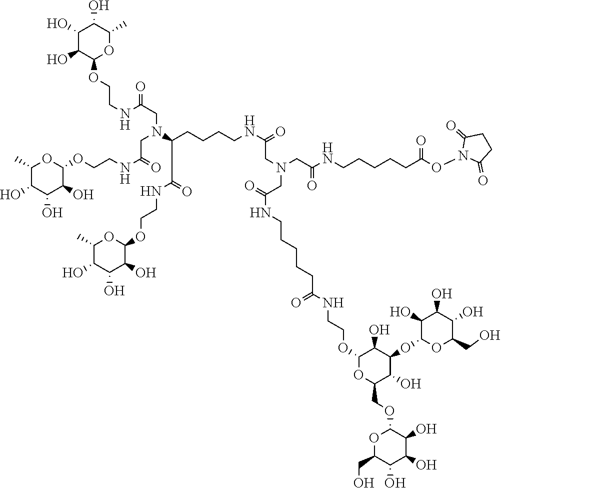 [SMILES: C[C@@H]1O[C@@H](OCCNC(=O)CN(CC(=O)NCCO[C@H]2O[C@@H](C)[C@@H](O)[C@@H](O)[C@@H]2O)[C@@H](CCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)C(=O)NCCO[C@@H]2O[C@@H](C)[C@@H](O)[C@@H](O)[C@@H]2O)[C@@H](O)[C@H](O)[C@@H]1O]

### Step 1: Benzyl (6-{[2-({α-D-mannopyranosyl-(1→3)-[α-D-mannopyranosyl-(1→6)]-α-D-mannopyranosyl}oxy)ethyl]amino}-6-oxohexyl)carbamate

To a solution of 2-({α-D-mannopyranosyl-(1-3)-[α-D-mannopyranosyl-(1→6)]-α-D-mannopyranosyl}oxy)ethan-1-amine (2.0 g, 3.65 mmol) and 2,5-dioxopyrrolidin-1-yl 6-{[(benzyloxy)carbonyl]amino}hexanoate (1.59 g, 4.38 mmol) in DMF (20 mL) at rt was added DIPEA (829 μL, 4.75 mmol). After stirring at rt for 16 h, the reaction mixture was concentrated, and the residue was purified by reverse phase chromatography to isolate the title compound. UPLC-MS Method A: m/z=795.4 (z=1); t_{R}=2.54 min.

### Step 2: 6-Amino-N-[2-({α-D-mannopyranosyl-(1→3)-[α-D-mannopyranosyl-(1→6)]-α-D-mannopyranosyl}oxy)ethyl]hexanamide

The title compound was prepared using the procedure analogous to that described for Example 1, Step 3, substituting benzyl (6-{[2-({α-D-mannopyranosyl-(1-3)-[α-D-mannopyranosyl-(1→6)]-α-D-mannopyranosyl}oxy)ethyl]amino}-6-oxohexyl)carbamate for benzyl [6-({2-[(α-D-mannopyranosyl)oxy]ethyl}amino)-6-oxohexyl]carbamate. UPLC-MS Method A: m/z=661.30 (z=1); t_{R}=0.85 min.

### Step 3: N-(2-{[6-(Benzyloxy)-6-oxohexyl]amino}-2-oxoethyl)-N-{2-[(6-{[2-({α-D-mannopyranosyl-(1→3)-[α-D-mannopyranosyl-(1→6)]-α-D-mannopyranosyl}oxy) ethyl]amino}-6-oxohexyl)amino]-2-oxoethyl}glycine

To a solution of 2,2′-[(2-{[6-(benzyloxy)-6-oxohexyl]amino}-2-oxoethyl)azanediyl]diacetic acid (1.1 g, 2.79 mmol) in DCM (24 mL) at 0° C. was added TFAA (473 μL, 3.35 mmol). After stirring at 0° C. for 2 h, the reaction mixture was cooled to −15° C. to which was added a solution of TEA (933 μL, 6.69 mmol) in DMF (12 mL) slowly over 20 min. After stirring at −15° C. for 10 min, the reaction mixture warmed up to rt over 30 min, to which then was added a solution of 6-amino-N-[2-({α-D-mannopyranosyl-(1-3)-[α-D-mannopyranosyl-(1→6)]-α-D-mannopyranosyl}oxy)ethyl]hexanamide (2.0 g, 3.07 mmol) in DMF (24 mL). After stirring for 90 min, the reaction mixture was concentrated, and the residue was purified by reverse phase chromatography to isolate the title compound. UPLC-MS Method A: m/z=1037.5 (z=1); t_{R}=2.84 min.

### Step 4: (S)-2,2′-[(6-{[(Benzyloxy)carbonyl]amino]-1-carboxyhexyl)azanediyl]diacetic acid

To a solution of bromoacetic acid (9.91 g, 71.3 mmol) in 1.0M NaOH (100 mL, 100 mmol) at 0° C. was added dropwise N⁶-benzyloxycarbonyl-L-lysine (5.0 g, 17.84 mmol) in 1.0M NaOH (50 mL, 50 mmol). After stirring at rt for 2 h, the reaction mixture was heated to 50° C. and stirred for 16 h. The reaction mixture was then cooled to 0° C. and acidified with 12M HCl until a solid formed, which was collected and dried in vacuo to give the title compound. UPLC-MS Method A: m/z=397.1 (z=1); t_{R}=4.30 min.

### Step 5: Benzyl (S)-[6-{bis[2-({2-[(α-L-fucopyranosyl)oxy]ethyl}amino)-2-oxoethyl]amino}-7-({2-[(α-L-fucopyranosyl)oxy]ethyl}amino)-7-oxoheptyl]carbamate

To a solution of (S)-2,2′-[(6-{[(benzyloxy)carbonyl]amino}-1-carboxyhexyl)azanediyl]diacetic acid (750 mg, 1.892 mmol) in DMF (15 mL) at 0° C. was added DMAP (901 mg, 7.38 mmol) and EDC (1.741 g, 9.08 mmol), and 30 min later 2-aminoethyl α-L-fucopyranoside (1.294 g, 6.24 mmol). After stirring at 0° C. for 30 min, the reaction mixture was brought to rt and stirred for 48 h. The reaction mixture was concentrated, and the residue was purified by reverse phase chromatography to isolate the title compound. UPLC-MS Method A: m/z=964.4 (z=1); t_{R}=3.00 min.

### Step 6: (S)-2,2′-{[7-Amino-1-({2-[(α-L-fucopyranosyl)oxy]ethyl}amino)-1-oxoheptan-2-yl]azanediyl}bis(N-{2-[(α-L-fucopyranosyl)oxy]ethyl}acetamide)

The title compound was prepared using the procedure analogous to that described for Example 1, Step 3, substituting benzyl (S)-[6-{bis[2-({2-[(α-L-fucopyranosyl)oxy]ethyl}amino)-2-oxoethyl]amino}-7-({2-[(α-L-fucopyranosyl)oxy]ethyl}amino)-7-oxoheptyl]carbamate for benzyl [6-({2-[(α-D-mannopyranosyl)oxy]ethyl}amino)-6-oxohexyl]carbamate. UPLC-MS Method A: m/z=830.3 (z=1); t_{R}=1.11 min.

### Step 7: Benzyl (S)-1-[(α-L-fucopyranosyl)oxy]-6-[2-({2-[(α-L-fucopyranosyl)oxy]ethyl}amino)-2-oxoethyl]-7-({2-[(α-L-fucopyranosyl)oxy]ethyl}carbamoyl)-4,13,17-trioxo-15-{2-oxo-2-[(6-oxo-6-{[2-({α-D-mannopyranosyl-(1→3)-[α-D-mannopyranosyl-(1→6)]-α-D-mannopyranosyl}oxy)ethyl]amino}hexyl)amino]ethyl}-3,6,12,15,18-pentaazatetracosan-24-oate

To a mixture of N-(2-{[6-(benzyloxy)-6-oxohexyl]amino}-2-oxoethyl)-N-{2-[(6-{[2-({α-D-mannopyranosyl-(1-3)-[α-D-mannopyranosyl-(1→6)]-α-D-mannopyranosyl}oxy)ethyl]amino}-6-oxohexyl)amino]-2-oxoethyl}glycine (200 mg, 0.193 mmol)) and (S)-2,2′-{[7-amino-1-({2-[(α-L-fucopyranosyl)oxy]ethyl}amino)-1-oxoheptan-2-yl]azanediyl}bis(N-{2-[(α-L-fucopyranosyl)oxy]ethyl}acetamide) (240 mg, 0.289 mmol) in DMF (3 mL) at rt was added EDC (73.9 mg, 0.386 mmol)) and DMAP (47.1 mg, 0.386 mmol). After stirring at rt overnight, the reaction mixture was concentrated, and the residue was purified by reverse phase chromatography to give the title compound. UPLC-MS Method A: m/z=1849.7 (z=1); t_{R}=2.88 min.

### Step 8: 2,5-Dioxopyrrolidin-1-yl (S)-1-[(α-L-fucopyranosyl)oxy]-6-[2-({2-[(α-L-fucopyranosyl) oxy]ethyl}amino)-2-oxoethyl]-7-({2-[(α-L-fucopyranosyl)oxy]ethyl}carbamoyl)-4,13,17-trioxo-15-{2-oxo-2-[(6-oxo-6-{[2-({α-D-mannopyranosyl-(1→3)-[α-D-mannopyranosyl-(1→6)]-α-D-mannopyranosyl}oxy)ethyl]amino}hexyl)amino]ethyl}-3,6,12,15,18-pentaazatetracosan-24-oate

The title compound was prepared using procedures analogous to those described for Example 1, Steps 9 to 10, substituting benzyl (S)-1-[(α-L-fucopyranosyl)oxy]-6-[2-({2-[(α-L-fucopyranosyl)oxy]ethyl}amino)-2-oxoethyl]-7-({2-[(α-L-fucopyranosyl)oxy]ethyl}carbamoyl)-4,13,17-trioxo-15-{2-oxo-2-[(6-oxo-6-{[2-({α-D-mannopyranosyl-(1-3)-[α-D-mannopyranosyl-(1→6)]-α-D-mannopyranosyl}oxy)ethyl]amino}hexyl)amino]ethyl}-3,6,12,15,18-pentaazatetracosan-24-oate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oate in Step 9. UPLC-MS Method A: m/z=1856.4 (z=1); t_{R}=2.14 min.

### Example 38: 2,5-Dioxopyrrolidin-1-yl (S)-1-[(α-L-fucopyranosyl)oxy]-6-[2-({2-[(α-L-fucopyranosyl)oxy]ethyl}amino)-2-oxoethyl]-7-({2-[(α-L-fucopyranosyl)oxy]ethyl}carbamoyl)-4,13,17-trioxo-15-(2-oxo-2-{[6-oxo-6-({2-[(α-L-fucopyranosyl)oxy]ethyl}amino)hexyl]amino}ethyl)-3,6,12,15,18-pentaazatetracosan-24-oate (ML-38)

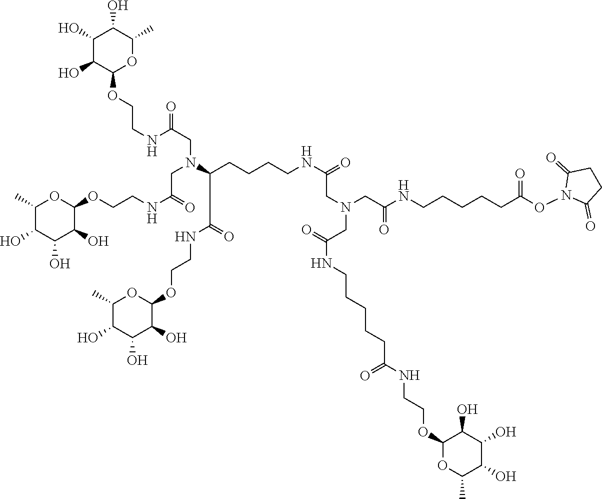 [SMILES: C[C@@H]1O[C@@H](OCCNC(=O)CCCCCNC(=O)CN(CC(=O)NCCCCCC(=O)ON2C(=O)CCC2=O)CC(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)[C@@H](O)[C@H](O)[C@@H]1O]

The title compound was prepared using procedures analogous to those described for Example 37, substituting 2-aminoethyl α-L-fucopyranoside for 2-({α-D-mannopyranosyl-(1→3)-[α-D-mannopyranosyl-(1→6)]-α-D-mannopyranosyl}oxy)ethan-1-amine in Step 1. UPLC-MS Method A: m/z=1515.5 (z=1); t_{R}=2.48 min.

### Example 39: 2,5-Dioxopyrrolidin-1-yl (S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-4,13,17-trioxo-15-(2-oxo-2-{[6-oxo-6-({2-[(α-D-mannopyranosyl)oxy]ethyl}amino)hexyl]amino}ethyl)-3,6,12,15,18-pentaazatetracosan-24-oate (ML-39)

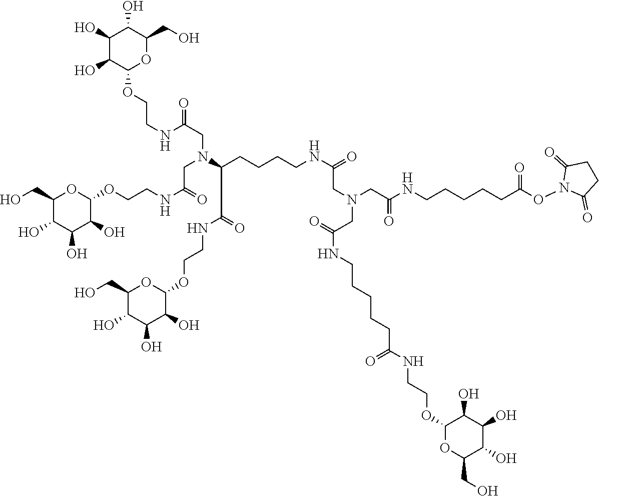 [SMILES: O=C(CCCCCNC(=O)CN(CC(=O)NCCCCCC(=O)ON1C(=O)CCC1=O)CC(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O)NCCO[C@H]1O[C@H](CO)[C@@H](O)[C@H](O)[C@@H]1O]

The title compound was prepared using procedures analogous to those described for Example 37, substituting 2-aminoethyl α-D-mannopyranoside for 2-({α-D-mannopyranosyl-(1→3)-[α-D-mannopyranosyl-(1→6)]-α-D-mannopyranosyl}oxy)ethan-1-amine in Step 1 and 2-aminoethyl α-D-mannopyranoside for 2-aminoethyl α-L-fucopyranoside in Step 5, respectively. UPLC-MS Method A: m/z=791.2 (z=2); t_{R}=2.20 min.

### Example 40: 2,5-Dioxopyrrolidin-1-yl (S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-4,13,17-trioxo-15-{2-oxo-2-[(6-oxo-6-{[2-({α-D-mannopyranosyl-(1→3)-[α-D-mannopyranosyl-(1→6)-[α-D-mannopyranosyl}oxy)ethyl]amino]hexyl)amino]ethyl}-3,6,12,15,18-pentaazatetracosan-24-oate (ML-40)

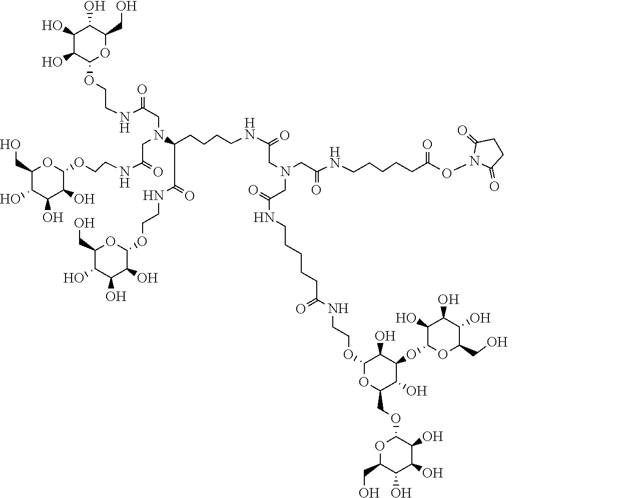 [SMILES: O=C(CCCCCNC(=O)CN(CC(=O)NCCCCCC(=O)ON1C(=O)CCC1=O)CC(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for Example 37, substituting 2-aminoethyl α-D-mannopyranoside for 2-aminoethyl α-L-fucopyranoside in Step 5. UPLC-MS Method A: m/z=952.2 (z=2); t_{R}=2.06 min.

### Example 41: 2,5-Dioxopyrrolidin-1-yl 13-[2-(bis{2-[(α-D-mannopyranosyl)oxy]ethyl}amino)-2-oxoethyl]-1-[(α-L-fucopyranosyl)oxy]-4,11,15,23,27-pentaoxo-25-{2-oxo-2-[(6-oxo-6-{[2-({α-D-mannopyranosyl-(1→3)-[α-D-mannopyranosyl-(1→6)]-α-D-mannopyranosyl}oxy)ethyl]amino}hexyl)amino]ethyl}-3,10,13,16,22,25,28-heptaazatetratriacontan-34-oate (ML-41)

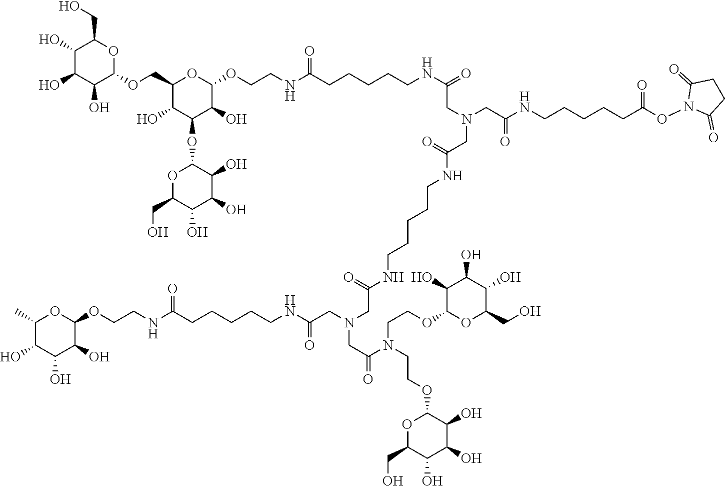 [SMILES: C[C@@H]1O[C@@H](OCCNC(=O)CCCCCNC(=O)CN(CC(=O)NCCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)N(CCO[C@H]2O[C@H](CO)[C@@H](O)[C@H](O)[C@@H]2O)CCO[C@H]2O[C@H](CO)[C@@H](O)[C@H](O)[C@@H]2O)[C@@H](O)[C@H](O)[C@@H]1O]

### Step 1: 13-[2-(bis{2-[(2,3,4,6-Tetra-O-acetyl-α-D-mannopyranosyl)oxy]ethyl}amino)-2-oxoethyl]-3,11-dioxo-1-phenyl-2-oxa-4,10,13-triazapentadecan-15-oic acid

To a suspension of 13-(carboxymethyl)-3,11-dioxo-1-phenyl-2-oxa-4,10,13-triazapentadecan-15-oic acid (750 mg, 1.832 mmol) in DCM (10 mL) at 0° C. was added TFAA (388 μL, 2.75 mmol). After stirring for 2 h at 0° C., the reaction mixture became clear, to which then was added TEA (638 μL, 4.58 mmol) in DMF (10 mL) dropwise over 30 min and bis{2-[(2,3,4,6-tetra-O-α-D-mannopyranosyl)oxy]ethyl}amine (1.403 g, 1.832 mmol, WO 2018/175272 A1) in DMF (10 mL). The reaction mixture was warmed to rt slowly and then stirred for 16 h, and then concentrated. The residue was taken into EtOAc (100 mL), washed with water (2×25 mL) and brine (25 mL), dried over Na₂SO₄, and concentrated to give the title compound. UPLC-MS Method A: m/z=1157.5 (z=1); t_{R}=1.35 min.

### Step 2: 13-[2-(bis{2-[(α-D-Mannopyranosyl)oxy]ethyl}amino)-2-oxoethyl]-3,11-dioxo-1-phenyl-2-oxa-4,10,13-triazapentadecan-15-oic acid

To a solution of 13-[2-(bis{2-[(2,3,4,6-tetra-O-acetyl-α-D-mannopyranosyl)oxy]ethyl}amino)-2-oxoethyl]-3,11-dioxo-1-phenyl-2-oxa-4,10,13-triazapentadecan-15-oic acid (1.76 g, 1.521 mmol) in MeOH (20 mL) at rt was added NaOMe (8.22 mg, 0.152 mmol). The reaction mixture was stirred at rt for 1 h, and then to which was added pre-washed ion exchange resin (DOWEX™) H⁺ form (10 g, 5:1 with respect to the substrate). After stirring for 5 min, the reaction mixture was filtered, and the filtrate was concentrated. The residue was purified by reverse phase chromatography to isolate the title compound. UPLC-MS Method A: m/z=821.4 (z=1); t_{R}=4.43 min.

### Step 3: Benzyl {13-[2-(bis{2-[(α-D-mannopyranosyl)oxy]ethyl}amino)-2-oxoethyl]-1-[(α-L-fucopyranosyl)oxy]-4,11,15-trioxo-3,10,13,16-tetraazahenicosan-21-yl}carbamate

To a solution of 13-[2-(bis{2-[(α-D-mannopyranosyl)oxy]ethyl}amino)-2-oxoethyl]-3,11-dioxo-1-phenyl-2-oxa-4,10,13-triazapentadecan-15-oic acid (100 mg, 0.122 mmol) in DMF (2 mL) at rt was added EDC (28.0 mg, 0.146 mmol), HOBt (18.66 mg, 0.122 mmol), TEA (42.5p L, 0.305 mmol), and 6-amino-N-{2-[(α-D-mannopyranosyl)oxy]ethyl} hexanamide (58.5 mg, 0.183 mmol). After stirring for 16 h, the reaction mixture was concentrated, and the residue was purified on reverse phase chromatography to isolate the title compound. UPLC-MS Method A: m/z=1123.6 (z=1); t_{R} 4.14 min.

### Step 4: 6-[2-({2-[(5-Aminopentyl)amino]-2-oxoethyl}[2-(bis{2-[(α-D-mannopyranosyl)oxy]ethyl}amino)-2-oxoethyl]amino)acetamido]-N-{2-[(α-L-fucopyranosyl)oxy]ethyl}hexanamide

The title compound was prepared using the procedure analogous to that described for Example 1, Step 3, substituting benzyl {13-[2-(bis{2-[(α-D-mannopyranosyl)oxy]ethyl} amino)-2-oxoethyl]-1-[(α-L-fucopyranosyl)oxy]-4,11,15-trioxo-3,10,13,16-tetraazahenicosan-21-yl}carbamate for benzyl [6-({2-[(α-D-mannopyranosyl)oxy]ethyl}amino)-6-oxohexyl]carbamate.

### Step 5: 6-Amino-N-[2-({α-D-mannopyranosyl-(1→3)-[α-D-mannopyranosyl-(1→6)]-α-D-mannopyranosyl}oxy)ethyl]hexanamide

The title compound was prepared using the procedures analogous to those described for Example 1, Steps 1 to 3, substituting 2-({α-D-mannopyranosyl-(1-3)-[α-D-mannopyranosyl-(1→6)]-α-D-mannopyranosyl}oxy)ethan-1-amine for 2-aminoethyl α-D-mannopyranoside in Step 2.

### Step 6: N-(2-{[6-(Benzyloxy)-6-oxohexyl]amino}-2-oxoethyl)-N-{2-[(6-{[2-({α-D-mannopyranosyl-(1→3)-[α-D-mannopyranosyl-(1→6)]-α-D-mannopyranosyl}oxy)ethyl]amino}-6-oxohexyl)amino]-2-oxoethyl}glycine

To a suspension of 2,2′-((2-((6-(benzyloxy)-6-oxohexyl)amino)-2-oxoethyl)azanediyl) diacetic acid for 13-(carboxymethyl)-3,11-dioxo-1-phenyl-2-oxa-4,10,13-triazapentadecan-15-oic acid (1.0 g, 2.54 mmol) in DCM (16 mL) at 0° C. was added TFAA (394 μL, 2.79 mmol). After stirring at 0° C. for 3 h, the reaction mixture was cooled to −30° C., to which was added a solution of TEA (848 μL, 6.08 mmol) in DMF (12 mL) dropwise over 30 min. The reaction mixture was stirred at −30° C. for 30 min, and then to which was added a mixture of 6-amino-N-[2-({α-D-mannopyranosyl-(1→3)-[α-D-mannopyranosyl-(1→6)]-α-D-mannopyranosyl}oxy)ethyl]hexanamide (1.256 g, 1.902 mmol) in DMF (16 mL). The resulting mixture was allowed to warm up to rt and stirred 16 h, and then concentrated. The residue was purified by reverse phase chromatography (C18 240 g), eluting 10-40% ACN in water, to give the title compound.

### Step 7: Benzyl 13-[2-(bis{2-[(α-D-mannopyranosyl)oxy]ethyl}amino)-2-oxoethyl]-1-[(α-L-fucopyranosyl)oxy]-4,11,15,23,27-pentaoxo-25-{2-oxo-2-[(6-oxo-6-{[2-({α-D-mannopyranosyl-(1→3)-[α-D-mannopyranosyl-(1→6)]-α-D-mannopyranosyl}oxy)ethyl]amino}hexyl)amino]ethyl}-3,10,13,16,22,25,28-heptaazatetratriacontan-34-oate

To a solution of N-(2-{[6-(benzyloxy)-6-oxohexyl]amino}-2-oxoethyl)-N-{2-[(6-{[2-({α-D-mannopyranosyl-(1-3)-[α-D-mannopyranosyl-(1→6)]-α-D-mannopyranosyl}oxy)ethyl]amino}-6-oxohexyl)amino]-2-oxoethyl}glycine (40 mg, 0.039 mmol) in DMF (3 mL) at rt was added EDC (11.09 mg, 0.058 mmol), HOBt (8.86 mg, 0.058 mmol), DIPEA (17 μL, 0.096 mmol), and 30 min later 6-[2-({2-[(5-aminopentyl)amino]-2-oxoethyl}[2-(bis{2-[(α-D-mannopyranosyl) oxy]ethyl}amino)-2-oxoethyl]amino)acetamido]-N-{2-[(α-L-fucopyranosyl)oxy]ethyl}hexanamide (38.1 mg, 0.039 mmol) in DMF (3 mL). After stirred at rt for overnight, the reaction mixture was concentrated, and the residue was purified by reverse phase HPLC (C18 BEH 30×250 mm column, eluting with 10-40 ACN (0.5% TFA) in water (0.1% TFA). The fractions containing the product were combined and freeze-dried to give the title compound. ¹H-NMR (500 MHz, CH₃OH-d4): δ 1.21 (d, 3H), 1.37 (s, 11H), 1.56 (s, 13H), 1.64 (p, 21H), 2.23-2.21 (m, 4H), 2.39-2.38 (m, 2H), 2.87 (s, 1H), 3.00 (s, 1H), 3.25 (dd, 10H), 3.81-3.72 (m, 58H), 4.73 (s, 1H), 4.77 (d, 3H), 4.82 (s, 1H), 5.08 (s, 1H), 5.12 (s, 2H), 7.36 (s, 5H). UPLC-MS Method A: m/z=1005.0 (z=2); t_{R}=4.37 min.

### Step 8: 2,5-Dioxopyrrolidin-1-yl 13-[2-(bis{2-[(α-D-mannopyranosyl)oxy]ethyl}amino)-2-oxoethyl]-1-[(α-L-fucopyranosyl)oxy]-4,11,15,23,27-pentaoxo-25-{2-oxo-2-[(6-oxo-6-{[2-({α-D-mannopyranosyl-(1→3)-[α-D-mannopyranosyl-(1→6)]-α-D-mannopyranosyl}oxy)ethyl]amino}hexyl)amino]ethyl}-3,10,13,16,22,25,28-heptaazatetratriacontan-34-oate

The title compound was prepared using procedures analogous to those described for Example 1, Steps 9 to 10, substituting benzyl 13-[2-(bis{2-[(α-D-mannopyranosyl)oxy]ethyl}amino)-2-oxoethyl]-1-[(α-L-fucopyranosyl)oxy]-4,11,15,23,27-pentaoxo-25-{2-oxo-2-[(6-oxo-6-{[2-({α-D-mannopyranosyl-(1→3)-[α-D-mannopyranosyl-(1→6)]-α-D-mannopyranosyl}oxy) ethyl]amino}hexyl)amino]ethyl}-3,10,13,16,22,25,28-heptaazatetratriacontan-34-oate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1→3)-[α-D-mannopyranosyl-(1→6)]-α-D-mannopyranosyl}oxy) ethyl]amino}ethyl)-3,10,13,16,22,25,28-heptaazatetratriacontan-34-oate in Step 9. UPLC-MS Method A: m/z=1007.9 (z=2); t_{R}=4.11 min.

### Example 42: 2,5-Dioxopyrrolidin-1-yl (S)-1-[(α-L-fucopyranosyl)oxy]-6-[2-({2-[(α-L-fucopyranosyl)oxy]ethyl}amino)-2-oxoethyl]-7-({2-[(α-L-fucopyranosyl)oxy]ethyl}carbamoyl)-22-{2-[(6-{[2-({α-D-mannopyranosyl-(1→3)-[α-D-mannopyranosyl-(1→6)]-α-D-mannopyranosyl}oxy)ethyl]amino}-6-oxohexyl)amino]-2-oxoethyl}-4,13,20,24-tetraoxo-3,6,12,19,22,25-hexaazahentriacontan-31-oate (ML-42)

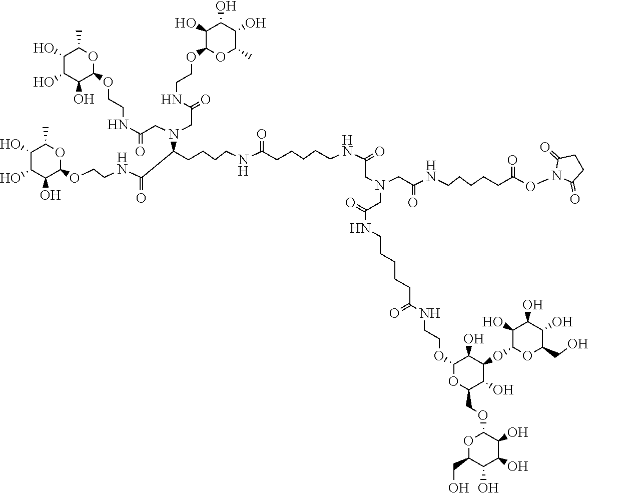 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)C(=O)NCCO[C@@H]2O[C@@H](C)[C@@H](O)[C@@H](O)[C@@H]2O)[C@@H](O)[C@H](O)[C@@H]1O]

### Step 1: (S)-2,2′-{[6-(6-Aminohexanamido)-1-({2-[(α-L-fucopyranosyl)oxy]ethyl}amino)-1-oxohexan-2-yl]azanediyl}bis(N-{2-[(α-L-fucopyranosyl)oxy]ethyl}acetamide)

The title compound was prepared using the procedures analogous to those described for Example 1, Steps 1 to 3, substituting (S)-2,2′-{[7-amino-1-({2-[(α-L-fucopyranosyl)oxy]ethyl}amino)-1-oxoheptan-2-yl]azanediyl}bis(N-{2-[(α-L-fucopyranosyl) oxy]ethyl}acetamide) for 2-aminoethyl α-D-mannopyranoside in Step 2. UPLC-MS Method A: m/z=943.5 (z=1); t_{R}=3.95 min.

### Step 2: 2,5-Dioxopyrrolidin-1-yl (S)-1-[(α-L-fucopyranosyl)oxy]-6-[2-({2-[(α-L-fucopyranosyl) oxy]ethyl}amino)-2-oxoethyl]-7-({2-[(α-L-fucopyranosyl)oxy]ethyl}carbamoyl)-22-{2-[(6-{[2-({α-D-mannopyranosyl-(1→3)-[α-D-mannopyranosyl-(1-+6)]-α-D-mannopyranosyl}oxy)ethyl]amino}-6-oxohexyl)amino]-2-oxoethyl}-4,13,20,24-tetraoxo-3, 6,12,19,22,25-hexaazahentriacontan-31-oate

The title compound was prepared using procedures analogous to those described for Example 37, Steps 7 to 8, substituting (S)-2,2′-{[6-(6-aminohexanamido)-1-({2-[(α-L-fucopyranosyl)oxy]ethyl}amino)-1-oxohexan-2-yl]azanediyl}bis(N-{2-[(α-L-fucopyranosyl)oxy]ethyl}acetamide) for (S)-2,2′-{[7-amino-1-({2-[(α-L-fucopyranosyl)oxy]ethyl amino)-1-oxoheptan-2-yl]azanediyl}bis(N-{2-[(α-L-fucopyranosyl)oxy]ethyl}acetamide) in Step 7. UPLC-MS Method A: m/z=984.9 (z=2); t_{R}=4.25 min.

### Example 43: 2,5-Dioxopyrrolidin-1-yl (7S,15S)-1-[(α-D-mannopyranosyl)oxy]-7-({2-[(α-D-mannopyranosyl)oxy]ethyl}carbamoyl)-4,9,14,17,21-pentaoxo-19-{2-oxo-2-[(6-oxo-6-{[2-({α-D-mannopyranosyl-(1→3)-[α-D-mannopyranosyl-(1→6)]-α-D-mannopyranosyl}oxy) ethyl]amino}hexyl)amino]ethyl}-15-(3-oxo-3-{[2-({α-D-mannopyranosyl-(1→3)-[α-D-mannopyranosyl-(1→6)]-α-D-mannopyranosyl}oxy)ethyl]amino}propyl)-3,8,13,16,19,22-hexaazaoctacosan-28-oate (ML-43)

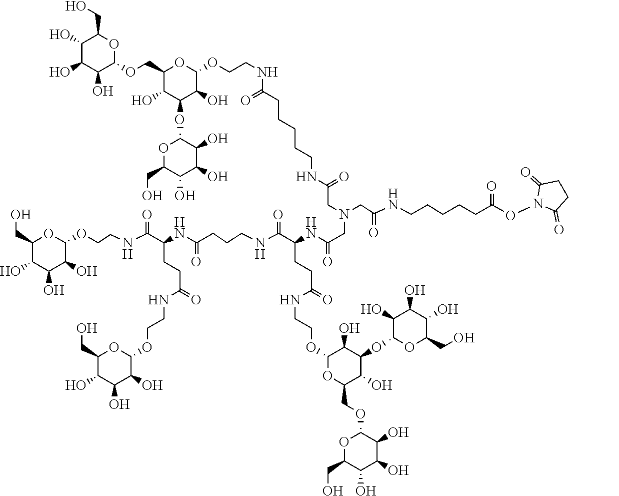 [SMILES: O=C(CCCCCNC(=O)CN(CC(=O)NCCCCCC(=O)ON1C(=O)CCC1=O)CC(=O)N[C@@H](CCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)C(=O)NCCCC(=O)N[C@@H](CCC(=O)NCCO[C@H]1O[C@H](CO)[C@@H](O)[C@H](O)[C@@H]1O)C(=O)NCCO[C@H]1O[C@H](CO)[C@@H](O)[C@H](O)[C@@H]1O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl (S)-(4-{[1,5-bis({2-[(α-D-mannopyranosyl)oxy]ethyl}amino)-1,5-dioxopentan-2-yl]amino}-4-oxobutyl)carbamate

To a solution of (2S)-2-amino-N¹,N⁵-bis{2-[(α-D-mannopyranosyl)oxy]ethyl}pentanediamide (1 g, 1.79 mmol) in DMF (7 mL) at rt was added 4-{[(benzyloxy)carbonyl]amino}butanoic acid (468 mg, 1.97 mmol), EDC (688 mg, 3.59 mmol), HOBt (27 mg, 0.18 mmol), and TEA (25 μL, 0.18 mmol). After stirring at rt for 16 h, the mixture was concentrated, and the residue was purified by reverse phase chromatography to isolate the title compound. UPLC-MS Method A: m/z=777.2 (z=1); t_{R}=3.55 min.

### Step 2: (S)-2-(4-Aminobutanamido)-N¹,N⁵-bis{2-[(α-D-mannopyranosyl)oxy]ethyl}pentanediamide

The title compound was prepared using the procedure analogous to that described for Example 1, Step 3, substituting benzyl (S)-(4-{[1,5-bis({2-[(α-D-mannopyranosyl)oxy]ethyl}amino)-1,5-dioxopentan-2-yl]amino}-4-oxobutyl)carbamate for benzyl [6-({2-[(α-D-mannopyranosyl)oxy]ethyl}amino)-6-oxohexyl]carbamate. UPLC-MS Method A: m/z=643.2 (z=2); t_{R}=2.95 min.

### Step 3: Benzyl (S)-4-{[(benzyloxy)carbonyl]amino}-5-[(4-{[(2S)-1,5-bis({2-[(α-D-mannopyranosyl)oxy]ethyl}amino)-1,5-dioxopentan-2-yl]amino}-4-oxobutyl)amino]-5-oxopentanoate

To a solution of N-α-benzyloxycarbonyl-D-glutamic acid-α-benzyl ester (360 mg, 0.97 mmol) in DMF (5 mL) at rt was added (S)-2-(4-aminobutanamido)-N¹,N⁵-bis{2-[(α-D-mannopyranosyl)oxy]ethyl}pentanediamide (810 mg, 1.26 mmol), EDC (186 mg, 0.97 mmol), HOBt (15 mg, 0.097 mmol), and DIPEA (17 μL, 0.097 mmol). After stirring at rt for 16 h, the mixture was concentrated, and the residue was purified by reverse phase chromatography to give the title compound. UPLC-MS Method A: m/z=997.0 (z=1); t_{R}=2.84 min.

### Step 4: (S)-4-{[(Benzyloxy)carbonyl]amino}-5-[(4-{[(2S)-1,5-bis({2-[(α-D-mannopyranosyl) oxy]ethyl}amino)-1,5-dioxopentan-2-yl]amino}-4-oxobutyl)amino]-5-oxopentanoic acid

To a solution of benzyl (S)-4-{[(benzyloxy)carbonyl]amino}-5-[(4-{[(2S)-1,5-bis({2-[(α-D-mannopyranosyl)oxy]ethyl}amino)-1,5-dioxopentan-2-yl]amino}-4-oxobutyl) amino]-5-oxopentanoate (797 mg, 0.8 mmol) in water (5 mL) at rt was added 1.0 N aq. NaOH solution (0.96 mL, 0.96 mmol). After stirring at rt for 2 h, the reaction mixture was treated with AcOH (69 μL, 1.2 mmol), stirred for additional 15 min and then concentrated. The residue was purified by reverse phase chromatography to isolate the title compound. UPLC-MS Method A: m/z=906.2 (z=1); t_{R}=3.64 min.

### Step 5: Benzyl {(7S,15S)-1-[(α-D-mannopyranosyl)oxy]-7-({2-[(α-D-mannopyranosyl)oxy]ethyl} carbamoyl)-21-({α-D-mannopyranosyl-(1→3)-[α-D-mannopyranosyl-(1→6)]-α-D-mannopyranosyl}oxy)-4,9,14,18-tetraoxo-3,8,13,19-tetraazahenicosan-15-yl}carbamate

To a solution of (S)-4-{[(benzyloxy)carbonyl]amino}-5-[(4-{[(2S)-1,5-bis({2-[(α-D-mannopyranosyl)oxy]ethyl}amino)-1,5-dioxopentan-2-yl]amino}-4-oxobutyl)amino]-5-oxopentanoic acid (297 mg, 0.328 mmol) and bis{2-[(α-D-mannopyranosyl)oxy]ethyl}amine (197 mg, 0.361 mmol) in DMF (3 mL) at rt was added EDC (126 mg, 0.656 mmol), HOBt (50 mg, 0.328 mmol), and DIPEA (86 μL, 0.492 mmol). After stirring at rt for 72 h, the reaction mixture was concentrated, and the residue was purified by reverse phase chromatography to isolate the title compound. UPLC-MS Method A: m/z=1435.3 (z=1); t_{R}=2.97 min.

### Step 6: (S)-2-Amino-N¹-(4-{[(S)-1,5-bis({2-[(α-D-mannopyranosyl)oxy]ethyl}amino)-1,5-dioxopentan-2-yl]amino}-4-oxobutyl)-N⁵-[2-({α-D-mannopyranosyl-(1→3)-[α-D-mannopyranosyl-(1→6)]-α-D-mannopyranosyl}oxy)ethyl]pentanediamide

The title compound was prepared using the procedure analogous to that described for Example 1, Step 3, substituting benzyl {(7S,15S)-1-[(α-D-mannopyranosyl)oxy]-7-({2-[(α-D-mannopyranosyl)oxy]ethyl}carbamoyl)-21-({α-D-mannopyranosyl-(1-3)-[α-D-mannopyranosyl-(1→6)]-α-D-mannopyranosyl}oxy)-4,9,14,18-tetraoxo-3,8,13,19-tetraazahenicosan-15-yl}carbamate for benzyl [6-({2-[(α-D-mannopyranosyl)oxy]ethyl}amino)-6-oxohexyl]carbamate. UPLC-MS Method A: m/z=1301.1 (z=1); t_{R}=2.49 min.

### Step 7: 2,5-Dioxopyrrolidin-1-yl (7S,15S)-1-[(α-D-mannopyranosyl)oxy]-7-({2-[(α-D-mannopyranosyl)oxy]ethyl}carbamoyl)-4,9,14,17,21-pentaoxo-19-{2-oxo-2-[(6-oxo-6-{[2-({α-D-mannopyranosyl-(1→3)-[α-D-mannopyranosyl-(1→6)]-α-D-mannopyranosyl}oxy)ethyl]amino} hexyl)amino]ethyl}-15-(3-oxo-3-{[2-({α-D-mannopyranosyl-(1→3)-[α-D-mannopyranosyl-(1→6)]-α-D-mannopyranosyl}oxy)ethyl]amino}propyl)-3,8,13,16,19,22-hexaazaoctacosan-28-oate

The title compound was prepared using the procedure analogous to that described for Example 37, Steps 7 to 8, substituting (S)-2-amino-N¹-(4-{[(S)-1,5-bis({2-[(α-D-mannopyranosyl)oxy]ethyl}amino)-1,5-dioxopentan-2-yl]amino}-4-oxobutyl)-N-[2-({α-D-mannopyranosyl-(1→3)-[α-D-mannopyranosyl-(1→6)]-α-D-mannopyranosyl}oxy)ethyl]pentanediamide for (S)-2,2′-{[7-amino-1-({2-[(α-L-fucopyranosyl)oxy]ethyl}amino)-1-oxoheptan-2-yl]azanediyl}bis(N-{2-[(α-L-fucopyranosyl)oxy]ethyl}acetamide) in Step 7. UPLC-MS Method A: m/z=1164.6 (z=2); t_{R}=3.06 min.

### Example 44: 2,5-Dioxopyrrolidin-1-yl (7S,14S)-1-[(α-L-fucopyranosyl)oxy]-6-[2-({2-[(α-L-fucopyranosyl)oxy]ethyl}amino)-2-oxoethyl]-14-{4-[6-({2-[(α-L-fucopyranosyl)oxy]ethyl}amino)-6-oxohexanamido]butyl}-7-({2-[(α-L-fucopyranosyl)oxy]ethyl}carbamoyl)-4,13,16-trioxo-3,6,12,15-tetraazahenicosan-21-oate (ML-44)

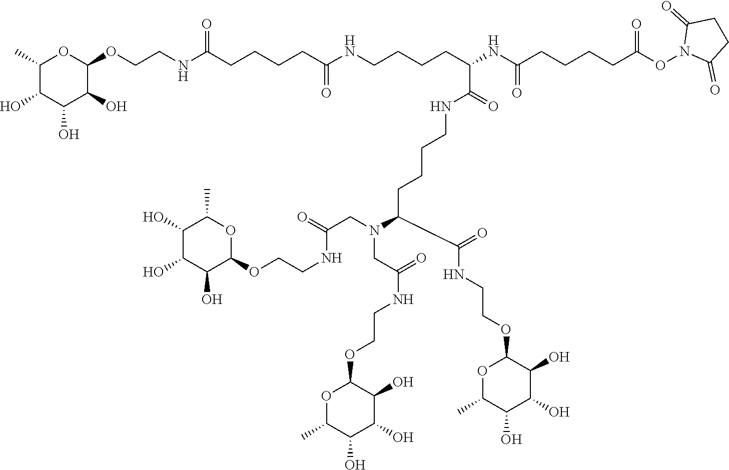 [SMILES: C[C@@H]1O[C@@H](OCCNC(=O)CCCCC(=O)NCCCC[C@H](NC(=O)CCCCC(=O)ON2C(=O)CCC2=O)C(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)[C@@H](O)[C@H](O)[C@@H]1O]

### Step 1: Benzyl 6-({2-[(α-L-fucopyranosyl)oxy]ethyl}amino)-6-oxohexanoate

To a stirred solution of 6-(benzyloxy)-6-oxohexanoic acid (3.42 g, 14.48 mmol) in DMF (5 mL) at rt was added EDC (4.163 g, 21.72 mmol), and HOBt (1.108 g, 7.24 mmol), and 20 min later, 2-aminoethyl α-L-fucopyranoside (3.0 g, 14.48 mmol). After stirring at rt overnight, the reaction mixture was concentrated, and the residue was purified by reverse phase HPLC (DELTA-PAK™ C4 300 A, 15 μm optical bed density (“OBD”), 50×250 mm column, gradient 8-25% in 25 min, ACN/water, flow rate 85 ml/min, to give the title compound.

### Step 2: 6-({2-[(α-L-Fucopyranosyl)oxy]ethyl}amino)-6-oxohexanoic acid

The title compound was prepared using the procedure analogous to that described for Example 1, Step 3, substituting benzyl 6-({2-[(α-L-fucopyranosyl)oxy]ethyl}amino)-6-oxohexanoate for benzyl [6-({2-[(α-D-mannopyranosyl)oxy]ethyl}amino)-6-oxohexyl]carbamate. UPLC-MS Method A: m/z=643.2 (z=2); t_{R}=2.95 min.

### Step 3: 2,5-Dioxopyrrolidin-1-yl 6-({2-[(α-L-fucopyranosyl)oxy]ethyl}amino)-6-oxohexanoate

To a stirred solution of 6-({2-[(α-L-fucopyranosyl)oxy]ethyl}amino)-6-oxohexanoic acid (1.2 g, 3.58 mmol) in DMF (10 mL) at 0° C. was added TSTU (1.131 g, 3.76 mmol) and DIPEA (687 μL, 3.94 mmol). The resulting mixture was allowed to warm to rt, stirred for overnight and concentrated to give the title compound, which was used without further purification.

### Step 4: N²-[(Benzyloxy)carbonyl]-N⁶-[6-({2-[(α-L-fucopyranosyl)oxy]ethyl}amino)-6-oxohexanoyl]-L-lysine

To a stirred solution of (S)-6-amino-2-{[(benzyloxy)carbonyl]amino}hexanoic acid (700 mg, 2.497 mmol) in DMF (5 mL) at 0° C. was added 2,5-dioxopyrrolidin-1-yl 6-({2-[(α-L-fucopyranosyl)oxy]ethyl}amino)-6-oxohexanoate (1.08 g, 2.497 mmol) and DIPEA (436 μL, 2.497 mmol). After stirring at rt for 18 h, the reaction mixture was concentrated, and the residue was purified by reverse phase HPLC (Delta Pak C4 300 Å, 15 m OBD, 50×250 mm column. Injection volume 3000 μL, gradient 8-25% in 25 min, ACN/water, flow rate 85 ml/min) to give the title compound.

### Step 5: 2,5-Dioxopyrrolidin-1-yl N²-[(benzyloxy)carbonyl]-N⁶-[6-({2-[(α-L-fucopyranosyl)oxy]ethyl}amino)-6-oxohexanoyl]-L-lysinate

To a stirred solution of N²-[(benzyloxy)carbonyl]-N⁶-[6-({2-[(α-L-fucopyranosyl)oxy]ethyl}amino)-6-oxohexanoyl]-L-lysine (220 mg, 0.368 mmol) in DMF (10 mL) at 0° C. was added TSTU (116 mg, 0.387 mmol) and DIPEA (64 μL, 0.368 mmol). After stirring at rt for 18 h, the reaction mixture was concentrated and dried in vacuo to give the title crude product to use in next step without purification.

### Step 6: Benzyl {(7S,14S)-1,28-bis[(α-L-fucopyranosyl)oxy]-6-[2-({2-[(α-L-fucopyranosyl)oxy]ethyl}amino)-2-oxoethyl]-7-({2-[(α-L-fucopyranosyl)oxy]ethyl}carbamoyl)-4,13,20,25-tetraoxo-3,6,12,19,26-pentaazaoctacosan-14-yl}carbamate

To a stirred solution of (S)-2,2′-{[7-amino-1-({2-[(α-L-fucopyranosyl)oxy]ethyl} amino)-1-oxoheptan-2-yl]azanediyl}bis(N-{2-[(α-L-fucopyranosyl)oxy]ethyl}acetamide) (220 mg, 0.265 mmol) in DMF (3 mL) at 0° C. was added 2,5-dioxopyrrolidin-1-yl N²-[(benzyloxy) carbonyl]-N⁶-[6-({2-[(α-L-fucopyranosyl)oxy]ethyl}amino)-6-oxohexanoyl]-L-lysinate (239 mg, 0.345 mmol) and DIPEA (46 μL, 0.265 mmol). After stirring at rt for 18 h, the reaction mixture was concentrated, and the residue was purified by reverse phase chromatography, eluting with 0-30% ACN in water, to give the title compound.

### Step 7: N¹—[(S)-5-Amino-6-{[(S)-5-{bis[2-({2-[(α-L-fucopyranosyl)oxy]ethyl}amino)-2-oxoethyl]amino}-6-({2-[(α-L-fucopyranosyl)oxy]ethyl}amino)-6-oxohexyl]amino}-6-oxohexyl]-N⁶-{2-[(α-L-fucopyranosyl)oxy]ethyl}adipamide

The title compound was prepared using the procedure analogous to that described for Example 1, Step 3, substituting benzyl {(7S,14S)-1,28-bis[(α-L-fucopyranosyl)oxy]-6-[2-({2-[(α-L-fucopyranosyl)oxy]ethyl}amino)-2-oxoethyl]-7-({2-[(α-L-fucopyranosyl)oxy]ethyl}carbamoyl)-4,13,20,25-tetraoxo-3,6,12,19,26-pentaazaoctacosan-14-yl}carbamate for benzyl [6-({2-[(α-D-mannopyranosyl)oxy]ethyl}amino)-6-oxohexyl]carbamate. UPLC-MS Method A: m/z=643.2 (z=2); t_{R}=2.95 min.

### Step 8: Benzyl (7S,14S)-1-[(α-L-fucopyranosyl)oxy]-6-[2-({2-[(α-L-fucopyranosyl)oxy]ethyl}amino)-2-oxoethyl]-14-{4-[6-({2-[(α-L-fucopyranosyl)oxy]ethyl}amino)-6-oxohexanamido]butyl}-7-({2-[(α-L-fucopyranosyl)oxy]ethyl}carbamoyl)-4,13,16-trioxo-3,6,12,15-tetraazahenicosan-21-oate

To a stirred solution of N¹—[(S)-5-amino-6-{[(S)-5-{bis[2-({2-[(α-L-fucopyranosyl)oxy]ethyl}amino)-2-oxoethyl]amino}-6-({2-[(α-L-fucopyranosyl)oxy]ethyl} amino)-6-oxohexyl]amino}-6-oxohexyl]-N⁶-{2-[(α-L-fucopyranosyl)oxy]ethyl}adipamide (100 mg, 0.078 mmol) in DMF (1.0 mL) at 0° C. was added benzyl (2,5-dioxopyrrolidin-1-yl) adipate (26.1 mg, 0.078 mmol) and DIPEA (13.69 μL, 0.078 mmol). The reaction mixture was warmed up to rt and stirred for 1 h. To the resulting mixture was added acetone (35 mL), and the precipitate was collected as pellet by discarding supernatant after centrifugation (30 min, 3500 rpm, 4° C.). The pellet was purified by reverse phase chromatography (C18, 130 g), eluting with 0-30% ACN in water, to give the title compound.

### Step 9: 2,5-Dioxopyrrolidin-1-yl (7S,14S)-1-[(α-L-fucopyranosyl)oxy]-6-[2-({2-[(α-L-fucopyranosyl)oxy]ethyl}amino)-2-oxoethyl]-14-{4-[6-({2-[(α-L-fucopyranosyl)oxy]ethyl}amino)-6-oxohexanamido]butyl}-7-({2-[(α-L-fucopyranosyl)oxy]ethyl}carbamoyl)-4,13,16-trioxo-3,6,12,15-tetraazahenicosan-21-oate

The title compound was prepared using procedures analogous to those described for Example 1, Steps 9 to 10, substituting benzyl (7S,14S)-1-[(α-L-fucopyranosyl)oxy]-6-[2-({2-[(α-L-fucopyranosyl)oxy]ethyl}amino)-2-oxoethyl]-14-{4-[6-({2-[(α-L-fucopyranosyl)oxy]ethyl}amino)-6-oxohexanamido]butyl}-7-({2-[(α-L-fucopyranosyl)oxy]ethyl}carbamoyl)-4,13,16-trioxo-3,6,12,15-tetraazahenicosan-21-oate for benzyl 1-[(α-D-mannopyranosyl)oxy]-25-{24-[(α-D-mannopyranosyl)oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15,23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino} ethyl)-3,10,13,16,22,25,28-heptaazatetratriacontan-34-oate in Step 9. UPLC-MS Method A: m/z=1007.9 (z=2); t_{R}=4.11 min.

### Example 45: 2,5-Dioxopyrrolidin-1-yl (7S,14S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-14-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,13,16-trioxo-3,6,12,15-tetraazahenicosan-21-oate (ML-45)

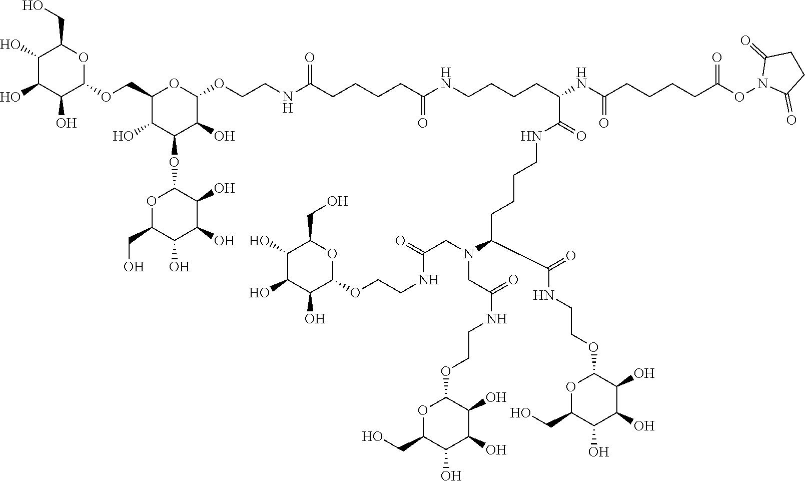 [SMILES: O=C(CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@H](NC(=O)CCCCC(=O)ON1C(=O)CCC1=O)C(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O]

The title compound was prepared using procedures analogous to those described for Example 44, substituting 2-({α-D-mannopyranosyl-(1-3)-[α-D-mannopyranosyl-(1→6)]-α-D-mannopyranosyl}oxy)ethan-1-amine for 2-aminoethyl α-L-fucopyranoside in Step 1, and (S)-2,2′-{[7-amino-1-({2-[(α-D-mannopyranosyl)oxy]ethyl}amino)-1-oxoheptan-2-yl]azanediyl}bis(N-{2-[(α-D-mannopyranosyl)oxy]ethyl}acetamide) for (S)-2,2′-{[7-amino-1-({2-[(α-L-fucopyranosyl)oxy]ethyl}amino)-1-oxoheptan-2-yl]azanediyl}bis(N-{2-[(α-L-fucopyranosyl) oxy]ethyl}acetamide) in Step 6, respectively. UPLC-MS Method A: m/z=1007.9 (z=2); t_{R}=4.11 min.

### Example 46: 2,5-Dioxopyrrolidin-1-yl (7S,14S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-14-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,13,16-trioxo-3,6,12,15-tetraazahenicosan-21-oate (ML-46)

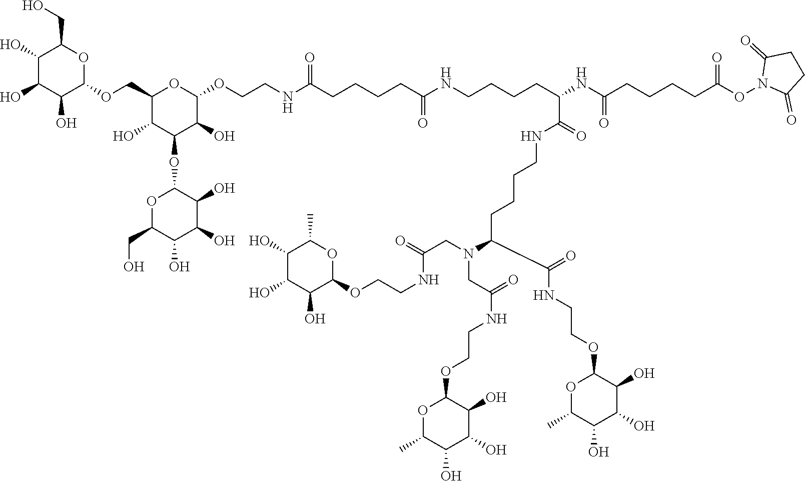 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)[C@H](CCCCNC(=O)CCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)NC(=O)CCCCC(=O)ON2C(=O)CCC2=O)C(=O)NCCO[C@@H]2O[C@@H](C)[C@@H](O)[C@@H](O)[C@@H]2O)[C@@H](O)[C@H](O)[C@@H]1O]

The title compound was prepared using procedures analogous to those described for Example 44, substituting 2-({α-D-mannopyranosyl-(1-3)-[α-D-mannopyranosyl-(1→6)]-α-D-mannopyranosyl}oxy)ethan-1-amine for 2-aminoethyl α-L-fucopyranoside in Step 1. UPLC-MS Method A: m/z=1007.9 (z=2); t_{R}=4.11 min.

### Example 47: 2,5-Dioxopyrrolidin-1-yl (7S,14S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-14-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,13,16-trioxo-3,6,12,15-tetraazatricosan-23-oate (ML-47)

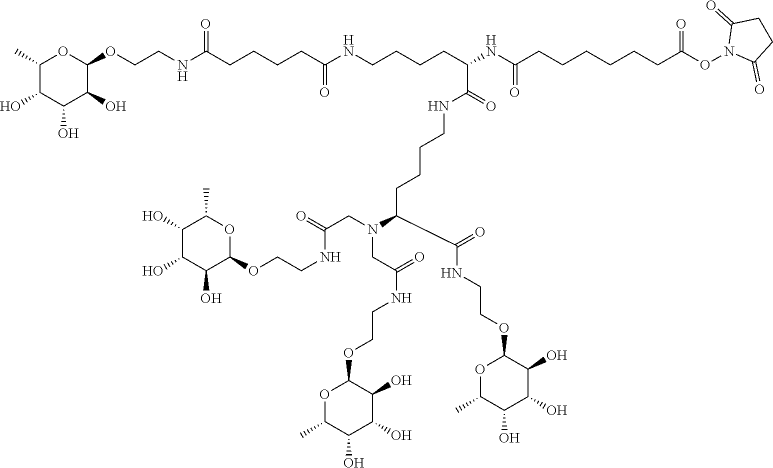 [SMILES: C[C@@H]1O[C@@H](OCCNC(=O)CCCCC(=O)NCCCC[C@H](NC(=O)CCCCCCC(=O)ON2C(=O)CCC2=O)C(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)[C@@H](O)[C@H](O)[C@@H]1O]

The title compound was prepared using procedures analogous to those described for Example 44, substituting 1-benzyl 8-(2,5-dioxopyrrolidin-1-yl) octanedioate for benzyl (2,5-dioxopyrrolidin-1-yl) adipate in Step 8. UPLC-MS Method A: m/z=1007.9 (z=2); t_{R}=4.11 min.

### Example 48: 2,5-Dioxopyrrolidin-1-yl (7S,21S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-21-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,13,20,23-tetraoxo-3,6,12,19,22-pentaazaoctacosan-28-oate (ML-48)

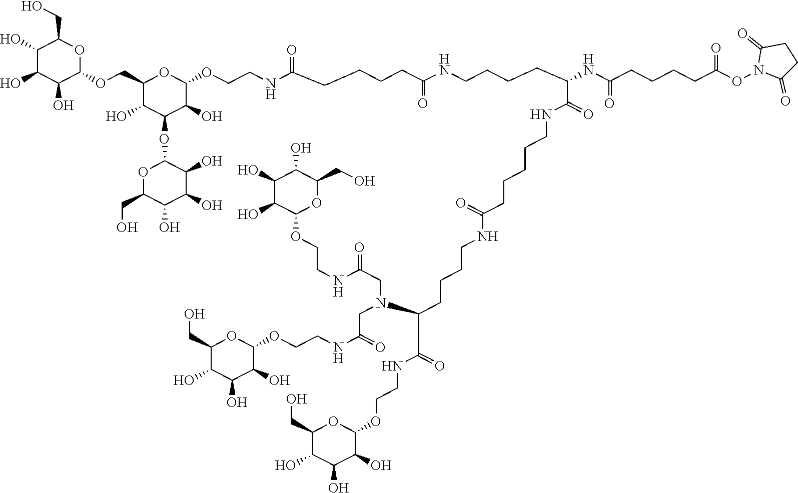 [SMILES: O=C(CCCCCNC(=O)[C@H](CCCCNC(=O)CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NC(=O)CCCCC(=O)ON1C(=O)CCC1=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O]

### Step 1: (S)-2,2′-{[6-(6-Aminohexanamido)-1-({2-[(α-D-mannopyranosyl)oxy]ethyl}amino)-1-oxohexan-2-yl]azanediyl}bis(N-{2-[(α-D-mannopyranosyl)oxy]ethyl}acetamide)

The title compound was prepared using the procedures analogous to those described for Example 1, Steps 1 to 3, substituting (S)-2,2′-{[7-amino-1-({2-[(α-D-mannopyranosyl)oxy]ethyl}amino)-1-oxoheptan-2-yl]azanediyl}bis(N-{2-[(α-D-mannopyranosyl)oxy]ethyl}acetamide) for 2-aminoethyl α-D-mannopyranoside in Step 2. UPLC-MS Method A: m/z=943.5 (z=1); t_{R}=3.95 min.

### Step 2: 2,5-Dioxopyrrolidin-1-yl (7S,21S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-21-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,13,20,23-tetraoxo-3,6,12,19,22-pentaazaoctacosan-28-oate

The title compound was prepared using procedures analogous to those described for Example 44, substituting 2-({α-D-mannopyranosyl-(1-3)-[α-D-mannopyranosyl-(1→6)]-α-D-mannopyranosyl}oxy)ethan-1-amine for 2-aminoethyl α-L-fucopyranoside in Step 1, and (S)-2,2′-{[6-(6-aminohexanamido)-1-({2-[(α-D-mannopyranosyl)oxy]ethyl}amino)-1-oxohexan-2-yl]azanediyl}bis(N-{2-[(α-D-mannopyranosyl)oxy]ethyl}acetamide) for (S)-2,2′-{[7-amino-1-({2-[(α-L-fucopyranosyl)oxy]ethyl}amino)-1-oxoheptan-2-yl]azanediyl}bis(N-{2-[(α-L-fucopyranosyl)oxy]ethyl}acetamide) in Step 6, respectively. UPLC-MS Method A: m/z=1007.9 (z=2); t_{R}=4.11 min.

### Example 49: 2,5-Dioxopyrrolidin-1-yl (7S,12S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-12-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,10,13-trioxo-3,6,11,14-tetraazaicosan-20-oate (ML-49)

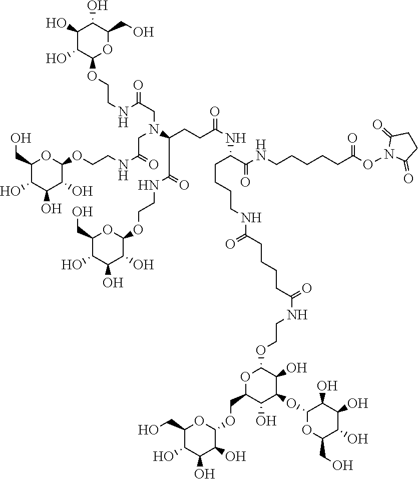 [SMILES: O=C(CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@H](NC(=O)CC[C@@H](C(=O)NCCO[C@@H]1O[C@H](CO)[C@@H](O)[C@H](O)[C@H]1O)N(CC(=O)NCCO[C@@H]1O[C@H](CO)[C@@H](O)[C@H](O)[C@H]1O)CC(=O)NCCO[C@@H]1O[C@H](CO)[C@@H](O)[C@H](O)[C@H]1O)C(=O)NCCCCCC(=O)ON1C(=O)CCC1=O]

### Step 1: Benzyl (S)-4-{bis[2-({2-[(α-D-mannopyranosyl)oxy]ethyl}amino)-2-oxoethyl]amino}-5-({2-[(α-D-mannopyranosyl)oxy]ethyl}amino)-5-oxopentanoate

To a stirred solution of (S)-2,2′-{[4-(benzyloxy)-1-carboxy-4-oxobutyl]azanediyl}diacetic acid (500 mg, 1.415 mmol, WO 2018/175272 A1) in DMF (15 mL) at rt was added EDC (1.085 g, 5.66 mmol), HOBt (217 mg, 1.415 mmol), and 20 min later, 2-aminoethyl α-D-mannopyranoside (500 mg, 1.415 mmol). After stirring at rt for 18 h, the reaction mixture was concentrated, and the residue was purified by reverse phase chromatography (C18), eluting with 0-40% ACN in water, to give the title product.

### Step 2: (S)-4-{bis[2-({2-[(α-D-Mannopyranosyl)oxy]ethyl}amino)-2-oxoethyl]amino}-5-({2-[(α-D-mannopyranosyl)oxy]ethyl}amino)-5-oxopentanoic acid

The title compound was prepared using the procedure analogous to that described for Example 1, Step 3, substituting benzyl (S)-4-{bis[2-({2-[(α-D-mannopyranosyl)oxy]ethyl}amino)-2-oxoethyl]amino}-5-({2-[(α-D-mannopyranosyl)oxy]ethyl}amino)-5-oxopentanoate for benzyl [6-({2-[(α-D-mannopyranosyl)oxy]ethyl}amino)-6-oxohexyl]carbamate.

### Step 3: 2,5-Dioxopyrrolidin-1-yl (S)-4-{bis[2-({2-[(α-D-mannopyranosyl)oxy]ethyl}amino)-2-oxoethyl]amino}-5-({2-[(α-D-mannopyranosyl)oxy]ethyl}amino)-5-oxopentanoate

To a stirred solution of (S)-4-{bis[2-({2-[(α-D-mannopyranosyl)oxy]ethyl} amino)-2-oxoethyl]amino}-5-({2-[(α-D-mannopyranosyl)oxy]ethyl}amino)-5-oxopentanoic acid (150 mg, 0.171 mmol) in DMF (3 mL) at 0° C. was added TSTU (56.5 mg, 0.188 mmol) and DIPEA (31 μL, 0.171 mmol). The reaction mixture was warmed up to rt, stirred for 2 h and pipetted to acetone (35 mL). The resulting precipitate was collected as pellet by discarding supernatant after centrifugation. The pellet was dried in vacuo to give desired product.

### Step 4: N²-[(Benzyloxy)carbonyl]-N⁶-(6-{[2-({α-D-mannopyranosyl-(1→3)-[α-D-mannopyranosyl-(1→6)]-α-D-mannopyranosyl}oxy)ethyl]amino}-6-oxohexanoyl)-L-lysine

The title compound was prepared using the procedures analogous to those described for Example 44, Steps 1 to 4, substituting 2-({α-D-mannopyranosyl-(1-3)-[α-D-mannopyranosyl-(1→6)]-α-D-mannopyranosyl}oxy)ethan-1-amine for 2-aminoethyl α-L-fucopyranoside in Step 1.

### Step 5: N⁶-(6-{[2-({α-D-Mannopyranosyl-(1→3)-[α-D-mannopyranosyl-(1→6)]-α-D-mannopyranosyl}oxy)ethyl]amino}-6-oxohexanoyl)-L-lysine

The title compound was prepared using the procedure analogous to that described for Example 1, Step 3, substituting N²-[(benzyloxy)carbonyl]-N⁶-(6-{[2-({α-D-mannopyranosyl-(1→3)-[α-D-mannopyranosyl-(1→6)]-α-D-mannopyranosyl}oxy)ethyl]amino}-6-oxohexanoyl)-L-lysine for benzyl [6-({2-[(α-D-mannopyranosyl)oxy]ethyl}amino)-6-oxohexyl]carbamate.

### Step 6: N²—[(S)-4-{bis[2-({2-[(α-D-Mannopyranosyl)oxy]ethyl}amino)-2-oxoethyl]amino}-5-({2-[(α-D-mannopyranosyl)oxy]ethyl}amino)-5-oxopentanoyl]-N⁶-(6-{[2-({α-D-mannopyranosyl-(1→3)-[α-D-mannopyranosyl-(1→6)]-α-D-mannopyranosyl}oxy)ethyl]amino}-6-oxohexanoyl)-L-lysine

To a stirred solution of N⁶-(6-{[2-({α-D-mannopyranosyl-(1-3)-[α-D-mannopyranosyl-(1→6)]-α-D-mannopyranosyl}oxy)ethyl]amino}-6-oxohexanoyl)-L-lysine (126 mg, 0.157 mmol) in DMF (3 mL) at 0° C. was added 2,5-dioxopyrrolidin-1-yl (S)-4-{bis[2-({2-[(α-D-mannopyranosyl)oxy]ethyl}amino)-2-oxoethyl]amino}-5-({2-[(α-D-mannopyranosyl)oxy]ethyl}amino)-5-oxopentanoate (153 mg, 0.157 mmol) in DMF (1.0 mL) dropwise and followed by TEA (22 μL, 0.157 mmol). The reaction mixture was warmed to rt, stirred for 4 h, and pipetted to acetone (35 mL) and centrifuged to give desired product.

### Step 7: Benzyl (7S,12S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-12-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,10,13-trioxo-3,6,11,14-tetraazaicosan-20-oate

To a stirred solution of N²—[(S)-4-{bis[2-({2-[(α-D-mannopyranosyl)oxy]ethyl} amino)-2-oxoethyl]amino}-5-({2-[(α-D-mannopyranosyl)oxy]ethyl}amino)-5-oxopentanoyl]-N⁶-(6-{[2-({α-D-mannopyranosyl-(1→3)-[α-D-mannopyranosyl-(1→6)]-α-D-mannopyranosyl}oxy)ethyl]amino}-6-oxohexanoyl)-L-lysine (140 mg, 0.083 mmol) in DMF (3 mL) at rt was added EDC (15.99 mg, 0.083 mmol), HOBt (12.77 mg, 0.083 mmol), and 6-(benzyloxy)-6-oxohexan-1-aminium 4-methylbenzenesulfonate (32.8 mg, 0.083 mmol). The reaction mixture was stirred at rt for 18 h and pipetted dropwise to acetone (30 mL) to generate a precipitate, which was, after centrifugation, collected and purified by reverse phase chromatography (C18, eluting with 0-50% ACN in water) to give the title compound.

### Step 8: 2,5-Dioxopyrrolidin-1-yl (7S,12S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-12-[4-(6-{[2-({α-D-mannopyranosyl-(1→3)-[α-D-mannopyranosyl-(1→6)]-α-D-mannopyranosyl}oxy)ethyl]amino}-6-oxohexanamido)butyl]-4,10,13-trioxo-3,6,11,14-tetraazaicosan-20-oate

The title compound was prepared using procedures analogous to those described for Example 1, Steps 9 to 10, substituting benzyl (7S,12S)-1-[(α-D-mannopyranosyl)oxy]-6-[2-({2-[(α-D-mannopyranosyl)oxy]ethyl}amino)-2-oxoethyl]-7-({2-[(α-D-mannopyranosyl)oxy]ethyl}carbamoyl)-12-[4-(6-{[2-({α-D-mannopyranosyl-(1-3)-[α-D-mannopyranosyl-(1→6)]-α-D-mannopyranosyl}oxy)ethyl]amino}-6-oxohexanamido)butyl]-4,10,13-trioxo-3,6,11,14-tetraazaicosan-20-oate for benzyl 1-[(α-D-mannopyranosyl)oxy]-25-{24-[(α-D-mannopyranosyl) oxy]-2,10,14,21-tetraoxo-12-(2-oxo-2-{[2-({α-D-mannopyranosyl-(1-3)-[α-D-mannopyranosyl-(1→6)]-α-D-mannopyranosyl}oxy)ethyl]amino}ethyl)-3,9,12,15,22-pentaazatetracosyl}-4,11,15, 23,27-pentaoxo-13-(2-oxo-2-{[2-({α-D-mannopyranosyl-(1-3)-[α-D-mannopyranosyl-(1→6)]-α-D-mannopyranosyl}oxy)ethyl]amino}ethyl)-3,10,13,16,22,25,28-heptaazatetratriacontan-34-oate in Step 9. UPLC-MS Method A: m/z=1007.9 (z=2); t_{R}=4.11 min.

### Example 50: 6-[(2,5-Dioxopyrrolidin-1-yl)oxy]-N-{2-[(α-L-fucopyranosyl)oxy]ethyl}-6-oxohexanamide (ML-50)

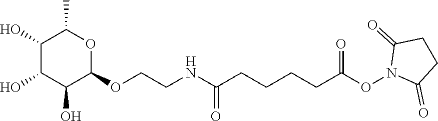 [SMILES: C[C@@H]1O[C@@H](OCCNC(=O)CCCCC(=O)ON2C(=O)CCC2=O)[C@@H](O)[C@H](O)[C@@H]1O]

### Step 1: Benzyl 6-oxo-6-((2-(((2R,3S,4R,5S,6S)-3,4,5-trihydroxy-6-methyltetrahydro-2H-pyran-2-yl)oxy)ethyl)amino)hexanoate

To a solution of 2-aminoethyl α-L-fucopyranoside (Robert Sardzik et al., Preparation of aminoethylglycosidesfor glycoconjugation, 6 BEILSTEIN J. ORG. CHEM. 699-703 (2010)) in DMF (30 mL) was added 6-(benzyloxy)-6-oxohexanoic acid (902 mg, 3.82 mmol), EDC (998 mg, 5.21 mmol), HOBt (797 mg, 5.21 mmol) and DIPEA [1.21 mL, 6.94 mmol). After stirring at 23° C. for 16 h the mixture evaporated and the residue was purified by flash chromatography on C18 reverse silica gel column (130 g), eluting with 10-50% AcCN in H₂O to give the title compound. UPLC Method B: observed m/e=426.1 [M+1]; t_{R}=2.9 min.

### Step 2: 6-Oxo-6-((2-(((2R,3S,4R,5S,6S)-3,4,5-trihydroxy-6-methyltetrahydro-2H-pyran-2-yl)oxy)ethyl)amino)hexanoic acid

A mixture of benzyl 6-oxo-6-((2-(((2R,3S,4R,5S,6S)-3,4,5-trihydroxy-6-methyltetrahydro-2H-pyran-2-yl)oxy)ethyl)amino)hexanoate (1.1 g, 2.59 mmol) and Pd/C (0.275 mg, 2.59 mmol) in water (80 mL) was allowed to stir under a balloon of H₂ at rt for 4 h. The catalyst was filtered off and washed with H₂O 20 mL). The filtrate was concentrated to give the title compound. UPLC Method B: observed m/e=335.8 [M+1]; t_{R}=1.35 min.

### Step 3: 2,5-Dioxopyrrolidin-1-yl 6-oxo-6-((2-(((2R,3S,4R,5S,6S)-3,4,5-trihydroxy-6-methyltetrahydro-2H-pyran-2-yl)oxy)ethyl)amino)hexanoate

To a solution of 6-oxo-6-((2-(((2R,3S,4R,5S,6S)-3,4,5-trihydroxy-6-methyltetrahydro-2H-pyran-2-yl)oxy)ethyl)amino)hexanoic acid (0.045 g, 0.134 mmol) in DMF (0.4 mL) at 0° C. was added TSTU (60.7 mg, 0.0148 mmol) and DIPEA (0.028 mL, 0.161 mmol). After stirring at 23° C. for 0.5 h, the solution was used directly for the insulin conjugation reaction. UPLC Method B: calculated for C₁₈H₂₈MN₂O₁₀, observed m/e=433.0 [M+1]; t_{R}=1.77 min.

### Example 51: 6-[(2,5-Dioxopyrrolidin-1-yl)oxy]-N-{2-[(α-L-fucopyranosyl)oxy]ethyl}-6-oxohexanamide (ML-51)

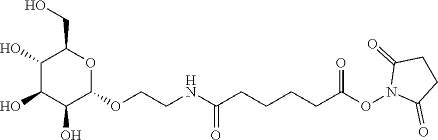 [SMILES: O=C(CCCCC(=O)ON1C(=O)CCC1=O)NCCO[C@H]1O[C@H](CO)[C@@H](O)[C@H](O)[C@@H]1O]

The title compound was prepared using procedures analogous to those described for ML-51, substituting 2-aminoethyl α-L-fucopyranoside (Robert Sardzik et al., Preparation of aminoethylglycosidesfor glycoconjugation, 6 BEILSTEIN J. ORG. CHEM. 699-703 (2010)) for 2-aminoethyl α-D-mannopyranoside in Step 2. UPLC Method B: m/e=449.14 [M+1], t_{R}=1.90 min.

### Example 52: 6-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-6-oxohexanamide (ML-52)

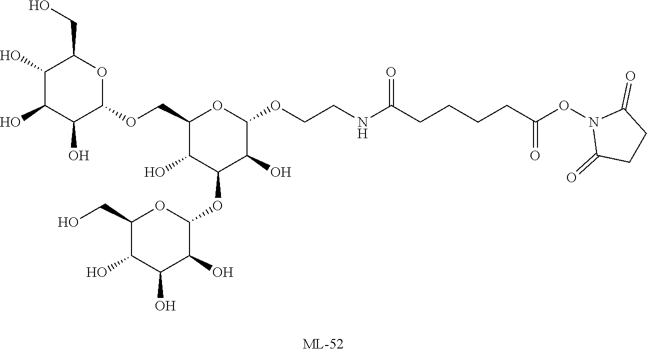 [SMILES: O=C(CCCCC(=O)ON1C(=O)CCC1=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl 6-[(2,5-dioxopyrrolidin-1-yl)oxy]-6-oxohexanoate

To a solution of 6-(benzyloxy)-6-oxohexanoic acid (3.3 g, 13.97 mmol) in DMF (50 mL) at 0° C. was added TSTU (4.3 g, 14.28 mmol) and DIPEA (2.5 mL, 14.31 mmol). After stirring at 0° C. for 1 h, the reaction mixture was partitioned between Et₂O and water. The organic layer was separated, and the aqueous layer was further extracted with Et₂O (2×150 mL). The combined organic phase was washed with brine, dried over Na₂SO₄, filtered, and concentrated to afford the title compound. UPLC Method B: calculated for C₁₇H₁₉NO₆ 333.12, observed m/e: 334.10 [M+1]; t_{R}=3.75 min. ¹H NMR (CDCl₃) δ 7.40-7.30 (5H, m), 5.10 (2H, s), 2.80 (4H, s), 2.62-2.58 (2H, m), 2.41-2.37 (2H, m), 1.80-1.72 (4H, m).

### Step 2: Benzyl 6-({2-[(α-D-mannopyranosyl-(1→3)-[α-D-mannopyranosyl-(1→6)-α-D-mannopyranosyl)oxy]ethyl}amino)-6-oxohexanoate

To a solution of 2-aminoethyl α-L-fucopyranoside (Robert Sardzik et al., Preparation of aminoethylglycosidesfor glycoconjugation, 6 BEILSTEIN J. ORG. CHEM. 699-703 (2010))) in DMF (20 mL) at 0° C. was added benzyl 6-[(2,5-dioxopyrrolidin-1-yl)oxy]-6-oxohexanoate (1.02 g, 3.06 mmol) and TEA (0.5 mL, 3.59 mmol). After stirring at 0° C. for 1 h, the reaction mixture was concentrated, and the residue was purified by flash chromatography on C18 reverse silica gel column, eluting with 0-40% ACN in H₂O to give the title compound. UPLC Method B: calculated for C₃₃H₅₁NO₁₉ 765.31, observed m/e=766.26 [M+1]; t_{R}=4.04 min. ¹H NMR (D₂O) 6 7.43-7.37 (5H, m), 5.14 (2H, s), 5.07-5.06 (1H, m), 4.82-4.81 (1H, m), 4.77-4.76 (1H, m), 4.06-4.01 (2H, m), 3.96-3.92 (2H, m), 3.87-3.81 (5H, m), 3.79-3.77 (1H, m), 3.74-3.67 (5H, m), 3.65-3.60 (4H, m), 3.53-3.49 (1H, m), 3.37-3.35 (2H, m), 2.43-2.40 (2H, m), 2.22-2.19 (2H, m), 1.62-1.52 (4H, m).

### Step 3: 6-({2-[(α-D-Mannopyranosyl-(1→3)-[α-D-mannopyranosyl-(1→6)-α-D-mannopyranosyl)oxy]ethyl}amino)-6-oxohexanoic acid

A mixture of benzyl 6-({2-[(α-D-mannopyranosyl-(1-3)-[α-D-mannopyranosyl-(1→6)]-α-D-mannopyranosyl)oxy]ethyl}amino)-6-oxohexanoate (1.15 g, 1.502 mmol) and Pd/C (80 mg, 0.075 mmol) in water (10 mL) was allowed to stir under H₂ at rt for 16 h. The catalyst was filtered off and washed with H₂O (3×10 mL). The filtrate was concentrated to give the title compound. UPLC Method B: calculated for C₂₆H₄₅NO₁₉ 675.26, observed m/e: 676.21 [M+1]; t_{R}=3.50 min.

### Step 4: 6-[(2,5-Dioxopyrrolidin-1-yl)oxy]-N-(2-{[α-D-mannopyranosyl-(1→3)-[α-D-mannopyranosyl-(1→6)]-β-D-mannopyranosylloxy}ethyl)-6-oxohexanamide

To a solution of 6-({2-[(α-D-mannopyranosyl-(1-3)-[α-D-mannopyranosyl-(1→6)]-α-D-mannopyranosyl)oxy]ethyl}amino)-6-oxohexanoic acid (1.55 g, 2.294 mmol) in DMF (22 mL) at 0° C. was added TSTU (760 mg, 2.52 mmol) and DIPEA (0.52 mL, 2.98 mmol). After stirring at 0° C. for 1 h, the reaction was quenched by the addition of TFA (371 μL, 4.82 mmol), and the resulting mixture was concentrated down to about 3 mL. The residue was transferred dropwise, via autopipette, to a tube containing anhydrous ACN (45 mL). The precipitate was collected through centrifugation (3000 rpm, 15 min, at 4° C.), washed with anhydrous ACN (1 mL) and dried to yield the title compound. UPLC Method B: calculated for C₃₀H₄₈N₂O₂₁ 772.27, observed m/e: 773.23 [M+1]; t_{R}=3.65 min. ¹H NMR (D₂O) δ 5.07-5.06 (1H, m), 4.84-4.83 (1H, m), 4.79-4.78 (1H, m), 4.06-4.01 (2H, m), 3.96-3.93 (2H, m), 3.87-3.83 (5H, m), 3.80-3.78 (1H, m), 3.75-3.69 (5H, m), 3.67-3.61 (4H, m), 3.57-3.52 (1H, m), 3.41-3.38 (2H, m), 2.91 (4H, s), 2.75-2.71 (2H, m), 2.29-2.25 (2H, m), 1.75-1.58 (4H, m).

### Example 53: 2,5-Dioxopyrrolidin-1-yl (S)-19-(2-((6-(((S)-6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrhydro-2H-pyran-2-yl)oxy)ethyl)amino)-1-((2-(((2S,4R,5S,6R)-5-hydroxy-4-((2-hydroxyethoxy)methoxy)-6-((((2S,3S,4S,5S,6R)-3,4,5-trihydroxy-6-(hydroxymethyl) tetrhydro-2H-pyran-2-yl)oxy)methyl)tetrahydro-2H-pyran-2yl)oxy)ethyl)amino)-6-oxohexan-2-yl)amino)-6-oxohexy)amino)ethyl)-8-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)6-((((2S,3S,4S,5S,6R)-3,4,5-trihydroxy-6-(hydroxymethyl)tetrahydroxy-6-(hydroxymethyl) tetrahydro-2H-pyran-2-yl)oxy)methyl)tetrahydro-2H-pyran-2-yl)oxy)ethyl)carbamoyl)-1-(((2S,3S,4S,5R,6R)-6-((((2S,3S,5S,6R)-3,5-dihydroxy-6-(hydroxymethyl)tetrahydro-2H-pyran-2-yl)oxy)tetrahydro-2H-pyran-2yl)oxy)-4,10,17,21-tetraoxo-3,9,16,19,22-pentaazaoctacosan-28-oate (ML-53)

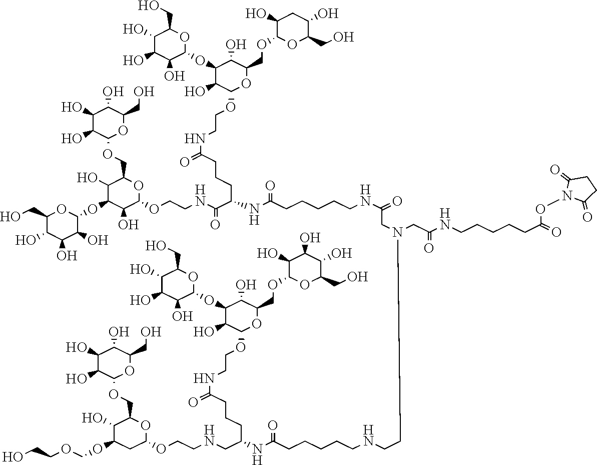

### Step 1: (S)-2-(6-(((benzyloxy)carbonyl)amino)hexanamido)hexanedioic acid

To 2,5-dioxopyrrolidin-1-yl 6-(((benzyloxy)carbonyl)amino)hexanoate (270 mg, 0.745 mmol) in DMF (4 ml) was added (S)-2-aminohexanedioic acid (100 mg, 0.621 mmol) in at 0° C. TEA (0.432 ml, 3.10 mmol) was added, and the mixture was stirred at rt overnight. The solvent was evaporated under reduced pressure. The residue was dissolved in water/acetone (5/1) and loaded on C18 column (43 g), purified by reverse column chromatography on silica gel, eluting with 0-50% ACN in H₂O over 50 min with flow rate of 30 ml/min. to give the desired product. UPLC Method B: m/e=409.0 [M+1], t_{R}=1.20 min.

### Step 2: Benzyl (6-(((S)-1,6-bis((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-1,6-dioxohexan-2-yl)amino)-6-oxohexyl)carbamate

To (S)-2-(6-(((benzyloxy)carbonyl)amino)hexanamido)hexanedioic acid (450 mg, 1.102 mmol) in DMF (40 ml) with stirring was added AETM (2413 mg, 4.41 mmol) neat at rt and Hunig's Base (2.309 ml, 13.22 mmol). HATU (1676 mg, 4.4 mmol) was added, and the mixture was stirred at rt overnight. The solvent was concentrated under reduced pressure. The residue was purified by C18 reverse phase column chromatography on silica gel 240 g, eluting with 0-50% ACN in H₂O over 1 h. UPLC Method B: m/e=1467.57 [M+1], t_{R}=1.50 min.

### Step 3: (S)-2-(6-aminohexanamido)-N1,N6-bis(2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)hexanediamide

Dihydroxypalladium (132 mg, 0.188 mmol) was added to a stirred solution of benzyl (6-(((S)-1,6-bis((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-1,6-dioxohexan-2-yl)amino)-6-oxohexyl)carbamate (920 mg, 0.627 mmol) in water (30 ml) at rt, and the mixture was degassed by reduced pressure and refilled with H₂. The reaction mixture was then placed under a balloon of H₂ at rt for 3 h. The reaction mixture was filtered through CELITE™ diatomaceous earth, washed with water, and lyophilized overnight to give the desired product. UPLC Method B: m/e=667.46 [M+1], t_{R}=1.32 min.

### Step 4: (S)-benzyl 19-(2-((6-(((S)-1,6-bis((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-1,6-dioxohexan-2-yl)amino)-6-oxohexyl)amino)-2-oxoethyl)-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)-8-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)carbamoyl)-4,10,17,21-tetraoxo-3,9,16,19,22-pentaazaoctacosan-28-oate

To a mixture of amine (608 mg, 0.456 mmol) and 2,2′-((2-((6-(benzyloxy)-6-oxohexyl)amino)-2-oxoethyl)azanediyl)diacetic acid (60 mg, 0.152 mmol) in DMF (3 mL) was added Hunig's Base (0.266 ml, 1.521 mmol) and HATU (174 mg, 0.456 mmol). The resulting mixture stirred at rt overnight. The solvent was removed by evaporation, and the residue was purified by reverse phase silica gel column chromatography (CombiFlash Teledyne: C18 86 g column) eluted with 0-40% ACN in H₂O over 1 h to give the desired product. UPLC Method B: m/e=1513.18 [M+1], t_{R}=1.50 min.

### Step 5: (S)-19-(2-((6-(((S)-1,6-bis((2-(((2S,3S,4S,5R,6R)-3,5-dihydroxy-4-(((2S,3S,4S,5S,6R) 3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-1,6-dioxohexan-2-yl)amino)-6-oxohexyl)amino)-2-oxoethyl)-1-(((2S,3S,4S,5R,6R)-3,5-dihydroxy-4-(((2S,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)-8-((2-(((2S,3S,4S,5R,6R)-3,5-dihydroxy-4-(((2S,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2yl)oxy)ethyl)carbamoyl)-4,10,17,21-tetraoxo-3,9,16,19,22-pentaazaoctacosan-28-oic acid

Dihydroxypalladium (20.89 mg, 0.030 mmol) was added to a stirred solution of the product of Step 4 (300 mg, 0.099 mol) in water (10 ml) at rt, and the mixture solution was degassed by reduced pressure and refilled with H₂. The reaction mixture was then placed under a balloon of H₂ at rt for 2.5 h. The residue was filtered through CELITE™ diatomaceous earth and washed with water 3 times. The crude product was freeze dried overnight and used as is. UPLC Method B: m/e=1468.27 [M+1], t_{R}=1.20 min.

### Step 6: 2,5-Dioxopyrrolidin-1-yl (S)-19-(2-((6-(((S)-6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-1-((2-(((2S,4R5S,6R)-5-hydroxy-4-((2-hydroxy-ethoxy)methoxy)-6-((((2S,3S,4S,5S,6R)-3,4,5-trihydroxy-6-(hydroxymethyl) tetrhydro-2H-pyran-2-yl)oxy)methyl)tetrahydro-2H-pyran-2yl)oxy)ethyl)amino)-6-oxohexan-2-yl)amino)-6-oxohexy)amino)ethyl)-8-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)₆-((((2S,3S,4S,5S,6R)-3,4,5-trihydroxy-6-(hydroxymethyl)tetrahydroxy-6-(hydroxymethyl) tetrahydro-2H-pyran-2-yl)oxy)methyl)tetrahydro-2H-pyran-2-yl)oxy)ethyl)carbamoyl)-1-(((2S,3S,4S,5R,6R)-6-((((2S,3S,5S,6R)-3,5-dihydroxy-6-(hydroxymethyl)tetrahydro-2H-pyran-2-yl)oxy)tetrahydro-2H-pyran-2yl)oxy)-4,10,17,21-tetraoxo-3,9,16,19,22-pentaazaoctacosan-28-oate

TSTU (35.7 mg, 0.119 mmol) was added to a stirred mixture of the product of Step 5 (290 mg, 0.099 mmol) in DMF (3 ml) at 0° C., and Hunig's Base (0.021 ml, 0.119 mmol) was added to the mixture. The mixture was stirred at rt for 3 h. The mixture was added dropwise to anhydrous acetone (35.00 ml) under stirring at rt. The suspension was centrifuged for 20 min. The solvent was decanted; the solid was dissolved in water (4 ml) and lyophilized overnight (300 mg, 100%). UPLC Method B: m/e=1516.76 [M+1], t_{R}=1.30 min.

### Example 54: Synthesis of IOC-1

Human insulin (90 mg, 0.015 mmol) was dissolved in aq Na₂CO₃ (0.682 mL, 0.1M) and ACN (2.0 mL). The pH of the resulting solution was adjusted to 10.5, to which ML-1 (52 mg, 0.02 mmol) in water (1.0 mL) in 4 portions over 80 min; the reaction mixture was quenched by adding 2-aminoethanol (4.8 μL, 0.079 mmol). After stirring at rt for 15 min, the reaction mixture was diluted with H₂O and pH was adjusted to about 2.5 using 1.0 N HCl solution and then concentrated. The resulting solution was purified by preparatory scale HPLC using a C4 50×250 mm column, gradient 24-28.5% ACN in H₂O with 0.1% TFA over 25 min, flow rate 85 mL/min. The combined desired fractions were lyophilized. The solids were dissolved in water, and the pH was adjusted to 7 using 0.1 N NaOH solution to provide a solution of B29 mono conjugated intermediate. UPLC-MS Method A: t_{R}=3.45 min; m/z=1661.72 (z=5).

Examples 55 through 87, conjugates IOC-2 to IOC-4, IOC-10, IOC-11, IOC-19, IOC-23, IOC-25 to IOC-30, IOC-33, IOC-36, IOC-39, IOC-42, IOC-50, IOC-51, IOC-53 to IOC-58, and IOC-61 to IOC-46, as listed in Table 1, were prepared according to procedures analogous to those described above with respect to Example 54, Synthesis of IOC-1, substituting the appropriate tetra-valent sugar clusters as indicated for ML-1.

TABLE 1

| Example | Conjugate | Tetra-Valent Sugar Cluster |
|---|---|---|
| 55 | IOC-2 | ML-2 |
| 56 | IOC-3 | ML-3 |
| 57 | IOC-4 | ML-4 |
| 58 | IOC-10 | ML-5 |
| 59 | IOC-11 | ML-6 |
| 60 | IOC-19 | ML-7 |
| 61 | IOC-23 | ML-16 |
| 62 | IOC-25 | ML-17 |
| 63 | IOC-26 | ML-18 |
| 64 | IOC-27 | ML-19 |
| 65 | IOC-28 | ML-20 |
| 66 | IOC-29 | ML-21 |
| 67 | IOC-30 | ML-22 |
| 68 | IOC-33 | ML-53 |
| 69 | IOC-36 | ML-26 |
| 70 | IOC-39 | ML-27 |
| 71 | IOC-42 | ML-28 |
| 72 | IOC-50 | ML-33 |
| 73 | IOC-51 | ML-34 |
| 74 | IOC-53 | ML-35 |
| 75 | IOC-54 | ML-36 |
| 76 | IOC-55 | ML-37 |
| 77 | IOC-56 | ML-38 |
| 78 | IOC-57 | ML-39 |
| 79 | IOC-58 | ML-40 |
| 80 | IOC-61 | ML-41 |
| 81 | IOC-62 | ML-42 |
| 82 | IOC-63 | ML-43 |
| 83 | IOC-64 | ML-44 |
| 84 | IOC-65 | ML-45 |
| 85 | IOC-66 | ML-46 |
| 86 | IOC-67 | ML-47 |
| 87 | IOC-68 | ML-48 |

### Example 88: Synthesis of IOC-5

To a 20 mL scintillation vial containing human insulin (400 mg, 0.069 mmol) at rt was added DMSO (4.0 mL) and TEA (67.2 μL, 0.482 mmol). The mixture was stirred gently until the human insulin dissolved. In a separate vial, ML-4 (152 mg, 0.138 mmol) was dissolved in DMSO (2.0 mL) at rt. To the solution containing human insulin was added the solution of ML-4 in three equal portions in 20 to 30 min intervals. The reaction was quenched by adding 2-aminoethanol (125 μL, 2.066 mmol). After stirring at rt for 15 min, the resulting mixture was carefully diluted with cold H₂O (70 mL) at 0° C. The pH of the resulting mixture was adjusted to a final pH of 2.5 using 1N HCl (or 0.1N NaOH). The resulting solution was purified by preparatory scale HPLC using a C8 10 μm, 100 Å, 50×250 mm column, eluted with Buffer A: 0.05-0.1% TFA in deionized water; Buffer B: 0.05-0.1% TFA in ACN. The combined desired fractions were lyophilized. The solids were dissolved in water, and the pH was adjusted to 7 using 0.1N NaOH solution to provide a solution of IOC-5. UPLC-MS Method A: t_{R}=3.24 min; m/z=1797.07 (z=4).

Examples 89 through 92, conjugates IOC-38 to IOC-41, IOC-45, and IOC-46, as listed in Table 2, were prepared according to procedures analogous to those described above with respect to Example 88, Synthesis of IOC-5, substituting the appropriate tetra-valent sugar clusters as indicated for ML-4.

TABLE 2

| Example | Conjugate | Tetra-Valent Sugar Cluster |
|---|---|---|
| 89 | IOC-38 | ML-27 |
| 90 | IOC-41 | ML-28 |
| 91 | IOC-45 | ML-30 |
| 92 | IOC-46 | ML-31 |

### Example 93: Synthesis of IOC-37

To a 20 mL scintillation vial containing human insulin (60 mg, 0.01 mmol) at rt was added DMSO (0.5 mL) and TEA (43 μL, 0.31 mmol). The mixture was stirred gently until the human insulin dissolved. In a separate vial, ML-27 (42.5 mg, 0.031 mmol) was dissolved in DMSO (0.5 mL) at rt. To the solution containing human insulin was added the solution of ML-27 in three equal portions in 20 to 30 min intervals. The reaction was quenched by adding 2-aminoethanol (19 μL, 0.31 mmol). After stirring at rt for 15 min, the resulting mixture was carefully diluted with cold H₂O (70 mL) at 0° C. The pH of the resulting mixture was adjusted to a final pH of 2.5 using 1N HCl (or 0.1N NaOH). The resulting solution was purified by preparatory scale HPLC using a C8 10 μm, 100 Å, 50×250 mm column, eluted with Buffer A: 0.05-0.1% TFA in deionized water; Buffer B: 0.05-0.1% TFA in ACN. The combined desired fractions were lyophilized. The solids were dissolved in water, and the pH was adjusted to 7 using 0.1N NaOH solution to provide a solution of IOC-37. UPLC-MS Method A: t_{R}=3.34 min; m/z=1916.54 (z=5).

### Example 94: Synthesis of IOC-12

To a solution of N^{A1}-Fmoc Human Insulin (50 mg, 0.0085 mmol; prepared according to the procedures disclosed in WO2015/051052) in DMSO (1 mL) at rt was added Hunig's Base (43 μL, 0.25 mmol) and a solution of ML-7 (41 mg, 0.029 mmol) in DMSO (1 mL). After stirring at rt for 2.5 h, the mixture was added to ACN (42 mL). The precipitate was collected through centrifugation and dissolved in water (5 mL, pH=3.00), and the mixture was cooled to 0° C., to which a solution of NH₄OH (5 mL, 28% in water) was added. The mixture was stirred at 0° C. for 2 h and then diluted with water (20 mL, pH=3.00). The resulting solution was concentrated and reduced to 5 mL, and was further diafiltrated with water (100 mL, pH=3.00) to final volume about 7.5 mL, which was purified by HPLC to give IOC-12. UPLC Method A: t_{R}=3.45 min; m/z=1683.21 (z=5).

Examples 95 through 113, conjugates IOC-13 to IOC-18, IOC-20, IOC-31, IOC-32, IOC-34, IOC-40, IOC-43, IOC-44, IOC-48, IOC-49, IOC-59, IOC-60 and IOC-69, as listed in Table 3, were prepared according to procedures analogous to those described above with respect to Example 94, Synthesis of IOC-12, substituting the appropriate tetra-valent sugar clusters as indicated for ML-7.

TABLE 3

| Example | Conjugate | Tetra-Valent Sugar Cluster |
|---|---|---|
| 95 | IOC-13 | ML-8 |
| 96 | IOC-14 | ML-9 |
| 97 | IOC-15 | ML-10 |
| 98 | IOC-16 | ML-11 |
| 99 | IOC-17 | ML-12 |
| 100 | IOC-18 | ML-13 |
| 101 | IOC-20 | ML-14 |
| 102 | IOC-31 | ML-23 |
| 103 | IOC-32 | ML-24 |
| 104 | IOC-34 | ML-53 |
| 105 | IOC-40 | ML-27 |
| 106 | IOC-43 | ML-28 |
| 107 | IOC-44 | ML-29 |
| 108 | IOC-47 | ML-31 |
| 109 | IOC-48 | ML-30 |
| 110 | IOC-49 | ML-32 |
| 111 | IOC-59 | ML-39 |
| 112 | IOC-60 | ML-38 |
| 113 | IOC-69 | ML-49 |

### Example 114: Synthesis of IOC-21

To a solution of N⁴-TFA Human Insulin (41.8 mg, 0.00708 mmol; prepared according to the procedures disclosed in WO2015/051052) in DMSO (1 mL) at rt was added Hunig's Base (38 μL, 0.218 mmol) and a solution of ML-15 (12 mg, 0.00827 mmol) in DMSO (120 L). After stirring at rt for 90 min, solution of ML-52 (9.4 mg, 0.012 mmol) in DMSO (100 L) was added. The resulting mixture was stirred at rt for 16 h and then added to ACN (45 mL), and the precipitate was collected through centrifugation and dissolved in water (5 mL, pH=3.00). The mixture was cooled to 0° C., to which a solution of NH₄OH (5 mL, 28% in water) was added. The mixture was stirred at 0° C. for 2 h and then diluted with water (20 mL, pH=3.00). The resulting solution was concentrated and reduced to 5 mL, and the solution was then further diafiltrated with water (100 mL, pH=3.00) to final volume about 6 mL, which was purified by HPLC to give IOC-21. UPLC Method A: t_{R}=3.32 min; m/z=1951.21 (z=4).

### Example 115: Synthesis of IOC-22

IOC-22 was prepared according to procedures analogous to those described above with respect to Example 114, Synthesis of IOC-21, substituting ML-15 for ML-52 and ML-52 for ML-15. UPLC Method A: t_{R}=3.34 min; m/z=1951.03 (z=4).

### Example 116: Synthesis of N^{A1}, N^{εB29}-Bis(trifluoroacetyl)Human Insulin

In a 100 mL round bottom flask was charged with human insulin (300 mg, 0.052 mmol), to which was added ACN (6.0 mL), water (6.0 mL), and DIPEA (1.5 mL, 8.59 mmol). To the resulting mixture at 0° C. was added ethyl trifluoroacetate (0.9 mL, 7.54 mml). After stirring at 0° C. for 2 h, the mixture was purified by HPLC (KROMASIL™ C8 10 μm, 100 Å, 50×250 mm column at 210 nm, flow rate at 85 mL/min, 0.05% TFA in ACN/H₂O, 27% ACN to 37% ACN in H₂O, 20 min ramp). The desired fractions were combined and freeze-dried to give the N^{A}I, N^{B29}-Bis(trifluoroacetyl) Human Insulin. UPLC Method A: m/e=1500.677 [(M+4)/4]; t_{R}=3.87 min.

### Example 117: Synthesis of N^{A1}, N^{εB29}-Bis[(9H-Fluoren-9-ylmethoxy)carbonyl]Human Insulin

In a 20 mL scintillation vial, human insulin (1.19 g, 0.205 mmol) and TEA (257 uL, 1.844 mmol) was dissolved in DMSO (10 mL). To this insulin solution was added 1-{[(9H-fluoren-9-ylmethoxy)carbonyl]oxy}pyrrolidine-2,5-dione (207 mg, 0.615 mmol) in DMSO (2 mL). After stirring at rt for 30 min, the reaction was quenched by the addition of HCl (1.84 mL, 1.844 mmol, 1.0M). The resulting mixture was purified by reverse phase HPLC chromatography. The desired fractions were collected and lyophilized to give the N^{A1}, N^{εB29}-bis[(9H-Fluoren-9-ylmethoxy) carbonyl]human insulin. UPLC Method A: m/e=1 564.04 [(M+4/4)]; t_{R}=4.41 min.

### Examples 118 and 119: Synthesis of IOC-6 and IOC-7

### Step 1: Synthesis of insulin B29 mono-conjugated intermediate

Human insulin (800 mg, 0.138 mmol) was dissolved in aq Na₂CO₃ (6.85 mL, 0.1M) and ACN (4.6 mL). The pH of the resulting solution was adjusted to 10.5, and ML-8 (157 mg, 0.207 mmol) in DMSO (2.25 mL) was added to the solution in 4 portions over 80 min. The reaction mixture was quenched by adding 2-aminoethanol (41.7 μL, 0.689 mmol). After stirring at rt for 15 min, the reaction mixture was diluted with H₂O, and the pH was adjusted to about 2.5 using 1.0 N HCl solution, concentrated. The resulting solution was purified by preparatory scale HPLC with a C4 50×250 mm column, using gradient 24-28.5% ACN in H₂O with 0.1% TFA over 25 min, flow rate 85 mL/min. The combined desired fractions were lyophilized. The solids were dissolved in water, and the pH adjusted to 7 using 0.1N NaOH solution to provide a solution of B29 mono-conjugated intermediate. UPLC-MS Method A: t_{R}=3.75 min; m/z=1613.72 (z=4).

### Step 2: A1 conjugation of B29 mono-conjugated intermediate

The procedures is analogous to those described in Example 118, substituting B29 mono conjugated intermediate from Step 1 for human insulin and ML-50 (1.2 eq) for ML-8, to give IOC-6 and IOC-7. HPLC-MS Method A (IOC-6): t_{R}=3.31 min; m/z=1928.71 (z=4). HPLC-MS Method A (IOC-7): t_{R}=3.36 min; m/z=2007.98 (z=4).

Examples 120 and 121, conjugates IOC-8 and IOC-9, as listed in Table 4, were prepared according to procedures analogous to those described above with respect to Examples 118 and 119: Synthesis of IOC-6 and IOC-7, substituting the appropriate tetra-valent sugar clusters as indicated for ML-4 in Step 1, ML-51 in Step 2.

TABLE 4

| | | Tetra-Valent Sugar |
|---|---|---|
| Example | Conjugate | Clusters |
| 120 | IOC-8 | ML-4 (Step 1), 51 (Step 2) |
| 121 | IOC-9 | ML-4 (Step 1), 51 (Step 2) |

### Example 122: Synthesis of IOC-24

N^{A1}, N^{εB29}-Bis(trifluoroacetyl)Human Insulin (100 mg, 0.017 mmol) was dissolved in DMSO (1 mL). To this solution was added ML-16 (32 mg, 0024 mmol in 1 ml DMSO) in three portions over 90 min. Then 2-aminoethanol (0.005 ml, 0.019 mmol) was added to the reaction mixture and stirred for 15 min to quench the reaction. To the mixture was added aqueous ammonium hydroxide (5 ml) and stored at 4° C. for 18 h. UPLC-MS showed fully deprotection of trifluoroacetate groups. The resulting solution was purified by preparatory scale HPLC with a C4 50×250 mm column, using gradient 27-31% ACN in H₂O with 0.1% TFA over 25 min, flow rate 85 mL/min. The combined desired fractions were lyophilized. The solids were dissolved in water, and the pH adjusted to 7 using 0.1N NaOH solution to provide a solution of B1 mono-conjugated product. UPLC-MS Method A: t_{R}=3.28 min; m/z=1705.63 (z=4)

Examples 123 and 124, conjugates IOC-35 and IOC-52, as listed in Table 5, were prepared according to procedures analogous to those described above with respect to Example 122, Synthesis of IOC-24, substituting the appropriate tetra-valent sugar clusters as indicated for ML-16.

TABLE 5

| Example | Conjugate | Tetra-Valent Sugar Cluster |
|---|---|---|
| 123 | IOC-35 | ML-53 |
| 124 | IOC-52 | ML-34 |

### Example 125: Insulin Receptor Phosphorylation Assays

The insulin receptor phosphorylation assays were performed using the commercially available Meso Scale Discovery (“MSD”) pIR assay (See Meso Scale Discovery, 9238 Gaithers Road, Gaithersburg, Md.). CHO cells stably expressing human IR(B) were in grown in in F12 cell media containing 10% FBS and antibiotics (G418, Penicillin/Strepavidin) for at least 8 h and then serum starved by switching to F12 media containing 0.5% BSA (insulin-free) in place of FBS for overnight growth. Cells were harvested and frozen in aliquots for use in the MSD pIR assay. Briefly, the frozen cells were plated in either 96-well (40,000 cells/well, Method A and Method B) or 384-well (10,000 cells/well, Method C) clear tissue culture plates and allowed to recover. IOC molecules at the appropriate concentrations were added, and the cells were incubated for 8 min at 37° C. The media was aspirated and chilled. MSD cell lysis buffer (cell lysis buffer formulation: 150 mM NaCl; 20 mM Tris, pH 7.5; 1 mM EDTA; 1 mM EGTA and 1% Triton X-100); MSD kit pIR detection plate containing insulin signaling panel) was added as per MSD kit instructions. The cells were lysed on ice for 40 min, and the lysate then mixed for 10 min at rt. The lysate was transferred to the MSD kit pIR detection plates. The remainder of the assay was carried out following the MSD kit recommended protocol.

### Example 126: Insulin Receptor Binding Assays

Insulin Receptor Binding Assays were performed as follows.

Two competition binding assays were utilized to determine IOC affinity for the human insulin receptor type B (IR(B)) against the endogenous ligand, insulin, labeled with ¹²⁵[I].

Method C: IR binding assay was a whole cell binding method using CHO cells overexpressing human IR(B). The cells were grown in F12 media (Ham's F-12 Nutrient Mixture, a nutrient mixture designed to cultivate a wide variety of mammalian and hybridoma cells when used with serum in combination with hormones and transferrin) containing 10% FBS and antibiotics (G418, Penicillin/Strepavidin), plated at 40,000 cells/well in a 96-well tissue culture plate for at least 8 h. The cells were then serum starved by switching to DMEM media containing 1% BSA (insulin-free) overnight. The cells were washed twice with chilled DMEM media containing 1% BSA (insulin-free) followed by the addition of IOC molecules at appropriate concentration in 90 μL of the same media. The cells were incubated on ice for 60 min. The ¹²⁵[I]-insulin (10 μL) was added at 0.015 nm final concentration and incubated on ice for 4 h. The cells were gently washed three times with chilled media and lysed with 30 μL of Cell Signaling lysis buffer (Cell Signal Technology, catalog #9803) with shaking for 10 min at rt. The lysate was added to scintillation liquid and counted to determine ¹²⁵[I]-insulin binding to IR and the titration effects of IOC molecules on this interaction.

Method D: IR binding assay was run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes were prepared in 50 mm Tris (tris(bydroxymethyl) aminornethane) buffer, pH 7.8 containing 5 mm MgCl₂. The assay buffer contained 50 mm Tris buffer, pH 7.5, 150 mm NaCl, 1 mm CaCl₂, 5 mm MgCl₂, 0.1% BSA and protease inhibitors (Complete-Mini-Roche). Cell membranes were added to WGA PVT PEI SPA beads (5 mg/ml final concentration) followed by addition of IOC molecules at appropriate concentrations. After 5-15 min incubation at rt, ¹²⁵[I]-insulin was added at 0.015 nm final concentration for a final total volume of 50 μL. The mixture was incubated with shaking at rt for 1 to 12 h followed by scintillation counting to determine ¹²⁵[I]-insulin binding to IR and the titration effects of IOC molecules on this interaction.

### Example 127: Human Macrophage Mannose Receptor 1 (MRC1) Binding Assays

Human macrophage mannose receptor 1 (“MRC1”) Binding Assays were performed as follows.

The competition binding assay for MRC1 utilized a ligand, mannosylated-BSA labeled with the DELFIA Eu-N1-ITC reagent (labeling kit for europium labeling of proteins and polypetides for use in dissociation-enhanced time-resolved fluorometric assay), as reported in the literature. Assay was performed either in a 96-well plate with 100 μL well volume (Method E) or in a 384-well plate with 25 μL well volume (Method F). Anti-MRC1 (Mannose Receptor C-Type 1) antibody (2 ng/μl) in PBS containing 1% stabilizer BSA was added to a Protein G plate that had been washed three times with 100 μl of 50 mm Tris buffer, pH 7.5 containing 100 mm NaCl, 5 mm CaCl₂, 1 mm MgCl₂ and 0.1% Tween-20 (wash buffer). The antibody was incubated in the plate for 1 h at rt with shaking. The plate was washed with wash buffer 3-5 times followed by addition of MRC1 (2 ng/μl final concentration) in PBS containing 1% stabilizer BSA. The plate was incubated at rt with gentle shaking for 1 h. The plate was washed three times with wash buffer. The IOC molecules in 12.5 μL (or 50 μL depending on plate format) buffer at appropriate concentrations were added followed by 12.5 μL (or 50 μL) Eu-mannosylated-BSA (0.1 nm final concentration) in 50 mm Tris, pH 7.5 containing 100 mm NaCl, 5 mm CaCl₂, 1 mm MgCl₂ and 0.2% stabilizer BSA. The plate was incubated for 2 h at rt with shaking followed by washing three times with wash buffer. Perkin Elmer Eu-inducer reagent was added and incubated for 30 min at rt prior to detection of the Eu signal (Excitation=340 nm: Emission=615 nm).

### Example 128: Assay Results

The following table lists conjugates that were prepared using appropriate intermediates following one of the General Methods described above. These conjugates were characterized using UPLC Method E or UPLC Method F noted by an asterisk (*), exhibiting either four charged, i.e. [(M+4)/4], (or five charged, i.e. [(M+5)/5]) species of parent compound at certain retention time (“t_{R}”). The in vitro biological activities towards insulin receptor (IR) were measured by either ligand competition assays or functional phosphorylation assays, as described above, labeled as following: Method A: IR phosphorylation assay based on 96-well; Method B: IR phosphorylation assay based on 384-well with automated liquid dispense; Method C: cell-based IR binding assay; Method D: SPA IR binding assay method E; Method E: MRC1 assay was performed in a 96-well plate; Method F: MRC1 assay was performed in a 384-well plate. The results are shown in Table 6.

TABLE 6

| | Human pIR | Human IR | Human MRC1 |
|---|---|---|---|
| Conjugate | EC₅₀ (nM) | IC₅₀ (nM) | IC₅₀ (nM) |
| IOC-1 | 0.76 | 2.04 | 7.22 |
| IOC-2 | 4.3 | 2.84 | 15.34 |
| IOC-3 | 1.13 | 3.46 | 2.38 |
| IOC-4 | 2.55 | 2.57 | 16.43 |
| IOC-5 | 13.74 | 24.18 | 2.59 |
| IOC-6 | 15.12 | 11.32 | 18.25 |
| IOC-7 | 13.05 | 9.07 | 31.78 |
| IOC-8 | 6.63 | 4.84 | 61.6 |
| IOC-9 | 9.71 | 11.67 | 41.95 |
| IOC-10 | 15.42 | 2.09 | 3.66 |
| IOC-11 | 10.62 | 2.16 | 18.91 |
| IOC-12 | 0.88 | 1.56 | 0.54 |
| IOC-13 | 0.53 | 2.16 | 13.47 |
| IOC-14 | 4.02 | 1.69 | 6.69 |
| IOC-15 | 7.53 | 5.36 | 46.8 |
| IOC-16 | 0.61 | 0.8 | 1801 |
| IOC-17 | 1.1 | 6.79 | 1.67 |
| IOC-18 | 1 | — | 18.64 |
| IOC-19 | 0.7 | — | 14.66 |
| IOC-20 | 1.08 | 2.77 | 74.26 |
| IOC-21 | 1.6 | 0.97 | 25.14 |
| IOC-22 | 0.76 | 0.44 | 26.23 |
| IOC-23 | 0.46 | 1.55 | 0.9 |
| IOC-24 | 0.43 | 0.84 | 2.71 |
| IOC-25 | 1.61 | 8.86 | 2.07 |
| IOC-26 | 1.92 | 7.64 | 9.1 |
| IOC-27 | 1.99 | 3.24 | 1.47 |
| IOC-28 | 2.57 | 10.1 | 9.33 |
| IOC-29 | 3.59 | 11.17 | 6.83 |
| IOC-30 | 1.43 | 1.74 | 2 |
| IOC-31 | 3.71 | 0.9 | 3.26 |
| IOC-32 | 0.53 | 3.72 | 2.58 |
| IOC-33 | 0.68 | 2.9 | 1.44 |
| IOC-34 | 10.97 | 24.5 | 0.69 |
| IOC-35 | 0.69 | 7.44 | 5.49 |
| IOC-36 | 0.65 | 0.5 | 34.64 |
| IOC-37 | 22.97 | 2.9 | 0.28 |
| IOC-38 | 19 | 2.71 | 0.92 |
| IOC-39 | 2.29 | 0.65 | 50.33 |
| IOC-40 | 0.44 | 0.94 | 0.68 |
| IOC-41 | 20.17 | 3.26 | 2.8 |
| IOC-42 | 2.47 | 0.99 | 55.45 |
| IOC-43 | 6.76 | 2.18 | 2.44 |
| IOC-44 | 3.36 | 2.73 | 4.25 |
| IOC-45 | 4.73 | 5.29 | 6.99 |
| IOC-46 | 7.99 | 4.75 | 3.33 |
| IOC-47 | 1.39 | 3.82 | 2.48 |
| IOC-48 | 0.68 | 2.39 | 12.51 |
| IOC-49 | 10.27 | 2.72 | 14.3 |
| IOC-50 | 11.85 | 4.4 | 13.94 |
| IOC-51 | 1.68 | 1.61 | 6.99 |
| IOC-52 | 0.94 | 0.63 | 9.28 |
| IOC-53 | 2.11 | 0.52 | 85.72 |
| IOC-54 | 4.51 | 0.51 | 29.5 |
| IOC-55 | 4.66 | 1.27 | 5 |
| IOC-56 | 1.81 | 0.82 | 18.18 |
| IOC-57 | 4.55 | 1 | 27.21 |
| IOC-58 | 4.41 | 1.55 | 13.85 |
| IOC-59 | 6.37 | 2.86 | 6.51 |
| IOC-60 | 6.69 | 3.13 | 0.32 |
| IOC-61 | 0.7 | 1.36 | 7.25 |
| IOC-62 | 1.2 | 0.61 | 11.91 |
| IOC-63 | 8.53 | 2.12 | 21.97 |
| IOC-64 | 7.5 | 1.55 | 26.09 |
| IOC-65 | 8.66 | 1.9 | 23.28 |
| IOC-66 | 2.93 | 0.53 | 1.2 |
| IOC-67 | 9.69 | 1.68 | 35.66 |
| IOC-68 | 0.9 | 1.54 | 33.28 |
| IOC-69 | 1.95 | — | 4.15 |

### Example 129: Effect of Methyl α-D-Mannopyranoside (αMM) on PK and PD of Various IOCs in Non-Diabetic Minipigs

Effect of Methyl α-D-Mannopyranoside (αMM) on PK and PD of various IOCs in Non-Diabetic Minipigs was evaluated.

Male Yucatan miniature pigs, non-diabetic, instrumented with two Jugular vein vascular access ports (“VAP”), were used in these studies. Animals were fasted overnight prior to the study. On the day of the study, animals were restrained in slings, and VAPs accessed for infusion and sampling. At t=−60 min, a constant infusion of PBS (n=3) or 21.2% αMM (n=3) is started, at a rate of 2.67 mL/kg/hr. This infusion was maintained for the duration of the study. At t=0 min, and after collecting a baseline blood sample for plasma glucose measurement, animals were administered IOC as a single bolus IV. Sampling continued for 90 min, with final readouts of plasma glucose and compound levels.

IOCs were formulated at 17-69 nmol/mL in sodium chloride (87 mm), phenol (21 mm), dibasic sodium phosphate (26.5 mm), Osmolality=275 mOsm/kg (milliosmoles per kilogram), pH=7.4; QS (Quantum satis) with Water for Injection.

Time points for sample collection: −60 min, 0 min, 1 min, 2 min, 4 min, 6 min, 8 min, 10 min, 15 min, 20 min, 25 min, 30 min, 35 min, 45 min, 60 min, and 90 min.

Blood was collected in K₃-EDTA (tripotassium ethyl enediaminetetraacetic acid) tubes, supplemented with 10 g/ml Aprotinin, and kept on an ice bath until processing, within 30 min of collection. After centrifugation at 3000 rpm, 4° C., for 8 min, plasma was collected and aliquoted for glucose measurement using a Beckman Coulter AU480 Chemistry analyzer and for compound levels measurement by LC-MS.

The IOCs evaluated were IOC-2, IOC-7, IOC-11, IOC-12, IOC-13, IOC-16, IOC-17, IOC-18, IOC-20, IOC-25, IOC-28, IOC-31, IOC-36, IOC-43, IOC-44, IOC-47, IOC-49, IOC-50, IOC-52, IOC-55, IOC-63, IOC-65, IOC-66, IOC-67, and IOC-69.

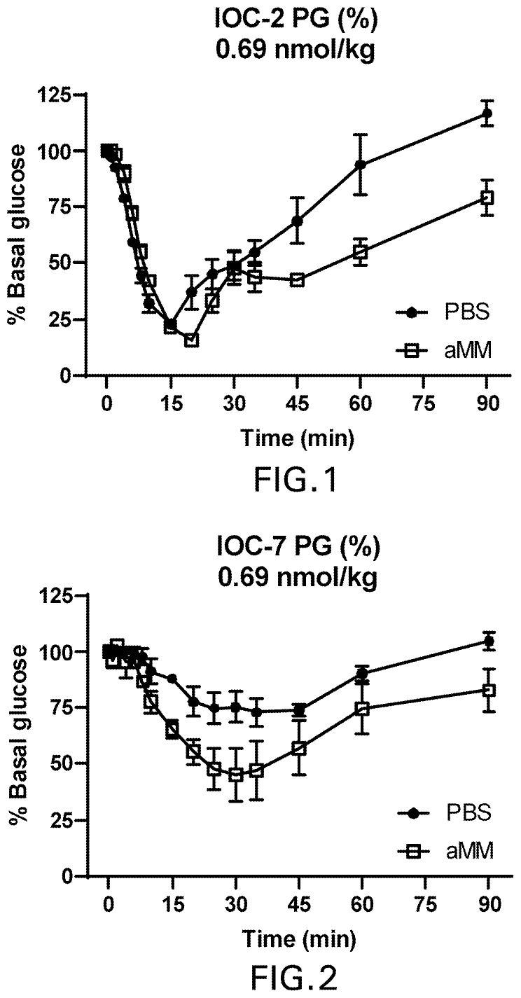

Glucose results are expressed as % changes over baseline values at t=0 min, and the results are shown in FIG. 1 through FIG. 25.

FIG. 1 shows blood glucose depression curves in non-diabetic male Yucatan minipigs equipped with dual vascular access ports (n=3 per study) following i.v. injection of conjugate IOC-2 at 0.69 nmol/kg under conditions of PBS infusion or i.v. αMM infusion.

FIG. 2 shows blood glucose depression curves in non-diabetic male Yucatan minipigs equipped with dual vascular access ports (n=3 per study) following i.v. injection of conjugate IOC-7 at 0.69 nmol/kg under conditions of PBS infusion or i.v. αMM infusion.

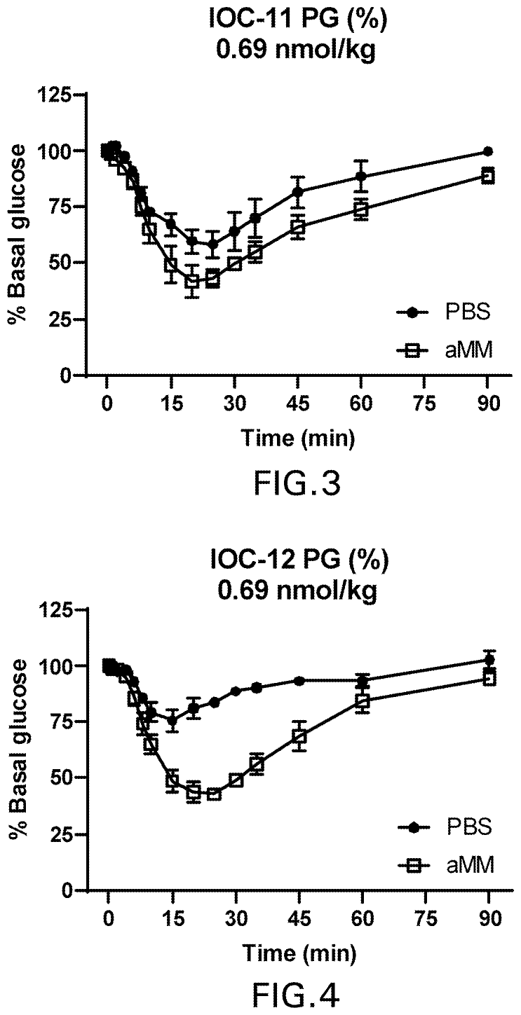

FIG. 3 shows blood glucose depression curves in non-diabetic male Yucatan minipigs equipped with dual vascular access ports (n=3 per study) following i.v. injection of conjugate IOC-11 at 0.69 nmol/kg under conditions of PBS infusion or i.v. αMM infusion.

FIG. 4 shows blood glucose depression curves in non-diabetic male Yucatan minipigs equipped with dual vascular access ports (n=3 per study) following i.v. injection of conjugate IOC-12 at 0.69 nmol/kg under conditions of PBS infusion or i.v. αMM infusion.

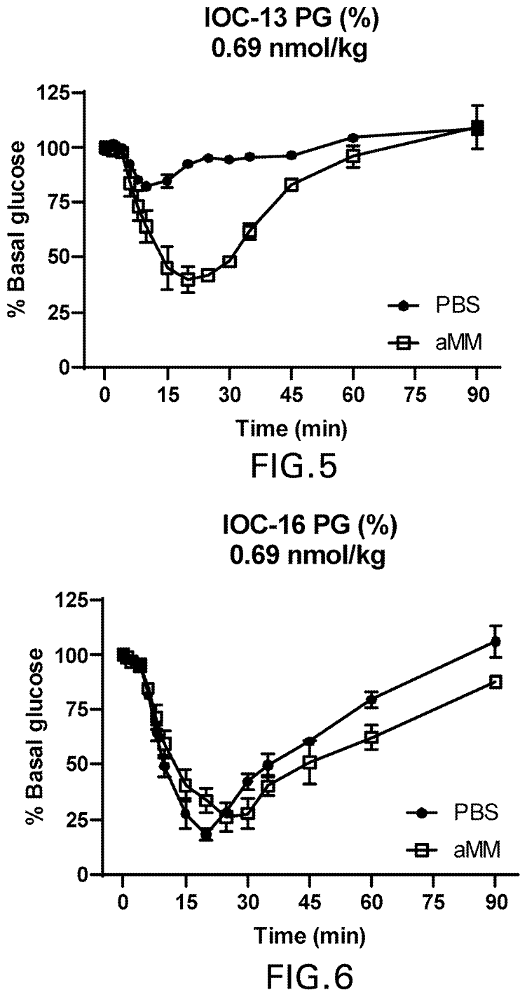

FIG. 5 shows blood glucose depression curves in non-diabetic male Yucatan minipigs equipped with dual vascular access ports (n=3 per study) following i.v. injection of conjugate IOC-13 at 0.69 nmol/kg under conditions of PBS infusion or i.v. αMM infusion.

FIG. 6 shows blood glucose depression curves in non-diabetic male Yucatan minipigs equipped with dual vascular access ports (n=3 per study) following i.v. injection of conjugate IOC-16 at 0.69 nmol/kg under conditions of PBS infusion or i.v. αMM infusion.

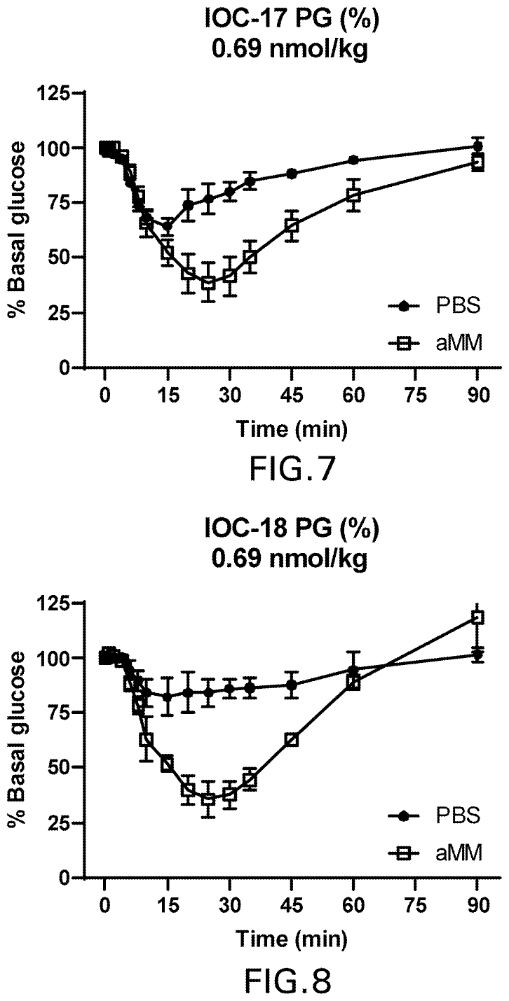

FIG. 7 shows blood glucose depression curves in non-diabetic male Yucatan minipigs equipped with dual vascular access ports (n=3 per study) following i.v. injection of conjugate IOC-17 at 0.69 nmol/kg under conditions of PBS infusion or i.v. αMM infusion.

FIG. 8 shows blood glucose depression curves in non-diabetic male Yucatan minipigs equipped with dual vascular access ports (n=3 per study) following i.v. injection of conjugate IOC-18 at 0.69 nmol/kg under conditions of PBS infusion or i.v. αMM infusion.

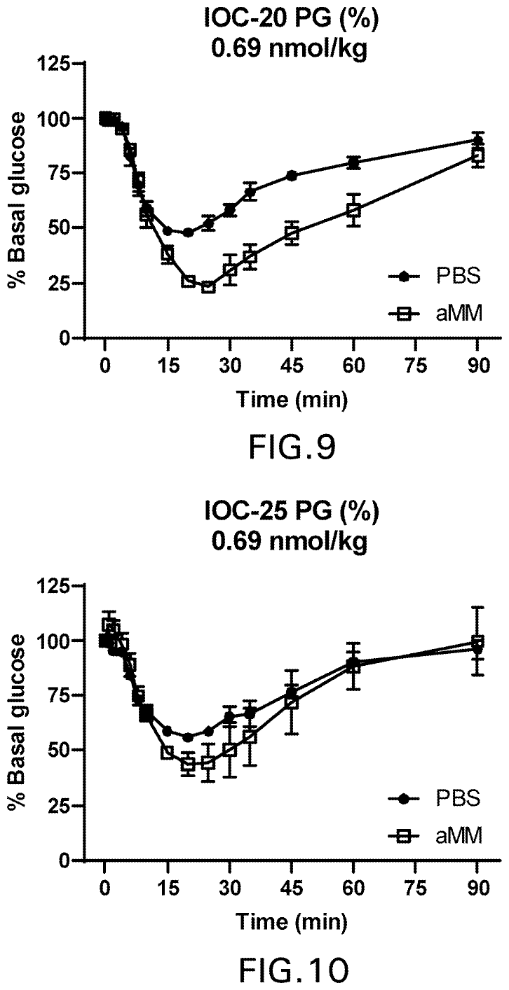

FIG. 9 shows blood glucose depression curves in non-diabetic male Yucatan minipigs equipped with dual vascular access ports (n=3 per study) following i.v. injection of conjugate IOC-20 at 0.69 nmol/kg under conditions of PBS infusion or i.v. αMM infusion.

FIG. 10 shows blood glucose depression curves in non-diabetic male Yucatan minipigs equipped with dual vascular access ports (n=3 per study) following i.v. injection of conjugate IOC-25 at 0.69 nmol/kg under conditions of PBS infusion or i.v. αMM infusion.

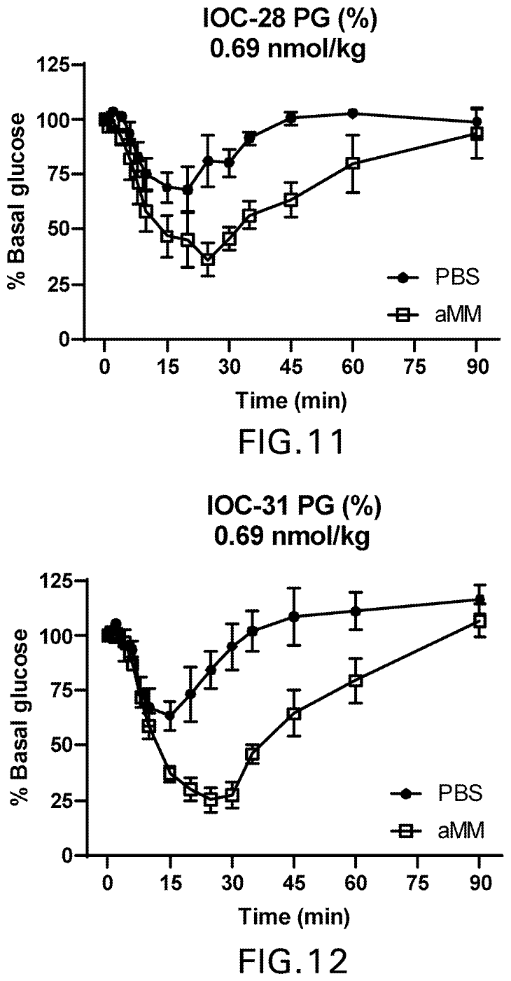

FIG. 11 shows blood glucose depression curves in non-diabetic male Yucatan minipigs equipped with dual vascular access ports (n=3 per study) following i.v. injection of conjugate IOC-28 at 0.69 nmol/kg under conditions of PBS infusion or i.v. αMM infusion.

FIG. 12 shows blood glucose depression curves in non-diabetic male Yucatan minipigs equipped with dual vascular access ports (n=3 per study) following i.v. injection of conjugate IOC-31 at 0.69 nmol/kg under conditions of PBS infusion or i.v. αMM infusion.

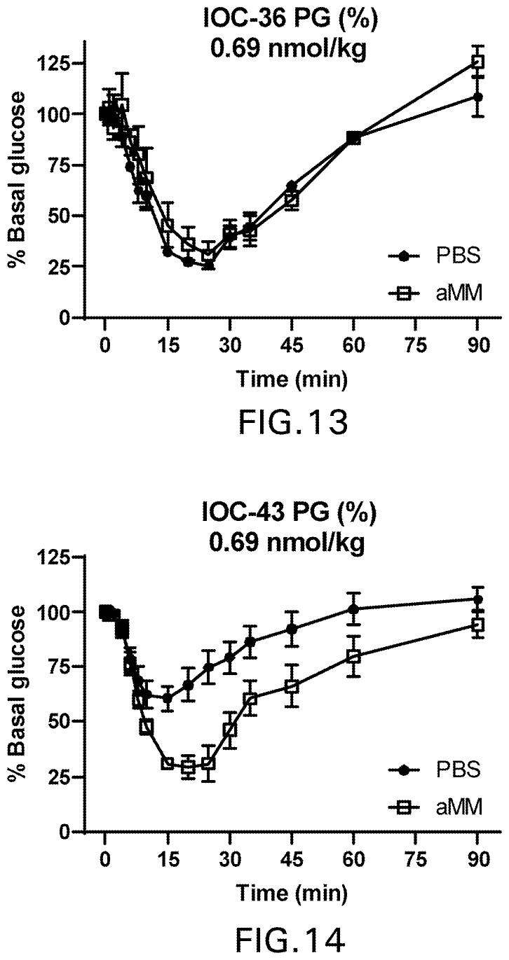

FIG. 13 shows blood glucose depression curves in non-diabetic male Yucatan minipigs equipped with dual vascular access ports (n=3 per study) following i.v. injection of conjugate IOC-36 at 0.69 nmol/kg under conditions of PBS infusion or i.v. αMM infusion.

FIG. 14 shows blood glucose depression curves in non-diabetic male Yucatan minipigs equipped with dual vascular access ports (n=3 per study) following i.v. injection of conjugate IOC-43 at 0.69 nmol/kg under conditions of PBS infusion or i.v. αMM infusion.

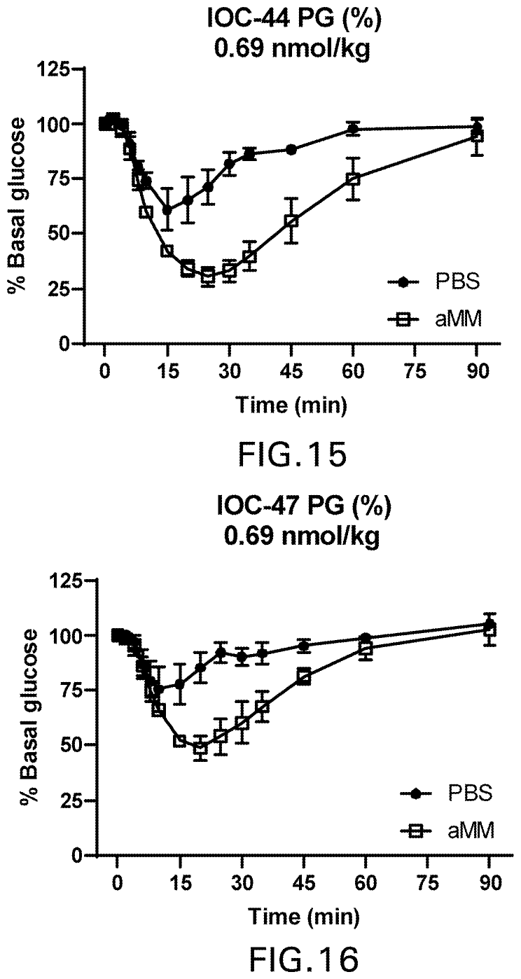

FIG. 15 shows blood glucose depression curves in non-diabetic male Yucatan minipigs equipped with dual vascular access ports (n=3 per study) following i.v. injection of conjugate IOC-44 at 0.69 nmol/kg under conditions of PBS infusion or i.v. αMM infusion.

FIG. 16 shows blood glucose depression curves in non-diabetic male Yucatan minipigs equipped with dual vascular access ports (n=3 per study) following i.v. injection of conjugate IOC-47 at 0.69 nmol/kg under conditions of PBS infusion or i.v. αMM infusion.

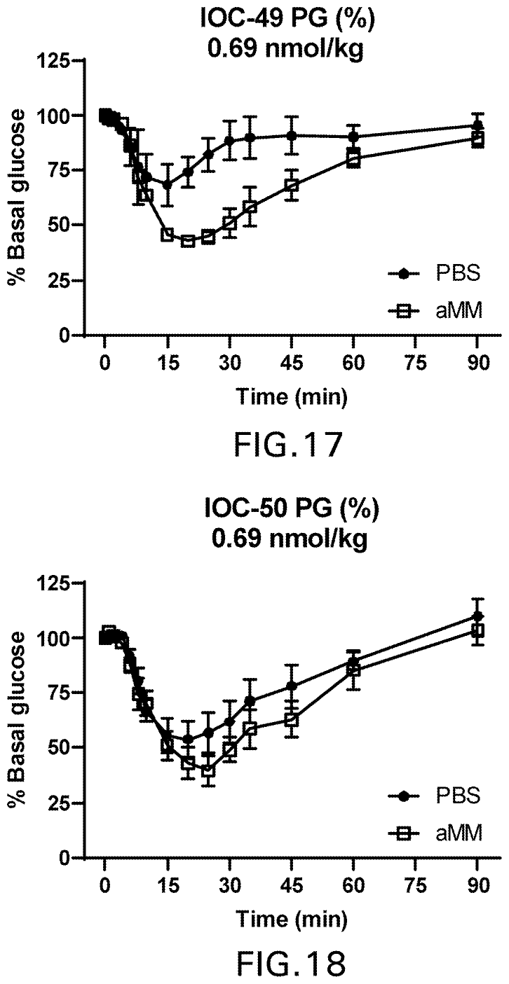

FIG. 17 shows blood glucose depression curves in non-diabetic male Yucatan minipigs equipped with dual vascular access ports (n=3 per study) following i.v. injection of conjugate IOC-49 at 0.69 nmol/kg under conditions of PBS infusion or i.v. αMM infusion.

FIG. 18 shows blood glucose depression curves in non-diabetic male Yucatan minipigs equipped with dual vascular access ports (n=3 per study) following i.v. injection of conjugate IOC-50 at 0.69 nmol/kg under conditions of PBS infusion or i.v. αMM infusion.

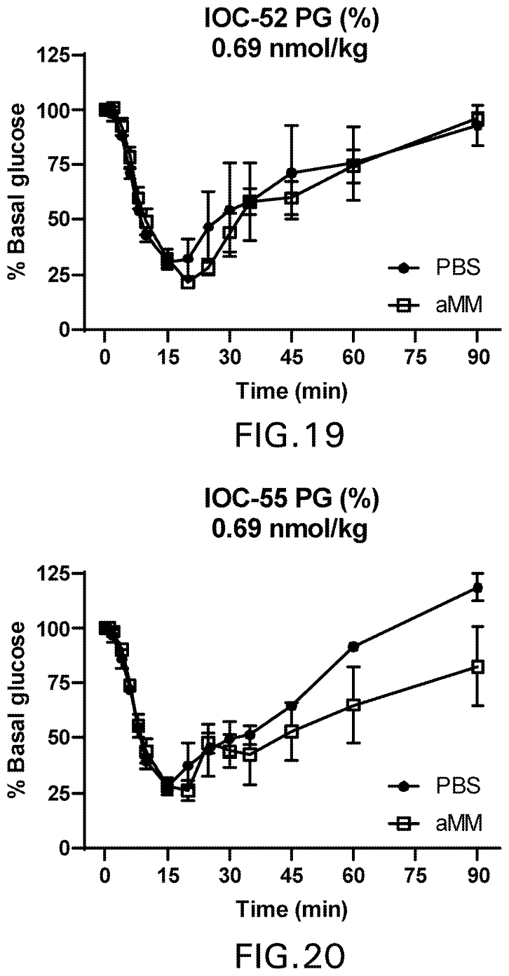

FIG. 19 shows blood glucose depression curves in non-diabetic male Yucatan minipigs equipped with dual vascular access ports (n=3 per study) following i.v. injection of conjugate IOC-52 at 0.69 nmol/kg under conditions of PBS infusion or i.v. αMM infusion.

FIG. 20 shows blood glucose depression curves in non-diabetic male Yucatan minipigs equipped with dual vascular access ports (n=3 per study) following i.v. injection of conjugate IOC-55 at 0.69 nmol/kg under conditions of PBS infusion or i.v. αMM infusion.

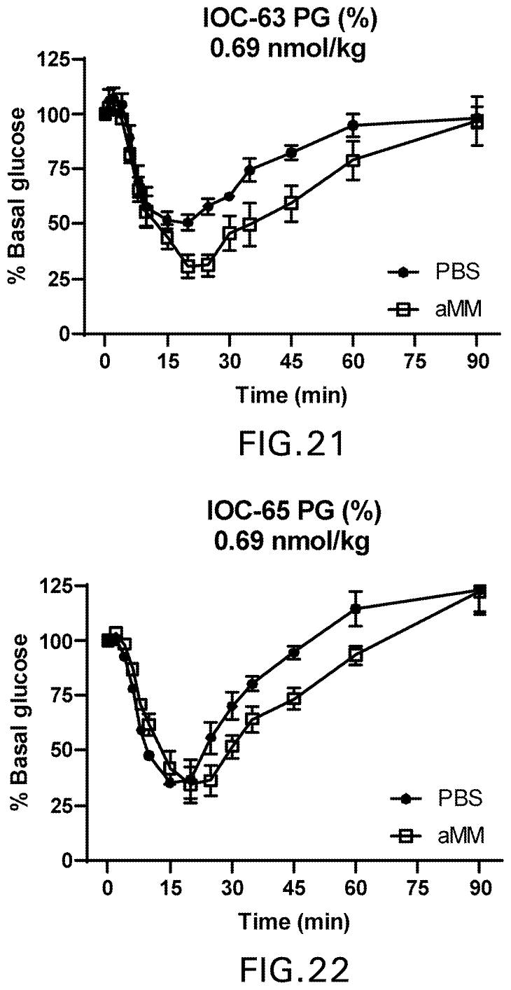

FIG. 21 shows blood glucose depression curves in non-diabetic male Yucatan minipigs equipped with dual vascular access ports (n=3 per study) following i.v. injection of conjugate IOC-63 at 0.69 nmol/kg under conditions of PBS infusion or i.v. αMM infusion.

FIG. 22 shows blood glucose depression curves in non-diabetic male Yucatan minipigs equipped with dual vascular access ports (n=3 per study) following i.v. injection of conjugate IOC-65 at 0.69 nmol/kg under conditions of PBS infusion or i.v. αMM infusion.

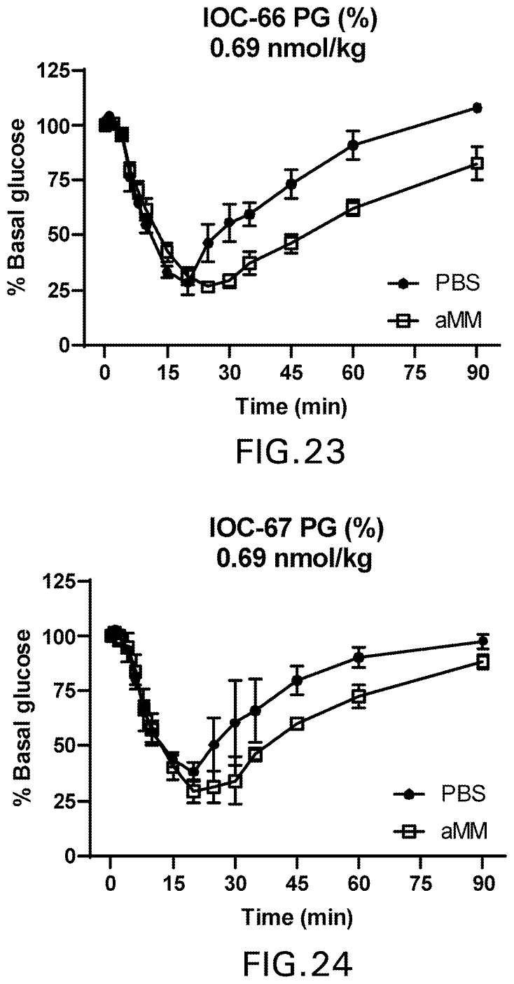

FIG. 23 shows blood glucose depression curves in non-diabetic male Yucatan minipigs equipped with dual vascular access ports (n=3 per study) following i.v. injection of conjugate IOC-66 at 0.69 nmol/kg under conditions of PBS infusion or i.v. αMM infusion.

FIG. 24 shows blood glucose depression curves in non-diabetic male Yucatan minipigs equipped with dual vascular access ports (n=3 per study) following i.v. injection of conjugate IOC-67 at 0.69 nmol/kg under conditions of PBS infusion or i.v. αMM infusion.

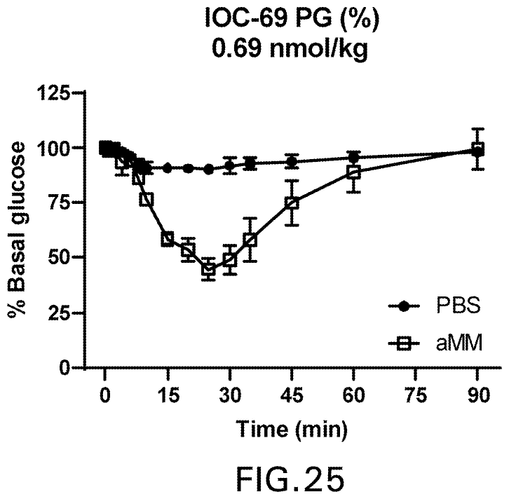

FIG. 25 shows blood glucose depression curves in non-diabetic male Yucatan minipigs equipped with dual vascular access ports (n=3 per study) following i.v. injection of conjugate IOC-69 at 0.69 nmol/kg under conditions of PBS infusion or i.v. αMM infusion.

It will be appreciated that various of the above-discussed and other features and functions, or alternatives thereof, may be desirably combined into many other different systems or applications. Also that various presently unforeseen or unanticipated alternatives, modifications, variations, or improvements therein may be subsequently made by those skilled in the art which are also intended to be encompassed by the following claims.

1. A conjugate comprising an insulin or insulin analog molecule covalently attached to at least one tetra-valent sugar cluster wherein the tetra-valent sugar cluster is provided by a tetra-dentate linker having four arms wherein each arm of the tetra-dentate linker is independently covalently linked to a ligand comprising a saccharide selected from the group consisting of monosaccharide, disaccharide, trisaccharide, tetrasaccharide, and branched trisaccharide.

2. The conjugate of claim 1, wherein the ligand comprises a saccharide selected from the group consisting of fucose, mannose, glucosamine, glucose, dimannose, trimannose, tetramannose, and branched trimannose.

3. The conjugate of claim 1, wherein the ligand comprises a saccharide selected from the group consisting of aminoethylglucose, aminoethylmannose, aminoethylbimannose, aminoethyltrimannose, O-aminoethyl-N-acetylglucosamine, and aminoethylfucose.

4. The conjugate of claim 1, wherein the tetra-valent sugar cluster is covalently linked to the amino acid at position A1 of the insulin or insulin analog molecule; position B1 of the insulin or insulin analog molecule; or position B29 of the insulin or insulin analog molecule.

5. The conjugate of claim 1, wherein the conjugate comprises an insulin or insulin analog molecule conjugated to at least two tetra-valent sugar clusters.

6. The conjugate of claim 1, wherein the conjugate comprises an insulin or insulin analog molecule conjugated to at least four tetra-valent sugar clusters.

7. The conjugate of claim 1, wherein the insulin analog is insulin lispro, insulin glargine, insulin aspart, insulin detemir, or insulin glulisine.

8. The conjugate of claim 1, wherein the conjugate displays a pharmacodynamic or pharmacokinetic profile that is sensitive to the serum concentration of a serum saccharide when administered to a subject in need thereof in the absence of an exogenous saccharide binding molecule.

9. The conjugate of claim 8, wherein the serum saccharide is glucose or alpha-methylmannose.

10. The conjugate of claim 1, wherein the conjugate binds an endogenous saccharide binding molecule at a serum glucose concentration of 60 mg/dL or less when administered to a subject in need thereof.

11. The conjugate of claim 10, wherein the endogenous saccharide binding molecule is human mannose receptor 1.

12. The conjugate of claim 1, wherein the conjugate has the general formula (I): 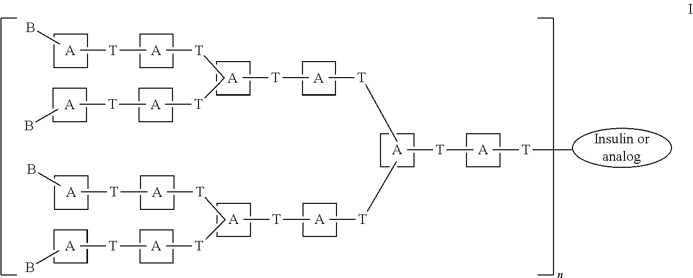
or has the general formula (II): 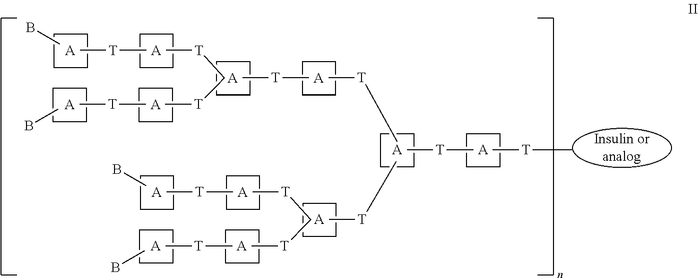
or has the general formula (III): 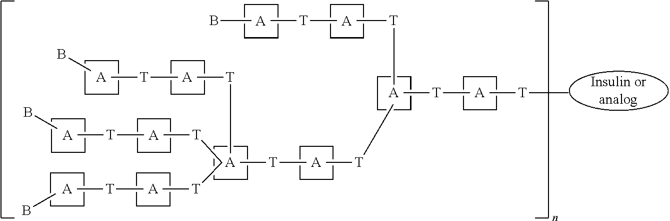
wherein:
(i) each occurrence of 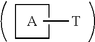
represents a repeat within a branch of the conjugate;
(ii) each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;
(iii) each occurrence of T is independently a covalent bond or a bivalent, straight or branched, saturated or unsaturated, optionally substituted C₁₋₃₀ hydrocarbon chain wherein one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;
(iv) each occurrence of R is independently hydrogen, a suitable protecting group, an acyl moiety, arylalkyl moiety, aliphatic moiety, aryl moiety, heteroaryl moiety, or heteroaliphatic moiety;
(v) —B is -T-L^{B}-X, wherein each occurrence of X is independently a ligand comprising a monosaccharide, disaccharide, trisaccharide, tetrasaccharide, or branched trisaccharide, and each occurrence of L^{B} is independently a covalent bond or a group derived from the covalent conjugation of a T with an X; and,
(vi) n is 1, 2, or 3.

13. The conjugate of claim 12, wherein the conjugate comprises the structure of conjugate (I) wherein the insulin or insulin analog is conjugated to a tetra-valent linker selected from the group consisting of: 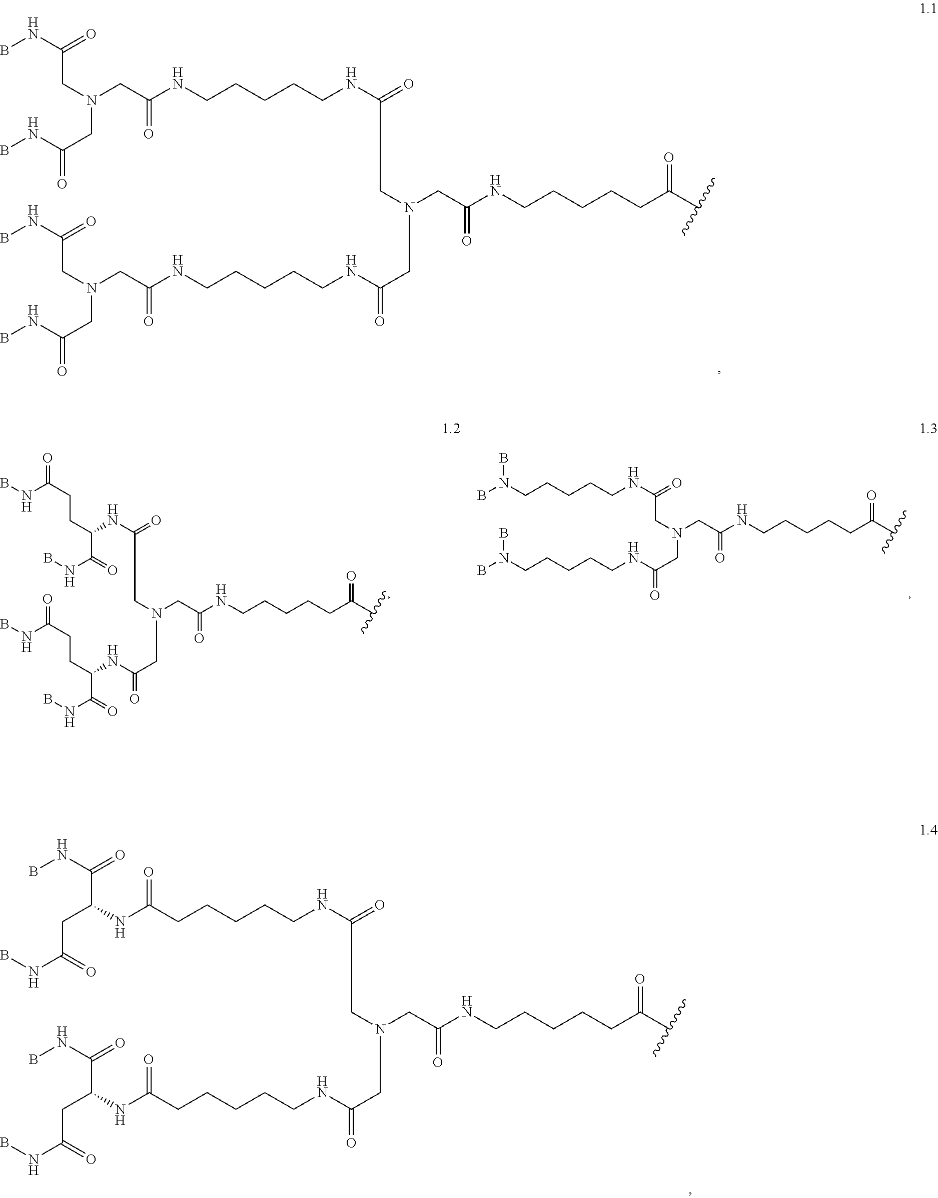 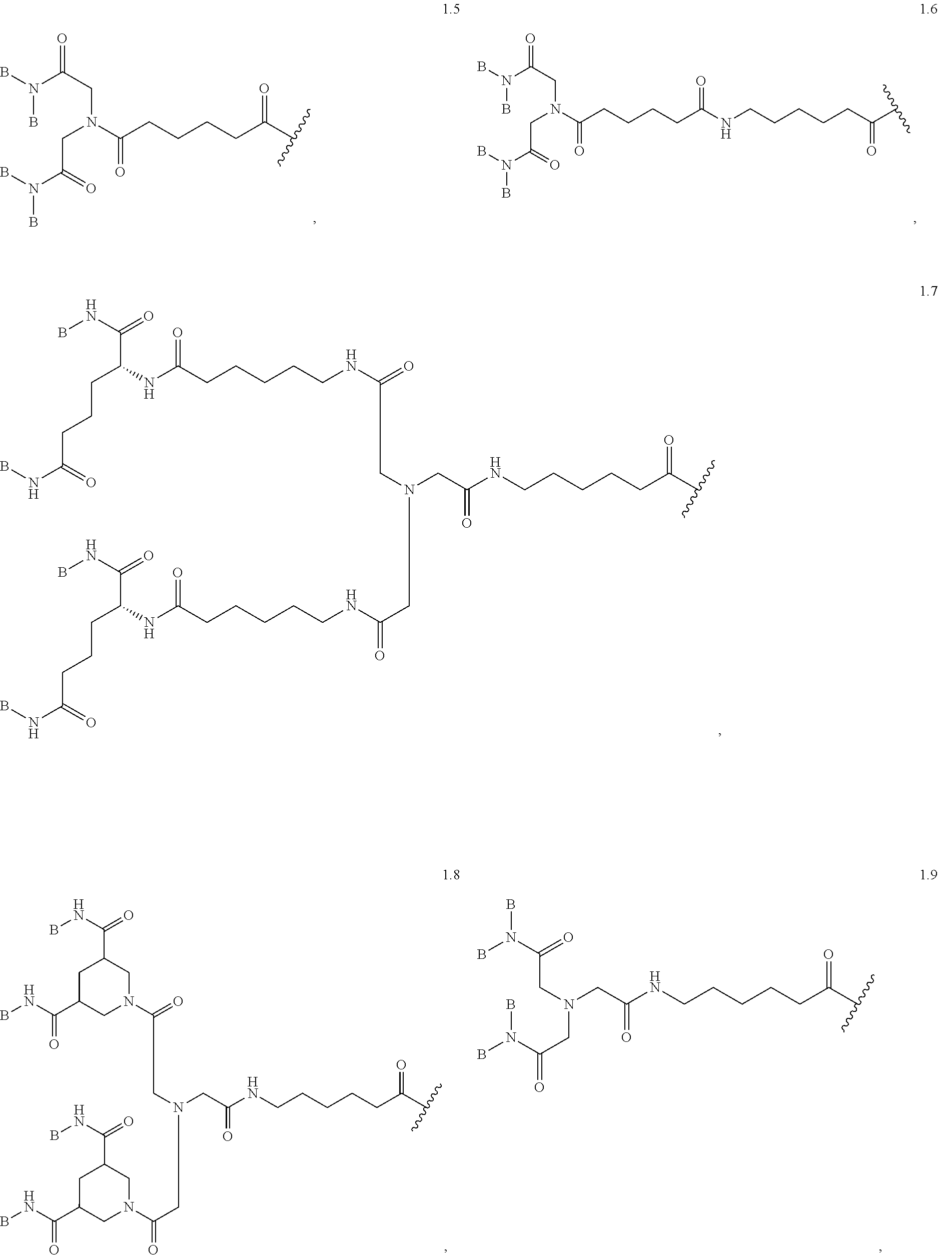 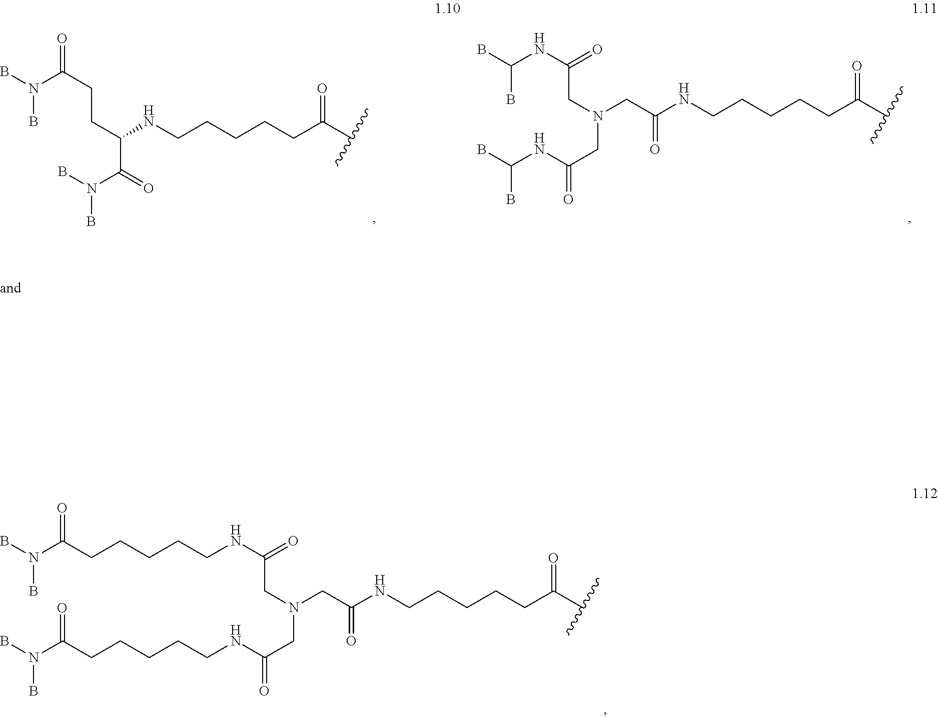
or the conjugate comprises the structure of conjugate II, wherein the insulin or insulin analog is conjugated to a tetra-valent linker selected from the group consisting of 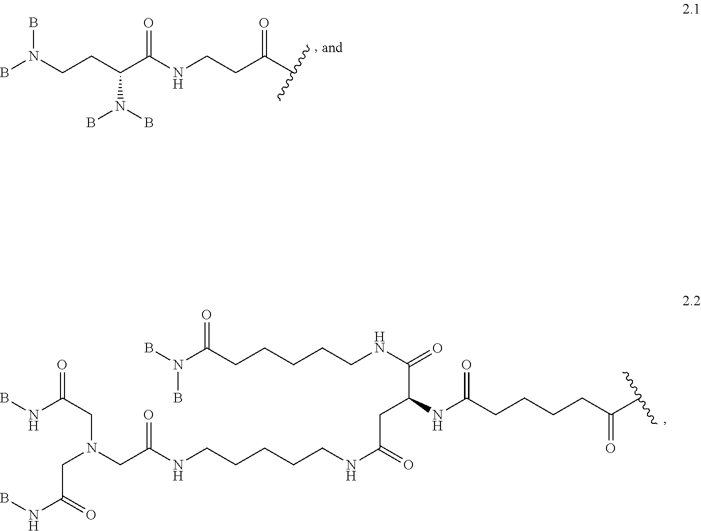
or the conjugate comprises the structure of conjugate III, wherein the insulin or insulin analog is conjugated to a tetra-valent linker selected from the group consisting of: 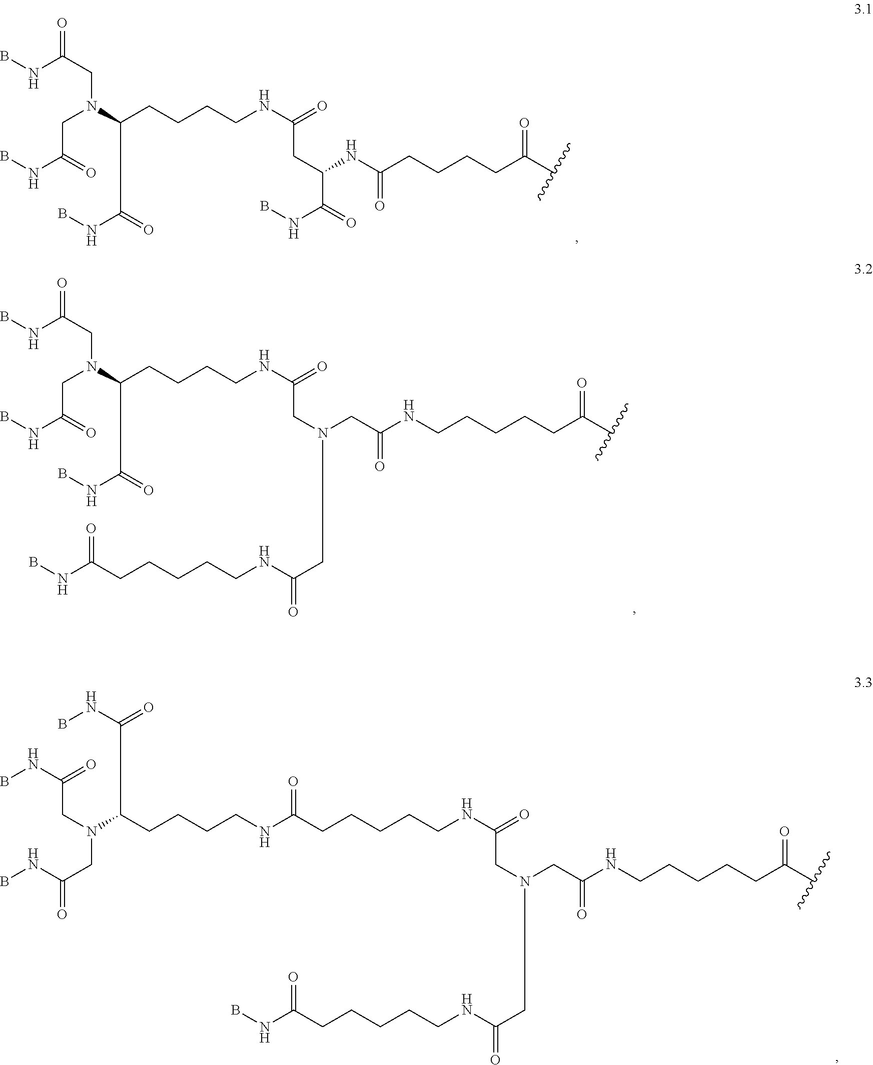 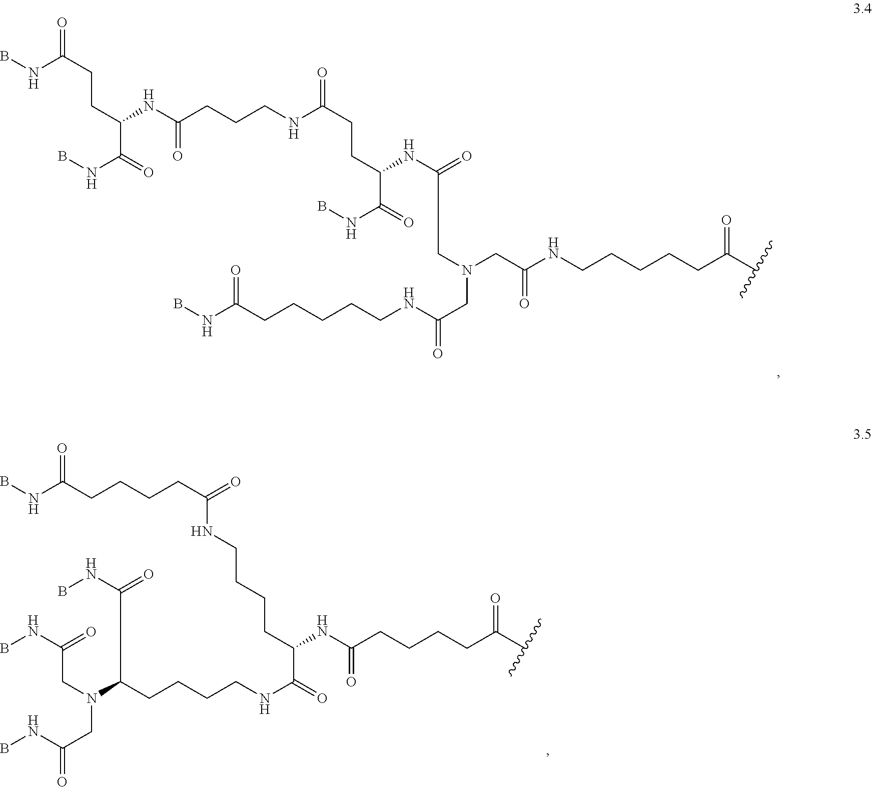 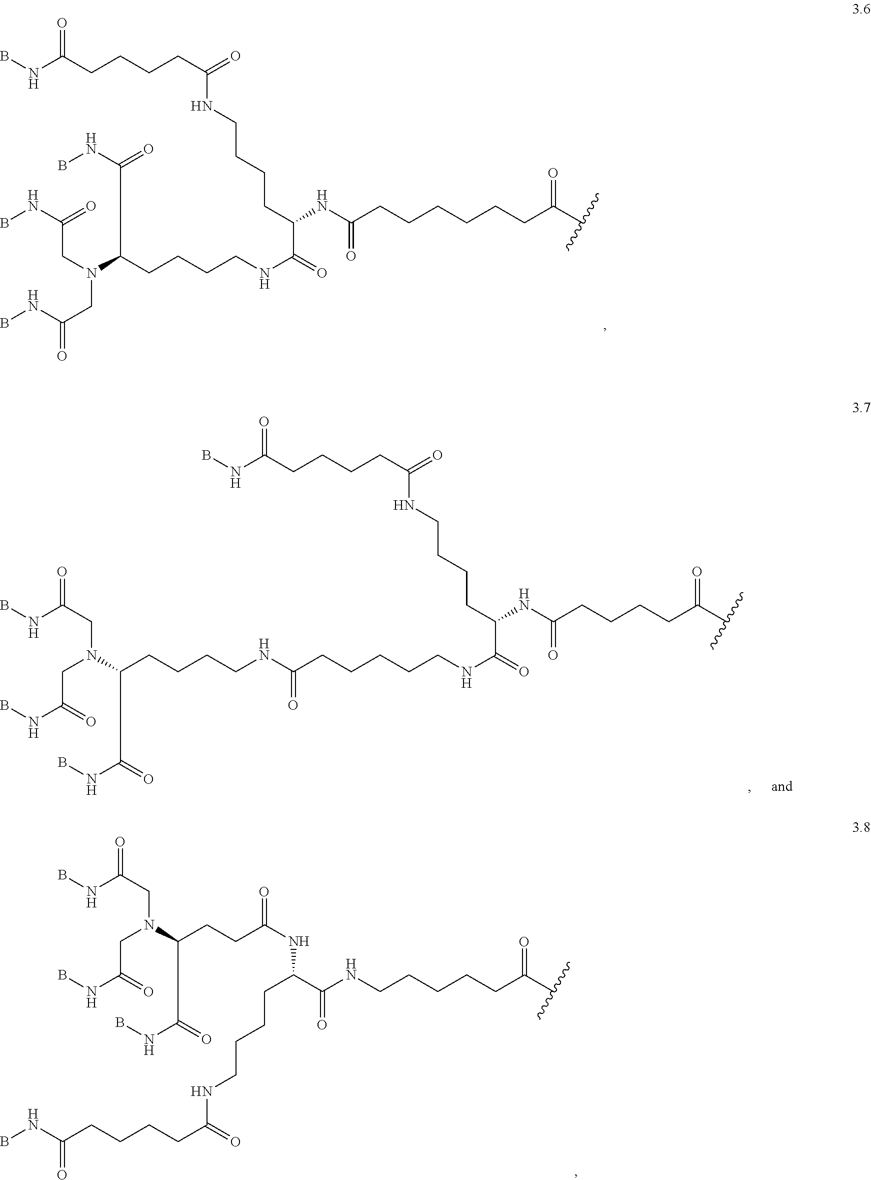
wherein a wavy line indicates the bond between the proximal end of the linker arm and amino acid on the insulin or insulin analog and wherein each B is independently -T-L^{B}-X, wherein each occurrence of X is independently the ligand comprising a monosaccharide, disaccharide, trisaccharide, tetrasaccharide, or branched trisaccharide, and each occurrence of L^{B} is independently a covalent bond or a group derived from the covalent conjugation of a T with an X.

14. A conjugate comprising an insulin or insulin analog conjugated to a tri-valent sugar cluster that comprises a structure selected from ML-1, ML-2, ML-3, ML-4, ML-5, ML-6, ML-7, ML-8, ML-9, ML-10, ML-11, ML-12, ML-13, ML-14, ML-15, ML-16, ML-17, ML-18, ML-19, ML-20, ML-21, ML-22, ML-23, ML-24, ML-25, ML-26, ML-27, ML-28, ML-29, ML-30, ML-31, ML-32, ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, ML-52, and ML-53.

15. A conjugate selected from IOC-1, IOC-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-40, IOC-41, IOC-42, IOC-43, IOC-44, IOC-45, IOC-46, IOC-47, IOC-48, IOC-49, IOC-50, IOC-51, IOC-52, IOC-53, IOC-54, IOC-55, IOC-56, IOC-57, IOC-58, IOC-59, IOC-60, IOC-61, IOC-62, IOC-63, IOC-64, IOC-65, IOC-66, IOC-67, IOC-68, and IOC-69.

16. A composition comprising a conjugate of claim 1 and a pharmaceutically acceptable carrier.

17. A method for treating diabetes comprising administering to an individual in need thereof a therapeutically effective amount of the conjugate of claim 1 to treat the diabetes.

18. The method of claim 17, wherein the diabetes is type I diabetes, type II diabetes, or gestational diabetes.

19. A method for treating diabetes comprising administering to an individual in need thereof a therapeutically effective amount of the composition of claim 16 to treat the diabetes.

20. The method of claim 19, wherein the diabetes is type I diabetes, type II diabetes, or gestational diabetes.

21-26. (canceled)
